SEQUENCE LISTING (317 sequences)

SEQ ID NO: 1 - protein, 23 aa, Xenopus laevis
GIGKFLHSAKKFGKAFVGEIMNS

SEQ ID NO: 2 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (21)..(21): Norleucine
XIGKXLHSAKKFGKAFVGEIXNS

SEQ ID NO: 3 - protein, 23 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
  MOD_RES (21)..(21): Norleucine
GXGKFXHSAKKFGKAFVGEIXNS

SEQ ID NO: 4 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (21)..(21): Norleucine
GIXKFLXSAKKFGKAFVGEIXNS

SEQ ID NO: 5 - protein, 23 aa, Artificial Sequence
  source: /note=“Description of Artificial Sequence: Synthetic peptide”
  MOD_RES (4)..(4): alpha-methyl, alpha-alkenyl non-natural amino acid
  SITE (4)..(8): /note=“May or may not be cross-linked via a side chain”
  MOD_RES (8)..(8): alpha-methyl, alpha-alkenyl non-natural amino acid
  MOD_RES (21)..(21): Norleucine
GIGXFLHXAKKFGKAFVGEIXNS

SEQ ID NO: 6 - protein, 23 aa, Artificial Sequence
  source: /note=“Description of Artificial Sequence: Synthetic peptide”
  MOD_RES (5)..(5): alpha-methyl, alpha-alkenyl non-natural amino acid
  SITE (5)..(9): /note=“May or may not be cross-linked via a side chain”
  MOD_RES (9)..(9): alpha-methyl, alpha-alkenyl non-natural amino acid
  MOD_RES (21)..(21): Norleucine
GIGKXLHSXKKFGKAFVGEIXNS

SEQ ID NO: 7 - protein, 23 aa, Artificial Sequence
  source: /note=“Description of Artificial Sequence: Synthetic peptide”
  MOD_RES (6)..(6): alpha-methyl, alpha-alkenyl non-natural amino acid
  SITE (6)..(10): /note=“May or may not be cross-linked via a side chain”
  MOD_RES (10)..(10): alpha-methyl, alpha-alkenyl non-natural amino acid
  MOD_RES (21)..(21): Norleucine
GIGKFXHSAXKFGKAFVGEIXNS

SEQ ID NO: 8 - protein, 23 aa, Artificial Sequence
  source: /note=“Description of Artificial Sequence: Synthetic peptide”
  MOD_RES (7)..(7): alpha-methyl, alpha-alkenyl non-natural amino acid
  SITE (7)..(11): /note=“May or may not be cross-linked via a side chain”
  MOD_RES (11)..(11): alpha-methyl, alpha-alkenyl non-natural amino acid
  MOD_RES (21)..(21): Norleucine
GIGKFLXSAKXFGKAFVGEIXNS

SEQ ID NO: 9 - protein, 23 aa, Artificial Sequence
  source: /note=“Description of Artificial Sequence: Synthetic peptide”
  MOD_RES (8)..(8): alpha-methyl, alpha-alkenyl non-natural amino acid
  SITE (8)..(12): /note=“May or may not be cross-linked via a side chain”
  MOD_RES (12)..(12): alpha-methyl, alpha-alkenyl non-natural amino acid
  MOD_RES (21)..(21): Norleucine
GIGKFLHXAKKXGKAFVGEIXNS

SEQ ID NO: 10 - protein, 23 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
  MOD_RES (21)..(21): Norleucine
GIGKFLHSXKKFXKAFVGEIXNS

SEQ ID NO: 11 - protein, 23 aa, Artificial Sequence
  source: /note=“Description of Artificial Sequence: Synthetic peptide”
  MOD_RES (10)..(10): alpha-methyl, alpha-alkenyl non-natural amino acid
  SITE (10)..(14): /note=“May or may not be cross-linked via a side chain”
  MOD_RES (14)..(14): alpha-methyl, alpha-alkenyl non-natural amino acid
  MOD_RES (21)..(21): Norleucine
GIGKFLHSAXKFGXAFVGEIXNS

SEQ ID NO: 12 - protein, 23 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
  MOD_RES (21)..(21): Norleucine
GIGKFLHSAKXFGKXFVGEIXNS

SEQ ID NO: 13 - protein, 23 aa, Artificial Sequence
  source: /note=“Description of Artificial Sequence: Synthetic peptide”
  MOD_RES (12)..(12): alpha-methyl, alpha-alkenyl non-natural amino acid
  SITE (12)..(16): /note=“May or may not be cross-linked via a side chain”
  MOD_RES (16)..(16): alpha-methyl, alpha-alkenyl non-natural amino acid
  MOD_RES (21)..(21): Norleucine
GIGKFLHSAKKXGKAXVGEIXNS

SEQ ID NO: 14 - protein, 23 aa, Artificial Sequence
  source: /note=“Description of Artificial Sequence: Synthetic peptide”
  MOD_RES (13)..(13): alpha-methyl, alpha-alkenyl non-natural amino acid
  SITE (13)..(17): /note=“May or may not be cross-linked via a side chain”
  MOD_RES (17)..(17): alpha-methyl, alpha-alkenyl non-natural amino acid
  MOD_RES (21)..(21): Norleucine
GIGKFLHSAKKFXKAFXGEIXNS

SEQ ID NO: 15 - protein, 23 aa, Artificial Sequence
  source: /note=“Description of Artificial Sequence: Synthetic peptide”
  MOD_RES (14)..(14): alpha-methyl, alpha-alkenyl non-natural amino acid
  SITE (14)..(18): /note=“May or may not be cross-linked via a side chain”
  MOD_RES (18)..(18): alpha-methyl, alpha-alkenyl non-natural amino acid
  MOD_RES (21)..(21): Norleucine
GIGKFLHSAKKFGXAFVXEIXNS

SEQ ID NO: 16 - protein, 23 aa, Artificial Sequence
  source: /note=“Description of Artificial Sequence: Synthetic peptide”
  MOD_RES (15)..(15): alpha-methyl, alpha-alkenyl non-natural amino acid
  SITE (15)..(19): /note=“May or may not be cross-linked via a side chain”
  MOD_RES (19)..(19): alpha-methyl, alpha-alkenyl non-natural amino acid
  MOD_RES (21)..(21): Norleucine
GIGKFLHSAKKFGKXFVGXIXNS

SEQ ID NO: 17 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVGEXXNS

SEQ ID NO: 18 - protein, 23 aa, Artificial Sequence
  source: /note=“Description of Artificial Sequence: Synthetic peptide”
  MOD_RES (17)..(17): alpha-methyl, alpha-alkenyl non-natural amino acid
  SITE (17)..(21): /note=“May or may not be cross-linked via a side chain”
  MOD_RES (21)..(21): alpha-methyl, alpha-alkenyl non-natural amino acid
GIGKFLHSAKKFGKAFXGEIXNS

SEQ ID NO: 19 - protein, 23 aa, Artificial Sequence
  source: /note=“Description of Artificial Sequence: Synthetic peptide”
  MOD_RES (18)..(18): alpha-methyl, alpha-alkenyl non-natural amino acid
  SITE (18)..(22): /note=“May or may not be cross-linked via a side chain”
  MOD_RES (21)..(21): Norleucine
  MOD_RES (22)..(22): alpha-methyl, alpha-alkenyl non-natural amino acid
GIGKFLHSAKKFGKAFVXEIXXS

SEQ ID NO: 20 - protein, 23 aa, Artificial Sequence
  source: /note=“Description of Artificial Sequence: Synthetic peptide”
  MOD_RES (19)..(19): alpha-methyl, alpha-alkenyl non-natural amino acid
  SITE (19)..(23): /note=“May or may not be cross-linked via a side chain”
  MOD_RES (21)..(21): Norleucine
  MOD_RES (23)..(23): alpha-methyl, alpha-alkenyl non-natural amino acid
GIGKFLHSAKKFGKAFVGXIXNX

SEQ ID NO: 21 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
KIGKFLHSAKKFGKAXVGEXXNS

SEQ ID NO: 22 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GKGKFLHSAKKFGKAXVGEXXNS

SEQ ID NO: 23 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIKKFLHSAKKFGKAXVGEXXNS

SEQ ID NO: 24 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKKLHSAKKFGKAXVGEXXNS

SEQ ID NO: 25 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFKHSAKKFGKAXVGEXXNS

SEQ ID NO: 26 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLKSAKKFGKAXVGEXXNS

SEQ ID NO: 27 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHKAKKFGKAXVGEXXNS

SEQ ID NO: 28 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSKKKFGKAXVGEXXNS

SEQ ID NO: 29 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKKGKAXVGEXXNS

SEQ ID NO: 30 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFKKAXVGEXXNS

SEQ ID NO: 31 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKKXVGEXXNS

SEQ ID NO: 32 - protein, 18 aa, Artificial Sequence
  source: /note=“Description of Artificial Sequence: Synthetic peptide”
  MOD_RES (16)..(16): Norleucine
LHSAKKFGKAFVGEIXNS

SEQ ID NO: 33 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXKGEXXNS

SEQ ID NO: 34 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVKEXXNS

SEQ ID NO: 35 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVGKXXNS

SEQ ID NO: 36 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVGEXXNK

SEQ ID NO: 37 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
GIGKFLHSAKKFGKAXVGEXKNS

SEQ ID NO: 38 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVGEXXKS

SEQ ID NO: 39 - protein, 4 aa, Artificial Sequence
  source: /note=“Description of Artificial Sequence: Synthetic peptide”
GIGK

SEQ ID NO: 40 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
EIGKFLHSAKKFGKAXVGEXXNS

SEQ ID NO: 41 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GEGKFLHSAKKFGKAXVGEXXNS

SEQ ID NO: 42 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIEKFLHSAKKFGKAXVGEXXNS

SEQ ID NO: 43 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGEFLHSAKKFGKAXVGEXXNS

SEQ ID NO: 44 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKELHSAKKFGKAXVGEXXNS

SEQ ID NO: 45 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFEHSAKKFGKAXVGEXXNS

SEQ ID NO: 46 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLESAKKFGKAXVGEXXNS

SEQ ID NO: 47 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHEAKKFGKAXVGEXXNS

SEQ ID NO: 48 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSEKKFGKAXVGEXXNS

SEQ ID NO: 49 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAEKFGKAXVGEXXNS

SEQ ID NO: 50 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKEFGKAXVGEXXNS

SEQ ID NO: 51 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKEGKAXVGEXXNS

SEQ ID NO: 52 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFEKAXVGEXXNS

SEQ ID NO: 53 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGEAXVGEXXNS

SEQ ID NO: 54 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKEXVGEXXNS

SEQ ID NO: 55 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXEGEXXNS

SEQ ID NO: 56 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVEEXXNS

SEQ ID NO: 57 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
GIGKFLHSAKKFGKAXVGEXENS

SEQ ID NO: 58 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVGEXXES

SEQ ID NO: 59 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFGKAXVGEXXNE

SEQ ID NO: 60 - protein, 23 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
GXGKFXHSKKKFGKAXVGEXAKK

SEQ ID NO: 61 - protein, 12 aa, Artificial Sequence
  source: /note=“Description of Artificial Sequence: Synthetic peptide”
FGKAFVGEIANS

SEQ ID NO: 62 - protein, 7 aa, Artificial Sequence
  source: /note=“Description of Artificial Sequence: Synthetic peptide”
VGEIANS

SEQ ID NO: 63 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
KIGKFLHSAKKFGKAFVGEIMNS

SEQ ID NO: 64 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GKGKFLHSAKKFGKAFVGEIMNS

SEQ ID NO: 65 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIKKFLHSAKKFGKAFVGEIMNS

SEQ ID NO: 66 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLKSAKKFGKAFVGEIMNS

SEQ ID NO: 67 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHKAKKFGKAFVGEIMNS

SEQ ID NO: 68 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKAFVGKIMNS

SEQ ID NO: 69 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKAFVGEIMKS

SEQ ID NO: 70 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIEKFLHSAKKFGKAFVGEIMNS

SEQ ID NO: 71 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLESAKKFGKAFVGEIMNS

SEQ ID NO: 72 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHEAKKFGKAFVGEIMNS

SEQ ID NO: 73 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKAFVEEIMNS

SEQ ID NO: 74 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKAFVGEIMES

SEQ ID NO: 75 - protein, 23 aa, Artificial Sequence
  source: /note=“Description of Artificial Sequence: Synthetic peptide”
GIGKFLHSKKKFGKAFVGEIAKK

SEQ ID NO: 76 - protein, 16 aa, Artificial Sequence
  source: /note=“Description of Artificial Sequence: Synthetic peptide”
  MOD_RES (14)..(14): Norleucine
SAKKFGKAFVGEIXNS

SEQ ID NO: 77 - protein, 14 aa, Artificial Sequence
  source: /note=“Description of Artificial Sequence: Synthetic peptide”
  MOD_RES (12)..(12): Norleucine
KKFGKAFVGEIXNS

SEQ ID NO: 78 - protein, 6 aa, Artificial Sequence
  source: /note=“Description of Artificial Sequence: Synthetic peptide”
GIGKFL

SEQ ID NO: 79 - protein, 12 aa, Artificial Sequence
  source: /note=“Description of Artificial Sequence: Synthetic peptide”
  MOD_RES (10)..(10): Norleucine
FGKAFVGEIXNS

SEQ ID NO: 80 - protein, 11 aa, Artificial Sequence
  source: /note=“Description of Artificial Sequence: Synthetic peptide”
GIGKFLHSAKK

SEQ ID NO: 81 - protein, 7 aa, Artificial Sequence
  source: /note=“Description of Artificial Sequence: Synthetic peptide”
  MOD_RES (5)..(5): Norleucine
VGEIXNS

SEQ ID NO: 82 - protein, 15 aa, Artificial Sequence
  source: /note=“Description of Artificial Sequence: Synthetic peptide”
GIGKFLHSAKKFGKA

SEQ ID NO: 83 - protein, 16 aa, Artificial Sequence
  source: /note=“Description of Artificial Sequence: Synthetic peptide”
GIGKFLHSAKKFGKAF

SEQ ID NO: 84 - protein, 15 aa, Artificial Sequence
  source: /note=“Description of Artificial Sequence: Synthetic peptide”
KIGKFLHSAKKFGKA

SEQ ID NO: 85 - protein, 15 aa, Artificial Sequence
  source: /note=“Description of Artificial Sequence: Synthetic peptide”
GKGKFLHSAKKFGKA

SEQ ID NO: 86 - protein, 15 aa, Artificial Sequence
  source: /note=“Description of Artificial Sequence: Synthetic peptide”
GIKKFLHSAKKFGKA

SEQ ID NO: 87 - protein, 15 aa, Artificial Sequence
  source: /note=“Description of Artificial Sequence: Synthetic peptide”
GIGKFLKSAKKFGKA

SEQ ID NO: 88 - protein, 15 aa, Artificial Sequence
  source: /note=“Description of Artificial Sequence: Synthetic peptide”
GIGKFLHKAKKFGKA

SEQ ID NO: 89 - protein, 18 aa, Artificial Sequence
  source: /note=“Description of Artificial Sequence: Synthetic peptide”
LHSAKKFGKAFVGEIANS

SEQ ID NO: 90 - protein, 17 aa, Artificial Sequence
  source: /note=“Description of Artificial Sequence: Synthetic peptide”
HSAKKFGKAFVGEIANS

SEQ ID NO: 91 - protein, 15 aa, Artificial Sequence
  source: /note=“Description of Artificial Sequence: Synthetic peptide”
GIEKFLHSAKKFGKA

SEQ ID NO: 92 - protein, 15 aa, Artificial Sequence
  source: /note=“Description of Artificial Sequence: Synthetic peptide”
GIGKFLESAKKFGKA

SEQ ID NO: 93 - protein, 15 aa, Artificial Sequence
  source: /note=“Description of Artificial Sequence: Synthetic peptide”
GIGKFLHEAKKFGKA

SEQ ID NO: 94 - protein, 16 aa, Artificial Sequence
  source: /note=“Description of Artificial Sequence: Synthetic peptide”
SAKKFGKAFVGEIANS

SEQ ID NO: 95 - protein, 14 aa, Artificial Sequence
  source: /note=“Description of Artificial Sequence: Synthetic peptide”
KKFGKAFVGEIANS

SEQ ID NO: 96 - protein, 17 aa, Artificial Sequence
  source: /note=“Description of Artificial Sequence: Synthetic peptide”
  MOD_RES (10)..(10): alpha-methyl, alpha-alkenyl non-natural amino acid
  SITE (10)..(14): /note=“May or may not be cross-linked via a side chain”
  MOD_RES (14)..(14): alpha-methyl, alpha-alkenyl non-natural amino acid
HSKKKFGKAXVGEXAKK

SEQ ID NO: 97 - protein, 15 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
GXGKFXHSKKKFGKA

SEQ ID NO: 98 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
XIGKXLHKAKKFGKAXVGEXXNS

SEQ ID NO: 99 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
XIGKXLHSKKKFGKAXVGEXXNS

SEQ ID NO: 100 - protein, 23 aa, Artificial Sequence
  source: /note=“Description of Artificial Sequence: Synthetic peptide”
  MOD_RES (21)..(21): Norleucine
GIGKFLHSAKKFGKAFVGEIXNS

SEQ ID NO: 101 - protein, 23 aa, Artificial Sequence
  source: /note=“Description of Artificial Sequence: Synthetic peptide”
  MOD_RES (1)..(1): (S)-2-(4′-pentenyl)Alanine
  SITE (1)..(5): /note=“May or may not be cross-linked via a side chain”
  MOD_RES (5)..(5): (S)-2-(4′-pentenyl)Alanine
  MOD_RES (21)..(21): Norleucine
XIGKXLHSAKKFGKAFVGEIXNS

SEQ ID NO: 102 - protein, 23 aa, Artificial Sequence
  source: /note=“Description of Artificial Sequence: Synthetic peptide”
  MOD_RES (2)..(2): (S)-2-(4′-pentenyl)Alanine
  SITE (2)..(6): /note=“May or may not be cross-linked via a side chain”
  MOD_RES (6)..(6): (S)-2-(4′-pentenyl)Alanine
  MOD_RES (21)..(21): Norleucine
GXGKFXHSAKKFGKAFVGEIXNS

SEQ ID NO: 103 - protein, 23 aa, Artificial Sequence
  source: /note=“Description of Artificial Sequence: Synthetic peptide”
  MOD_RES (3)..(3): (S)-2-(4′-pentenyl)Alanine
  SITE (3)..(7): /note=“May or may not be cross-linked via a side chain”
  MOD_RES (7)..(7): (S)-2-(4′-pentenyl)Alanine
  MOD_RES (21)..(21): Norleucine
GIXKFLXSAKKFGKAFVGEIXNS

SEQ ID NO: 104 - protein, 23 aa, Artificial Sequence
  source: /note=“Description of Artificial Sequence: Synthetic peptide”
  MOD_RES (5)..(5): (S)-2-(4′-pentenyl)Alanine
  SITE (5)..(9): /note=“May or may not be cross-linked via a side chain”
  MOD_RES (9)..(9): (S)-2-(4′-pentenyl)Alanine
  MOD_RES (21)..(21): Norleucine
GIGKXLHSXKKFGKAFVGEIXNS

SEQ ID NO: 105 - protein, 23 aa, Artificial Sequence
  source: /note=“Description of Artificial Sequence: Synthetic peptide”
  MOD_RES (7)..(7): (S)-2-(4′-pentenyl)Alanine
  SITE (7)..(11): /note=“May or may not be cross-linked via a side chain”
  MOD_RES (11)..(11): (S)-2-(4′-pentenyl)Alanine
  MOD_RES (21)..(21): Norleucine
GIGKFLXSAKXFGKAFVGEIXNS

SEQ ID NO: 106 - protein, 23 aa, Artificial Sequence
  source: /note=“Description of Artificial Sequence: Synthetic peptide”
  MOD_RES (12)..(12): (S)-2-(4′-pentenyl)Alanine
  SITE (12)..(16): /note=“May or may not be cross-linked via a side chain”
  MOD_RES (16)..(16): (S)-2-(4′-pentenyl)Alanine
  MOD_RES (21)..(21): Norleucine
GIGKFLHSAKKXGKAXVGEIXNS

SEQ ID NO: 107 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKAXVGEXXNS

SEQ ID NO: 108 - protein, 23 aa, Artificial Sequence
  source: /note=“Description of Artificial Sequence: Synthetic peptide”
  MOD_RES (17)..(17): (S)-2-(4′-pentenyl)Alanine
  SITE (17)..(21): /note=“May or may not be cross-linked via a side chain”
  MOD_RES (21)..(21): (S)-2-(4′-pentenyl)Alanine
GIGKFLHSAKKFGKAFXGEIXNS

SEQ ID NO: 109 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
KIGKFLHSAKKFGKAXVGEXXNS

SEQ ID NO: 110 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GKGKFLHSAKKFGKAXVGEXXNS

SEQ ID NO: 111 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIKKFLHSAKKFGKAXVGEXXNS

SEQ ID NO: 112 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLKSAKKFGKAXVGEXXNS

SEQ ID NO: 113 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHKAKKFGKAXVGEXXNS

SEQ ID NO: 114 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKAXVGKXXNS

SEQ ID NO: 115 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKAXVGEXXKS

SEQ ID NO: 116 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIEKFLHSAKKFGKAXVGEXXNS

SEQ ID NO: 117 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLESAKKFGKAXVGEXXNS

SEQ ID NO: 118 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHEAKKFGKAXVGEXXNS

SEQ ID NO: 119 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKAXVEEXXNS

SEQ ID NO: 120 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKAXVGEXXES

SEQ ID NO: 121 - protein, 23 aa, Artificial Sequence
  source: /note=“Description of Artificial Sequence: Synthetic peptide”
  MOD_RES (2)..(2): (S)-2-(4′-pentenyl)Alanine
  SITE (2)..(6): /note=“May or may not be cross-linked via a side chain”
  MOD_RES (6)..(6): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
GXGKFXHSKKKFGKAXVGEXAKK

SEQ ID NO: 122 - protein, 17 aa, Artificial Sequence
  source: /note=“Description of Artificial Sequence: Synthetic peptide”
  MOD_RES (15)..(15): Norleucine
HSAKKFGKAFVGEIXNS

SEQ ID NO: 123 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKKFVGEIMNS

SEQ ID NO: 124 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKAFVKEIMNS

SEQ ID NO: 125 - protein, 23 aa, Artificial Sequence
  source: /note=“Description of Artificial Sequence: Synthetic peptide”
GIGKFLHSAKKFGKAFVGEIKNS

SEQ ID NO: 126 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKEFVGEIMNS

SEQ ID NO: 127 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKKFGKAFVGEIMNE

SEQ ID NO: 128 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKKXVGEXXNS

SEQ ID NO: 129 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKAXVKEXXNS

SEQ ID NO: 130 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
GIGKFLHSAKKFGKAXVGEXKNS

SEQ ID NO: 131 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKEXVGEXXNS

SEQ ID NO: 132 - protein, 23 aa, Artificial Sequence
  source: /note=“Description of Artificial Sequence: Synthetic peptide”
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIGKFLHSAKKFGKAXVGEXXNE

SEQ ID NO: 133 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
XKGKXLHSAKKFGKAXVGEXXNS

SEQ ID NO: 134 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
XIEKXLHSAKKFGKAXVGEXXNS

SEQ ID NO: 135 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
XIGKXLESAKKFGKAXVGEXXNS

SEQ ID NO: 136 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
XIGKXLHSAKKFGKAXVEEXXNS

SEQ ID NO: 137 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (21)..(21): Norleucine
XIGKXLHSAKKFGKAFVGEIXES

SEQ ID NO: 138 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (21)..(21): Norleucine
GKXKFLXSAKKFGKAFVGEIXNS

SEQ ID NO: 139 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (21)..(21): Norleucine
GIXKFLXSAKKFGKAFVEEIXNS

SEQ ID NO: 140 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (21)..(21): Norleucine
GKXKFLXSAKKFGKAFVGEIXES

SEQ ID NO: 141 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
XIGKXLHSAKKFGKAXVGEXXNS

SEQ ID NO: 142 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIXKFLXSAKKFGKAXVGEXXNS

SEQ ID NO: 143 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIXKFLXKAKKFGKAXVGEXXNS

SEQ ID NO: 144 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GIXKFLXSKKKFGKAXVGEXXNS

SEQ ID NO: 145 - protein, 23 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
  MOD_RES (21)..(21): Norleucine
GXGKFXHSKKKFGKAXVGEXXNS

SEQ ID NO: 146 - protein, 23 aa, Artificial Sequence
  source: /note=“Description of Artificial Sequence: Synthetic peptide”
  MOD_RES (1)..(1): (S)-2-(4′-pentenyl)Alanine
  SITE (1)..(5): /note=“May or may not be cross-linked via a side chain”
  MOD_RES (5)..(5): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
XKGKXLHSAKKFGKAXVGEXXNS

SEQ ID NO: 147 - protein, 23 aa, Artificial Sequence
  source: /note=“Description of Artificial Sequence: Synthetic peptide”
  MOD_RES (1)..(1): (S)-2-(4′-pentenyl)Alanine
  SITE (1)..(5): /note=“May or may not be cross-linked via a side chain”
  MOD_RES (5)..(5): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
XIEKXLHSAKKFGKAXVGEXXNS

SEQ ID NO: 148 - protein, 23 aa, Artificial Sequence
  source: /note=“Description of Artificial Sequence: Synthetic peptide”
  MOD_RES (1)..(1): (S)-2-(4′-pentenyl)Alanine
  SITE (1)..(5): /note=“May or may not be cross-linked via a side chain”
  MOD_RES (5)..(5): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
XIGKXLESAKKFGKAXVGEXXNS

SEQ ID NO: 149 - protein, 23 aa, Artificial Sequence
  source: /note=“Description of Artificial Sequence: Synthetic peptide”
  MOD_RES (1)..(1): (S)-2-(4′-pentenyl)Alanine
  SITE (1)..(5): /note=“May or may not be cross-linked via a side chain”
  MOD_RES (5)..(5): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
XIGKXLHSAKKFGKAXVEEXXNS

SEQ ID NO: 150 - protein, 23 aa, Artificial Sequence
  source: /note=“Description of Artificial Sequence: Synthetic peptide”
  MOD_RES (1)..(1): (S)-2-(4′-pentenyl)Alanine
  SITE (1)..(5): /note=“May or may not be cross-linked via a side chain”
  MOD_RES (5)..(5): (S)-2-(4′-pentenyl)Alanine
  MOD_RES (21)..(21): Norleucine
XIGKXLHSAKKFGKAFVGEIXES

SEQ ID NO: 151 - protein, 23 aa, Artificial Sequence
  source: /note=“Description of Artificial Sequence: Synthetic peptide”
  MOD_RES (3)..(3): (S)-2-(4′-pentenyl)Alanine
  SITE (3)..(7): /note=“May or may not be cross-linked via a side chain”
  MOD_RES (7)..(7): (S)-2-(4′-pentenyl)Alanine
  MOD_RES (21)..(21): Norleucine
GKXKFLXSAKKFGKAFVGEIXNS

SEQ ID NO: 152 - protein, 23 aa, Artificial Sequence
  source: /note=“Description of Artificial Sequence: Synthetic peptide”
  MOD_RES (3)..(3): (S)-2-(4′-pentenyl)Alanine
  SITE (3)..(7): /note=“May or may not be cross-linked via a side chain”
  MOD_RES (7)..(7): (S)-2-(4′-pentenyl)Alanine
  MOD_RES (21)..(21): Norleucine
GIXKFLXSAKKFGKAFVEEIXNS

SEQ ID NO: 153 - protein, 23 aa, Artificial Sequence
  source: /note=“Description of Artificial Sequence: Synthetic peptide”
  MOD_RES (3)..(3): (S)-2-(4′-pentenyl)Alanine
  SITE (3)..(7): /note=“May or may not be cross-linked via a side chain”
  MOD_RES (7)..(7): (S)-2-(4′-pentenyl)Alanine
  MOD_RES (21)..(21): Norleucine
GKXKFLXSAKKFGKAFVGEIXES

SEQ ID NO: 154 - protein, 23 aa, Artificial Sequence
  source: /note=“Description of Artificial Sequence: Synthetic peptide”
  MOD_RES (1)..(1): (S)-2-(4′-pentenyl)Alanine
  SITE (1)..(5): /note=“May or may not be cross-linked via a side chain”
  MOD_RES (5)..(5): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
XIGKXLHSAKKFGKAXVGEXXNS

SEQ ID NO: 155 - protein, 23 aa, Artificial Sequence
  source: /note=“Description of Artificial Sequence: Synthetic peptide”
  MOD_RES (3)..(3): (S)-2-(4′-pentenyl)Alanine
  SITE (3)..(7): /note=“May or may not be cross-linked via a side chain”
  MOD_RES (7)..(7): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIXKFLXSAKKFGKAXVGEXXNS

SEQ ID NO: 156 - protein, 23 aa, Artificial Sequence
  source: /note=“Description of Artificial Sequence: Synthetic peptide”
  MOD_RES (3)..(3): (S)-2-(4′-pentenyl)Alanine
  SITE (3)..(7): /note=“May or may not be cross-linked via a side chain”
  MOD_RES (7)..(7): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIXKFLXKAKKFGKAXVGEXXNS

SEQ ID NO: 157 - protein, 23 aa, Artificial Sequence
  source: /note=“Description of Artificial Sequence: Synthetic peptide”
  MOD_RES (3)..(3): (S)-2-(4′-pentenyl)Alanine
  SITE (3)..(7): /note=“May or may not be cross-linked via a side chain”
  MOD_RES (7)..(7): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GIXKFLXSKKKFGKAXVGEXXNS

SEQ ID NO: 158 - protein, 23 aa, Artificial Sequence
  source: /note=“Description of Artificial Sequence: Synthetic peptide”
  MOD_RES (2)..(2): (S)-2-(4′-pentenyl)Alanine
  SITE (2)..(6): /note=“May or may not be cross-linked via a side chain”
  MOD_RES (6)..(6): (S)-2-(4′-pentenyl)Alanine
  MOD_RES (16)..(16): (S)-2-(4′-pentenyl)Alanine
  SITE (16)..(20): /note=“May or may not be cross-linked via a side chain”
  MOD_RES (20)..(20): (S)-2-(4′-pentenyl)Alanine
  MOD_RES (21)..(21): Norleucine
GXGKFXHSKKKFGKAXVGEXXNS

SEQ ID NO: 159 - skipped

SEQ ID NO: 160 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
  MOD_RES (16)..(16): Norleucine
LHSAKKFGKAXVGEXXNS

SEQ ID NO: 161 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
LHSAKKFGKAXVGEXANS

SEQ ID NO: 162 - protein, 15 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
XKGKXLHSAKKFGKA

SEQ ID NO: 163 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
  MOD_RES (16)..(16): Norleucine
LHSAKKFGKAXVGEXXNS

SEQ ID NO: 164 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
LHSAKKFGKAXVGEXANS

SEQ ID NO: 165 - protein, 15 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
XIEKXLHSAKKFGKA

SEQ ID NO: 166 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
  MOD_RES (16)..(16): Norleucine
LESAKKFGKAXVGEXXNS

SEQ ID NO: 167 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
LESAKKFGKAXVGEXANS

SEQ ID NO: 168 - protein, 18 aa, Artificial Sequence
  source: /note=“Description of Artificial Sequence: Synthetic peptide”
  MOD_RES (16)..(16): Norleucine
LHSAKKFGKAFVGEIXES

SEQ ID NO: 169 - protein, 18 aa, Artificial Sequence
  source: /note=“Description of Artificial Sequence: Synthetic peptide”
LHSAKKFGKAFVGEIAES

SEQ ID NO: 170 - protein, 16 aa, Artificial Sequence
  source: /note=“Description of Artificial Sequence: Synthetic peptide”
  MOD_RES (14)..(14): Norleucine
SAKKFGKAFVGEIXNS

SEQ ID NO: 171 - protein, 16 aa, Artificial Sequence
  source: /note=“Description of Artificial Sequence: Synthetic peptide”
SAKKFGKAFVGEIANS

SEQ ID NO: 172 - protein, 16 aa, Artificial Sequence
  source: /note=“Description of Artificial Sequence: Synthetic peptide”
  MOD_RES (14)..(14): Norleucine
SAKKFGKAFVEEIXNS

SEQ ID NO: 173 - protein, 16 aa, Artificial Sequence
  source: /note=“Description of Artificial Sequence: Synthetic peptide”
SAKKFGKAFVEEIANS

SEQ ID NO: 174 - protein, 16 aa, Artificial Sequence
  source: /note=“Description of Artificial Sequence: Synthetic peptide”
  MOD_RES (14)..(14): Norleucine
SAKKFGKAFVGEIXES

SEQ ID NO: 175 - protein, 16 aa, Artificial Sequence
  source: /note=“Description of Artificial Sequence: Synthetic peptide”
SAKKFGKAFVGEIAES

SEQ ID NO: 176 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
  MOD_RES (16)..(16): Norleucine
LHSAKKFGKAXVGEXXNS

SEQ ID NO: 177 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
LHSAKKFGKAXVGEXANS

SEQ ID NO: 178 - protein, 15 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
XIGKXLHSAKKFGKA

SEQ ID NO: 179 - protein, 16 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
  MOD_RES (14)..(14): Norleucine
SAKKFGKAXVGEXXNS

SEQ ID NO: 180 - protein, 16 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
SAKKFGKAXVGEXANS

SEQ ID NO: 181 - protein, 15 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
GIXKFLXSAKKFGKA

SEQ ID NO: 182 - protein, 16 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
  MOD_RES (14)..(14): Norleucine
KAKKFGKAXVGEXXNS

SEQ ID NO: 183 - protein, 16 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
KAKKFGKAXVGEXANS

SEQ ID NO: 184 - protein, 15 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
GIXKFLXKAKKFGKA

SEQ ID NO: 185 - protein, 16 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
  MOD_RES (14)..(14): Norleucine
SKKKFGKAXVGEXXNS

SEQ ID NO: 186 - protein, 16 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
SKKKFGKAXVGEXANS

SEQ ID NO: 187 - protein, 15 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
GIXKFLXSKKKFGKA

SEQ ID NO: 188 - protein, 17 aa, Artificial Sequence
  source: /note=“Description of Artificial Sequence: Synthetic peptide”
  MOD_RES (10)..(10): alpha-methyl, alpha-alkenyl non-natural amino acid
  SITE (10)..(14): /note=“May or may not be cross-linked via a side chain”
  MOD_RES (14)..(14): alpha-methyl, alpha-alkenyl non-natural amino acid
  MOD_RES (15)..(15): Norleucine
HSKKKFGKAXVGEXXNS

SEQ ID NO: 189 - protein, 17 aa, Artificial Sequence
  source: /note=“Description of Artificial Sequence: Synthetic peptide”
  MOD_RES (10)..(10): alpha-methyl, alpha-alkenyl non-natural amino acid
  SITE (10)..(14): /note=“May or may not be cross-linked via a side chain”
  MOD_RES (14)..(14): alpha-methyl, alpha-alkenyl non-natural amino acid
HSKKKFGKAXVGEXANS

SEQ ID NO: 190 - protein, 15 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
GXGKFXHSKKKFGKA

SEQ ID NO: 191 - protein, 15 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
XIGKXLESAKKFGKA

SEQ ID NO: 192 - protein, 23 aa, Artificial Sequence
  source: /note=“Description of Artificial Sequence: Synthetic peptide”
  MOD_RES (4)..(4): (S)-2-(4′-pentenyl)Alanine
  SITE (4)..(8): /note=“May or may not be cross-linked via a side chain”
  MOD_RES (8)..(8): (S)-2-(4′-pentenyl)Alanine
  MOD_RES (21)..(21): Norleucine
GIGXFLHXAKKFGKAFVGEIXNS

SEQ ID NO: 193 - protein, 23 aa, Artificial Sequence
  source: /note=“Description of Artificial Sequence: Synthetic peptide”
  MOD_RES (8)..(8): (S)-2-(4′-pentenyl)Alanine
  SITE (8)..(12): /note=“May or may not be cross-linked via a side chain”
  MOD_RES (12)..(12): (S)-2-(4′-pentenyl)Alanine
  MOD_RES (21)..(21): Norleucine
GIGKFLHXAKKXGKAFVGEIXNS

SEQ ID NO: 194 - protein, 23 aa, Artificial Sequence
  source: /note=“Description of Artificial Sequence: Synthetic peptide”
  MOD_RES (9)..(9): (S)-2-(4′-pentenyl)Alanine
  SITE (9)..(13): /note=“May or may not be cross-linked via a side chain”
  MOD_RES (13)..(13): (S)-2-(4′-pentenyl)Alanine
  MOD_RES (21)..(21): Norleucine
GIGKFLHSXKKFXKAFVGEIXNS

SEQ ID NO: 195 - protein, 23 aa, Artificial Sequence
  source: /note=“Description of Artificial Sequence: Synthetic peptide”
  MOD_RES (10)..(10): (S)-2-(4′-pentenyl)Alanine
  SITE (10)..(14): /note=“May or may not be cross-linked via a side chain”
  MOD_RES (14)..(14): (S)-2-(4′-pentenyl)Alanine
  MOD_RES (21)..(21): Norleucine
GIGKFLHSAXKFGXAFVGEIXNS

SEQ ID NO: 196 - protein, 23 aa, Artificial Sequence
  source: /note=“Description of Artificial Sequence: Synthetic peptide”
  MOD_RES (14)..(14): (S)-2-(4′-pentenyl)Alanine
  SITE (14)..(18): /note=“May or may not be cross-linked via a side chain”
  MOD_RES (18)..(18): (S)-2-(4′-pentenyl)Alanine
  MOD_RES (21)..(21): Norleucine
GIGKFLHSAKKFGXAFVXEIXNS

SEQ ID NO: 197 - protein, 23 aa, Artificial Sequence
  source: /note=“Description of Artificial Sequence: Synthetic peptide”
  MOD_RES (15)..(15): (S)-2-(4′-pentenyl)Alanine
  SITE (15)..(19): /note=“May or may not be cross-linked via a side chain”
  MOD_RES (19)..(19): (S)-2-(4′-pentenyl)Alanine
  MOD_RES (21)..(21): Norleucine
GIGKFLHSAKKFGKXFVGXIXNS

SEQ ID NO: 198 - protein, 23 aa, Artificial Sequence
  source: /note=“Description of Artificial Sequence: Synthetic peptide”
  MOD_RES (18)..(18): (S)-2-(4′-pentenyl)Alanine
  SITE (18)..(22): /note=“May or may not be cross-linked via a side chain”
  MOD_RES (21)..(21): Norleucine
  MOD_RES (22)..(22): (S)-2-(4′-pentenyl)Alanine
GIGKFLHSAKKFGKAFVXEIXXS

SEQ ID NO: 199 - protein, 23 aa, Artificial Sequence
  source: /note=“Description of Artificial Sequence: Synthetic peptide”
  MOD_RES (19)..(19): (S)-2-(4′-pentenyl)Alanine
  SITE (19)..(23): /note=“May or may not be cross-linked via a side chain”
  MOD_RES (21)..(21): Norleucine
  MOD_RES (23)..(23): (S)-2-(4′-pentenyl)Alanine
GIGKFLHSAKKFGKAFVGXIXNX

SEQ ID NO: 200 - protein, 7 aa, Artificial Sequence
  source: /note=“Description of Artificial Sequence: Synthetic peptide”
GIGKFLH

SEQ ID NO: 201 - protein, 8 aa, Artificial Sequence
  source: /note=“Description of Artificial Sequence: Synthetic peptide”
GIGKFLHS

SEQ ID NO: 202 - protein, 9 aa, Artificial Sequence
  source: /note=“Description of Artificial Sequence: Synthetic peptide”
GIGKFLHSA

SEQ ID NO: 203 - protein, 13 aa, Artificial Sequence
  source: /note=“Description of Artificial Sequence: Synthetic peptide”
GIGKFLHSAKKFG

SEQ ID NO: 204 - protein, 14 aa, Artificial Sequence
  source: /note=“Description of Artificial Sequence: Synthetic peptide”
GIGKFLHSAKKFGK

SEQ ID NO: 205 - protein, 17 aa, Artificial Sequence
  source: /note=“Description of Artificial Sequence: Synthetic peptide”
GIGKFLHSAKKFGKAFV

SEQ ID NO: 206 - protein, 18 aa, Artificial Sequence
  source: /note=“Description of Artificial Sequence: Synthetic peptide”
GIGKFLHSAKKFGKAFVG

SEQ ID NO: 207 - protein, 15 aa, Artificial Sequence
  source: /note=“Description of Artificial Sequence: Synthetic peptide”
  MOD_RES (13)..(13): Norleucine
AKKFGKAFVGEIXNS

SEQ ID NO: 208 - protein, 11 aa, Artificial Sequence
  source: /note=“Description of Artificial Sequence: Synthetic peptide”
  MOD_RES (9)..(9): Norleucine
GKAFVGEIXNS

SEQ ID NO: 209 - protein, 10 aa, Artificial Sequence
  source: /note=“Description of Artificial Sequence: Synthetic peptide”
  MOD_RES (8)..(8): Norleucine
KAFVGEIXNS

SEQ ID NO: 210 - protein, 9 aa, Artificial Sequence
  source: /note=“Description of Artificial Sequence: Synthetic peptide”
  MOD_RES (7)..(7): Norleucine
AFVGEIXNS

SEQ ID NO: 211 - protein, 5 aa, Artificial Sequence
  source: /note=“Description of Artificial Sequence: Synthetic peptide”
  MOD_RES (3)..(3): Norleucine
EIXNS

SEQ ID NO: 212 - protein, 4 aa, Artificial Sequence
  source: /note=“Description of Artificial Sequence: Synthetic peptide”
  MOD_RES (2)..(2): Norleucine
IXNS

SEQ ID NO: 213 - protein, 15 aa, Artificial Sequence
  source: /note=“Description of Artificial Sequence: Synthetic peptide”
AKKFGKAFVGEIANS

SEQ ID NO: 214 - protein, 11 aa, Artificial Sequence
  source: /note=“Description of Artificial Sequence: Synthetic peptide”
GKAFVGEIANS

SEQ ID NO: 215 - protein, 10 aa, Artificial Sequence
  source: /note=“Description of Artificial Sequence: Synthetic peptide”
KAFVGEIANS

SEQ ID NO: 216 - protein, 9 aa, Artificial Sequence
  source: /note=“Description of Artificial Sequence: Synthetic peptide”
AFVGEIANS

SEQ ID NO: 217 - protein, 5 aa, Artificial Sequence
  source: /note=“Description of Artificial Sequence: Synthetic peptide”
EIANS

SEQ ID NO: 218 - protein, 4 aa, Artificial Sequence
  source: /note=“Description of Artificial Sequence: Synthetic peptide”
IANS

SEQ ID NO: 219 - protein, 25 aa, Pleuronectes americanus
GWGSFFKKAAHVGKHVGKAALTHYL

SEQ ID NO: 220 - protein, 37 aa, Unknown
  source: /note=“Description of Unknown: CAP18 sequence”
GLRKRLRKFRNKIKEKLKKIGQKIQGLLPKLAPRTDY

SEQ ID NO: 221 - protein, 21 aa, Unknown
  source: /note=“Description of Unknown: Esculentin sequence”
GIFSKLAGKKIKNLLISGLKG

SEQ ID NO: 222 - protein, 25 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
GXGSFXKKKAHVGKHXGKAXLTHYL

SEQ ID NO: 223 - protein, 25 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
GXGSFXKKAAHVGKHXGKAXLTHYL

SEQ ID NO: 224 - protein, 25 aa, Artificial Sequence
  source: /note=“Description of Artificial Sequence: Synthetic peptide”
  MOD_RES (13)..(13): alpha-methyl, alpha-alkenyl non-natural amino acid
  SITE (13)..(17): /note=“May or may not be cross-linked via a side chain”
  MOD_RES (17)..(17): alpha-methyl, alpha-alkenyl non-natural amino acid
GWGSFFKKAAHVXKHVXKAALTHYL

SEQ ID NO: 225 - protein, 32 aa, Artificial Sequence
  source: /note=“Description of Artificial Sequence: Synthetic polypeptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
  MOD_RES (24)..(24): alpha-methyl, alpha-alkenyl non-natural amino acid
  SITE (24)..(28): /note=“May or may not be cross-linked via a side chain”
  MOD_RES (28)..(28): alpha-methyl, alpha-alkenyl non-natural amino acid
GXRKRXRKFRNKIKEKKKKIGQKXQGLXPKLA

SEQ ID NO: 226 - protein, 21 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
  MOD_RES (15)..(15): alpha-methyl, alpha-alkenyl non-natural amino acid
  SITE (15)..(19): /note=“May or may not be cross-linked via a side chain”
  MOD_RES (19)..(19): alpha-methyl, alpha-alkenyl non-natural amino acid
GXFSKXKGKKIKNLXISGXKG

SEQ ID NO: 227 - protein, 25 aa, Artificial Sequence
  source: /note=“Description of Artificial Sequence: Synthetic peptide”
GWGSFFKKKAHVGKHVGKAALTHYL

SEQ ID NO: 228 - protein, 32 aa, Unknown
  source: /note=“Description of Unknown: CAP18 sequence”
GLRKRLRKFRNKIKEKLKKIGQKIQGLLPKLA

SEQ ID NO: 229 - protein, 37 aa, Artificial Sequence
  source: /note=“Description of Artificial Sequence: Synthetic polypeptide”
  VARIANT (17)..(17): /replace=“L”
  VARIANT (28)..(28): /replace=“L”
  SITE (1)..(37): /note=“Variant residues given in the sequence have no preference with respect to those in the annotations for variant positions”
GLRKRLRKFRNKIKEKKKKIGQKIQGLVPKLAPRTDY

SEQ ID NO: 230 - protein, 21 aa, Artificial Sequence
  source: /note=“Description of Artificial Sequence: Synthetic peptide”
GIFSKLKGKKIKNLLISGLKG

SEQ ID NO: 231 - protein, 37 aa, Unknown
  source: /note=“Description of Unknown: CAP18 sequence”
GLRKRLRKFRNKIKEKLKKIGQKIQGLVPKLAPRTDY

SEQ ID NO: 232 - protein, 32 aa, Unknown
  source: /note=“Description of Unknown: CAP18 sequence”
GLRKRLRKFRNKIKEKLKKIGQKIQGLVPKLA

SEQ ID NO: 233 - protein, 23 aa, Artificial Sequence
  source: /note=“Description of Artificial Sequence: Synthetic peptide”
  VARIANT (1)..(1): /replace=“a hydrophobic amino acid” or “a hydrophilic amino acid” or “a stapling amino acid”
  VARIANT (2)..(2): /replace=“a hydrophobic amino acid” or “a stapling amino acid”
  VARIANT (3)..(3): /replace=“a hydrophobic amino acid” or “a hydrophilic amino acid” or “a stapling amino acid”
  VARIANT (4)..(4): /replace=“a positively charged amino acid”
  VARIANT (5)..(5): /replace=“a hydrophobic amino acid” or “a stapling amino acid”
  VARIANT (6)..(6): /replace=“a hydrophobic amino acid” or “a stapling amino acid”
  VARIANT (7)..(7): /replace=“a positively charged amino acid” or “a stapling amino acid”
  VARIANT (8)..(8): /replace=“a hydrophobic amino acid” or “a hydrophilic amino acid” or “a stapling amino acid”
  VARIANT (9)..(9): /replace=“a hydrophobic amino acid” or “a stapling amino acid”
  VARIANT (10)..(10): /replace=“a positively charged amino acid” or “a stapling amino acid”
  VARIANT (11)..(11): /replace=“a positively charged amino acid” or “a stapling amino acid”
  VARIANT (12)..(12): /replace=“a hydrophobic amino acid” or “a stapling amino acid”
  VARIANT (13)..(13): /replace=“a hydrophobic amino acid” or “a stapling amino acid”
  VARIANT (14)..(14): /replace=“a positively charged amino acid” or “a stapling amino acid”
  VARIANT (15)..(15): /replace=“a hydrophobic amino acid” or “a hydrophilic amino acid” or “a stapling amino acid”
  VARIANT (16)..(16): /replace=“a hydrophobic amino acid” or “a stapling amino acid”
  VARIANT (17)..(17): /replace=“a hydrophobic amino acid” or “a stapling amino acid”
  VARIANT (18)..(18): /replace=“a hydrophobic amino acid” or “a hydrophilic amino acid” or “a stapling amino acid”
  VARIANT (19)..(19): /replace=“a hydrophobic amino acid” or “a hydrophilic amino acid” or “a stapling amino acid”
  VARIANT (20)..(20): /replace=“a hydrophobic amino acid” or “a stapling amino acid”
  VARIANT (21)..(21): /replace=“Norleucine” or “a hydrophobic amino acid” or “a stapling amino acid”
  VARIANT (22)..(22): /replace=“a hydrophilic amino acid” or “a positively charged amino acid” or “a stapling amino acid”
  VARIANT (23)..(23): /replace=“a hydrophobic amino acid” or “a hydrophilic amino acid” or “a stapling amino acid”
  SITE (1)..(23): /note=“Variant residues given in the sequence have no preference with respect to those in the annotations for variant positions”
  source: /note=“See specification as filed for detailed description of substitutions and preferred embodiments”
GIGKFLHSAKKFGKAFVGEIMNS

SEQ ID NO: 234 - protein, 5 aa, Artificial Sequence
  source: /note=“Description of Artificial Sequence: Synthetic peptide”
GIGKF

SEQ ID NO: 235 - protein, 10 aa, Artificial Sequence
  source: /note=“Description of Artificial Sequence: Synthetic peptide”
GIGKFLHSAK

SEQ ID NO: 236 - protein, 12 aa, Artificial Sequence
  source: /note=“Description of Artificial Sequence: Synthetic peptide”
GIGKFLHSAKKF

SEQ ID NO: 237 - protein, 6 aa, Artificial Sequence
  source: /note=“Description of Artificial Sequence: Synthetic peptide”
IGKFLH

SEQ ID NO: 238 - protein, 6 aa, Artificial Sequence
  source: /note=“Description of Artificial Sequence: Synthetic peptide”
GKFLHS

SEQ ID NO: 239 - protein, 6 aa, Artificial Sequence
  source: /note=“Description of Artificial Sequence: Synthetic peptide”
KFLHSA

SEQ ID NO: 240 - protein, 6 aa, Artificial Sequence
  source: /note=“Description of Artificial Sequence: Synthetic peptide”
FLHSAK

SEQ ID NO: 241 - protein, 6 aa, Artificial Sequence
  source: /note=“Description of Artificial Sequence: Synthetic peptide”
LHSAKK

SEQ ID NO: 242 - protein, 6 aa, Artificial Sequence
  source: /note=“Description of Artificial Sequence: Synthetic peptide”
HSAKKF

SEQ ID NO: 243 - protein, 6 aa, Artificial Sequence
  source: /note=“Description of Artificial Sequence: Synthetic peptide”
SAKKFG

SEQ ID NO: 244 - protein, 6 aa, Artificial Sequence
  source: /note=“Description of Artificial Sequence: Synthetic peptide”
AKKFGK

SEQ ID NO: 245 - protein, 6 aa, Artificial Sequence
  source: /note=“Description of Artificial Sequence: Synthetic peptide”
KKFGKA

SEQ ID NO: 246 - protein, 6 aa, Artificial Sequence
  source: /note=“Description of Artificial Sequence: Synthetic peptide”
KFGKAF

SEQ ID NO: 247 - protein, 6 aa, Artificial Sequence
  source: /note=“Description of Artificial Sequence: Synthetic peptide”
FGKAFV

SEQ ID NO: 248 - protein, 6 aa, Artificial Sequence
  source: /note=“Description of Artificial Sequence: Synthetic peptide”
GKAFVG

SEQ ID NO: 249 - protein, 6 aa, Artificial Sequence
  source: /note=“Description of Artificial Sequence: Synthetic peptide”
KAFVGE

SEQ ID NO: 250 - protein, 6 aa, Artificial Sequence
  source: /note=“Description of Artificial Sequence: Synthetic peptide”
AFVGEI

SEQ ID NO: 251 - protein, 6 aa, Artificial Sequence
  source: /note=“Description of Artificial Sequence: Synthetic peptide”
  MOD_RES (6)..(6): Norleucine
FVGEIX

SEQ ID NO: 252 - protein, 6 aa, Artificial Sequence
  source: /note=“Description of Artificial Sequence: Synthetic peptide”
  MOD_RES (5)..(5): Norleucine
VGEIXN

SEQ ID NO: 253 - protein, 15 aa, Artificial Sequence
  source: /note=“Description of Artificial Sequence: Synthetic peptide”
  MOD_RES (13)..(13): Norleucine
AKKFGKAFVGEIXNS

SEQ ID NO: 254 - protein, 14 aa, Artificial Sequence
  source: /note=“Description of Artificial Sequence: Synthetic peptide”
  MOD_RES (12)..(12): Norleucine
KKFGKAFVGEIXNS

SEQ ID NO: 255 - protein, 13 aa, Artificial Sequence
  source: /note=“Description of Artificial Sequence: Synthetic peptide”
  MOD_RES (11)..(11): Norleucine
KFGKAFVGEIXNS

SEQ ID NO: 256 - protein, 12 aa, Artificial Sequence
  source: /note=“Description of Artificial Sequence: Synthetic peptide”
  MOD_RES (10)..(10): Norleucine
FGKAFVGEIXNS

SEQ ID NO: 257 - protein, 11 aa, Artificial Sequence
  source: /note=“Description of Artificial Sequence: Synthetic peptide”
  MOD_RES (9)..(9): Norleucine
GKAFVGEIXNS

SEQ ID NO: 258 - protein, 10 aa, Artificial Sequence
  source: /note=“Description of Artificial Sequence: Synthetic peptide”
  MOD_RES (8)..(8): Norleucine
KAFVGEIXNS

SEQ ID NO: 259 - protein, 9 aa, Artificial Sequence
  source: /note=“Description of Artificial Sequence: Synthetic peptide”
  MOD_RES (7)..(7): Norleucine
AFVGEIXNS

SEQ ID NO: 260 - protein, 8 aa, Artificial Sequence
  source: /note=“Description of Artificial Sequence: Synthetic peptide”
  MOD_RES (6)..(6): Norleucine
FVGEIXNS

SEQ ID NO: 261 - protein, 7 aa, Artificial Sequence
  source: /note=“Description of Artificial Sequence: Synthetic peptide”
  MOD_RES (5)..(5): Norleucine
VGEIXNS

SEQ ID NO: 262 - protein, 6 aa, Artificial Sequence
  source: /note=“Description of Artificial Sequence: Synthetic peptide”
  MOD_RES (4)..(4): Norleucine
GEIXNS

SEQ ID NO: 263 - protein, 5 aa, Artificial Sequence
  source: /note=“Description of Artificial Sequence: Synthetic peptide”
  MOD_RES (3)..(3): Norleucine
EIXNS

SEQ ID NO: 264 - protein, 4 aa, Artificial Sequence
  source: /note=“Description of Artificial Sequence: Synthetic peptide”
  MOD_RES (2)..(2): Norleucine
IXNS

SEQ ID NO: 265 - protein, 15 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
GXGSFXKKKAHVGKH

SEQ ID NO: 266 - protein, 15 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
GXGSFXKKAAHVGKH

SEQ ID NO: 267 - protein, 12 aa, Artificial Sequence
  source: /note=“Description of Artificial Sequence: Synthetic peptide”
GWGSFFKKAAHV

SEQ ID NO: 268 - protein, 23 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
GXRKRXRKFRNKIKEKKKKIGQK

SEQ ID NO: 269 - protein, 14 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(6): /note=“May or may not be cross-linked via a side chain”
  MOD_RES (6)..(6): alpha-methyl, alpha-alkenyl non-natural amino acid
GXFSKXKGKKIKNL

SEQ ID NO: 270 - protein, 19 aa, Artificial Sequence
  source: /note=“Description of Artificial Sequence: Synthetic peptide”
  MOD_RES (10)..(10): alpha-methyl, alpha-alkenyl non-natural amino acid
  SITE (10)..(14): /note=“May or may not be cross-linked via a side chain”
  MOD_RES (14)..(14): alpha-methyl, alpha-alkenyl non-natural amino acid
KKKAHVGKHXGKAXLTHYL

SEQ ID NO: 271 - protein, 5 aa, Artificial Sequence
  source: /note=“Description of Artificial Sequence: Synthetic peptide”
LTHYL

SEQ ID NO: 272 - protein, 5 aa, Artificial Sequence
  source: /note=“Description of Artificial Sequence: Synthetic peptide”
LTHYL

SEQ ID NO: 273 - protein, 8 aa, Artificial Sequence
  source: /note=“Description of Artificial Sequence: Synthetic peptide”
KAALTHYL

SEQ ID NO: 274 - protein, 26 aa, Artificial Sequence
  source: /note=“Description of Artificial Sequence: Synthetic peptide”
  MOD_RES (18)..(18): alpha-methyl, alpha-alkenyl non-natural amino acid
  SITE (18)..(22): /note=“May or may not be cross-linked via a side chain”
  MOD_RES (22)..(22): alpha-methyl, alpha-alkenyl non-natural amino acid
RKFRNKIKEKKKKIGQKXQGLXPKLA

SEQ ID NO: 275 - protein, 4 aa, Artificial Sequence
  source: /note=“Description of Artificial Sequence: Synthetic peptide”
PKLA

SEQ ID NO: 276 - protein, 15 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(13): /note=“May or may not be cross-linked via a side chain”
  MOD_RES (13)..(13): alpha-methyl, alpha-alkenyl non-natural amino acid
KGKKIKNLXISGXKG

SEQ ID NO: 277 - protein, 6 aa, Artificial Sequence
  source: /note=“Description of Artificial Sequence: Synthetic peptide”
FVGEIA

SEQ ID NO: 278 - protein, 6 aa, Artificial Sequence
  source: /note=“Description of Artificial Sequence: Synthetic peptide”
VGEIAN

SEQ ID NO: 279 - protein, 15 aa, Artificial Sequence
  source: /note=“Description of Artificial Sequence: Synthetic peptide”
AKKFGKAFVGEIANS

SEQ ID NO: 280 - protein, 14 aa, Artificial Sequence
  source: /note=“Description of Artificial Sequence: Synthetic peptide”
KKFGKAFVGEIANS

SEQ ID NO: 281 - protein, 13 aa, Artificial Sequence
  source: /note=“Description of Artificial Sequence: Synthetic peptide”
KFGKAFVGEIANS

SEQ ID NO: 282 - protein, 12 aa, Artificial Sequence
  source: /note=“Description of Artificial Sequence: Synthetic peptide”
FGKAFVGEIANS

SEQ ID NO: 283 - protein, 11 aa, Artificial Sequence
  source: /note=“Description of Artificial Sequence: Synthetic peptide”
GKAFVGEIANS

SEQ ID NO: 284 - protein, 10 aa, Artificial Sequence
  source: /note=“Description of Artificial Sequence: Synthetic peptide”
KAFVGEIANS

SEQ ID NO: 285 - protein, 9 aa, Artificial Sequence
  source: /note=“Description of Artificial Sequence: Synthetic peptide”
AFVGEIANS

SEQ ID NO: 286 - protein, 8 aa, Artificial Sequence
  source: /note=“Description of Artificial Sequence: Synthetic peptide”
FVGEIANS

SEQ ID NO: 287 - protein, 7 aa, Artificial Sequence
  source: /note=“Description of Artificial Sequence: Synthetic peptide”
VGEIANS

SEQ ID NO: 288 - protein, 6 aa, Artificial Sequence
  source: /note=“Description of Artificial Sequence: Synthetic peptide”
GEIANS

SEQ ID NO: 289 - protein, 5 aa, Artificial Sequence
  source: /note=“Description of Artificial Sequence: Synthetic peptide”
EIANS

SEQ ID NO: 290 - protein, 4 aa, Artificial Sequence
  source: /note=“Description of Artificial Sequence: Synthetic peptide”
IANS

SEQ ID NO: 291 - protein, 19 aa, Artificial Sequence
  source: /note=“Description of Artificial Sequence: Synthetic peptide”
  MOD_RES (10)..(10): alpha-methyl, alpha-alkenyl non-natural amino acid
  SITE (10)..(14): /note=“May or may not be cross-linked via a side chain”
  MOD_RES (14)..(14): alpha-methyl, alpha-alkenyl non-natural amino acid
KKAAHVGKHXGKAXLTHYL

SEQ ID NO: 292 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(8): /note=“May or may not be cross-linked via a side chain”
  MOD_RES (8)..(8): alpha-methyl, alpha-alkenyl non-natural amino acid
  MOD_RES (21)..(21): Norleucine
XIGKFLHXAKKFGKAFVGEIXNS

SEQ ID NO: 293 - protein, 23 aa, Artificial Sequence
  source: /note=“Description of Artificial Sequence: Synthetic peptide”
  MOD_RES (2)..(2): alpha-methyl, alpha-alkenyl non-natural amino acid
  SITE (2)..(9): /note=“May or may not be cross-linked via a side chain”
  MOD_RES (9)..(9): alpha-methyl, alpha-alkenyl non-natural amino acid
  MOD_RES (21)..(21): Norleucine
GXGKFLHSXKKFGKAFVGEIXNS

SEQ ID NO: 294 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(10): /note=“May or may not be cross-linked via a side chain”
  MOD_RES (10)..(10): alpha-methyl, alpha-alkenyl non-natural amino acid
  MOD_RES (21)..(21): Norleucine
GIXKFLHSAXKFGKAFVGEIXNS

SEQ ID NO: 295 - protein, 23 aa, Artificial Sequence
  source: /note=“Description of Artificial Sequence: Synthetic peptide”
  MOD_RES (4)..(4): alpha-methyl, alpha-alkenyl non-natural amino acid
  SITE (4)..(11): /note=“May or may not be cross-linked via a side chain”
  MOD_RES (11)..(11): alpha-methyl, alpha-alkenyl non-natural amino acid
  MOD_RES (21)..(21): Norleucine
GIGXFLHSAKXFGKAFVGEIXNS

SEQ ID NO: 296 - protein, 23 aa, Artificial Sequence
  source: /note=“Description of Artificial Sequence: Synthetic peptide”
  MOD_RES (5)..(5): alpha-methyl, alpha-alkenyl non-natural amino acid
  SITE (5)..(12): /note=“May or may not be cross-linked via a side chain”
  MOD_RES (12)..(12): alpha-methyl, alpha-alkenyl non-natural amino acid
  MOD_RES (21)..(21): Norleucine
GIGKXLHSAKKXGKAFVGEIXNS

SEQ ID NO: 297 - protein, 23 aa, Artificial Sequence
  source: /note=“Description of Artificial Sequence: Synthetic peptide”
  MOD_RES (6)..(6): alpha-methyl, alpha-alkenyl non-natural amino acid
  SITE (6)..(13): /note=“May or may not be cross-linked via a side chain”
  MOD_RES (13)..(13): alpha-methyl, alpha-alkenyl non-natural amino acid
  MOD_RES (21)..(21): Norleucine
GIGKFXHSAKKFXKAFVGEIXNS

SEQ ID NO: 298 - protein, 23 aa, Artificial Sequence
  source: /note=“Description of Artificial Sequence: Synthetic peptide”
  MOD_RES (7)..(7): alpha-methyl, alpha-alkenyl non-natural amino acid
  SITE (7)..(14): /note=“May or may not be cross-linked via a side chain”
  MOD_RES (14)..(14): alpha-methyl, alpha-alkenyl non-natural amino acid
  MOD_RES (21)..(21): Norleucine
GIGKFLXSAKKFGXAFVGEIXNS

SEQ ID NO: 299 - protein, 23 aa, Artificial Sequence
  source: /note=“Description of Artificial Sequence: Synthetic peptide”
  MOD_RES (8)..(8): alpha-methyl, alpha-alkenyl non-natural amino acid
  SITE (8)..(15): /note=“May or may not be cross-linked via a side chain”
  MOD_RES (15)..(15): alpha-methyl, alpha-alkenyl non-natural amino acid
  MOD_RES (21)..(21): Norleucine
GIGKFLHXAKKFGKXFVGEIXNS

SEQ ID NO: 300 - protein, 23 aa, Artificial Sequence
  source: /note=“Description of Artificial Sequence: Synthetic peptide”
  MOD_RES (9)..(9): alpha-methyl, alpha-alkenyl non-natural amino acid
  SITE (9)..(16): /note=“May or may not be cross-linked via a side chain”
  MOD_RES (16)..(16): alpha-methyl, alpha-alkenyl non-natural amino acid
  MOD_RES (21)..(21): Norleucine
GIGKFLHSXKKFGKAXVGEIXNS

SEQ ID NO: 301 - protein, 23 aa, Artificial Sequence
  source: /note=“Description of Artificial Sequence: Synthetic peptide”
  MOD_RES (10)..(10): alpha-methyl, alpha-alkenyl non-natural amino acid
  SITE (10)..(17): /note=“May or may not be cross-linked via a side chain”
  MOD_RES (17)..(17): alpha-methyl, alpha-alkenyl non-natural amino acid
  MOD_RES (21)..(21): Norleucine
GIGKFLHSAXKFGKAFXGEIXNS

SEQ ID NO: 302 - protein, 23 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(18): /note=“May or may not be cross-linked via a side chain”
  MOD_RES (18)..(18): alpha-methyl, alpha-alkenyl non-natural amino acid
  MOD_RES (21)..(21): Norleucine
GIGKFLHSAKXFGKAFVXEIXNS

SEQ ID NO: 303 - protein, 23 aa, Artificial Sequence
  source: /note=“Description of Artificial Sequence: Synthetic peptide”
  MOD_RES (12)..(12): alpha-methyl, alpha-alkenyl non-natural amino acid
  SITE (12)..(19): /note=“May or may not be cross-linked via a side chain”
  MOD_RES (19)..(19): alpha-methyl, alpha-alkenyl non-natural amino acid
  MOD_RES (21)..(21): Norleucine
GIGKFLHSAKKXGKAFVGXIXNS

SEQ ID NO: 304 - protein, 23 aa, Artificial Sequence
  source: /note=“Description of Artificial Sequence: Synthetic peptide”
  MOD_RES (13)..(13): alpha-methyl, alpha-alkenyl non-natural amino acid
  SITE (13)..(20): /note=“May or may not be cross-linked via a side chain”
  MOD_RES (20)..(20): alpha-methyl, alpha-alkenyl non-natural amino acid
  MOD_RES (21)..(21): Norleucine
GIGKFLHSAKKFXKAFVGEXXNS

SEQ ID NO: 305 - protein, 23 aa, Artificial Sequence
  source: /note=“Description of Artificial Sequence: Synthetic peptide”
  MOD_RES (14)..(14): alpha-methyl, alpha-alkenyl non-natural amino acid
  SITE (14)..(21): /note=“May or may not be cross-linked via a side chain”
  MOD_RES (21)..(21): alpha-methyl, alpha-alkenyl non-natural amino acid
GIGKFLHSAKKFGXAFVGEIXNS

SEQ ID NO: 306 - protein, 23 aa, Artificial Sequence
  source: /note=“Description of Artificial Sequence: Synthetic peptide”
  MOD_RES (15)..(15): alpha-methyl, alpha-alkenyl non-natural amino acid
  SITE (15)..(22): /note=“May or may not be cross-linked via a side chain”
  MOD_RES (21)..(21): Norleucine
  MOD_RES (22)..(22): alpha-methyl, alpha-alkenyl non-natural amino acid
GIGKFLHSAKKFGKXFVGEIXXS

SEQ ID NO: 307 - protein, 23 aa, Artificial Sequence
  source: /note=“Description of Artificial Sequence: Synthetic peptide”
  MOD_RES (16)..(16): alpha-methyl, alpha-alkenyl non-natural amino acid
  SITE (16)..(23): /note=“May or may not be cross-linked via a side chain”
  MOD_RES (21)..(21): Norleucine
  MOD_RES (23)..(23): alpha-methyl, alpha-alkenyl non-natural amino acid
GIGKFLHSAKKFGKAXVGEIXNX

SEQ ID NO: 308 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
XIGKXLHKAKKFGKAXVGEXAKK

SEQ ID NO: 309 - protein, 23 aa, Artificial Sequence
  source: /note=“Description of Artificial Sequence: Synthetic peptide”
  MOD_RES (1)..(1): alpha-methyl, alpha-alkenyl non-natural amino acid
  SITE (1)..(5): /note=“May or may not be cross-linked via a side chain”
  MOD_RES (5)..(5): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
XIGKXLHSKKKFGKAXVGEXAKK

SEQ ID NO: 310 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
GIXKFLXKAKKFGKAXVGEXAKK

SEQ ID NO: 311 - protein, 23 aa, Artificial Sequence
  source: /note=“Description of Artificial Sequence: Synthetic peptide”
  MOD_RES (3)..(3): alpha-methyl, alpha-alkenyl non-natural amino acid
  SITE (3)..(7): /note=“May or may not be cross-linked via a side chain”
  MOD_RES (7)..(7): alpha-methyl, alpha-alkenyl non-natural amino acid
  MOD_RES (16)..(16): alpha-methyl, alpha-alkenyl non-natural amino acid
  SITE (16)..(20): /note=“May or may not be cross-linked via a side chain”
  MOD_RES (20)..(20): alpha-methyl, alpha-alkenyl non-natural amino acid
GIXKFLXSKKKFGKAXVGEXAKK

SEQ ID NO: 312 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSKKKFGKAFVGEIMNS

SEQ ID NO: 313 - protein, 23 aa, Artificial Sequence
  source: /note=“Description of Artificial Sequence: Synthetic peptide”
  VARIANT (21)..(21): /replace=“Norleucine”
  SITE (1)..(23): /note=“Variant residues given in the sequence have no preference with respect to those in the annotations for variant positions”
GIGKFLHSAKEFGKAFVGEIMNS

SEQ ID NO: 314 - protein, 23 aa, Artificial Sequence
  source: /note=“Description of Artificial Sequence: Synthetic peptide”
GIGKFLHKAKKFGKAFVGEIAKK

SEQ ID NO: 315 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
  MOD_RES (16)..(16): Norleucine
LHSAKKFGKAXVEEXXNS

SEQ ID NO: 316 - protein, 18 aa, Artificial Sequence
  source: /note=“Description of Artificial Sequence: Synthetic peptide”
  MOD_RES (11)..(11): alpha-methyl, alpha-alkenyl non-natural amino acid
  SITE (11)..(15): /note=“May or may not be cross-linked via a side chain”
  MOD_RES (15)..(15): alpha-methyl, alpha-alkenyl non-natural amino acid
LHSAKKFGKAXVEEXANS

SEQ ID NO: 317 - protein, 23 aa, Artificial Sequence
  source: /note=“Description of Artificial Sequence: Synthetic peptide”
  MOD_RES (21)..(21): Norleucine
GIGKFLHSAKKFGKAFVGEIXNS

(19) United States
(12) Patent Application Publication
(10) Pub. No.: US 2023/0116760 A1
(43) Pub. Date: Apr. 13, 2023

(54) STRUCTURALLY-STABILIZED ONCOLYTIC PEPTIDES AND USES THEREOF

(71) Applicant: Dana-Farber Cancer Institute, Inc., Boston, MA (US)

(72) Inventors: Loren D. Walensky, Newton, MA (US); Rida Mourtada, Brookline, MA (US); Henry D. Herce, Brookline, MA (US)

(21) Appl. No.: 17/785,561

(22) PCT Filed: Dec. 15, 2020
(86) PCT No.: PCT/US2020/065078

§ 371 Date: Jun. 15, 2022

Related U.S. Application Data
(60) Provisional application No. 62/948,582, filed on Dec. 16, 2019.

(51) Int. Cl. A61K 38/16; C07K 14/46; A61P 35/00

(52) U.S. Cl. CPC A61K 38/16; C07K 14/463; A61P 35/00

(57) ABSTRACT

This disclosure features structurally-stabilized oncolytic peptides and related compositions and methods of making same. Also disclosed are methods of using such structurally-stabilized peptides in the treatment of cancer, e.g., a hematological cancer, e.g., a leukemia, a lymphoma, or multiple myeloma, and/or in the inhibition of proliferation of a cancer cell, e.g., a hematological cancer, e.g., a leukemia and/or a lymphoma and/or a multiple myeloma cell. Also disclosed are methods of using such structurally-stabilized peptides for killing cancer cells or dual-killing cancer cell and micro-organisms, and thus providing therapeutics to treat cancer with or without simultaneously preventing or treating infection, while avoiding toxicity to normal, non-cancerous cells.

### CROSS-REFERENCE TO RELATED APPLICATIONS

This application is a U.S. National Stage application, and claims the priority benefit of International Application No. PCT/US2020/065078, filed Dec. 15, 2020, which claims the priority benefit of U.S. Provisional Application No. 62/948,582, filed Dec. 16, 2019, the contents of each of which are hereby incorporated by reference herein in their entireties.

### SEQUENCE LISTING

The instant application contains a Sequence Listing which has been submitted electronically in ASCII format and is hereby incorporated by reference in its entirety. Said ASCII copy, created on Nov. 21, 2022, is named 00530-0375WO1_SL.txt and is 183,797 bytes in size.

### TECHNICAL FIELD

This disclosure features structurally-stabilized oncolytic peptides and related compositions and methods of making same. Also disclosed are methods of using such structurally-stabilized peptides in the treatment of cancer (e.g., a hematological cancer, e.g., a leukemia, a lymphoma, a multiple myeloma) and/or in the inhibition of proliferation of a cancer cell (e.g., a hematological cancer cell, e.g., a leukemia cell, a lymphoma cell, a multiple myeloma cell). Also disclosed are methods of using such structurally-stabilized peptides for killing cancer cells or for dual-killing of cancer cells and micro-organisms, and thus providing therapeutics to treat cancer with or without simultaneously preventing or treating infection, while avoiding toxicity to normal, non-cancerous cells.

### BACKGROUND

The development of resistance to treatment is a common problem in oncology and infectious disease. Cancer cells readily develop resistance to kinase inhibitors or antibodies that target certain surface proteins. Thus, there remains a need for the development of therapeutics for the treatment of cancer.

Similar to the resistance that readily develops in cancer cells, antibiotics that target enzymatic or ribosomal processes are associated with high rates of resistance. However, resistance rates to antibiotics that lyse bacterial membranes, such as antimicrobial peptides and daptomycin, are much lower due to the difficulty in modulating membrane composition to thwart these agents.

Anti-microbial peptides (AMPs) are an evolutionarily conserved class of proteins that form an essential line of defense against microbial invasion. These peptides are produced by many disparate organisms and have been found to exhibit a wide spectrum of activity against bacteria, fungi (including yeasts), protozoa (including parasites), and viruses. AMPs can be divided into four main structural groups: stabilized p-sheet peptides with two to four disulfide bridges; loop peptides with a single disulfide bridge; α-helical peptides; and extended structures rich in arginine, glycine, proline, tryptophan, and histidine. Typically 12 to 50 amino acids in length, these peptides are usually cationic with an amphipathic character. These biophysical properties allow them to interact with bacterial membranes resulting in either disruption of membrane integrity or translocation into bacterial cells and disruption of intracellular processes. The alpha-helical structural motif of AMPs can be important to the ability of AMPs to interact with bacterial membranes. Upon binding to the membrane, AMPs can either translocate or insert themselves and permeabilize the membrane through a barrel-stove mechanism, a carpet-like mechanism or a toroidal pore mechanism. This process of permeabilization and disruption of membrane integrity can account for the antimicrobial properties of alpha-helical AMPs.

### SUMMARY

This disclosure features novel Stapled Oncolytic Peptides (StOPs). Also, Applicant has surprisingly shown that exemplary StOPs—stabilized (e.g., stapled) magainin peptides—can be used to selectively kill cancer cells (e.g., hematological cancer cells, e.g., leukemia cells, lymphoma cells, multiple myeloma cells). In addition, also described herein are stabilized (e.g., stapled) magainin peptides that can lyse both cancer cells and bacterial cells. The StOPs disclosed herein do not have, or have reduced, lytic activity on non-cancerous cells (e.g., red blood cells, endothelial cells). The StOPs disclosed herein can be used for treating cancer. In some instances, the cancer is a hematological cancer. In some instances, the cancer is a leukemia. In some instances, the cancer is a lymphoma. In some instances, the cancer is multiple myeloma. In certain instances, the cancer is acute myeloid leukemia. In some instances, the cancer is a histiocytic lymphoma. In certain instances, the cancer is a mixed lineage leukemia. The StOPs disclosed herein can be used to lyse cells comprising an anionic outer leaflet of the cell membrane (e.g., of a malignant/tumor cell), lyse cancer cells, kill cancer cells (e.g., drug-resistant cancer cells), inhibit proliferation of cancer cells, and/or increase cancer cell cytotoxicity (e.g., in response to other chemotherapeutic agents or cancer treatments (e.g. immunotherapy, radiation)). In some instances, the StOPs disclosed herein are used to kill a human cell that has increased net negative charge on the outer leaflet surface of its cell membrane relative to the counterpart normal cell (e.g., a cell that is becoming, or that is already, tumorigenic). This disclosure also features methods of treating cancer while prophylaxing or treating bacterial infections that arise during cancer therapy, e.g., as a result of immune suppression following chemotherapy or radiotherapy, using the StOPs disclosed herein.

In one aspect, the disclosure features a method of killing a cancer cell in a human subject in need thereof, the method comprising administering to the subject a therapeutically effective amount of a stabilized antimicrobial peptide. In one instance, the stabilized antimicrobial peptide is a stabilized magainin (e.g., any one disclosed herein). In one instance, the stabilized antimicrobial peptide is a stabilized esculentin. In one instance, the stabilized antimicrobial peptide is a stabilized CAP18. In one instance, the stabilized antimicrobial peptide is a stabilized pleurocidin. In one instance, the stabilized antimicrobial peptide is a peptide depicted in the Figures. In some instances, the stabilized AMP is a peptide with a stabilized amphipathic helix with hydrophobic face and positively charged face. In one instance, the stabilized antimicrobial peptide is a stabilized amphipathic helix with hydrophobic face and positively charged face. In some instances, the cancer is a hematological cancer. In some instances, the cancer is a leukemia. In some instances, the cancer is a lymphoma. In some instances, the cancer is multiple myeloma. In certain instances, the cancer is acute myeloid leukemia. In some instances, the cancer is a histiocytic lymphoma. In certain instances, the cancer is a mixed lineage leukemia. In one instance, the cancer cell has an increased net negative charge on the outer leaflet surface of its cell membrane relative to the counterpart normal cell.

In another aspect, the disclosure features a method of treating a cancer in a human subject in need thereof, the method comprising administering to the subject a therapeutically effective amount of a stabilized antimicrobial peptide. In one instance, the stabilized antimicrobial peptide is a stabilized magainin (e.g., any one disclosed herein). In one instance, the stabilized antimicrobial peptide is a stabilized esculentin. In one instance, the stabilized antimicrobial peptide is a stabilized CAP18. In one instance, the stabilized antimicrobial peptide is a stabilized pleurocidin. In one instance, the stabilized antimicrobial peptide is a peptide depicted in the Figures. In some instances, the stabilized AMP is a stabilized amphipathic helix with hydrophobic face and positively charged face. In one instance, the stabilized antimicrobial peptide is a peptide with a stabilized amphipathic helix with hydrophobic face and positively charged face. In some instances, the cancer is a hematological cancer. In some instances, the cancer is a leukemia. In some instances, the cancer is a lymphoma. In some instances, the cancer is multiple myeloma. In certain instances, the cancer is acute myeloid leukemia. In some instances, the cancer is a histiocytic lymphoma. In certain instances, the cancer is a mixed lineage leukemia. In one instance, the cancer cell has an increased net negative charge on the outer leaflet surface of its cell membrane relative to the counterpart normal cell.

In yet another aspect, the disclosure features a method of killing a cell that has an increased net negative charge on the outer leaflet surface of its cell membrane (relative to the counterpart normal cell), the method comprising contacting the cell with a stabilized antimicrobial peptide. In one instance, the stabilized antimicrobial peptide is a stabilized magainin (e.g., any one disclosed herein). In one instance, the stabilized antimicrobial peptide is a stabilized esculentin. In one instance, the stabilized antimicrobial peptide is a stabilized CAP18. In one instance, the stabilized antimicrobial peptide is a stabilized pleurocidin. In one instance, the stabilized antimicrobial peptide is a peptide depicted in the Figures. In some instances, the stabilized AMP is a stabilized amphipathic helix with hydrophobic face and positively charged face. In one instance, the stabilized antimicrobial peptide is a peptide with a stabilized amphipathic helix with hydrophobic face and positively charged face.

In another aspect, the disclosure provides method of killing a cancer cell in a human subject in need thereof. The method comprises administering to the subject a therapeutically effective amount of a structurally-stabilized peptide, wherein the structurally-stabilized peptide is 5 to 50 amino acids in length and has at least 50% (at least 55%, at least 60%, at least 65%, at least 70%, at least 75%, at least 80%, at least 85%, at least 90%, or at least 95%) identity over the full length of the amino acid sequence GIGKFLHSAKKFGKAFVGEIMNS (SEQ ID NO:1), wherein the structurally-stabilized peptide specifically lyses the cancer cells. In some instances, the structurally-stabilized peptide is an amphipathic helix. In some instances, the cancer cell is a leukemia cancer cell, a lymphoma cancer cell, or a multiple myeloma cancer cell.

In another aspect, the disclosure features a method of inhibiting proliferation of a cancer cell in a human subject in need thereof. The method comprising administering to the subject a therapeutically effective amount of a structurally-stabilized peptide, wherein the structurally-stabilized peptide is 5 to 50 amino acids in length and has at least 50% (at least 55%, at least 60%, at least 65%, at least 70%, at least 75%, at least 80%, at least 85%, at least 90%, or at least 95%) identity over the full length of the amino acid sequence GIGKFLHSAKKFGKAFVGEIMNS (SEQ ID NO:1), wherein the structurally-stabilized peptide specifically lyses the cancer cells. In some instances, the structurally-stabilized peptide is an amphipathic helix. In some instances, the cancer cell is a leukemia cancer cell, a lymphoma cancer cell, or a multiple myeloma cancer cell.

In yet another aspect, the disclosure features a method of treating a hematological cancer in a human subject in need thereof. The method comprises administering to the subject a therapeutically effective amount of a structurally-stabilized peptide, wherein the structurally-stabilized peptide is 5 to 50 amino acids in length and has at least 50% (at least 55%, at least 60%, at least 65%, at least 70%, at least 75%, at least 80%, at least 85%, at least 90%, or at least 95%) identity over the full length of the amino acid sequence GIGKFLHSAKKFGKAFVGEIMNS (SEQ ID NO:1). In some instances, the structurally-stabilized peptide is an amphipathic helix. In some instances, the structurally-stabilized peptide specifically lyses leukemia, lymphoma, and/or multiple myeloma cells. In certain instances, the hematological cancer is leukemia, lymphoma, or multiple myeloma.

In some instances of each of the above aspects, the structurally-stabilized peptide is a structurally stabilized peptide disclosed herein (e.g., magainin, pleurocidin, CAP18, esculentin, or any other antimicrobial peptide having an amphipathic helix). In one instance, the structurally-stabilized peptide is any one of SEQ ID NOs. 2-60, 133-145, or 222-226, wherein each of X₁, X₂, X₃, and X₄ is independently a stapling amino acid, B is norleucine, and the peptide is stapled.

In one instance, the structurally-stabilized peptide comprises or consists of an amino acid sequence depicted in FIG. 1A, FIG. 5A, FIG. 5B, FIG. 9, FIG. 11, FIG. 16, FIG. 17A, or FIG. 17B. In another instance, the structurally-stabilized peptide comprises or consists of an amino acid sequence depicted in FIG. 1A, FIG. 5A, FIG. 5B, FIG. 9, FIG. 11, FIG. 16, FIG. 17A, or FIG. 17B but having 1, 2, 3, 4, 5, or 6 amino acid substititutions (e.g., conservative substitutions) and wherein the structurally-stabilized peptide selectively kills cancer cells (e.g., hematological cancer cells, e.g., leukemia cells, lymphoma cells, multiple myeloma cells).

In one instance, the structurally-stabilized peptide comprises the formula Formula (I), or a pharmaceutically acceptable salt thereof, wherein: each R₁ and R₂ is H or a C1 to C10 alkyl, alkenyl, alkynyl, arylalkyl, cycloalkylalkyl, heteroarylalkyl, or heterocyclylalkyl, any of which is substituted or unsubstituted; each R₃ is independently alkylene, alkenylene, or alkynylene, any of which is substituted or unsubstituted; z is 1, 2, 3, 4, 5, 6, 7, 8, 9, or 10, and [Xaa]_{w}, [Xaa]ₓ, and [Xaa]_{y} are based on the sequences disclosed herein. In some cases, R₁ is an alkyl or a methyl group. In some cases, R₂ is an alkyl or a methyl group. In some cases, R₃ is an alkenyl or is —CH2-CH2-CH2-CH═CH—CH2-CH2-CH2-. In some cases, R₁ is a methyl group, R₂ is a methyl group, and R₃ is an alkenyl. In certain instances z in Formula (I) is 1.

In some instances, the structurally-stabilized peptide comprises the amino acid sequence set forth in any one of SEQ ID NOs: 2-6, 8-11, 13, 15-23, 26, 27, 31, 34, 35, 37, 38, 42, 46, 47, 54, 56, 58-60, 101-121, 128-158, and 222-226. In some instances, the structurally-stabilized peptide is 26 to 50 (26, 27, 28, 29, 30, 31, 32, 33, 34, 35, 36, 37, 38, 39, 40, 41, 42, 43, 44, 45, 46, 47, 48, 49, 50) amino acids in length.

In some instances, the structurally-stabilized peptide comprises the amino acid sequence set forth in any one of SEQ ID NOs: 292-311. In some instances, the structurally-stabilized peptide is 26 to 50 (26, 27, 28, 29, 30, 31, 32, 33, 34, 35, 36, 37, 38, 39, 40, 41, 42, 43, 44, 45, 46, 47, 48, 49, 50) amino acids in length.

In one aspect, the disclosure features a method of treating a hematological cancer and prophylaxing or treating a bacterial infection in a human subject in need thereof. The method involves administering to the subject a structurally stabilized peptide comprising the amino acid sequence set forth in any one of SEQ ID NOs: 17, 27, 28, 98, 99, 141-145, and 60, or a variant thereof. In some instances, the hematological cancer is a leukemia, a lymphoma, or a multiple myeloma.

This disclosure also features a peptide comprising an amino acid sequence set forth in 30 any one of SEQ ID NOs:37, 98, 99, 133-145, and 222-226 with 0 to 4 (0, 1, 2, 3, 4) amino acid substitutions therein, wherein B is norleucine, and wherein the peptide lyses leukemia, lymphoma, and/or multiple myeloma cells. In some instances, the peptide is 26 to 50 (26, 27, 28, 29, 30, 31, 32, 33, 34, 35, 36, 37, 38, 39, 40, 41, 42, 43, 44, 45, 46, 47, 48, 49, 50) amino acids in length.

Also provided is a structurally-stabilized peptide comprising an amino acid sequence set forth in any one of SEQ ID NOs.: 37, 98, 99, 133-145, and 222-226 with 0 to 4 (0, 1, 2, 3, 4) amino acid substitutions therein, wherein B is norleucine, and wherein the peptide lyses leukemia, lymphoma, and/or multiple myeloma cells. In some instances, the peptide is 26 to 50 (26, 27, 28, 29, 30, 31, 32, 33, 34, 35, 36, 37, 38, 39, 40, 41, 42, 43, 44, 45, 46, 47, 48, 49, 50) amino acids in length. In some instances, the structurally-stabilized peptide is stapled.

In one aspect, the disclosure relates to a pharmaceutical composition comprising a peptide or structurally-stabilized peptide disclosed herein and a pharmaceutically acceptable carrier.

In another aspect, the disclosure relates to a pharmaceutical composition comprising: (a) a means for selectively killing cancer cells; and (b) a pharmaceutically acceptable carrier. In some instances, the cancer cells are hematological cancer cells, and optionally leukemia cells, lymphoma cells, or multiple myeloma cells. In some instances, the cancer cells are from a hematological cancer, a leukemia, a lymphoma, a multiple myeloma, an acute myeloid leukemia, a histiocytic lymphoma, or a mixed lineage leukemia. In some instances, the means for selectively killing cancer cells are hydrocarbon stapled Magainin II peptides, and optionally wherein the means do not have, or have reduced, lytic activity on non-cancerous cells (e.g., red blood cells, endothelial cells).

In another aspect, the disclosure relates to a pharmaceutical composition comprising: (a) a means for killing both cancer cells and bacterial cells; and (b) a pharmaceutically acceptable carrier. In some instances, the means for killing cancer cells and bacterial cells are hydrocarbon stapled Magainin II peptides. hydrocarbon stapled Pleurocidin peptides, hydrocarbon stapled CAP18 peptides, or hydrocarbon stapled esculentin peptides.

Also featured is a method of making a structurally stabilized peptide herein by providing an unstapled peptide (e.g., a peptide of magainin, pleurocidin, CAP18, esculentin, or any antimicrobial peptide having an amphipathic helix) and cross-linking the peptide. In some instances, the peptide has the sequence set forth in any one of SEQ ID NOs: 37, 98, 99, 133-145, and 222-226.

Where a structurally stabilized peptide described herein contains a methionine (M), it may be interchanged for a norleucine (B). Where a structurally stabilized peptide described herein contains a norleucine (B), it may be interchanged for a methionine (M).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1A shows a staple scanning library of magainin II, with pairs of natural amino acids sequentially replaced by an i, i+4 all-hydrocarbon staple along the length of the antimicrobial peptide sequence. X₁ and X₂ are independently stapling amino acids (e.g., α-methyl, α-alkenyl non-natural amino acids) for each of the recited SEQ ID NOs. Experiments using these peptides were carried out with peptides in which X₁ and X₂ of each of the SEQ ID NOs are both (S)-2-(4′-pentenyl)Alanine.

FIG. 1B shows a helical wheel representation of the amphipathic alpha-helical magainin II peptide. M21 can be replaced by norleucine.

FIG. 2 shows the percent lysis of red blood cells and MV4;11 leukemia cells treated with the i, i+4 staple scanning library of magainin II.

FIG. 3 shows the EC₅₀ for LDH release from (cell lysis of) OCI-AML3 and U937 acute myeloid leukemia cells for the library of i, i+4 stapled magainin II peptides. X-axis: 0-18 correspond to Mag(i+4)0-Mag(i+4)18, respectively.

FIG. 4 shows the comparative lysis of OCI-AML3, U937, and red blood cells (RBCs) by the i, i+4 stapled magainin II peptide. X-axis: 0-18 correspond to Mag(i+4)0-Mag(i+4)18, respectively.

FIG. 5A and FIG. 5B show lysine and glutamate scanning libraries, respectively, of the Mag(i+4)15 stapled peptide, which showed cancer cell lysis without causing RBC lysis. X₁ and X₂ are independently stapling amino acids (e.g., α-methyl, α-alkenyl non-natural amino acids) for each of the recited SEQ ID NOs. Experiments using these peptides were carried out with peptides in which X₁ and X₂ of each of the SEQ ID NOs are both (S)-2-(4′-pentenyl)Alanine.

FIG. 6 shows the percent cancer cell lysis at 90 minutes for OCI-AML3 and U937 cells treated with 10 or 25 μg/mL of each peptide in the Mag(i+4)15 lysine scanning library, as measured by LDH release assay. The x-axis refers to the position in Mag(i+4)15 where lysine (K) is introduced (SEQ ID NOs: 21-31, 33-35, 37, 38, 36, and 17, from left to right, respectively).

FIG. 7 shows the percent cancer cell lysis at 90 minutes for OCI-AML3 and U937 cells treated with 10 or 25 μg/mL of each peptide in the Mag(i+4)15 glutamate scanning library, as measured by LDH release assay. The x-axis refers to the position in Mag(i+4)15 where glutamic acid (E) is introduced (SEQ ID NOs: 40-59 and 17, from left to right, respectively).

FIG. 8 shows that HUVEC cells are unaffected by select Mag(i+4) compounds at doses that are otherwise profoundly lytic to cancer cells.

FIG. 9 shows that a lead stapled antimicrobial peptide, Mag(i+4)1,15(A9K, B21A, N22K, S23K) (SEQ ID NO:60), shown to kill multidrug resistant bacteria in mouse models without causing RBC or kidney toxicity is also capable of lysing a series of leukemia cells at 90 min and 24 hour time points with EC50s ranging from 11.5-36.6 μg/mL for leukemia cells (e.g. U937, OCI-AML3 and MV4;11 cells).

FIG. 10 shows the effect of Mag(i+4)1,15(A9K, B21A, N22K, S23K) (SEQ ID NO:60) on breast cancer and osteosarcoma cells.

FIG. 11 shows that integrating the staple scanning, lysine scanning, and glutamate scanning data can lead to the identification of stapled peptides that are selectively toxic to cancer cells or that exhibit dual anti-cancer cell and anti-bacterial cell lytic activity. The schematic at top right shows that the fifteen peptides to its left are designed to specifically lyse cancer cells without lysing bacterial cells, RBCs, and endothelial cells. The schematic at bottom right shows that the eleven peptides to its left are designed to lyse both cancer cells and bacterial cells without lysing RBCs and endothelial cells.

FIG. 12 shows the variety of stapling amino acids that can be used to generate all-hydrocarbon stapled oncolytic peptides, and the placement of such non-natural amino acids at [i, i+3], [i, i+4], or [i, i+7] positions along the length of the peptide sequence followed by crosslinking by ruthenium-catalyzed olefin metathesis.

FIG. 13 illustrates how in addition to single staple insertion and scanning to identify optimal stapled oncolytic peptides, double and triple stapling can also be accomplished using combinations of [i, i+3], [i, i+4], and [i, i+7] staples.

FIG. 14 illustrates that all-hydrocarbon stitching by use of a Bis-pentenyl glycine moiety can enable two contiguous staples to emerge from a single amino acid position, yielding a diversity of “stitched” oncolytic peptides.

FIG. 15 illustrates how hydrocarbon-stapling and mutagenesis can be integrated to yield optimal oncolytic peptides for therapeutic application.

FIG. 16 illustrates how hydrocarbon-stapling and mutagenesis can be integrated to yield optimal oncolytic peptides for therapeutic application based on a diversity of natural AMP sequences, including pleurocidin, CAP18, and esculentin.

FIG. 17A depicts the amino acid sequences of designed and synthesized i, i+7 staple scanning StOPs of Magainin II. 8 and X are independently stapling amino acids (e.g., α-methyl, α-alkenyl non-natural amino acids) for each of the recited SEQ ID NOs. Experiments using these peptides were carried out with peptides in which 8 is (R)-2-(7′-octenyl)Alanine and X is (S)-2-(4′-pentenyl)Alanine.

FIG. 17B depicts the amino acid sequences of designed and synthesized integrated StOPs (iStOPs) that combine discrete staple types, staple positions, and point mutation(s) that were individually shown to confer favorable therapeutic and selectivity properties. Each X is independently a stapling amino acid (e.g., α-methyl, α-alkenyl non-natural amino acids) for each of the recited SEQ ID NOs. Experiments using these peptides were carried out with peptides in which each X is (S)-2-(4′-pentenyl)Alanine.

FIG. 18 illustrates the differential susceptibility of OC-AML3 leukemia cells to an i, i+7 staple scanning library of Magainin II. From left to right: SEQ ID NOs:292-307, respectively.

FIG. 19 illustrates the differential susceptibility of OCI-AML3 leukemia cells to an iStOP series of Magainin II peptides bearing a combination of discrete staple types, staple locations and point mutation(s). From left to right: SEQ ID NOs: 42, 46, 47, 50, 54, 56, 58, 59, 27, 98, 99, 308-311, and 22, respectively.

FIG. 20 illustrates the differential susceptibility of HeLa cervical cancer cells to an i, i+4 staple scanning library of Magainin II. From left to right, SEQ ID NOs:317 and 2-20, respectively.

FIG. 21 illustrates the differential susceptibility of HeLa cervical cancer cells to an i, i+7 staple scanning library of Magainin II. From left to right: SEQ ID NOs:292-307, respectively.

FIG. 22 illustrates the differential susceptibility of HeLa cervical cancer cells to an iStOP series of Magainin II peptides bearing a combination of discrete staple types, staple locations and point mutation(s). From left to right: SEQ ID NOs: 42, 46, 47, 50, 54, 56, 58, 59, 27, 98, 99, 308-311, and 22, respectively.

FIG. 23 illustrates the dose-responsive cytotoxic response of over 750 human cancer cell lines representing more than 45 distinct lineages clustered by 23 human tissues of origin to treatment with a StOP corresponding to SEQ ID NO:2.

FIG. 24 illustrates the dose-responsive cytotoxic response of over 750 human cancer cell lines representing more than 45 distinct lineages clustered by 23 human tissues of origin to treatment with a StOP corresponding to SEQ ID NO:17.

FIG. 25 illustrates the dose-responsive cytotoxic response of over 750 human cancer cell lines representing more than 45 distinct lineages clustered by 23 human tissues of origin to treatment with a StOP corresponding to SEQ ID NO:60.

FIG. 26 illustrates the differential dose-dependent lytic response of a primary human B-acute lymphoblastic leukemia specimen obtained from the peripheral blood of a pediatric patient (06078-686) at diagnosis to StOPs of SEQ ID NOs:2, 17, and 60 (sequences on x-axis from left to right, respectively).

FIG. 27 illustrates the dose-dependent lytic response of a primary human B-acute lymphoblastic leukemia specimen obtained from the peripheral blood of a pediatric patient (06078-689) at diagnosis to a StOP of SEQ ID NO:60, which has been safely administered to mice without evidence of non-specific red blood cell hemolysis or kidney toxicity.

### DETAILED DESCRIPTION

A reservoir of natural and non-natural alpha-helical peptides, such as anti-microbial peptides, have the capacity to rupture membranes to induce killing of infectious organisms but are largely non-specific, causing the destruction of mammalian cells as well. The application of hydrocarbon stapling technology has recently identified critical biophysical determinants that confer membrane-specific lytic activity. This disclosure provides membrane-selective stapled AMPs that specifically lyse cancer cell membranes, causing cancer cell cytotoxicity. Such membrane-selective stapled AMPs that specifically lyse cancer cell membranes are referred to herein as Stapled Oncolytic Peptides (StOPs).

Normal mammalian cells are surrounded by a bilayer membrane with an asymmetric distribution of phospholipids. Under homeostatic conditions, the outer leaflet contains mostly zwitterionic lipids and has an overall neutral charge, while the inner leaflet contains mainly negatively charged lipids and thus an overall net negative charge. During certain biological processes, like apoptosis, loss of this asymmetry due to the translocation of negatively charged phospholipids, such as phosphatidylserine, to the outer leaflet results in a dramatic change in the net charge on the outside of mammalian cells. Interestingly, similar changes in phospholipid distribution occur in various kinds of cancer. In addition, changes in the glycosylation pattern of glycoproteins on the surface of cancer cells can result in a higher level of sialic acid content, further increasing the net negative charge on the outer leaflet surface. This disclosure features stapled peptides that can be used to kill human cells that have a cell membrane comprising an anionic outer leaflet or that have a cell membrane comprising an outer leaflet having an increased net negative charge relative to the outer leaflet of a cell membrane of a normal counterpart human cell. In one instance, the human cell that has a cell membrane comprising an anionic outer leaflet or that has a cell membrane comprising an outer leaflet having an increased negative charge relative to the outer leaflet of a cell membrane of a normal counterpart human cell is a cancer cell.

Staple scanning identified AMP-based constructs with specific cancer-cell killing activity, both with and without anti-bacterial activity, while avoiding toxicity to normal mammalian cells, such as red blood cells and endothelial cells. The constructs can be used for cancer treatment and dual anti-cancer/anti-bacterial compounds. With respect to the latter use, the peptides disclosed herein can, for example, be used to treat cancer while prophylaxing or treating bacterial infections that often complicate chemotherapy treatment due to immune suppression, including neutropenia.

### Oncolytic Peptides

Disclosed herein are stabilized antimicrobial peptides that can be used to kill cancer cells. In one instance, the stabilized antimicrobial peptide is a stabilized magainin. In one instance, the stabilized antimicrobial peptide is a stabilized esculentin. In one instance, the stabilized antimicrobial peptide is a stabilized CAP18. In one instance, the stabilized antimicrobial peptide is a stabilized pleurocidin. In some instances, the stabilized AMP is a peptide with a stabilized amphipathic helix with hydrophobic face and positively charged face

Magainins are a class of antimicrobial peptides found in the African clawed frog (Xenopus laevis). The peptides are cationic and generally lack a stable conformation in water but form an amphipathic α-helix in membranes. Their mechanism against micro-organisms involves direct disruption of the cell membranes of a broad spectrum of bacteria, protozoa, and fungi. The amino acid sequence of magainin II (Xenopus species) is GIGKFLHSAKKFGKAFVGEIMNS (SEQ ID NO:1). The amino acid sequence of a modified magainin II is GIGKFLHSAKKFGKAFVGEIBNS (SEQ ID NO:100), wherein B is norleucine.

Pleurocidin is an antimicrobial peptide found in the mucus secreted by the skin of the winter flounder that has broad-spectrum antimicrobial activity. It has potent activity against Gram-positive and Gram-negative bacteria. It inhibits nucleic acid and protein synthesis. The amino acid sequence of pleurocidin is GWGSFFKKAAHVGKHVGKAALTHYL (SEQ ID NO:219). In some instances, the pleurocidin peptide is a modified pleurocidin peptide comprising a lysine (lys, K) at the amino acid corresponding to position 9 of SEQ ID NO:219 (i.e., corresponding to Ala9 of SEQ ID NO:219). For example, in some instances, the modified pleurocidin peptide comprises the amino acid sequence

(SEQ ID NO: 227)
GWGSFFKKKAHVGKHVGKAALTHYL.

CAP18 is an antimicrobial protein that binds to the lipid A moiety of bacterial lipopolysaccharides (LPS), a glycolipid present in the outer membrane of all Gram-negative bacteria. The amino acid sequence of CAP18 is GLRKRLRKFRNKIKEKLKKIGQKIQGLLPKLAPRTDY (SEQ ID NO:220). In some instances, the amino acid sequence of CAP18 is GLRKRLRKFRNKIKEKLKKIGQKIQGLLPKLA (SEQ ID NO:228) In some instances, the amino acid sequence of CAP18 is GLRKRLRKFRINKIKEKLKKIGQKIQGLVPKLAPRTDY (SEQ ID NO:231). In some instances, the amino acid sequence of CAP18 is GLRKRLRKFRNKIKEKLKKIGQKIQGLVPKLA (SEQ ID NO:232). In some instances, the CAP18 peptide is a modified CAP18 peptide comprising: (i) a lysine (lys, K) at the amino acid corresponding to position 17 of SEQ ID NO:220 (i.e., corresponding to Leu17 of SEQ ID NO:220); and/or (ii) a valine (val, V) at the amino acid corresponding to position 28 of SEQ ID NO:220 (i.e., corresponding to Leu28 of SEQ ID NO:220). For example, in some instances, the modified CAP18 peptide comprises the amino acid sequence GLRKRLRKFRNKIKEKX₁KKIGQKIQGLX₂PKLAPRTDY, wherein X₁ is K or L and X₂ is V or L (SEQ ID NO:229).

Esculentin is an antimicrobial peptide located outside the epithelial cell's membrane of the skin of many species of amphibians, such as Rana chiricahuensis. The amino acid sequence of esculentin is GIFSKLAGKKIKNLLISGLKG (SEQ ID NO:221). In some instances, the esculentin peptide is a modified esculentin peptide comprising a lysine (lys, K) at the amino acid corresponding to position 7 of SEQ ID NO:221 (i.e., corresponding to Ala7 of SEQ ID NO:221). For example, in some instances, the modified esculentin peptide comprises the amino acid sequence GIFSKLKGKKIKNLLISGLKG (SEQ ID NO:230).

Provided herein are peptides comprising a modified amino acid sequence of a magainin II peptide described herein. The magainin II peptides are modified to introduce structural-stabilization to the peptide (e.g., to maintain alpha-helicity of the peptide). The structural-stabilization may be by, e.g., “stapling” the peptide. In some cases, the staple is a hydrocarbon staple. In some instances, the modifications to introduce structural-stabilization (e.g., internal cross-linking, e.g., stapling) into the magainin II peptides described herein are positioned at the amino acid positions in the magainin II peptide corresponding to residues:

(i) 1 and 5 of SEQ ID NO:1 or 100;

(ii) 2 and 6 of SEQ ID NO:1 or 100;

(iii) 3 and 7 of SEQ ID NO:1 or 100;

(iv) 4 and 8 of SEQ ID NO:1 or 100;

(v) 5 and 9 of SEQ ID NO:1 or 100;

(vi) 6 and 10 of SEQ ID NO:1 or 100;

(vii) 7 and 11 of SEQ ID NO:1 or 100;

(viii) 8 and 12 of SEQ ID NO:1 or 100;

(ix) 9 and 13 of SEQ ID NO:1 or 100;

(x) 10 and 14 of SEQ ID NO:1 or 100;

(xi) 11 and 15 of SEQ ID NO:1 or 100;

(xii) 12 and 16 of SEQ ID NO:1 or 100;

(xiii) 13 and 17 of SEQ ID NO:1 or 100;

(xiv) 14 and 18 of SEQ ID NO:1 or 100;

(xv) 15 and 19 of SEQ ID NO:1 or 100;

(xvi) 16 and 20 of SEQ ID NO:1 or 100;

(xvii) 17 and 21 of SEQ ID NO:1 or 100;

(xviii) 18 and 22 of SEQ ID NO:1 or 100;

(xix) 19 and 23 of SEQ ID NO:1 or 100;

(xx) 2, 6, 16, and 20 of SEQ ID NO:1 or 100; or

(xxi) 1, 5, 16, and 20 of SEQ ID NO:1 or 100.

In some cases, the staple is a hydrocarbon staple. In some instances, the modifications to introduce structural-stabilization (e.g., internal cross-linking, e.g., stapling) into the magainin II peptides described herein are positioned at the amino acid positions in the magainin II peptide corresponding to residues:

(i) 1 and 8 of SEQ ID NO:1 or 100;

(ii) 2 and 9 of SEQ ID NO:1 or 100;

(iii) 3 and 10 of SEQ ID NO:1 or 100;

(iv) 4 and 11 of SEQ ID NO:1 or 100;

(v) 5 and 12 of SEQ ID NO:1 or 100;

(vi) 6 and 13 of SEQ ID NO:1 or 100;

(vii) 7 and 14 of SEQ ID NO:1 or 100;

(viii) 8 and 15 of SEQ ID NO:1 or 100;

(ix) 9 and 16 of SEQ ID NO:1 or 100;

(x) 10 and 17 of SEQ ID NO:1 or 100;

(xi) 11 and 18 of SEQ ID NO:1 or 100;

(xii) 12 and 19 of SEQ ID NO:1 or 100;

(xiii) 13 and 20 of SEQ ID NO:1 or 100;

(xiv) 14 and 21 of SEQ ID NO:1 or 100;

(xv) 15 and 22 of SEQ ID NO:1 or 100; or

(xvi) 16 and 23 of SEQ ID NO:1 or 100.

In some instances, the modifications to introduce structural-stabilization (e.g., internal cross-linking, e.g., stapling or stitching) into the magainin II peptides described herein are positioned at the amino acid positions in the magainin II peptide corresponding to residues:

(i) 1 and 5 of SEQ ID NO:1 or 100;

(ii) 2 and 6 of SEQ ID NO:1 or 100;

(iii) 3 and 7 of SEQ ID NO:1 or 100;

(iv) 5 and 9 of SEQ ID NO:1 or 100;

(v) 7 and 11 of SEQ ID NO:1 or 100;

(vi) 12 and 16 of SEQ ID NO:1 or 100;

(vii) 16 and 20 of SEQ ID NO:1 or 100;

(viii) 17 and 21 of SEQ ID NO:1 or 100; or

(ix) 1, 5, 16, and 20 of SEQ ID NO:1 or 100;

(x) 2, 6, 16, and 20 of SEQ ID NO:1 or 100; or

(xi) 3, 7, 16, and 20 of SEQ ID NO:1 or 100.

In certain instances, the modifications to introduce structural-stabilization (e.g., internal cross-linking, e.g., stapling or stitching) into the magainin II peptides described herein are positioned at the amino acid positions in the magainin II peptide corresponding to residues 16 and 20 of SEQ ID NO:1. In certain instances, the structurally-stabilized (e.g., stapled) peptides described herein may also contain one or more (e.g., 1, 2, 3, 4, 5, 6, 7, 8) additional amino acid substitutions (relative to the wild type magainin II peptide sequence), e.g., one or more (e.g., 1, 2, 3, 4, 5, 6, 7, 8) conservative and/or non-conservative amino acid substitutions (i.e., one or more amino acid substitutions in addition to the amino acid substitutions made to the magainin II to impart the structural-stabilization). In certain instances, the additional substitution(s) comprise substitution(s) with Lysine (K) at one or more (e.g., 1, 2, 3, 4, 5, 6, 7, 8, 9, 10, 11) of amino acids corresponding to Gly1, Ile2, Gly3, His7, Ser8, Ala9, Ala15, Gly18, Glu19, Met21, Asn22 or Ser23 of SEQ ID NO:1. In certain instances, the additional substitution(s) comprise substitution(s) with Glutamate (E) at one or more (e.g., 1, 2, 3, 4, 5, 6, 7) of amino acids corresponding to Gly3, His7, Ser8, Ala9, Ala15, Gly18, Asn22, or Ser23 of SEQ ID NO:1. In certain instances, the additional substitutions comprise substitutions with norleucine (B) at an amino acid corresponding to Met21 of SEQ ID NO:1. In certain instances, the additional substitutions comprise substitutions with alanine (A) at an amino acid corresponding to Met21 of SEQ ID NO:1. In certain instances, the additional substitution(s) comprise substitution with Alanine (A) at an amino acid corresponding to Met21 of SEQ ID NO:1 and substitution with Lysine (K) at amino acids corresponding to Ala9, Asn22, and Ser23 of SEQ ID NO:1. In certain instances, the structurally-stabilized (e.g., internally cross-linked, e.g., stapled) peptides described herein may also contain one or more (e.g., 1, 2, 3, 4, 5, 6, 7, 8, 9, 10) deletions from the N- and/or C-terminus of the magainin II peptide. For example, the structurally-stabilized (e.g., internally cross-linked, e.g., stapled) peptides are 5 or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In a specific instance, the structurally-stabilized (e.g., internally cross-linked, e.g., stapled) peptides are 5-30 (i.e., 5, 6, 7, 8, 9, 10, 11, 12, 13, 14, 15, 16, 17, 18, 19, 20, 21, 22, 23, 24, 25, 26, 27, 28, 29, 30) in length. In a specific instance, the structurally-stabilized (e.g., internally cross-linked, e.g., stapled) peptides are 10-30 (i.e., 10, 11, 12, 13, 14, 15, 16, 17, 18, 19, 20, 21, 22, 23, 24, 25, 26, 27, 28, 29, 30) amino acids in length. In a specific instance, the structurally-stabilized (e.g., internally cross-linked, e.g., stapled) peptides are 15-30 (i.e., 15, 16, 17, 18, 19, 20, 21, 22, 23, 24, 25, 26, 27, 28, 29, 30) amino acids in length. In a specific instance, the structurally-stabilized (e.g., internally cross-linked, e.g., stapled) peptides are 5-23 (i.e., 5, 6, 7, 8, 9, 10, 11, 12, 13, 14, 15, 16, 17, 18, 19, 20, 21, 22, 23) in length. In a specific instance, the structurally-stabilized (e.g., internally cross-linked, e.g., stapled) peptides are 10-30 (i.e., 10, 11, 12, 13, 14, 15, 16, 17, 18, 19, 20, 21, 22, 23) amino acids in length. In a specific instance, the structurally-stabilized (e.g., internally cross-linked, e.g., stapled) peptides are 15-23 (i.e., 15, 16, 17, 18, 19, 20, 21, 22, 23) amino acids in length. In a specific instance, the structurally-stabilized (e.g., internally cross-linked, e.g., stapled) peptides are 23-30 (i.e., 23, 24, 25, 26, 27, 28, 29, or 30) amino acids in length. In a specific instance, the structurally-stabilized (e.g., internally cross-linked, e.g., stapled) peptides are 23 amino acids in length.

In certain instances, the magainin II peptides of this disclosure can have 1, 2, 3, 4, 5, 6, 7, 8, 9, 10, 11, 12, or 13 amino acid substitutions in any one of SEQ ID NOs:1, 63-75, 100, 123-127, and 312-314 (e.g., 1, 2, 3, 4, 5, 6, 7, 8, 9, 10, 11, 12, or 13 amino acids are conservatively or non-conservatively substituted, and wherein the amino acid substitution is with a naturally or non-naturally occurring amino acid). In some instances, two or more (e.g., 2, 3, 4) amino acids of these peptides are substituted with stapling amino acids (e.g., α-methyl, α-alkenyl non-natural amino acids). For example, in certain instances, the magainin II peptide of this disclosure comprises a modified amino acid sequence of the sequence set forth in SEQ ID NO:1 or SEQ ID NO:100, wherein the modified amino acid sequence comprises SEQ ID NO:1 or SEQ ID NO:100 having 1, 2, 3, 4, 5, 6, 7, 8, 9, 10, 11, 12, or 13 amino acid substitutions in the SEQ ID NO:1 or SEQ ID NO:100 sequence (e.g., the modified amino acid sequence comprises SEQ ID NO:1 or SEQ ID NO:100, except that 1, 2, 3, 4, 5, 6, 7, 8, 9, 10, 11, 12, or 13 amino acids of SEQ ID NO:1 or SEQ ID NO:100 are conservatively or non-conservatively substituted, and wherein the amino acid substitution is with a naturally or non-naturally occurring amino acid). In some instances, two or more (e.g., 2, 3, 4) amino acids of these peptides are substituted with stapling amino acids (e.g., α-methyl, α-alkenyl non-natural amino acids). In another example, in certain instances, the magainin II peptide of this disclosure comprises a modified amino acid sequence of the sequence set forth in SEQ ID NO:75, wherein the modified amino acid sequence comprises SEQ ID NO:75 having 1, 2, 3, 4, 5, 6, 7, 8, 9, 10, 11, 12, or 13 amino acid substitutions in the SEQ ID NO:75 sequence (e.g., the modified amino acid sequence comprises SEQ ID NO:75, except that 1, 2, 3, 4, 5, 6, 7, 8, 9, 10, 11, 12, or 13 amino acids of SEQ ID NO:75 are conservatively or non-conservatively substituted, and wherein the amino acid substitution is with a naturally or non-naturally occurring amino acid). In some instances, two or more (e.g., 2, 3, 4) amino acids of these peptides are substituted with stapling amino acids (e.g., α-methyl, α-alkenyl non-natural amino acids).

Also, provided herein are peptides comprising a modified amino acid sequence of a pleurocidin, CAP18, and esculentin peptides described herein.

A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), and acidic side chains and their amides (e.g., aspartic acid, glutamic acid, asparagine, glutamine). By “naturally occurring” amino acids refers to the known 20 amino acids that appear in the genetic code. “Non-naturally occurring” are those not found in the genetic code, e.g., norleucine is a “non-naturally occurring” amino acid. In some instances, one to five amino acids of any one of SEQ ID NOs:1, 63-75, 100, 123-127, 219-221, 227-232, and 312-314 are substituted (in addition to any substitutions introducing structural-stabilization, e.g., substitutions with stapling or stitching amino acids). In some instances, the substitution(s) is/are a conservative amino acid substitution. In other instances, the substitution(s) is/are a non-conservative amino acid substitution. In some instances, where there are more than one amino acid substitutions, the substitutions are both conservative and non-conservative amino acid substitutions. In certain instances, the substituted amino acid(s) are selected from the group consisting of L-Ala, D-Ala, Aib, Sar, Ser, a substituted alanine, or a substituted glycine derivative.

In certain instances, the peptides of this disclosure can have 1, 2, 3, 4, or 5, amino acids removed/deleted from the C-terminus of the sequence set forth in any one of SEQ ID NOs:1, 63-75, 100, 123-127, 219-221, 227-232, and 312-314. In certain instances, the peptides of this disclosure can have 1, 2, 3, 4, or 5, amino acid removed/deleted from the N-terminus of the sequence set forth in any one of SEQ ID NOs:1, 63-75, 100, 123-127, and 312-314. In certain instances, these removed amino acids can be replaced with 1-6 (e.g., 1, 2, 3, 4, 5, or 6) amino acids selected from the group consisting of L-Ala, D-Ala, Aib, Sar, Ser, a substituted alanine, or a substituted glycine derivative. These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

In some instances, the peptides of this disclosure can have one or more (e.g., 1, 2, 3, 4, 5, 6, 7, 8) amino acids corresponding to positions 2, 5, 6, 12, 16, 17, 20, and 21 of SEQ ID NO:1 (i.e., corresponding to Ile2, Phe5, Leu6, Phe12, Phe16, Val17, Ile20, and Met21 of SEQ ID NO:1) substituted with a different hydrophobic amino acid. For example, in some instances, the amino acid corresponding to Ile2 of SEQ ID NO:1 is substituted with Phe (F). Examples of hydrophobic amino acids include leucine (L), isoleucine (I), phenylalanine (F), tryptophan (W), valine (V), methionine (M), cysteine (C), tyrosine (T), and alanine (A). In certain instances, Met21 is replaced with B (norleucine)21. These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

In some instances, the peptides of this disclosure can have one or more (e.g., 1, 2, 3, 4) amino acids corresponding to positions 4, 10, 11, and 14 of SEQ ID NO:1 (i.e., corresponding to Lys4, Lys10, Lys11, and Lys14 of SEQ ID NO:1) substituted with a different positively charged amino acid. For example, in some instances, the amino acid corresponding to Lys4 of SEQ ID NO:1 is substituted with His (H), or Arginine (R), or Ornithine. Examples of positively charged amino acids include lysine (K), arginine (R), and histidine (H). These peptides kill human hematological cancer cells (e.g., leukemia, lymphoma, multiple myeloma cells) or human cells with a cell membrane comprising an anionic outer leaflet or with a cell membrane comprising an outer leaflet having an increased net negative charge relative to the outer leaflet of a cell membrane of a normal counterpart human cell.

In some instances, the peptides of this disclosure can have one or more (e.g., 1, 2, 3, 4) amino acids corresponding to positions 7, 8, 22, and 23 of SEQ ID NO:1 (i.e., corresponding to His7, Ser8, Asn22, and Ser23 of SEQ ID NO:1) substituted with a different hydrophilic amino acid. For example, in some instances, the amino acid corresponding to His7 of SEQ ID NO:1 is substituted with Ser (S). Examples of hydrophilic amino acids include arginine (R), lysine (K), asparagine (N), histidine (H), and proline (P). These peptides kill human hematological cancer cells (e.g., leukemia, lymphoma, multiple myeloma cells) or human cells with a cell membrane comprising an anionic outer leaflet or with a cell membrane comprising an outer leaflet having an increased net negative charge relative to the outer leaflet of a cell membrane of a normal counterpart human cell.

In some instances, the peptides of this disclosure can have the amino acid corresponding to position 19 of SEQ ID NO:1 (i.e., corresponding to Glu19 of SEQ ID NO:1) substituted with another negatively charged amino acid. For example, in some instances, the amino acid corresponding to Glu19 of SEQ ID NO:1 is substituted with Asp (D). Examples of negatively charged amino acids include aspartate (D) and glutamate (E). These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

In certain instances, the disclosure features a peptide that kills human hematological cancer cells (e.g., leukemia, lymphoma, multiple myeloma cells) or human cells with a cell membrane comprising an anionic outer leaflet or with a cell membrane comprising an outer leaflet having an increased net negative charge relative to the outer leaflet of a cell membrane of a normal counterpart human cell. The peptide comprises the sequence:

X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃, wherein:
X₁ is G, a hydrophobic or hydrophilic amino acid, or a stapling amino acid
X₂ is I, a hydrophobic amino acid, or a stapling amino acid
X₃ is G, a hydrophobic or hydrophilic amino acid, or a stapling amino acid
X₄ is K, or a positively charged amino acid such as R or H
X₅ is F, or a hydrophobic amino acid, or a stapling amino acid
X₆ is L, or a hydrophobic amino acid, or a stapling amino acid
X₇ is H, or a positively charged amino acids such as R or H, or a stapling amino acid
X₈ is S, a hydrophobic or hydrophilic amino acid, or a stapling amino acid
X₉ is A, or a hydrophobic amino acid, or a stapling amino acid
X₁₀ is K, or a positively charged amino acid such as R or H, or a stapling amino acid.
X₁₁ is K, or a positively charged amino acid such as R or H, or a stapling amino acid
X₁₂ is F, a hydrophobic amino acid, or a stapling amino acid
X₁₃ is G, a hydrophobic amino acid, or a stapling amino acid
X₁₄ is K, or a positively charged amino acid such as R or H, or a stapling amino acid
X₁₅ is A, a hydrophobic or hydrophilic amino acid, or a stapling amino acid
X₁₆ is F, a hydrophobic amino acid, or a stapling amino acid
X₁₇ is V, a hydrophobic amino acid, or a stapling amino acid
X₁₈ is G, a hydrophobic or hydrophilic amino acid, or a stapling amino acid
X₁₉ is E, or a hydrophobic or hydrophilic amino acid, or a stapling amino acid
X₂₀ is I, a hydrophobic amino acid, or a stapling amino acid
X₂₁ is M or B, or a conservative amino acid substitution thereof, a hydrophobic amino acid, or a stapling amino acid;
X₂₂ is N, a hydrophilic amino acid, or a positively charged amino acid such as K, R or H, or a stapling amino acid.
X₂₃ is S, a hydrophobic or hydrophilic amino acid, or a stapling amino acid (SEQ ID NO:233).

In some instances of SEQ ID NO:233, only two of any one of X₁ to X₂₃ are stapling amino acids, wherein the two stapling amino acids are at positions i and i+4 or i and i+7. For example, in one instance of SEQ ID NO:233, only X₁₆ and X₂₀ are stapling amino acids, and X₁ is G, or a hydrophobic or hydrophilic amino acid, X₂ is I or a hydrophobic amino acid, X₃ is G, a hydrophobic amino acid, or a hydrophilic amino acid, X₄ is K or a positively charged amino acid such as R or H, X₅ is F or a hydrophobic amino acid, X₆ is L or a hydrophobic amino acid, X₇ is H or a positively charged amino acid such as R or H, X₈ is S, a hydrophobic amino acid, or a hydrophilic amino acid, X₉ is A or a hydrophobic amino acid, X₁₀ is K or a positively charged amino acid such as R or H, X₁₁ is K or a positively charged amino acid such as R or H, X₁₂ is F or a hydrophobic amino acid, X₁₃ is G or a hydrophobic amino acid, X₁₄ is K or a positively charged amino acid such as R or H, X₁₅ is A, a hydrophobic amino acid, or a hydrophilic amino acid, X₁₆ is a stapling amino acid, X₁₇ is V or a hydrophobic amino acid, X₁₈ is G, a hydrophobic amino acid, or a hydrophilic amino acid, X₁₉ is E, a hydrophobic amino acid, or a hydrophilic amino acid, X₂₀ is a stapling amino acid, X₂₁ is M or B, or a conservative amino acid substitution thereof, or a hydrophobic amino acid, X₂₂ is N, a hydrophilic amino acid, or a positively charged amino acid such as K, R or H, and X₂₃ is S, a hydrophobic amino acid, or a hydrophilic amino acid, or a stapling amino acid. In some instances of SEQ ID NO:233, only four of any one of X₁ to X₂₃ are stapling amino acids, wherein the four stapling amino acids consist of two pairs of stapling amino acids, wherein the two amino acids in each pair are independently at positions i and i+4 or i and i+7. For example, in one instance of SEQ ID NO:233, only X₂, X₆, X₁₆ and X₂₀ are stapling amino acids, and X₁ is G, or a hydrophobic or hydrophilic amino acid, X₂ is a stapling amino acid, X₃ is G, a hydrophobic amino acid, or a hydrophilic amino acid, X₄ is K or a positively charged amino acid such as R or H, X₅ is F or a hydrophobic amino acid, X₆ is a stapling amino acid, X₇ is H or a positively charged amino acid such as R or H, X₈ is S, a hydrophobic amino acid, or a hydrophilic amino acid, X₉ is A or a hydrophobic amino acid, X₁₀ is K or a positively charged amino acid such as R or H, X₁₁ is K or a positively charged amino acid such as R or H, X₁₂ is F or a hydrophobic amino acid, X₁₃ is G or a hydrophobic amino acid, X₁₄ is K or a positively charged amino acid such as R or H, X₁₅ is A, a hydrophobic amino acid, or a hydrophilic amino acid, X₁₆ is a stapling amino acid, X₁₇ is V or a hydrophobic amino acid, X₁₈ is G, a hydrophobic amino acid, or a hydrophilic amino acid, X₁₉ is E, a hydrophobic amino acid, or a hydrophilic amino acid, X₂₀ is a stapling amino acid, X₂₁ is M or B, or a conservative amino acid substitution thereof, or a hydrophobic amino acid, X₂₂ is N, a hydrophilic amino acid, or a positively charged amino acid such as K, R or H, and X₂₃ is S, a hydrophobic amino acid, or a hydrophilic amino acid, or a stapling amino acid.

The disclosure also encompasses magainin II, pleurocidin, CAP18, or esculentin peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of any one of SEQ ID NOs:1, 63-75, 100, 123-127, 219-221, 227-232, or 312-314. These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell. In specific instances, the magainin II, pleurocidin, CAP18, or esculentin peptide comprises an amino acid sequence that is at least 60%, at least 65%, at least 70%, at least 75%, at least 80%, at least 85%, at least 90%, at least 95%, at least 96%, at least 97%, at least 98%, at least 99% identical to the amino acid sequence of any one of SEQ ID NOs:1, 63-75, 100, 123-127, 219-221, 227-232, or 312-314. In some instances, the magainin II peptide is at least 60% identical to the amino acid sequence of SEQ ID NO:1. In some instances, the magainin II peptide is at least 65% identical to the amino acid sequence of SEQ ID NO:1. In some instances, the magainin II peptide is at least 70% identical to the amino acid sequence of SEQ ID NO:1. In some instances, the magainin II peptide is at least 75% identical to the amino acid sequence of SEQ ID NO:1. In some instances, the magainin II peptide is at least 80% identical to the amino acid sequence of SEQ ID NO:1. In some instances, the magainin II peptide is at least 85% identical to the amino acid sequence of SEQ ID NO:1. In some instances, the magainin II peptide is at least 90% identical to the amino acid sequence of SEQ ID NO:1. In some instances, the magainin II peptide is at least 95% identical to the amino acid sequence of SEQ ID NO:1. In some instances, the magainin II peptide is at least 98% identical to the amino acid sequence of SEQ ID NO:1. In specific instances, the magainin II peptide consists of the amino acid sequence of any one of SEQ ID NOs: 1, 63-75, 100, 123-127, and 312-314. Methods for determining percent identity between amino acid sequences are known in the art. For example, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred instance, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences is accomplished using the BLAST 2.0 program. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al. (Nucleic Acids Res. 25:3389-3402, 1997).

In certain instances, the magainin II peptide is a variant having an amino acid sequence set forth in Table 1 below. These magainin II peptide variants kill hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell. This disclosure also features stabilized versions (e.g., internally cross-linked, e.g., stapled) of these variant magainin II peptides. For example, two (or more) residues of these variants separated by, e.g., 3 or 6 amino acids, are replaced with non-natural amino acids (e.g., α-methyl, α-alkenyl non-natural amino acids, e.g., (S)-2-(4′-pentenyl)Alanine) that can form a cross-link by olefin methathesis. In some instances, the variant magainin II peptides are stabilized by a hydrocarbon staple, a lactam staple; a UV-cycloaddition staple; an oxime staple; a thioether staple; a double-click staple; a bis-lactam staple; a bis-arylation staple; or a combination of any two or more thereof. In some instances, the variant magainin II peptides are stabilized by a hydrocarbon staple. These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

TABLE 1
Exemplary Magainin II peptide variants. B is norleucine.

| SEQ | | |
|---|---|---|
| ID NO | Description | Sequence |
| 63 | Magainin 11 (G1K) | KIGKFLHSAKKFGKAFVGEI(M/B)NS |
| 64 | Magainin 11 (I2K) | GKGKFLHSAKKFGKAFVGEI(M/B)NS |
| 65 | Magainin II (G3K) | GIKKFLHSAKKFGKAFVGEI(M/B)NS |
| 66 | Magainin II (H7K) | GIGKFLKSAKKFGKAFVGEI(M/B)NS |
| 67 | Magainin II (S8K) | GIGKFLHKAKKFGKAFVGEI(M/B)NS |
| 68 | Magainin II (E19K) | GIGKFLHSAKKFGKAFVGKI(M/B)NS |
| 69 | Magainin II (N22K) | GIGKFLKSAKKFGKAFVGEI(M/B)KS |
| 70 | Magainin II (G3E) | GIEKFLHSAKKFGKAFVGEI(M/B)NS |
| 71 | Magainin II (H7E) | GIGKFLESAKKFGKAFVGEI(M/B)NS |
| 72 | Magainin II (S8E) | GIGKFLHEAKKFGKAFVGEI(M/B)NS |
| 73 | Magainin II (G18E) | GIGKFLHSAKKFGKAFVEEI(M/B)NS |
| 74 | Magainin II (N22E) | GIGKFLKSAKKFGKAFVGEI(M/B)ES |
| 75 | Magainin II (A9K, B21A, N22K, | GIGKFLHSKKKFGKAFVGEIAKK |
| | S23K) | |
| 100 | Magainin II (M2IB) | GIGKFLHSAKKFGKAFVGEIBNS |
| 123 | Magainin II (A15K) | GIGKFLHSAKKFGKKFVGEI(M/B)NS |
| 124 | Magainin II (G18K) | GIGKFLHSAKKFGKAFVKEI(M/B)NS |
| 125 | Magainin II (M21K) | GIGKFLHSAKKFGKAFVGEIKNS |
| 126 | Magainin II (A15E) | GIGKFLHSAKKFGKEFVGEI(M/B)NS |
| 127 | Magainin II (S23E) | GIGKFLKSAKKFGKAFVGEI(M/B)NE |
| 312 | Magainin II (A9K) | GIGKFLHSKKKFGKAFVGEI(M/B)NS |
| 313 | Magainin II (KI IE) | GIGKFLHSAKEFGKAFVGEI(M/B)NS |
| 314 | Magainin II (S8K, B21A, N22K, | GIGKFLHKAKKFGKAFVGEIAKK |
| | S23K) | |

The magainin II, pleurocidin, CAP18, and esculentin peptides described herein can be optimized for therapeutic use. For example, if any of the above-described magainin II, pleurocidin, CAP18, or esculentin peptides cause undesired membrane disruption (cell lysis of, e.g., red blood cells), the peptides can be optimized by lowering the overall peptide hydrophobicity. This can for example be achieved by substituting especially hydrophobic residues with an amino acid with lower hydrophobicity (e.g., lysine or glutamate). Membrane disruption can also be lowered by reducing the overall positive charge of the peptide. This can be accomplished by substituting basic residues with uncharged or acidic residues. In certain instances, both the overall peptide hydrophobicity and the overall positive charge of the peptide are lowered. In some instances, the minimum overall positive charge of the peptide is +3.

### Structurally-Stabilized Oncolytic Peptides

A peptide helix is an important mediator of key protein-protein interactions that regulate many important biological processes such as apoptosis; however, when such a helix is taken out of its context within a protein and prepared in isolation, it usually adopts a random coil conformation, leading to a drastic reduction in biological activity and thus diminished therapeutic potential. The present disclosure provides structurally-stabilized antimicrobial peptides that can function as oncolytic peptides. The present disclosure includes structurally-stabilized oncolytic peptides (such as structurally-stabilized versions of the magainin II, pleurocidin, CAP18, or esculentin peptides described above) comprising at least two (e.g., 2, 3, 4, 5, 6) modified amino acids joined by an internal (intramolecular) cross-link (e.g., a staple or stitch). Stabilized peptides as described herein include stapled peptides and stitched peptides as well as peptides containing multiple stitches, multiple staples or a mix of staples and stitches, or any other chemical strategies for structural reinforcement (see. e.g., Balaram P. Cur. Opin. Struct. Biol. 1992; 2:845; Kemp D S, et al., J. Am. Chem. Soc. 1996; 118:4240; Orner B P, et al., J. Am. Chem. Soc. 2001; 123:5382; Chin J W, et al., Int. Ed. 2001; 40:3806; Chapman R N, et al., J. Am. Chem. Soc. 2004; 126:12252; Horne W S, et al., Chem., Int. Ed. 2008; 47:2853; Madden et al., Chem Commun (Camb). 2009 Oct. 7; (37): 5588-5590; Lau et al., Chem. Soc. Rev., 2015, 44:91-102; and Gunnoo et al., Org. Biomol. Chem., 2016, 14:8002-8013; all of which are incorporated by reference herein in its entirety).

In certain instances, one or more of the magainin II, pleurocidin, CAP18, or esculentin peptides described herein can be structurally-stabilized by peptide stapling (see, e.g., Walensky, J. Med. Chem., 57:6275-6288 (2014), the contents of which are incorporated by reference herein in its entirety). A peptide is “structurally-stabilized” in that it maintains its native secondary structure. For example, stapling allows a peptide, predisposed to have an α-helical secondary structure, to maintain its native α-helical conformation. This secondary structure increases resistance of the peptide to proteolytic cleavage and heat, and also may increase target binding affinity, hydrophobicity, and cell permeability. Accordingly, the stapled (cross-linked) peptides described herein have improved biological activity relative to a corresponding non-stapled (un-cross-linked) peptide.

“Peptide stapling” is a term coined from a synthetic methodology wherein two olefin-containing side-chains (e.g., cross-linkable side chains) present in a peptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chain, which may be present in a peptide chain, using any number of reaction conditions and/or catalysts to facilitate such a reaction, to provide a singly “stapled” peptide. The term “multiply stapled” peptides refers to those peptides containing more than one individual staple, and may contain two, three, or more independent staples of various spacing. Additionally, the term “peptide stitching,” as used herein, refers to multiple and tandem “stapling” events in a single peptide chain to provide a “stitched” (e.g., tandem or multiply stapled) peptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced.

In certain instances, one or more of the oncolytic peptides described herein can be structurally-stabilized. In some instances, the oncolytic peptides of this disclosure are structurally-stabilized by a hydrocarbon staple or stitch, a lactam staple or stitch; a UV-cycloaddition staple or stitch; an oxime staple or stitch; a thioether staple or stitch; a double-click staple or stitch; a bis-lactam staple or stitch; a bis-arylation staple or stitch; or a combination of any two or more thereof. In some instances, the oncolytic peptides of this disclosure are structurally-stabilized by a hydrocarbon staple. In some instances, the oncolytic peptides of this disclosure are structurally-stabilized by a hydrocarbon stitch. In some instances, the structurally-stabilized (e.g., stapled) peptide is a cross-linked version of a peptide comprising or consisting of the amino acid sequence of any one of SEQ ID NOs:1, 63-75, 100, 123-127, 219-221, 227-232, and 312-314. For example, two or more amino acids of these peptides are replaced by a stapling amino acid (e.g., an α-methyl, α-alkenyl non-natural amino acid). In some instances, the stapled peptide is a hydrocarbon stapled version of a peptide comprising or consisting of the amino acid sequence of any one of SEQ ID NOs:1, 63-75, 100, 123-127, 219-221, 227-232, and 312-314. In some instances, the stapled peptide is a peptide comprising or consisting of the amino acid sequence of any one SEQ ID NOs:1, 63-75, 100, 123-127, 219-221, 227-232, and 312-314, except that at least two (e.g., 2, 3, 4, 5, 6) amino acids of the amino acid sequence of any one of SEQ ID NOs: 1, 63-75, 100, 123-127, 219-221, 227-232, and 312-314, respectively, are replaced with a non-natural amino acid capable of forming a staple or stitch (e.g., non-natural amino acids with olefinic side chains, e.g., (S)-2-(4′-pentenyl)Alanine, (R)-2-(4′-pentenyl)Alanine, (R)-2-(7′-octenyl)Alanine, (S)-2-(7′-octenyl)Alanine). In some instances, the stapled peptide is a peptide comprising or consisting of the amino acid sequence of any one of SEQ ID NOs:1, 63-75, 100, 123-127, 219-221, 227-232, and 312-314 or comprising 1 to 13 (i.e., 1, 2, 3, 4, 5, 6, 7, 8, 9, 10, 11, 12, or 13) amino acid substitutions, deletions and/or insertions therein. In certain instances, the stapled peptide includes at least two (e.g., 2, 3, 4, 5, 6) amino acid substitutions, wherein the substituted amino acids are separated by two, three, or six amino acids, and wherein the substituted amino acids are non-natural amino acids with olefinic side chains (e.g., (S)-2-(4′-pentenyl)Alanine, (R)-2-(4′-pentenyl)Alanine, (R)-2-(7′-octenyl)Alanine, (S)-2-(7′-octenyl)Alanine). There are many known non-natural amino acids that may be used as stapling amino acids or stitching amino acids, any of which may be included in the peptides of the present invention. One example of a non-natural amino acid that may be used as a stapling amino acid is an α-methyl, α-alkenyl non-natural amino acid. Some additional examples of non-natural amino acids that may be used as stapling amino acids or stitching amino acids are: (R)-2-(7′-octenyl)Alanine, (S)-2-(7′-octenyl)Alanine, (S)-2-(4′-pentenyl)Alanine, (R)-2-(4′-pentenyl)Alanine, (R)-2-(2′-propenyl)alanine 2,2-Bis(4′-pentenyl)glycine, and 2,2-Bis(7′-octenyl)glycine. Additionally, amino acids can be derivatized to include amino acid residues that are hydroxylated, phosphorylated, sulfonated, acylated, or glycosylated. In some instances, the amino acids forming the staple or stitch (also referred to as the “stapling amino acids” or the “stitching amino acids”, respectively) are (R)-2-(2′-propenyl)alanine and (S)-2-(4′-pentenyl)alanine at positions i and i+3, respectively, of the staple. In some instances, the amino acids forming the staple or stitch (also referred to as the “stapling amino acids” or the “stitching amino acids”, respectively) are (S)-2-(4′-pentenyl)Alanine and (R)-2-(2′-propenyl)alanine at positions i and i+3, respectively, of the staple. In some instances, the amino acids forming the staple or stitch (also referred to as the “stapling amino acids” or the “stitching amino acids”, respectively) are (R)-2-(4′-pentenyl)Alanine and (S)-2-(4′-pentenyl)Alanine at positions i and i+3, respectively, of the staple. In some instances, the amino acids forming the staple or stitch (also referred to as the “stapling amino acids” or the “stitching amino acids”, respectively) are (S)-2-(4′-pentenyl)Alanine and (R)-2-(4′-pentenyl)Alanine at positions i and i+3, respectively, of the staple. In some instances, the amino acids forming the staple or stitch (also referred to as the “stapling amino acids” or the “stitching amino acids”, respectively) are (S)-2-(4′-pentenyl)Alanine at each of positions i and i+4 of the staple. In some instances, the amino acids forming the staple or stitch (also referred to as the “stapling amino acids” or the “stitching amino acids”, respectively) are (R)-2-(4′-pentenyl)Alanine at each of positions i and i+4 of the staple. In some instances, the amino acids forming the staple or stitch are (R)-2-(7′-octenyl)Alanine and (S)-2-(4′-pentenyl)Alanine at positions i and i+7, respectively, of the staple. In some instances, the amino acids forming the staple or stitch are (S)-2-(7′-octenyl)Alanine and (R)-2-(4′-pentenyl)Alanine at positions i and i+7, respectively, of the staple. When a stitch is present instead, or in addition to, a staple, a central bridging amino acid with two alkenyl residues is employed. When forming stitches, the central bridging non-natural amino acid can contain two alkenyl residues, such as in 2,2-Bis(4′-pentenyl)glycine and 2,2-Bis(7′-octenyl)glycine.

In certain instances when a stitch is at the i, i+4, and i+11 residues, (S)-2-(4′-pentenyl)Alanine, 2,2-Bis(4′-pentenyl)glycine, and (S)-2-(7′-octenyl)Alanine are substituted for the amino acids at i, i+4, and i+11 positions, respectively. In certain instances when a stitch is at the i, i+7, and i+11 residues, (R)-2-(7′-octenyl)Alanine, 2,2-Bis(4′-pentenyl)glycine, and (S)-2-(4′-pentenyl)Alanine are substituted for the amino acids at i, i+7, and i+11 positions, respectively. In certain instances when a stitch is at the i, i+7, and i+14 residues, (R)-2-(7′-octenyl)Alanine, 2,2-Bis(4′-pentenyl)glycine, and (S)-2-(7′-octenyl)Alanine are substituted for the amino acids at i, i+7, and i+14, respectively. In certain instances when a stitch is at the i, i+7, and i+14 residues, (S)-2-(7′-octenyl)Alanine, 2,2-Bis(4′-pentenyl)glycine, and (R)-2-(7′-octenyl)Alanine are substituted for the amino acids at i, i+7, and i+14, respectively. In certain instances when a stitch is at the i, i+7, and i+14 residues, (R)-2-(4′-pentenyl)Alanine, 2,2-Bis(7′-octenyl)glycine, and (S)-2-(4′-pentenyl)Alanine are substituted for the amino acids at i, i+7, and i+14, respectively. In certain instances when a stitch is at the i, i+7, and i+14 residues, (S)-2-(4′-pentenyl)Alanine, 2,2-Bis(7′-octenyl)glycine, and (R)-2-(4′-pentenyl)Alanine are substituted for the amino acids at i, i+7, and i+14, respectively.

Hydrocarbon stapled peptides include one or more tethers (linkages) between two non-natural amino acids, which tether significantly enhances the α-helical secondary structure of the peptide. Generally, the tether extends across the length of one or two helical turns (i.e., about 3.4 or about 7 amino acids). Accordingly, amino acids positioned at i and i+3; i and i+4; or i and i+7 are ideal candidates for chemical modification and cross-linking. Thus, for example, where a peptide has the sequence . . . X1, X2, X3, X4, X5, X6, X7, X8, X9 . . . , cross-links between X1 and X4, or between X1 and X5, or between X1 and X8 are useful hydrocarbon stapled forms of that peptide, as are cross-links between X2 and X5, or between X2 and X6, or between X2 and X9, etc. (i.e., forming an “i, i+3 staple”, an “i, i+4 staple”, or an “i, i+7 staple”, respectively). The use of multiple cross-links (e.g., 2, 3, 4, or more) is also contemplated. The use of multiple cross-links is very effective at stabilizing and optimizing the peptide, especially with increasing peptide length. Thus, the disclosure encompasses the incorporation of more than one cross-link within the peptide sequence to either further stabilize the sequence or facilitate the structural-stabilization, proteolytic resistance, acid stability, thermal stability, cellular permeability, and/or biological activity enhancement of longer peptide stretches. Additional description regarding making and use of hydrocarbon stapled peptides can be found, e.g., in U.S. Patent Publication Nos. 2012/0172285, 2010/0286057, and 2005/0250680, the contents of all of which are incorporated by reference herein in their entireties.

In certain instances when a staple is at the i and i+3 residues, (R)-2-(2′-propenyl)alanine and (S)-2-(4′-pentenyl)Alanine are substituted for the amino acids at those positions, respectively. In certain instances when a staple is at the i and i+3 residues, (S)-2-(4′-pentenyl)Alanine and (R)-2-(2′-propenyl)alanine are substituted for the amino acids at those positions, respectively. In certain instances when a staple is at the i and i+3 residues, (R)-2-(4′-pentenyl)Alanine and (S)-2-(4′-pentenyl)Alanine are substituted for the amino acids at those positions, respectively. In certain instances when a staple is at the i and i+3 residues, (S)-2-(4′-pentenyl)Alanine and (R)-2-(4′-pentenyl)Alanine are substituted for the amino acids at those positions, respectively. In certain instances when a staple is at the i and i+4 residues, (S)-2-(4′-pentenyl)Alanine is substituted for the amino acids at each of i and i+4. In certain instances when a staple is at the i and i+7 residues, (S)-2-(4′-pentenyl)Alanine and (R)-2-(7′-octenyl)Alanine are substituted for the amino acids at i and i+7, respectively. In certain instances when a staple is at the i and i+7 residues, (R)-2-(7′-octenyl)Alanine and (S)-2-(4′-pentenyl)Alanine are substituted for the amino acids at i and i+7, respectively. In certain instances when a stitch is at the i, i+4, and i+11 residues, (S)-2-(4′-pentenyl)Alanine, 2,2-Bis(4′-pentenyl)glycine, and (S)-2-(7′-octenyl)Alanine are substituted for the amino acids at i, i+4, and i+11 positions, respectively. In certain instances when a stitch is at the i, i+7, and i+11 residues, (R)-2-(7′-octenyl)Alanine, 2,2-Bis(4′-pentenyl)glycine, and (S)-2-(4′-pentenyl)Alanine are substituted for the amino acids at i, i+7, and i+11 positions, respectively. In certain instances when a stitch is at the i, i+7, and i+14 residues, (R)-2-(7′-octenyl)Alanine, 2,2-Bis(4′-pentenyl)glycine, and (S)-2-(7′-octenyl)Alanine are substituted for the amino acids at i, i+7, and i+14, respectively. In certain instances when a stitch is at the i, i+7, and i+14 residues, (S)-2-(7′-octenyl)Alanine, 2,2-Bis(4′-pentenyl)glycine, and (R)-2-(7′-octenyl)Alanine are substituted for the amino acids at i, i+7, and i+14, respectively. In certain instances when a stitch is at the i, i+7, and i+14 residues, (R)-2-(4′-pentenyl)Alanine, 2,2-Bis(7′-octenyl)glycine, and (S)-2-(4′-pentenyl)Alanine are substituted for the amino acids at i, i+7, and i+14, respectively. In certain instances when a stitch is at the i, i+7, and i+14 residues, (S)-2-(4′-pentenyl)Alanine, 2,2-Bis(7′-octenyl)glycine, and (R)-2-(4′-pentenyl)Alanine are substituted for the amino acids at i, i+7, and i+14, respectively.

In a peptide to be stapled or stitched, amino acids that interfere with (e.g., inhibit or reduce the efficiency of) the stapling/stitching reaction should be substituted with amino acids that do not interfere with (e.g., do not inhibit or do not substantially reduce the efficiency of) the stapling/stitching reaction. For example, methionine (Met, M) may interfere with the stapling reaction; thus, in certain instances, the methionine(s) in a peptide to be stapled is replaced with, e.g., norleucine(s). For example, in certain instances, when stapling magainin II, Met21 (numbered according to the amino acid sequence of SEQ ID NO:1) is replaced with norleucine (B). In some instances, when stapling magainin II, Met21 (numbered according to the amino acid sequence of SEQ ID NO:1) is replaced with an alanine.

In some instances, the staple(s) is located at the amino acid positions in a magainin II peptide corresponding to positions (i) 1 and 5, (ii) 2 and 6, (iii) 3 and 7, (iv) 4 and 8, (v) 5 and 9, (vi) 6 and 10, (vii) 7 and 11, (viii) 8 and 12, (ix) 9 and 13, (x) 10 and 14, (xi) 11 and 15, (xii) 12 and 16, (xiii) 13 and 17, (xiv) 14 and 18, (xv) 15 and 19, (xvi) 16 and 20, (xvii) 17 and 21, (xviii) 18 and 22, (xix) 19 and 23, (xx) (a) 2 and 6 and (b) 16 and 20, (xxi) (a) 1 and 5 and (b) 16 and 20 of the amino acid sequence of SEQ ID NO:1 or 100, or (xxii) (a) 3 and 7 and (b) 16 and 20 of the amino acid sequence of SEQ ID NO:1 or 100. In some instances, the staple is located at the amino acid positions in a magainin II peptide corresponding to positions 1 and 5 of the amino acid sequence of SEQ ID NO:1 or 100. In some instances, the staple is located at the amino acid positions in a magainin II peptide corresponding to positions 2 and 6 of the amino acid sequence of SEQ ID NO:1 or 100. In some instances, the staple is located at the amino acid positions in a magainin II peptide corresponding to positions 3 and 7 of the amino acid sequence of SEQ ID NO:1 or 100. In some instances, the staple is located at the amino acid positions in a magainin II peptide corresponding to positions 5 and 10 of the amino acid sequence of SEQ ID NO:1 or 100. In some instances, the staple is located at the amino acid positions in a magainin II peptide corresponding to positions 7 and 11 of the amino acid sequence of SEQ ID NO:1 or 100. In some instances, the staple is located at the amino acid positions in a peptide corresponding to positions 12 and 16 of the amino acid sequence of SEQ ID NO:1 or 100. In some instances, the staple is located at the amino acid positions in a magainin II peptide corresponding to positions 16 and 20 of the amino acid sequence of SEQ ID NO:1 or 100. In some instances, the staple is located at the amino acid positions in a magainin II peptide corresponding to positions 17 and 21 of the amino acid sequence of SEQ ID NO:1 or 100. In some instances, a first staple is located at the amino acid positions in a magainin II peptide corresponding to positions 2 and 6 of the amino acid sequence of SEQ ID NO:1 or 100 and a second staple is located at the amino acid positions in a magainin II peptide corresponding to positions 16 and 20 of the amino acid sequence of SEQ ID NO:1 or 100. In some instances, a first staple is located at the amino acid positions in a magainin II peptide corresponding to positions 1 and 5 of the amino acid sequence of SEQ ID NO:1 or 100 and a second staple is located at the amino acid positions in a magainin II peptide corresponding to positions 16 and 20 of the amino acid sequence of SEQ ID NO:1 or 100. In some instances, a first staple is located at the amino acid positions in a magainin II peptide corresponding to positions 3 and 7 of the amino acid sequence of SEQ ID NO:1 or 100 and a second staple is located at the amino acid positions in a magainin II peptide corresponding to positions 16 and 20 of the amino acid sequence of SEQ ID NO:1 or 100.

In some instances in which the magainin II peptide comprises or consists of the amino acid sequence of any one of SEQ ID NOs: 63-75, 100, 123-127, and 312-314, the staple(s) is located at the amino acid positions corresponding to positions (i) 1 and 5, (ii) 2 and 6, (iii) 3 and 7, (iv) 4 and 8, (v) 5 and 9, (vi) 6 and 10, (vii) 7 and 11, (viii) 8 and 12, (ix) 9 and 13, (x) 10 and 14, (xi) 11 and 15, (xii) 12 and 16, (xiii) 13 and 17, (xiv) 14 and 18, (xv) 15 and 19, (xvi) 16 and 20, (xvii) 17 and 21, (xviii) 18 and 22, (xix) 19 and 23, (xx) (a) 2 and 6 and (b) 16 and 20, (xxi) (a) 1 and 5 and (b) 16 and 20, or (xxii) (a) 3 and 7 and (b) 16 and 20 of the amino acid sequence of SEQ ID NO:1 or 100. In some instances in which the magainin II peptide comprises or consists of the amino acid sequence of any one of SEQ ID NOs: 63-75, 100, 123-127, and 312-314 the staple(s) is located at the amino acid positions corresponding to positions 3 and 7 and 16 and 20 of the amino acid sequence of SEQ ID NO:1 or 100.

In some instances in which the magainin II peptide comprises or consists of the amino acid sequence of any one of SEQ ID NOs: 63-75, 100, 123-127, and 312-314, the staple(s) is located at the amino acid positions corresponding to positions (i) 1 and 8, (ii) 2 and 9, (iii) 3 and 10, (iv) 4 and 11, (v) 5 and 12, (vi) 6 and 13, (vii) 7 and 14, (viii) 8 and 15, (ix) 9 and 16, (x) 10 and 17, (xi) 11 and 18, (xii) 12 and 19, (xiii) 13 and 20, (xiv) 14 and 21, (xv) 15 and 22, (xvi) 16 and 23. As needed, any non-specific toxicity of these i+7 stapled peptides can be mitigated with one or more amino substitutions.

In some instances in which the magainin II peptide comprises or consists of the amino acid sequence of any one of SEQ ID NOs: 63-75, 100, 123-127, and 312-314, the staple is located at the amino acid positions corresponding to positions 1 and 5 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 2 and 6 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 3 and 7 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 4 and 9 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 5 and 9 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 7 and 11 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 8 and 12 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 9 and 13 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 10 and 14 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 12 and 16 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 14 and 18 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 15 and 19 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 16 and 20 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 17 and 21 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 18 and 22 of the amino acid sequence of SEQ ID NO:1 or 100. In some instances in which the magainin II peptide comprises or consists of the amino acid sequence of any one of SEQ ID NOs: 63-75, 100, 123-127, and 312-314, the staple is located at the amino acid positions corresponding to positions 19 and 23 of the amino acid sequence of SEQ ID NO:1 or 100. In some instances in which the magainin II peptide comprises or consists of the amino acid sequence of SEQ ID NO:75, a first staple is located at the amino acid positions corresponding to positions 2 and 6 of the amino acid sequence of SEQ ID NO:1 or 100 and a second staple is located at the amino acid positions corresponding to positions 16 and 20 of the amino acid sequence of SEQ ID NO:1 or 100. In some instances in which the magainin II peptide comprises or consists of the amino acid sequence of SEQ ID NO:75, a first staple is located at the amino acid positions corresponding to positions 1 and 5 of the amino acid sequence of SEQ ID NO:1 or 100 and a second staple is located at the amino acid positions corresponding to positions 16 and 20 of the amino acid sequence of SEQ ID NO:1 or 100. In some instances in which the magainin II peptide comprises or consists of the amino acid sequence of SEQ ID NO:75, a first staple is located at the amino acid positions corresponding to positions 3 and 7 of the amino acid sequence of SEQ ID NO:1 or 100 and a second staple is located at the amino acid positions corresponding to positions 16 and 20 of the amino acid sequence of SEQ ID NO:1 or 100.

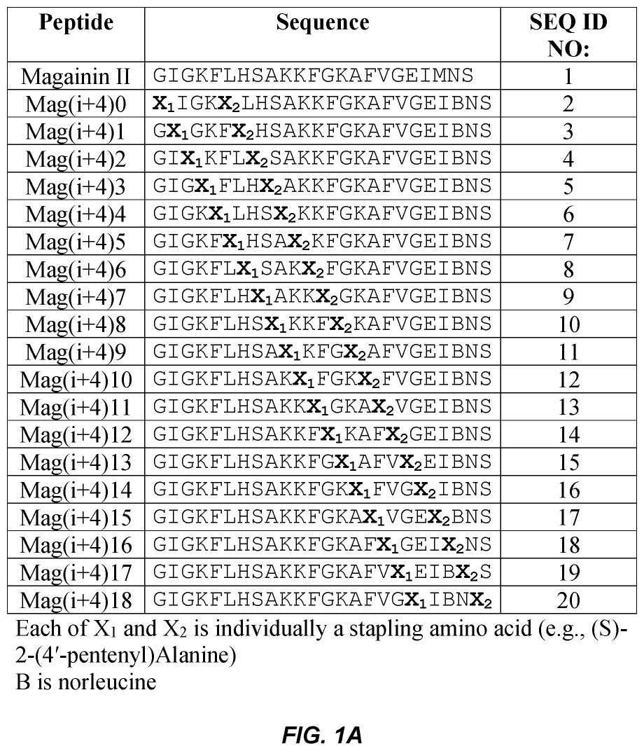

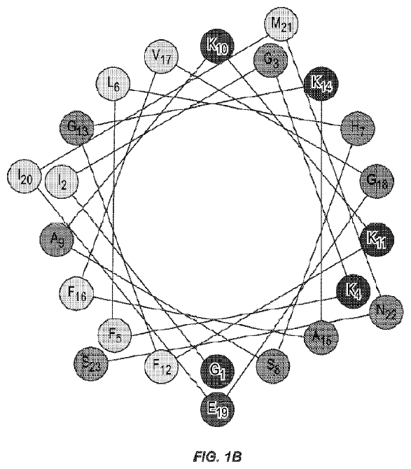

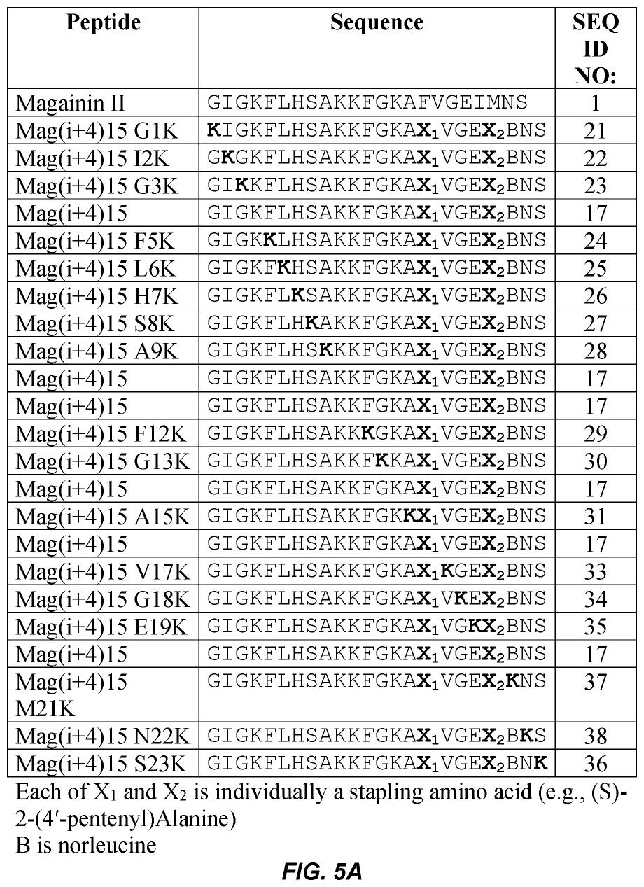

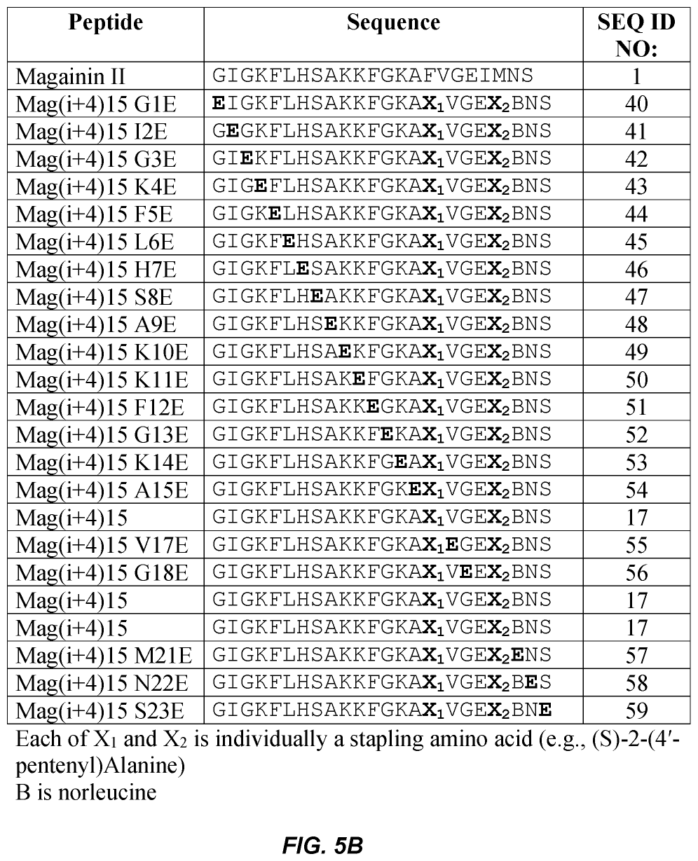

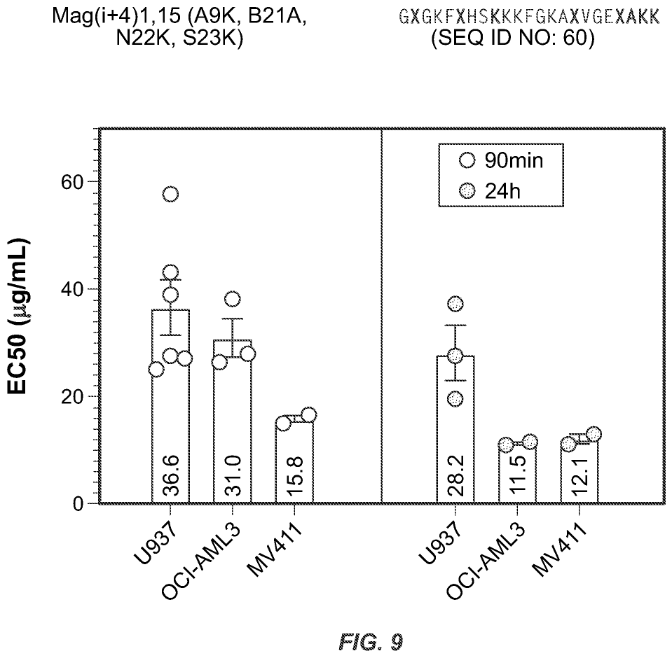

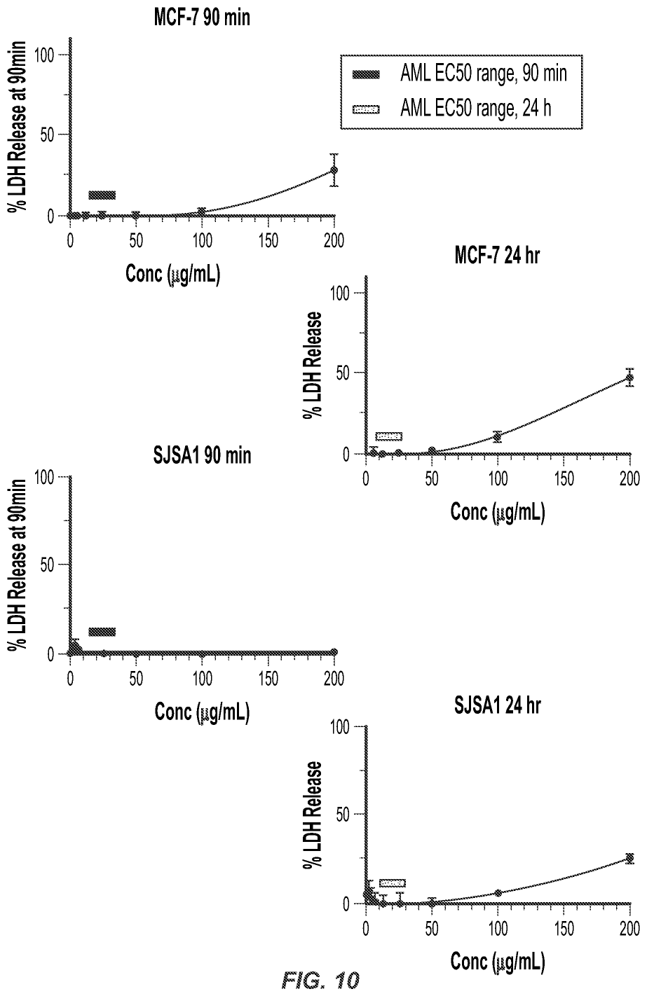

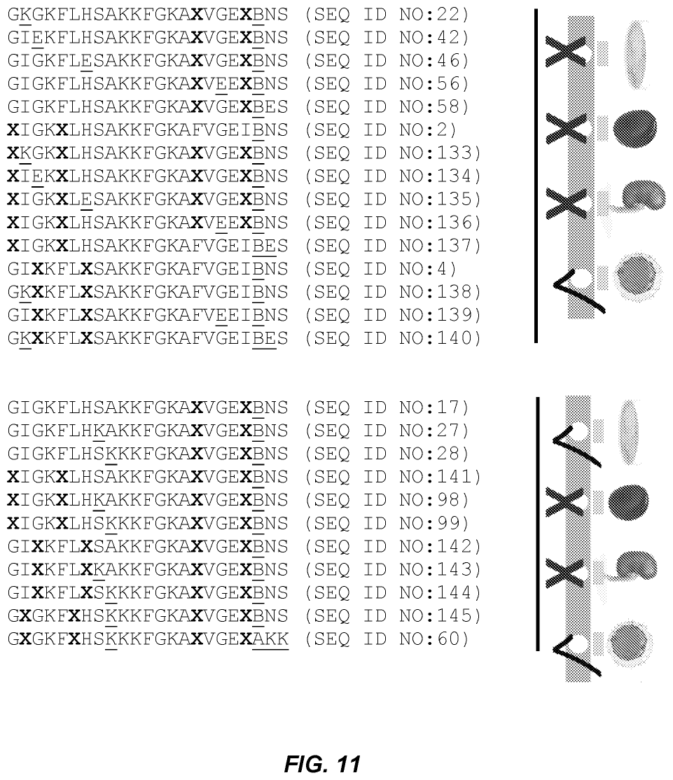

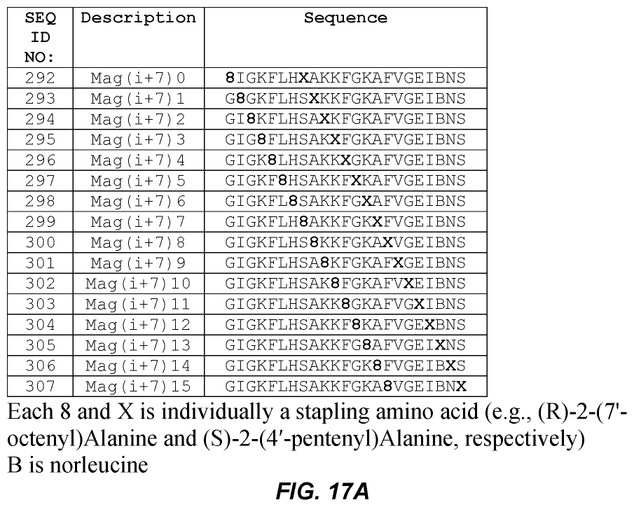

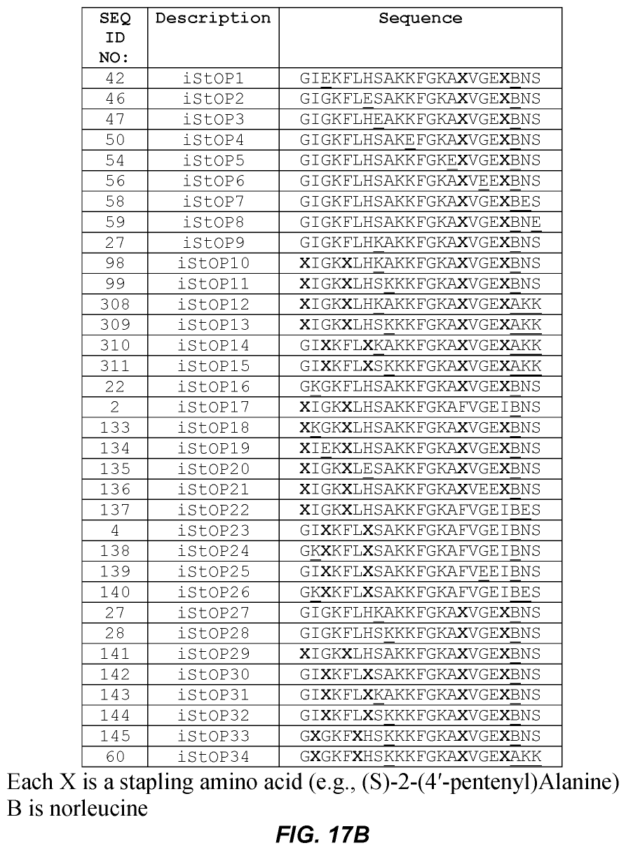

In some instances, the structurally-stabilized peptide comprises or consists of an amino acid sequence set forth in FIG. 1A (e.g., the amino acid sequence of any one of SEQ ID NOs:2-20) with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in FIG. 5A (e.g., the amino acid sequence of any one of SEQ ID NOs:21-31 and 33-38) with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in FIG. 5B (e.g., the amino acid sequence of any one of SEQ ID NOs: 40-59) with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in FIG. 9 (i.e., the amino acid sequence of SEQ ID NO:60) with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in SEQ ID NO: 98 or 99 with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in SEQ ID NO: 98 or 99 with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in FIG. 11 with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in FIG. 17A with 0 to 8 (e.g., 0, 1, 2, 3, 4, 5, 6, 7, or 8) amino acid substitutions, wherein the structurally-stabilized peptide kills cancer cells. In some instances, the structurally-stabilized peptide comprises or consists of an amino acid sequence set forth in FIG. 17B with 0 to 8 (e.g., 0, 1, 2, 3, 4, 5, 6, 7, or 8) amino acid substitutions, wherein the structurally-stabilized peptide kills cancer cells.

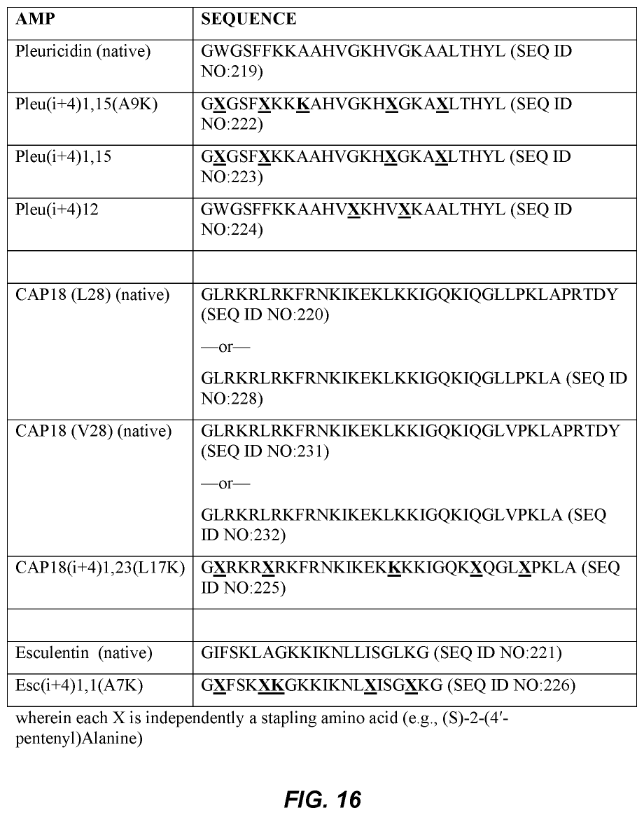

In some instances, the structurally-stabilized peptide comprises or consists of a stapled form of a peptide described in FIG. 1A (i.e., the stapled peptide is the product of one or more ring-closing metathesis reaction(s) on a peptide comprising or consisting of an amino acid sequence described in FIG. 1A, e.g., the amino acid sequence of any one of SEQ ID NOs:2-20). In some instances, the structurally-stabilized peptide comprises or consists of a stapled form of a peptide described in FIG. 5A (i.e., the stapled peptide is the product of one or more ring-closing metathesis reaction(s) on a peptide comprising or consisting of an amino acid sequence described in FIG. 5A, e.g., the amino acid sequence of any one of SEQ ID NOs:21-31 and 33-38). In some instances, the structurally-stabilized peptide comprises or consists of a stapled form of a peptide described in FIG. 5B (i.e., the stapled peptide is the product of one or more ring-closing metathesis reaction(s) on a peptide comprising or consisting of an amino acid sequence described in FIG. 5B, e.g., the amino acid sequence of any one of SEQ ID NOs: 40-59). In some instances, the structurally-stabilized peptide comprises or consists of a stapled form of a peptide described in FIG. 9 (i.e., the stapled peptide is the product of one or more ring-closing metathesis reaction(s) on a peptide comprising or consisting of an amino acid sequence described in FIG. 9, i.e., the amino acid sequence of SEQ ID NO:60). In some instances, the structurally-stabilized peptide comprises or consists of a stapled form of a peptide described in FIG. 11 (i.e., the stapled peptide is the product of one or more ring-closing metathesis reaction(s) on a peptide comprising or consisting of an amino acid sequence described in FIG. 11, e.g., the amino acid sequence of any one of SEQ ID NOs: 2, 4, 17, 22, 27, 28, 42, 46, 56, 58, 98, 99, and 133-145). In some instances, the structurally-stabilized peptide comprises or consists of a stapled form of a peptide described in FIG. 16 (i.e., the stapled peptide is the product of one or more ring-closing metathesis reaction(s) on a peptide comprising or consisting of an amino acid sequence described in FIG. 16, e.g., the amino acid sequence of any one of SEQ ID NOs: 222-226). In some instances, the structurally-stabilized peptide comprises or consists of a stapled form of a peptide described in FIG. 17A (i.e., the stapled peptide is the product of one or more ring-closing metathesis reaction(s) on a peptide comprising or consisting of an amino acid sequence described in FIG. 17A, e.g., the amino acid sequence of any one of SEQ ID NOs: 292-307). In some instances, the structurally-stabilized peptide comprises or consists of a stapled form of a peptide described in FIG. 17B (i.e., the stapled peptide is the product of one or more ring-closing metathesis reaction(s) on a peptide comprising or consisting of an amino acid sequence described in FIG. 17B, e.g., the amino acid sequence of any one of SEQ ID NOs: 2, 4, 22, 27, 28, 42, 46, 47, 50, 54, 56, 58-60, 98, 99, 133-145, and 308-311).

In some instances, the stabilized magainin II peptide comprises a stapled form of a peptide described in Table 2 (i.e., the stapled peptide is the product of one or more ring-closing metathesis reaction(s) on a peptide of Table 2). In some instances, the stabilized magainin II peptide comprises a stapled form of a peptide described in Table 2 (i.e., the stapled peptide is the product of one or more ring-closing metathesis reaction(s) on a peptide of Table 2) with 0 to 8 amino acid substitutions. In some instances, the stabilized magainin II peptide comprises a stapled form of a peptide described in Table 2 (i.e., the stapled peptide is the product of one or more ring-closing metathesis reaction(s) on a peptide of Table 2) with 0 to 4 amino acid substitutions. These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

TABLE 2
Exemplary stapled magainin II peptides. B is norleucine.

| SEQ ID | | |
|---|---|---|
| NO | DESCRIPTION | SEQUENCE |
| 2 | Mag(i + 4)0 | X₁IGKX₂LHSAKKFGKAFVGEIBNS, wherein each of X₁ |
| | | and X₂ is independently a stapling amino acid |
| 3 | Mag(i + 4)1 | GX₁GKFX₂HSAKKFGKAFVGEIBNS, wherein each of X₁ |
| | | and X₂ is independently a stapling amino acid |
| 4 | Mag(i + 4)2 | GIX₁KFLX₂SAKKFGKAFVGEIBNS, wherein each of X₁ and |
| | | X₂ is independently a stapling amino acid |
| 5 | Mag(i + 4)3 | GIGX₁FLHX₂AKKFGKAFVGEIBNS, wherein each of X₁ |
| | | and X₂ is independently a stapling amino acid |
| 6 | Mag(i + 4)4 | GIGKX₁LHSX₂KKFGKAFVGEIBNS, wherein each of X₁ |
| | | and X₂ is independently a stapling amino acid |
| 8 | Mag(i + 4)6 | GIGKFLX₁SAKX₂FGKAFVGEIBNS, wherein each of X₁ and |
| | | X₂ is independently a stapling amino acid |
| 9 | Mag(i + 4)7 | GIGKFLHX₁AKKX₂GKAFVGEIBNS, wherein each of X₁ |
| | | and X₂ is independently a stapling amino acid |
| 10 | Mag(i + 4)8 | GIGKFLHSX₁KKFX₂KAFVGEIBNS, wherein each of X₁ and |
| | | X₂ is independently a stapling amino acid |
| 11 | Mag(i + 4)9 | GIGKFLHSAX₁KFGX₂AFVGEIBNS, wherein each of X₁ and |
| | | X₂ is independently a stapling amino acid |
| 13 | Mag(i + 4)11 | GIGKFLHSAKKX₁GKAX₂VGEIBNS, wherein each of X₁ |
| | | and X₂ is independently a stapling amino acid |
| 15 | Mag(i + 4)13 | GIGKFLHSAKKFGX₁AFVX₂EIBNS, wherein each of X₁ and |
| | | X₂ is independently a stapling amino acid |
| 16 | Mag(i + 4)14 | GIGKFLHSAKKFGKX₁FVGX₂IBNS, wherein each of X₁ |
| | | and X₂ is independently a stapling amino acid |
| 17 | Mag(i + 4)15 | GIGKFLHSAKKFGKAX₁VGEX₂BNS, wherein each of X₁ |
| | | and X₂ is independently a stapling amino acid |
| 18 | Mag(i + 4)16 | GIGKFLHSAKKFGKAFX₁GEIX₂NS, wherein each of X₁ and |
| | | X₂ is independently a stapling amino acid |
| 19 | Mag(i + 4)17 | GIGKFLHSAKKFGKAFVX₁EIBX₂S, wherein each of X₁ and |
| | | X₂ is independently a stapling amino acid |
| 20 | Mag(i + 4)18 | GIGKFLHSAKKFGKAFVGX₁IBNX₂, wherein each of X₁ |
| | | and X₂ is independently a stapling amino acid |
| 21 | Mag(i + 4)15 (G1K) | KIGKFLHSAKKFGKAX₁VGEX₂BNS, wherein each of X₁ |
| | | and X₂ is independently a stapling amino acid |
| 22 | Mag(i + 4)15 (I2K) | GKGKFLHSAKKFGKAX₁VGEX₂BNS, wherein each of X₁ |
| | | and X₂ is independently a stapling amino acid |
| 23 | Mag(i + 4)15 (G3K) | GIKKFLHSAKKFGKAX₁VGEX₂BNS, wherein each of X₁ |
| | | and X₂ is independently a stapling amino acid |
| 26 | Mag(i + 4)15 (H7K) | GIGKFLKSAKKFGKAX₁VGEX₂BNS, wherein each of X₁ |
| | | and X₂ is independently a stapling amino acid |
| 27 | Mag(i + 4)15 (S8K) | GIGKFLHKAKKFGKAX₁VGEX₂BNS, wherein each of X₁ |
| | | and X₂ is independently a stapling amino acid |
| 31 | Mag(i + 4)15 | GIGKFLHSAKKFGKKX₁VGEX₂BNS, wherein each of X₁ |
| | (A15K) | and X₂ is independently a stapling amino acid |
| 34 | Mag(i + 4)15 | GIGKFLHSAKKFGKAX₁VKEX₂BNS, wherein each of X₁ |
| | (G18K) | and X₂ is independently a stapling amino acid |
| 35 | Mag(i + 4)15 | GIGKFLHSAKKFGKAX₁VGKX₂BNS, wherein each of X₁ |
| | (E19K) | and X₂ is independently a stapling amino acid |
| 37 | Mag(i + 4)15 | GIGKFLHSAKKFGKAX₁VGEX₂KNS, wherein each of X₁ |
| | (M21K) | and X₂ is independently a stapling amino acid |
| 38 | Mag(i + 4)15 | GIGKFLHSAKKFGKAX₁VGEX₂BKS, wherein each of X₁ |
| | (N22K) | and X₂ is independently a stapling amino acid |
| 42 | Mag(i + 4)15 (G3E) | GIEKFLHSAKKFGKAX₁VGEX₂BNS, wherein each of X₁ |
| | | and X₂ is independently a stapling amino acid |
| 46 | Mag(i + 4)15 (H7E) | GIGKFLESAKKFGKAX₁VGEX₂BNS, wherein each of X₁ |
| | | and X₂ is independently a stapling amino acid |
| 47 | Mag(i + 4)15 (S8E) | GIGKFLHEAKKFGKAX₁VGEX₂BNS, wherein each of X₁ |
| | | and X₂ is independently a stapling amino acid |
| 54 | Mag(i + 4)15 | GIGKFLHSAKKFGKEX₁VGEX₂BNS, wherein each of X₁ |
| | (A15E) | and X₂ is independently a stapling amino acid |
| 56 | Mag(i + 4)15 | GIGKFLHSAKKFGKAX₁VEEX₂BNS, wherein each of X₁ |
| | (G18E) | and X₂ is independently a stapling amino acid |
| 58 | Mag(i + 4)15 | GIGKFLHSAKKFGKAX₁VGEX₂BES, wherein each of X₁ |
| | (N22E) | and X₂ is independently a stapling amino acid |
| 59 | Mag(i + 4)15 (S23E) | GIGKFLHSAKKFGKAX₁VGEX₂BNE, wherein each of X₁ |
| | | and X₂ is independently a stapling amino acid |
| 60 | Mag(i + 4)15 (A9K, | GX₁GKFX₂HSKKKFGKAX₃VGEX₄AKK, wherein each of |
| | B21A, N22K, | X₁, X₂, X₃, and X₄ is independently a stapling amino acid |
| | S23K) | |
| 133 | Mag(i + 4)0, 15 | X₁KGKX₂LHSAKKFGKAX₃VGEX₄BNS, wherein each of |
| | (I2K) | X₁, X₂, X₃, and X₄ is independently a stapling amino acid |
| 134 | Mag(i + 4)0, 15 | X₁IEKX₂LHSAKKFGKAX₃VGEX₄BNS, wherein each of X₁, |
| | (G3E) | X₂, X₃, and X₄ is independently a stapling amino acid |
| 135 | Mag(i + 4)0, 15 | X₁IGKX₂LESAKKFGKAX₃VGEX₄BNS, wherein each of X₁, |
| | (H7E) | X₂, X₃, and X₄ is independently a stapling amino acid |
| 136 | Mag(i + 4)0, 15 | X₁IGKX₂LHSAKKFGKAX₃VEEX₄BNS, wherein each of X₁, |
| | (G18E) | X₂, X₃, and X₄ is independently a stapling amino acid |
| 137 | Mag(i + 4)0 (N22E) | X₁IGKX₂LHSAKKFGKAFVGEIBES, wherein each of X₁, |
| | | and X₂ is independently a stapling amino acid |
| 138 | Mag(i + 4)2 (I2K) | GKX₁KFLX₂SAKKFGKAFVGEIBNS, wherein each of X₁, |
| | | and X₂ is independently a stapling amino acid |
| 139 | Mag(i + 4)2 (G18E) | GIX₁KFLX₂SAKKFGKAFVEEIBNS, wherein each of X₁ and |
| | | X₂ is independently a stapling amino acid |
| 140 | Mag(i + 4)0, 15 | GKX₁KFLX₂SAKKFGKAFVGEIBES, wherein each of X₁ |
| | (I2K, N22E) | and X₂ is independently a stapling amino acid |
| 141 | Mag(i + 4)2, 15 | X₁IGKX₂LHSAKKFGKAX₃VGEX₄BNS, wherein each of X₁, |
| | (S8K) | X₂, X₃, and X₄ is independently a stapling amino acid |
| 142 | Mag(i + 4)2, 15 | GIX₁KFLX₂SAKKFGKAX₃VGEX₄BNS, wherein each of X₁, |
| | | X₂, X₃, and X₄ is independently a stapling amino acid |
| 143 | Mag(i + 4)2, 15 | GIX₁KFLX₂KAKKFGKAX₃VGEX₄BNS, wherein each of X₁, |
| | (S8K) | X₂, X₃, and X₄ is independently a stapling amino acid |
| 144 | Mag(i + 4)2, 15 | GIX₁KFLX₂SKKKFGKAX₃VGEX₄BNS, wherein each of X₁, |
| | (A9K) | X₂, X₃, and X₄ is independently a stapling amino acid |
| 145 | Mag(i + 4)1, 15 | GX₁GKFX₂HSKKKFGKAX₃VGEX₄BNS, wherein each of |
| | (A9K) | X₁, X₂, X₃, and X₄ is independently a stapling amino acid |
| 101 | Mag(i + 4)0 | X₁IGKX₂LHSAKKFGKAFVGEIBNS, wherein each of X₁ |
| | | and X₂ is (S)-2-(4′-pentenyl)Alanine |
| 102 | Mag(i + 4)1 | GX₁GKFX₂HSAKKFGKAFVGEIBNS, wherein each of X₁ |
| | | and X₂ is (S)-2-(4′-pentenyl)Alanine |
| 103 | Mag(i + 4)2 | GIX₁KFLX₂SAKKFGKAFVGEIBNS, wherein each of X₁ and |
| | | X₂ is (S)-2-(4′-pentenyl)Alanine |
| 104 | Mag(i + 4)4 | GIGKX₁LHSX₂KKFGKAFVGEIBNS, wherein each of X₁ |
| | | and X₂ is (S)-2-(4′-pentenyl)Alanine |
| 105 | Mag(i + 4)6 | GIGKFLX₁SAKX₂FGKAFVGEIBNS, wherein each of X₁ and |
| | | X₂ is (S)-2-(4′-pentenyl)Alanine |
| 106 | Mag(i + 4)11 | GIGKFLHSAKKX₁GKAX₂VGEIBNS, wherein each of X₁ |
| | | and X₂ is (S)-2-(4′-pentenyl)Alanine |
| 107 | Mag(i + 4)15 | GIGKFLHSAKKFGKAX₁VGEX₂BNS, wherein each of X₁ |
| | | and X₂ is (S)-2-(4′-pentenyl)Alanine |
| 108 | Mag(i + 4)16 | GIGKFLHSAKKFGKAFX₁GEIX₂NS, wherein each of X₁ and |
| | | X₂ is (S)-2-(4′-pentenyl)Alanine |
| 109 | Mag(i + 4)15 (G1K) | KIGKFLHSAKKFGKAX₁VGEX₂BNS, wherein each of X₁ |
| | | and X₂ is (S)-2-(4′-pentenyl)Alanine |
| 110 | Mag(i + 4)15 (I2K) | GKGKFLHSAKKFGKAX₁VGEX₂BNS, wherein each of X₁ |
| | | and X₂ is (S)-2-(4′-pentenyl)Alanine |
| 111 | Mag(i + 4)15 (G3K) | GIKKFLHSAKKFGKAX₁VGEX₂BNS, wherein each of X₁ |
| | | and X₂ is (S)-2-(4′-pentenyl)Alanine |
| 112 | Mag(i + 4)15 (H7K) | GIGKFLKSAKKFGKAX₁VGEX₂BNS, wherein each of X₁ |
| | | and X₂ is (S)-2-(4′-pentenyl)Alanine |
| 113 | Mag(i + 4)15 (S8K) | GIGKFLHKAKKFGKAX₁VGEX₂BNS, wherein each of X₁ |
| | | and X₂ is (S)-2-(4′-pentenyl)Alanine |
| 114 | Mag(i + 4)15 | GIGKFLHSAKKFGKAX₁VGKX₂BNS, wherein each of X₁ |
| | (E19K) | and X₂ is (S)-2-(4′-pentenyl)Alanine |
| 115 | Mag(i + 4)15 | GIGKFLHSAKKFGKAX₁VGEX₂BKS, wherein each of X₁ |
| | (N22K) | and X₂ is (S)-2-(4′-pentenyl)Alanine |
| 116 | Mag(i + 4)15 (G3E) | GIEKFLHSAKKFGKAX₁VGEX₂BNS, wherein each of X₁ |
| | | and X₂ is (S)-2-(4′-pentenyl)Alanine |
| 117 | Mag(i + 4)15 (H7E) | GIGKFLESAKKFGKAX₁VGEX₂BNS, wherein each of X₁ |
| | | and X₂ is (S)-2-(4′-pentenyl)Alanine |
| 118 | Mag(i + 4)15 (S8E) | GIGKFLHEAKKFGKAX₁VGEX₂BNS, wherein each of X₁ |
| | | and X₂ is (S)-2-(4′-pentenyl)Alanine |
| 119 | Mag(i + 4)15 | GIGKFLHSAKKFGKAX₁VEEX₂BNS, wherein each of X₁ |
| | (G18E) | and X₂ is (S)-2-(4′-pentenyl)Alanine |
| 120 | Mag(i + 4)15 | GIGKFLHSAKKFGKAX₁VGEX₂BES, wherein each of X₁ |
| | (N22E) | and X₂ is (S)-2-(4′-pentenyl)Alanine |
| 121 | Mag(i + 4)15 (A9K, | GX₁GKFX₂HSKKKFGKAX₃VGEX₄AKK, wherein each of |
| | B21A, N22K, S23E) | X₁, X₂, X₃, and X₄ is (S)-2-(4′-pentenyl)Alanine |
| 128 | Mag(i + 4)15 | GIGKFLHSAKKFGKKX₁VGEX₂BNS, wherein each of X₁ |
| | (A15K) | and X₂ is (S)-2-(4′-pentenyl)Alanine |
| 129 | Mag(i + 4)15 | GIGKFLHSAKKFGKAX₁VKEX₂BNS, wherein each of X₁ |
| | (G18K) | and X₂ is (S)-2-(4′-pentenyl)Alanine |
| 130 | Mag(i + 4)15 | GIGKFLHSAKKFGKAX₁VGEX₂KNS, wherein each of X₁ |
| | (M21K) | and X₂ is (S)-2-(4′-pentenyl)Alanine |
| 131 | Mag(i + 4)15 | GIGKFLHSAKKFGKEX₁VGEX₂BNS, wherein each of X₁ |
| | (A15E) | and X₂ is (S)-2-(4′-pentenyl)Alanine |
| 132 | Mag(i + 4)15 (S23E) | GIGKFLHSAKKFGKAX₁VGEX₂BNE, wherein each of X₁ |
| | | and X₂ is (S)-2-(4′-pentenyl)Alanine |
| 146 | Mag(i + 4)0, 15 | X₁KGKX₂LHSAKKFGKAX₃VGEX₄BNS, wherein each of |
| | (I2K) | X₁, X₂, X₃, and X₄ is (S)-2-(4′-pentenyl)Alanine |
| 147 | Mag(i + 4)0, 15 | X₁IEKX₂LHSAKKFGKAX₃VGEX₄BNS, wherein each of X₁, |
| | (G3E) | X₂, X₃, and X₄ is (S)-2-(4′-pentenyl)Alanine |
| 148 | Mag(i + 4)0, 15 | X₁IGKX₂LESAKKFGKAX₃VGEX₄BNS, wherein each of X₁, |
| | (H7E) | X₂, X₃, and X₄ is (S)-2-(4′-pentenyl)Alanine |
| 149 | Mag(i + 4)0, 15 | X₁IGKX₂LHSAKKFGKAX₃VEEX₄BNS, wherein each of X₁, |
| | (G18E) | X₂, X₃, and X₄ is (S)-2-(4′-pentenyl)Alanine |
| 150 | Mag(i + 4)0 (N22E) | X₁IGKX₂LHSAKKFGKAFVGEIBES, wherein each of X₁, |
| | | and X₂ is (S)-2-(4′-pentenyl)Alanine |
| 151 | Mag(i + 4)2 (I2K) | GKX₁KFLX₂SAKKFGKAFVGEIBNS, wherein each of X₁, |
| | | and X₂ is (S)-2-(4′-pentenyl)Alanine |
| 152 | Mag(i + 4)2 (G18E) | GIX₁KFLX₂SAKKFGKAFVEEIBNS, wherein each of X₁ and |
| | | X₂ is (S)-2-(4′-pentenyl)Alanine |
| 153 | Mag(i + 4)0, 15 | GKX₁KFLX₂SAKKFGKAFVGEIBES, wherein each of X₁ |
| | (I2K, N22E) | and X₂ is (S)-2-(4′-pentenyl)Alanine |
| 154 | Mag(i + 4)2, 15 | X₁IGKX₂LHSAKKFGKAX₃VGEX₄BNS, wherein each of X₁, |
| | (S8K) | X₂, X₃, and X₄ is (S)-2-(4′-pentenyl)Alanine |
| 155 | Mag(i + 4)2, 15 | GIX₁KFLX₂SAKKFGKAX₃VGEX₄BNS, wherein each of X₁, |
| | | X₂, X₃, and X₄ is (S)-2-(4′-pentenyl)Alanine |
| 156 | Mag(i + 4)2, 15 | GIX₁KFLX₂KAKKFGKAX₃VGEX₄BNS, wherein each of X₁, |
| | (S8K) | X₂, X₃, and X₄ is (S)-2-(4′-pentenyl)Alanine |
| 157 | Mag(i + 4)2, 15 | GIX₁KFLX₂SKKKFGKAX₃VGEX₄BNS, wherein each of X₁, |
| | (A9K) | X₂, X₃, and X₄ is (S)-2-(4′-pentenyl)Alanine |
| 158 | Mag(i + 4)1, 15 | GX₁GKFX₂HSKKKFGKAX₃VGEX₄BNS, wherein each of |
| | (A9K) | X₁, X₂, X₃, and X₄ is (S)-2-(4′-pentenyl)Alanine |
| 192 | Mag(i + 4)3 | GIGX₁FLHX₂AKKFGKAFVGEIBNS, wherein each of X₁ |
| | | and X₂ is (S)-2-(4′-pentenyl) Alanine |
| 193 | Mag(i + 4)7 | GIGKFLHX₁AKKX₂GKAFVGEIBNS, wherein each of X₁ |
| | | and X₂ is (S)-2-(4′-pentenyl)Alanine |
| 194 | Mag(i + 4)8 | GIGKFLHSX₁KKFX₂KAFVGEIBNS, wherein each of X₁ and |
| | | X₂ is (S)-2-(4′-pentenyl)Alanine |
| 195 | Mag(i + 4)9 | GIGKFLHSAX₁KFGX₂AFVGEIBNS, wherein each of X₁ and |
| | | X₂ is (S)-2-(4′-pentenyl)Alanine |
| 196 | Mag(i + 4)13 | GIGKFLHSAKKFGX₁AFVX₂EIBNS, wherein each of X₁ and |
| | | X₂ is (S)-2-(4′-pentenyl)Alanine |
| 197 | Mag(i + 4)14 | GIGKFLHSAKKFGKX₁FVGX₂IBNS, wherein each of X₁ |
| | | and X₂ is (S)-2-(4′-pentenyl)Alanine |
| 198 | Mag(i + 4)17 | GIGKFLHSAKKFGKAFVX₁EIBX₂S, wherein each of X₁ and |
| | | X₂ is (S)-2-(4′-pentenyl)Alanine |
| 199 | Mag(i + 4)18 | GIGKFLHSAKKFGKAFVGX₁IBNX₂, wherein each of X₁ |
| | | and X₂ is (S)-2-(4′-pentenyl)Alanine |

In some instances, the disclosure features structurally-stabilized (e.g., stapled) peptides comprising or consisting of the amino acid sequence set forth in SEQ ID NO:2 or 101, wherein a side chain of the stapling amino acid at position 1 of the amino acid sequence set forth in SEQ ID NO:2 or 101, respectively, is cross-linked to a side chain of the stapling amino acid at position 5 of the amino acid sequence set forth in SEQ ID NO:2 or 101, respectively. In some instances, the disclosure features structurally-stabilized (e.g., stapled) peptides comprising or consisting of the amino acid sequence set forth in SEQ ID NO:3 or 102, wherein a side chain of the stapling amino acid at position 2 of the amino acid sequence set forth in SEQ ID NO:3 or 102, respectively, is cross-linked to a side chain of the stapling amino acid at position 6 of the amino acid sequence set forth in SEQ ID NO:3 or 102, respectively. In some instances, the disclosure features structurally-stabilized (e.g., stapled) peptides comprising or consisting of the amino acid sequence set forth in SEQ ID NO:4 or 103, wherein a side chain of the stapling amino acid at position 3 of the amino acid sequence set forth in SEQ ID NO:4 or 103, respectively, is cross-linked to a side chain of the stapling amino acid at position 7 of the amino acid sequence set forth in SEQ ID NO:4 or 103, respectively. In some instances, the disclosure features structurally-stabilized (e.g., stapled) peptides comprising or consisting of the amino acid sequence set forth in SEQ ID NO:6 or 104, wherein a side chain of the stapling amino acid at position 5 of the amino acid sequence set forth in SEQ ID NO:6 or 104, respectively, is cross-linked to a side chain of the stapling amino acid at position 9 of the amino acid sequence set forth in SEQ ID NO:6 or 104, respectively. In some instances, the disclosure features structurally-stabilized (e.g., stapled) peptides comprising or consisting of the amino acid sequence set forth in SEQ ID NO:8 or 105, wherein a side chain of the stapling amino acid at position 7 of the amino acid sequence set forth in SEQ ID NO:8 or 105, respectively, is cross-linked to a side chain of the stapling amino acid at position 11 of the amino acid sequence set forth in SEQ ID NO:8 or 105, respectively. In some instances, the disclosure features structurally-stabilized (e.g., stapled) peptides comprising or consisting of the amino acid sequence set forth in SEQ ID NO:13 or 106, wherein a side chain of the stapling amino acid at position 12 of the amino acid sequence set forth in SEQ ID NO:13 or 106, respectively, is cross-linked to a side chain of the stapling amino acid at position 16 of the amino acid sequence set forth in SEQ ID NO:13 or 106, respectively. In some instances, the disclosure features structurally-stabilized (e.g., stapled) peptides comprising or consisting of the amino acid sequence set forth in any one of SEQ ID NOs:17, 21-23, 26, 27, 35, 38, 42, 46, 47, 56, 58, 107, and 109-120, wherein a side chain of the stapling amino acid at position 16 of the amino acid sequence set forth in any one of SEQ ID NOs:17, 21-23, 26, 27, 35, 38, 42, 46, 47, 56, 58, 107, and 109-120, respectively, is cross-linked to a side chain of the stapling amino acid at position 20 of the amino acid sequence set forth in any one of SEQ ID NOs:17, 21-23, 26, 27, 35, 38, 42, 46, 47, 56, 58, 107, and 109-120, respectively. In some instances, the disclosure features structurally-stabilized (e.g., stapled) peptides comprising or consisting of the amino acid sequence set forth in SEQ ID NO:18 or 108, wherein a side chain of the stapling amino acid at position 17 of the amino acid sequence set forth in SEQ ID NO:18 or 108, respectively, is cross-linked to a side chain of the stapling amino acid at position 21 of the amino acid sequence set forth in SEQ ID NO:18 or 108, respectively. In some instances, the disclosure features structurally-stabilized (e.g., stapled) peptides comprising or consisting of the amino acid sequence set forth in SEQ ID NO:60 or 121, wherein a side chain of the stapling amino acid at position 2 of the amino acid sequence set forth in SEQ ID NO:60 or 121, respectively, is cross-linked to a side chain of the stapling amino acid at position 6 of the amino acid sequence set forth in SEQ ID NO:60 or 121, respectively, and a side chain of the stapling amino acid at position 16 of the amino acid sequence set forth in SEQ ID NO:60 or 121, respectively, is cross-linked to a side chain of the stapling amino acid at position 20 of the amino acid sequence set forth in SEQ ID NO:60 or 121, respectively. In some instances, the disclosure features structurally-stabilized (e.g., stapled) peptides comprising or consisting of the amino acid sequence set forth in SEQ ID NO:5 or 192, wherein a side chain of the stapling amino acid at position 4 of the amino acid sequence set forth in SEQ ID NO:2 or 101, respectively, is cross-linked to a side chain of the stapling amino acid at position 8 of the amino acid sequence set forth in SEQ ID NO:5 or 192, respectively. In some instances, the disclosure features structurally-stabilized (e.g., stapled) peptides comprising or consisting of the amino acid sequence set forth in SEQ ID NO:9 or 193, wherein a side chain of the stapling amino acid at position 8 of the amino acid sequence set forth in SEQ ID NO:2 or 101, respectively, is cross-linked to a side chain of the stapling amino acid at position 12 of the amino acid sequence set forth in SEQ ID NO:9 or 193, respectively. In some instances, the disclosure features structurally-stabilized (e.g., stapled) peptides comprising or consisting of the amino acid sequence set forth in SEQ ID NO:10 or 194, wherein a side chain of the stapling amino acid at position 9 of the amino acid sequence set forth in SEQ ID NO:2 or 101, respectively, is cross-linked to a side chain of the stapling amino acid at position 13 of the amino acid sequence set forth in SEQ ID NO:10 or 194, respectively. In some instances, the disclosure features structurally-stabilized (e.g., stapled) peptides comprising or consisting of the amino acid sequence set forth in SEQ ID NO:11 or 195, wherein a side chain of the stapling amino acid at position 10 of the amino acid sequence set forth in SEQ ID NO:2 or 101, respectively, is cross-linked to a side chain of the stapling amino acid at position 14 of the amino acid sequence set forth in SEQ ID NO:11 or 195, respectively. In some instances, the disclosure features structurally-stabilized (e.g., stapled) peptides comprising or consisting of the amino acid sequence set forth in SEQ ID NO:15 or 196, wherein a side chain of the stapling amino acid at position 14 of the amino acid sequence set forth in SEQ ID NO:2 or 101, respectively, is cross-linked to a side chain of the stapling amino acid at position 18 of the amino acid sequence set forth in SEQ ID NO:15 or 196, respectively. In some instances, the disclosure features structurally-stabilized (e.g., stapled) peptides comprising or consisting of the amino acid sequence set forth in SEQ ID NO:16 or 197, wherein a side chain of the stapling amino acid at position 15 of the amino acid sequence set forth in SEQ ID NO:2 or 101, respectively, is cross-linked to a side chain of the stapling amino acid at position 19 of the amino acid sequence set forth in SEQ ID NO:16 or 197, respectively. In some instances, the disclosure features structurally-stabilized (e.g., stapled) peptides comprising or consisting of the amino acid sequence set forth in SEQ ID NO:10 or 194, wherein a side chain of the stapling amino acid at position 18 of the amino acid sequence set forth in SEQ ID NO:19 or 198, respectively, is cross-linked to a side chain of the stapling amino acid at position 22 of the amino acid sequence set forth in SEQ ID NO:19 or 198, respectively. In some instances, the disclosure features structurally-stabilized (e.g., stapled) peptides comprising or consisting of the amino acid sequence set forth in SEQ ID NO:10 or 194, wherein a side chain of the stapling amino acid at position 19 of the amino acid sequence set forth in SEQ ID NO:20 or 199, respectively, is cross-linked to a side chain of the stapling amino acid at position 23 of the amino acid sequence set forth in SEQ ID NO:20 or 190, respectively.

In some instances, the disclosure features structurally-stabilized (e.g., stapled) peptides comprising or consisting of an amino acid sequence described in FIG. 17A, wherein a side chain of the first (N-terminal) stapling amino acid is cross-linked to a side chain of the second (C-terminal) stapling amino acid. In some instances, the disclosure features structurally-stabilized (e.g., stapled) peptides comprising or consisting of an amino acid sequence described in FIG. 17B, wherein a side chain of the first (from N-terminus to C-terminus) stapling amino acid is cross-linked to a side chain of the second (from N-terminus to C-terminus) stapling amino acid, and where a third and fourth stapling amino acid is present, a side chain of the third (from N-terminus to C-terminus) stapling amino acid is cross-linked to a side chain of the fourth (from N-terminus to C-terminus) stapling amino acid.

This disclosure also features stabilized pleurocidin antimicrobial peptides. Exemplary stabilized pleurocidin antimicrobial peptides include:

GX₁GSFX₂KKKAHVGKHX₃GKAX₄LTHYL, wherein each of X₁, X₂, X₃, and X₄ is independently a stapling amino acid (SEQ ID NO:222)

GX₁GSFX₂KKAAHVGKHX₃GKAX₄LTHYL, wherein each of X₁, X₂, X₃, and X₄ is independently a stapling amino acid (SEQ ID NO:223); and

GWGSFFKKAAHVX₁KHVX₂KAALTHYL, wherein each of X₁ and X₂ is independently a stapling amino acid (SEQ ID NO:224).

In some instances of SEQ ID NO:222, each of each of X₁, X₂, X₃, and X₄ is (S)-2-(4′-pentenyl)Alanine. In some instances of SEQ ID NO:222, a side chain of X₁ is cross-linked to a side chain of X₂ and a side chain of X₃ is cross-linked to a side chain of X₄. In some instances of SEQ ID NO:223, each of each of X₁, X₂, X₃, and X₄ is (S)-2-(4′-pentenyl)Alanine. In some instances of SEQ ID NO:223, a side chain of X₁ is cross-linked to a side chain of X₂ and a side chain of X₃ is cross-linked to a side chain of X₄. In some instances of SEQ ID NO:224, each of each of X₁ and X₂ is (S)-2-(4′-pentenyl)Alanine. In some instances of SEQ ID NO:224, a side chain of X₁ is cross-linked to a side chain of X₂ and a side chain of X₃ is cross-linked to a side chain of X₄. In some instances, the stabilized pleurocidin antimicrobial peptide has one or more (e.g., 1, 2, 3, 4, 5, 6, 7, 8, 9, 10) amino acid substitutions in the above listed sequences. The substitutions may be conservative and/or non-conservative. The peptides are amphipathic.

These peptides are able to kill cancer cells (e.g., leukemia, lymphoma, multiple myeloma cells) or any human cell with a net negative charge on the outer leaflet surface of its cell membrane.

This disclosure also features stabilized CAP18 antimicrobial peptides. An exemplary stabilized CAP18 antimicrobial peptide includes:

GX₁RKRX₂RKFRNKIKEKKKKIGQKX₃QGLX₄PKLA, wherein each of X₁, X₂, X₃, and X₄ is independently a stapling amino acid (SEQ ID NO:225).

In some instances of SEQ ID NO:225, each of each of X₁, X₂, X₃, and X₄ is (S)-2-(4′-pentenyl)Alanine. In some instances of SEQ ID NO:225, a side chain of X₁ is cross-linked to a side chain of X₂ and a side chain of X₃ is cross-linked to a side chain of X₄. In some instances, the stabilized CAP18 antimicrobial peptide has one or more (e.g., 1, 2, 3, 4, 5, 6, 7, 8, 9, 10) amino acid substitutions in the above listed sequence. The substitutions may be conservative and/or non-conservative. The peptides are amphipathic. These peptides are able to kill cancer cells (e.g., leukemia, lymphoma, multiple myeloma cells) or any human cell with a net negative charge on the outer leaflet surface of its cell membrane.

This disclosure also features stabilized esculentin antimicrobial peptides. An exemplary stabilized esculentin antimicrobial peptide includes:

GX₁FSKX₂KGKKIKNLX₃ISGX₄KG, wherein each of X₁, X₂, X₃, and X₄ is independently a stapling amino acid (SEQ ID NO:226).

In some instances of SEQ ID NO:226, each of each of X₁, X₂, X₃, and X₄ is (S)-2-(4′-pentenyl)Alanine. In some instances of SEQ ID NO:226, a side chain of X₁ is cross-linked to a side chain of X₂ and a side chain of X₃ is cross-linked to a side chain of X₄. In some instances, the stabilized CAP18 antimicrobial peptide has one or more (e.g., 1, 2, 3, 4, 5, 6, 7, 8, 9, 10) amino acid substitutions in the above listed sequence. The substitutions may be conservative and/or non-conservative. The peptides are amphipathic. These peptides are able to kill cancer cells (e.g., leukemia, lymphoma, multiple myeloma cells) or any human cell with a net negative charge on the outer leaflet surface of its cell membrane.

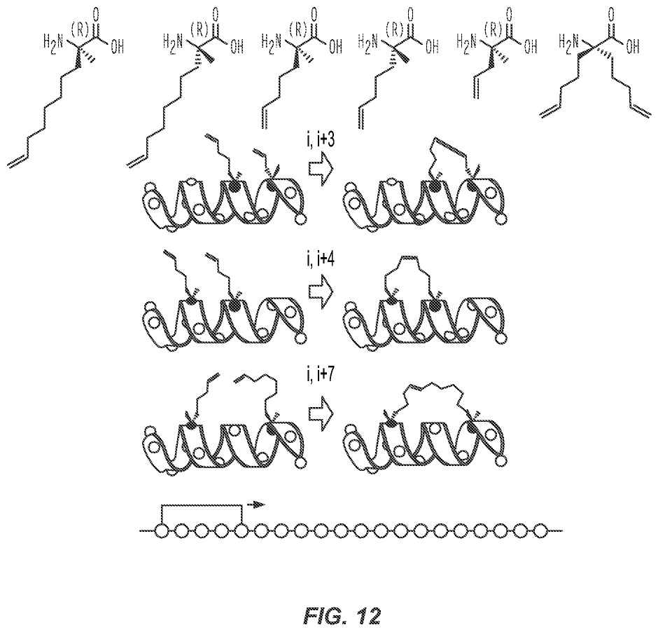

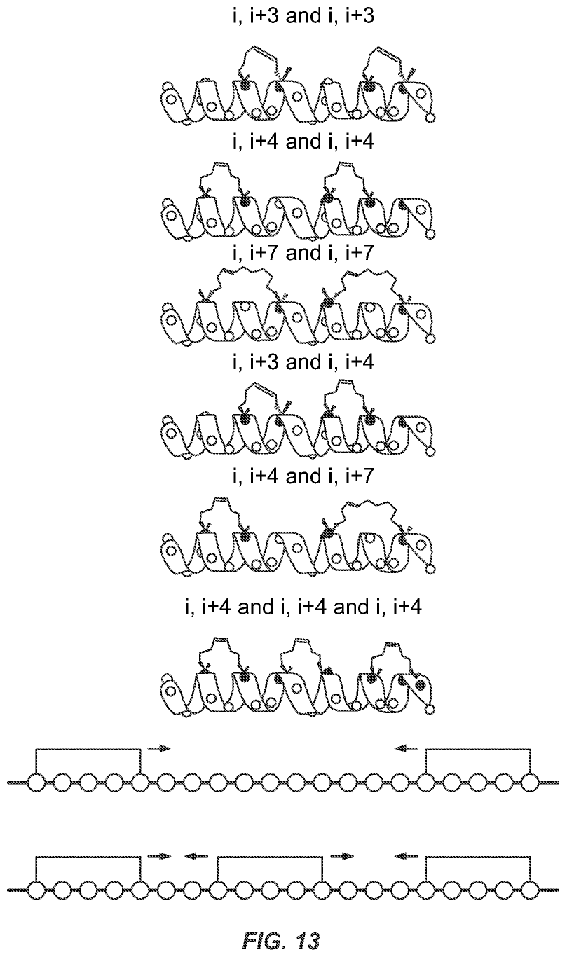

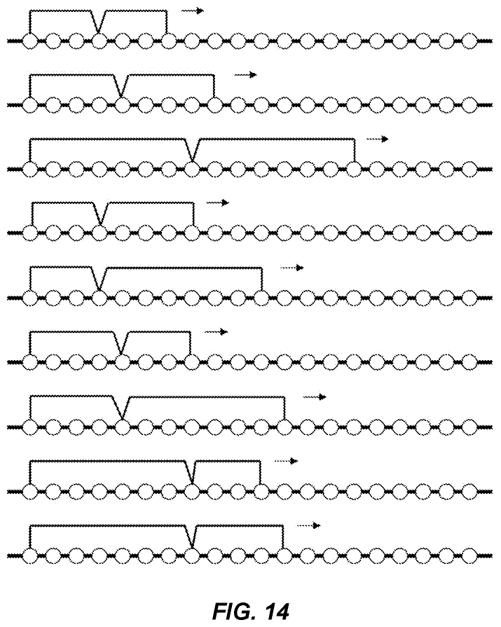

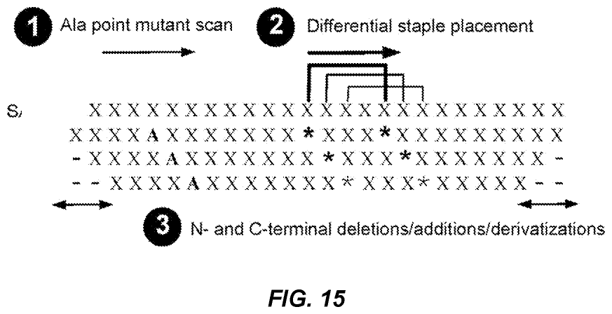

FIG. 12 top panel shows exemplary chemical structures of non-natural amino acids that can be used to generate various cross-linked compounds (i.e., “stapling amino acids” or “stitching amino acids”). FIG. 12 middle panel illustrates peptides with hydrocarbon cross-links between positions i and i+3; i and i+4; and i and i+7 residues. FIG. 12 bottom panel illustrates a staple walk along a peptide sequence. FIG. 13 shows various peptide sequences with double and triple stapling strategies, and exemplary staple walks. FIG. 14 illustrates exemplary staple walks using various lengths of branched stitched moieties. FIG. 15 illustrates peptide variants based on point mutant and staple scans, and N- and C-terminal deletions, additions, and/or derivatizations. FIG. 16 further shows specific examples of a variety of AMPs that can be modified to produce StOPs.

In one aspect, the structurally-stabilized magainin II peptide comprises Formula (I),

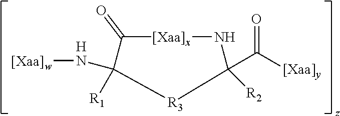

or a pharmaceutically acceptable salt thereof, wherein:
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄—K—R₄]ₙ; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

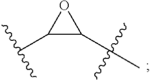 [SMILES: *C1OC1*]

R₆ is H, alkyl, or a therapeutic agent;
n is an integer from 1-4;
x is an integer from 2-10;
each y is independently an integer from 0-100;
z is an integer from 1-10 (e.g., 1, 2, 3, 4, 5, 6, 7, 8, 9, 10);

and each Xaa is independently an amino acid, and wherein the structurally-stabilized peptide specifically lyses hematological cancer cells (e.g., leukemia, lymphoma, and/or multiple myeloma cells). In some instances, each of the [Xaa]_{w} of Formula (I), [Xaa]ₓ of Formula (I), and [Xaa]_{y} of Formula (I) is as described for any one of constructs 1-78 of Table 3. For example, for a stabilized peptide comprising the [Xaa]_{w}, the [Xaa]ₓ, and the [Xaa]_{y} of construct 1 of Table 3, [Xaa]_{w} is absent, [Xaa]ₓ is IGK, and [Xaa]_{y} is LHSAKKFGKAFVGEIBNS (SEQ ID NO:32). As another example, for a stabilized peptide comprising the [Xaa]_{w}, the [Xaa]ₓ, and the [Xaa]_{y} of construct 2 of Table 3, [Xaa]_{w} is G, [Xaa]ₓ is GFK, and [Xaa]_{y} is HSAKKFGKAFVGEIBNS (SEQ ID NO:122).

Formula (I) may be applied to any amino acid sequence described herein comprising two stapling amino acids. For instance, when applying Formula (I) to the amino acid sequence X₁IGKX₂LHSAKKFGKAFVGEIBNS (SEQ ID NO: 2), [Xaa]_{w} is absent, [Xaa]ₓ is IGK, and [Xaa]_{y} is LHSAKKFGKAFVGEIBNS (SEQ ID NO:32).

TABLE 3
[Xaa]_{w}, [Xaa]ₓ, [Xaa]_{y} sequences for Formula (I) constructs 1-78.
B is norleucine.

| Construct | [Xaa]_{w} | [Xaa]ₓ | [Xaa]_{y} |
|---|---|---|---|
| 1 | 8 Absent 9 | IGK | LHSAKKFGKAFVGEIBNS (SEQ |
| | | | ID NO: 32) or |
| | | | LHSAKKFGKAFVGEIANS (SEQ |
| | | | ID NO: 89) |
| 2 | G | GFK | HSAKKFGKAFVGEIBNS (SEQ |
| | | | ID NO: 122) or |
| | | | HSAKKFGKAFVGEIANS (SEQ |
| | | | ID NO: 90) |
| 3 | GI | KFL | SAKKFGKAFVGEIBNS (SEQ ID |
| | | | NO: 76) or |
| | | | SAKKFGKAFVGEIANS (SEQ ID |
| | | | NO: 94) |
| 4 | GIGK (SEQ ID NO: 39) | LHS | KKFGKAFVGEIBNS (SEQ ID |
| | | | NO: 77) or KKFGKAFVGEIANS |
| | | | (SEQ ID NO: 95) |
| 5 | GIGKFL (SEQ ID | SAK | FGKAFVGEIBNS (SEQ ID |
| | NO: 78) | | NO: 79) or FGKAFVGEIANS |
| | | | (SEQ ID NO: 61) |
| 6 | GIGKFLHSAKK (SEQ | GKA | VGEIBNS (SEQ ID NO: 81) or |
| | ID NO: 80) | | VGEIANS (SEQ ID NO: 62) |
| 7 | GIGKFLHSAKKFGKA | VGE | BNS or ANS |
| | (SEQ ID NO: 82) | | |
| 8 | GIGKFLHSAKKFGKAF | GEI | NS |
| | (SEQ ID NO: 83) | | |
| 9 | KIGKFLHSAKKFGKA | VGE | BNS or ANS |
| | (SEQ ID NO: 84) | | |
| 10 | GKGKFLHSAKKFGKA | VGE | BNS or ANS |
| | (SEQ ID NO: 85) | | |
| 11 | GIKKFLHSAKKFGKA | VGE | BNS or ANS |
| | (SEQ ID NO: 86) | | |
| 12 | GIGKFLKSAKKFGKA | VGE | BNS or ANS |
| | (SEQ ID NO: 87) | | |
| 13 | GIGKFLHKAKKFGKA | VGE | BNS or ANS |
| | (SEQ ID NO: 88) | | |
| 14 | GIGKFLHSAKKFGKA | VGK | BNS or ANS |
| | (SEQ ID NO: 82) | | |
| 15 | GIGKFLHSAKKFGKA | VGE | BKS or AKS |
| | (SEQ ID NO: 82) | | |
| 16 | GIEKFLHSAKKFGKA | VGE | BNS or ANS |
| | (SEQ ID NO: 91) | | |
| 17 | GIGKFLESAKKFGKA | VGE | BNS or ANS |
| | (SEQ ID NO: 92) | | |
| 18 | GIGKFLHEAKKFGKA | VGE | BNS or ANS |
| | (SEQ ID NO: 93) | | |
| 19 | GIGKFLHSAKKFGKA | VEE | BNS or ANS |
| | (SEQ ID NO: 82) | | |
| 20 | GIGKFLHSAKKFGKA | VGE | BES or AES |
| | (SEQ ID NO: 82) | | |
| 21 | G | GKF | HSKKKFGKAX₁VGEX₂AKK |
| | | | (SEQ ID NO: 96), wherein each of |
| | | | X₁ and X₂ is a stapling amino acid, |
| | | | and wherein a side chain of X₁ is |
| | | | cross-linked to a side chain of X₂ |
| 22 | GX₁GKFX₂HSKKKFGK | VGE | AKK |
| | A (SEQ ID NO: 97), | | |
| | wherein each of X₁ and X₂ | | |
| | is a stapling amino acid, | | |
| | and wherein a side chain | | |
| | of X₁ is cross-linked to a | | |
| | side chain of X₂ | | |
| 23 | GIGKFLHSAKKFGKA | VGE | BNE or ANE |
| | (SEQ ID NO: 82) | | |
| 24 | Absent | KGK | LHSAKKFGKAX₃VGEX₄BNS |
| | | | (SEQ ID NO: 160) or |
| | | | LHSAKKFGKAX₃VGEX₄ANS |
| | | | (SEQ ID NO: 161), wherein each of |
| | | | X₃ and X₄ is a stapling amino acid, |
| | | | and wherein a side chain of X₃ is |
| | | | cross-linked to a side chain of X₄ |
| 25 | X₁KGKX₂LHSAKKFGK | VGE | BNS or ANS |
| | A, wherein each of X₁ and | | |
| | X₂ is a stapling amino | | |
| | acid (SEQ ID NO: 162), | | |
| | and wherein a side chain | | |
| | of X₁ is cross-linked to a | | |
| | side chain of X₂ | | |
| 26 | Absent | IEK | LHSAKKFGKAX₃VGEX₄BNS |
| | | | (SEQ ID NO: 163) or |
| | | | LHSAKKFGKAX₃VGEX₄ANS |
| | | | (SEQ ID NO: 164), wherein each of |
| | | | X₃ and X₄ is a stapling amino acid, |
| | | | and wherein a side chain of X₃ is |
| | | | cross-linked to a side chain of X₄ |
| 27 | X₁IEKX₂LHSAKKFGKA, | VGE | BNS or ANS |
| | wherein each of X₁ and | | |
| | X₂ is a stapling amino | | |
| | acid (SEQ ID NO: 165), | | |
| | and wherein a side chain | | |
| | of X₁ is cross-linked to a | | |
| | side chain of X₂ | | |
| 28 | Absent | IGK | LESAKKFGKAX₃VGEX₄BNS |
| | | | (SEQ ID NO: 166) or |
| | | | LESAKKFGKAX₃VGEX₄ANS |
| | | | (SEQ ID NO: 167), wherein each of |
| | | | X₃ and X₄ is a stapling amino acid, |
| | | | and wherein a side chain of X₃ is |
| | | | cross-linked to a side chain of X₄ |
| 29 | X₁IGKX₂LESAKKFGKA, | VGE | BNS or ANS |
| | wherein each of X₁ and | | |
| | X₂ is a stapling amino | | |
| | acid (SEQ ID NO: 191), | | |
| | and wherein a side chain | | |
| | of X₁ is cross-linked to a | | |
| | side chain of X₂ | | |
| 30 | Absent | IGK | LHSAKKFGKAX₃VEEX₄BNS |
| | | | (SEQ ID NO: 315) or |
| | | | LHSAKKFGKAX₃VEEX₄ANS |
| | | | (SEQ ID NO: 316), wherein each of |
| | | | X₃ and X₄ is a stapling amino acid, |
| | | | and wherein a side chain of X₃ is |
| | | | cross-linked to a side chain of X₄ |
| 31 | X₁IGKX₂LHSAKKFGKA, | VEE | BNS or ANS |
| | wherein each of X₁ and | | |
| | X₂ is a stapling amino | | |
| | acid (SEQ ID NO: 178), | | |
| | and wherein a side chain | | |
| | of X₁ is cross-linked to a | | |
| | side chain of X₂ | | |
| 32 | Absent | IGK | LHSAKKFGKAFVGEIBES (SEQ |
| | | | ID NO: 168) or |
| | | | LHSAKKFGKAFVGEIAES (SEQ |
| | | | ID NO: 169) |
| 33 | GK | KFL | SAKKFGKAFVGEIBNS (SEQ ID |
| | | | NO: 170) or |
| | | | SAKKFGKAFVGEIANS (SEQ ID |
| | | | NO: 171) |
| 34 | GI | KFL | SAKKFGKAFVEEIBNS (SEQ ID |
| | | | NO: 172) or |
| | | | SAKKFGKAFVEEIANS (SEQ ID |
| | | | NO: 173) |
| 35 | GK | KFL | SAKKFGKAFVGEIBES (SEQ ID |
| | | | NO: 174) or |
| | | | SAKKFGKAFVGEIAES (SEQ ID |
| | | | NO: 175) |
| 36 | Absent | IGK | LHSAKKFGKAX₃VGEX₄BNS |
| | | | (SEQ ID NO: 176) or |
| | | | LHSAKKFGKAX₃VGEX₄ANS |
| | | | (SEQ ID NO: 177), wherein each of |
| | | | X₃ and X₄ is a stapling amino acid, |
| | | | and wherein a side chain of X₃ is |
| | | | cross-linked to a side chain of X₄ |
| 37 | X₁IGKXLHSAKKFGKA, | VGE | BNS or ANS |
| | wherein each of X₁ and | | |
| | X₂ is a stapling amino | | |
| | acid (SEQ ID NO: 178), | | |
| | and wherein a side chain | | |
| | of X₁ is cross-linked to a | | |
| | side chain of X₂ | | |
| 38 | GI | KFL | SAKKFGKAX₃VGEX₄BNS (SEQ |
| | | | ID NO: 179) or |
| | | | SAKKFGKAX₃VGEX₄ANS (SEQ |
| | | | ID NO: 180), wherein each of X₃ |
| | | | and X₄ is a stapling amino acid, and |
| | | | wherein a side chain of X₃ is cross- |
| | | | linked to a side chain of X₄ |
| 39 | GIX₁KFLX₂SAKKFGKA, | VGE | BNS or ANS |
| | wherein each of X₁ and | | |
| | X₂ is a stapling amino | | |
| | acid (SEQ ID NO: 181), | | |
| | and wherein a side chain | | |
| | of X₁ is cross-linked to a | | |
| | side chain of X₂ | | |
| 40 | GI | KFL | KAKKFGKAX₃VGEX₄BNS (SEQ |
| | | | ID NO: 182) or |
| | | | KAKKFGKAX₃VGEX₄ANS (SEQ |
| | | | ID NO: 183), wherein each of X₃ |
| | | | and X₄ is a stapling amino acid, and |
| | | | wherein a side chain of X₃ is cross- |
| | | | linked to a side chain of X₄ |
| 41 | GIX₁KFLX₂KAKKFGKA, | VGE | BNS or ANS |
| | wherein each of X₁ and | | |
| | X₂ is a stapling amino | | |
| | acid (SEQ ID NO: 184), | | |
| | and wherein a side chain | | |
| | of X₁ is cross-linked to a | | |
| | side chain of X₂ | | |
| 42 | GI | KFL | SKKKFGKAX₃VGEX₄BNS (SEQ |
| | | | ID NO: 185) or |
| | | | SKKKFGKAX₃VGEX₄ANS (SEQ |
| | | | ID NO: 186), wherein each of X₃ |
| | | | and X₄ is a stapling amino acid, and |
| | | | wherein a side chain of X₃ is cross- |
| | | | linked to a side chain of X₄ |
| 43 | GIX₁KFLX₂SKKKFGKA, | VGE | BNS or ANS |
| | wherein each of X₁ and | | |
| | X₂ is a stapling amino | | |
| | acid (SEQ ID NO: 187), | | |
| | wherein a side chain of X₁ | | |
| | is cross-linked to a side | | |
| | chain of X₂ | | |
| 44 | G | GKF | HSKKKFGKAX₃VGEX₄BNS |
| | | | (SEQ ID NO: 188) or |
| | | | HSKKKFGKAX₃VGEX₄ANS |
| | | | (SEQ ID NO: 189), wherein each of |
| | | | X₃ and X₄ is a stapling amino acid, |
| | | | and wherein a side chain of X₃ is |
| | | | cross-linked to a side chain of X₄ |
| 45 | GX₁GKFX₂HSKKKFGKA, | VGE | BNS or ANS |
| | wherein each of X₁ and | | |
| | X₂ is a stapling amino | | |
| | acid (SEQ ID NO: 190), | | |
| | and wherein a side chain | | |
| | of X₁ is cross-linked to a | | |
| | side chain of X₂ | | |
| 46 | GIG | FLH | AKKFGKAFVGEIBNS (SEQ ID |
| | | | NO: 207) or |
| | | | AKKFGKAFVGEIANS (SEQ ID |
| | | | NO: 213) |
| 47 | GIGKFLH (SEQ ID | AKK | GKAFVGEIBNS (SEQ ID |
| | NO: 200) | | NO: 208) or GKAFVGEIANS |
| | | | (SEQ ID NO: 214) |
| 48 | GIGKFLHS (SEQ ID | KKF | KAFVGEIBNS (SEQ ID NO: 209) |
| | NO: 201) | | or KAFVGEIANS (SEQ ID |
| | | | NO: 215) |
| 49 | GIGKFLHSA (SEQ ID | KFG | AFVGEIBNS (SEQ ID NO: 210) or |
| | NO: 202) | | AFVGEIANS (SEQ ID NO: 216) |
| 50 | GIGKFLHSAKKFG | AFV | EIBNS (SEQ ID NO: 211) or |
| | (SEQ ID NO: 203) | | EIANS (SEQ ID NO 217) |
| 51 | GIGKFLHSAKKFGK | FVG | IBNS (SEQ ID NO: 212) or IANS |
| | (SEQ ID NO: 204) | | (SEQ ID NO: 218) |
| 52 | GIGKFLHSAKKFGKAF | EIB | S |
| | V (SEQ ID NO: 205) | | |
| 53 | GIGKFLHSAKKFGKAF | IBN | Absent |
| | VG (SEQ ID NO: 206) | | |
| 54 | Absent | IGKFLH | AKKFGKAFVGEIBNS (SEQ ID |
| | | (SEQ ID | NO: 253) or |
| | | NO: 237) | AKKFGKAFVGEIANS (SEQ ID |
| | | | NO: 279) |
| 55 | G | GKFLHS | KKFGKAFVGEIBNS (SEQ ID |
| | | (SEQ ID | NO: 254) or KKFGKAFVGEIANS |
| | | NO: 238) | (SEQ ID NO: 280) |
| 56 | GI | KFLHSA | KFGKAFVGEIBNS (SEQ ID |
| | | (SEQ ID | NO: 255) or KFGKAFVGEIANS |
| | | NO: 239) | (SEQ ID NO: 281) |
| 57 | GIG | FLHSAK | FGKAFVGEIBNS (SEQ ID |
| | | (SEQ ID | NO: 256) or FGKAFVGEIANS |
| | | NO: 240) | (SEQ ID NO: 282) |
| 58 | GIGK (SEQ ID NO: 39) | LHSAKK | GKAFVGEIBNS (SEQ ID |
| | | (SEQ ID | NO: 257) or GKAFVGEIANS |
| | | NO: 241) | (SEQ ID NO: 283) |
| 59 | GIGKF (SEQ ID NO: 234) | HSAKKF | KAFVGEIBNS (SEQ ID NO: 258) |
| | | (SEQ ID | or KAFVGEIANS (SEQ ID |
| | | NO: 242) | NO: 284) |
| 60 | GIGKFL (SEQ ID | SAKKFG | AFVGEIBNS (SEQ ID NO: 259) or |
| | NO: 78) | (SEQ ID | AFVGEIANS (SEQ ID NO: 285) |
| | | NO: 243) | |
| 61 | GIGKFLH (SEQ ID | AKKFGK | FVGEIBNS (SEQ ID NO: 260) or |
| | NO: 200) | (SEQ ID | FVGEIANS (SEQ ID NO: 286) |
| | | NO: 244) | |
| 62 | GIGKFLHS (SEQ ID | KKFGKA | VGEIBNS (SEQ ID NO: 261) or |
| | NO: 201) | (SEQ ID | VGEIANS (SEQ ID NO: 287) |
| | | NO: 245) | |
| 63 | GIGKFLHSA (SEQ ID | KFGKAF | GEIBNS (SEQ ID NO: 262) or |
| | NO: 202) | (SEQ ID | GEIANS (SEQ ID NO: 288) |
| | | NO: 246) | |
| 64 | GIGKFLHSAK (SEQ ID | FGKAFV | EIBNS (SEQ ID NO: 263) or |
| | NO: 235) | (SEQ ID | EIANS (SEQ ID NO: 289) |
| | | NO: 247) | |
| 65 | GIGKFLHSAKK (SEQ | GKAFVG | IBNS (SEQ ID NO: 264) or IANS |
| | ID NO: 80) | (SEQ ID | (SEQ ID NO: 290) |
| | | NO: 248) | |
| 66 | GIGKFLHSAKKF (SEQ | KAFVGE | BNS or ANS |
| | IDNO: 236) | (SEQ ID | |
| | | NO: 249) | |
| 67 | GIGKFLHSAKKFG | AFVGEI | NS |
| | (SEQ ID NO: 203) | (SEQ ID | |
| | | NO: 250) | |
| 68 | GIGKFLHSAKKFGK | FVGEIB | S |
| | (SEQ ID NO: 204) | (SEQ ID | |
| | | NO: 251) or | |
| | | FVGEIA | |
| | | (SEQ ID | |
| | | NO: 277) | |
| 69 | GIGKFLHSAKKFGKA | VGEIBN | Absent |
| | (SEQ ID NO: 82) | (SEQ ID | |
| | | NO: 252) or | |
| | | VGEIAN | |
| | | (SEQ ID | |
| | | NO: 278) | |
| 70 | G | GSF | KKKAHVGKH_{X}1GKA_{X}2LTHYL, |
| | | | wherein each of X₁ and X₂ is a |
| | | | stapling amino acid (SEQ ID |
| | | | NO: 270), wherein a side chain of |
| | | | X₁ is cross-linked to a side chain of |
| | | | X₂ |
| 71 | GX₁GSFX₂KKKAHVGKH, | GKA | LTHYL (SEQ ID NO: 271) |
| | wherein each of X₁ and | | |
| | X₂ is a stapling amino | | |
| | acid (SEQ ID NO: 265), | | |
| | wherein a side chain of X₁ | | |
| | is cross-linked to a side | | |
| | chain of X₂ | | |
| 72 | G | GSF | KKAAHVGKHX₁GKAX₂LTHYL, |
| | | | wherein each of X₁ and X₂ is a |
| | | | stapling amino acid (SEQ ID |
| | | | NO: 291), wherein a side chain of |
| | | | X₁ is cross-linked to a side chain of |
| | | | X₂ |
| 73 | GX₁GSFX₂KKAAHVGKH, | GKA | LTHYL (SEQ ID NO: 272) |
| | wherein each of X₁ and | | |
| | X₂ is a stapling amino | | |
| | acid (SEQ ID NO: 266), | | |
| | wherein a side chain of X₁ | | |
| | is cross-linked to a side | | |
| | chain of X₂ | | |
| 74 | GWGSFFKKAAHV | KHV | KAALTHYL (SEQ ID NO: 273) |
| | (SEQ ID NO: 267) | | |
| 75 | G | RKR | RKFRNKIKEKKKKIGQKX₁QGLX₂PKLA, |
| | | | wherein each of X₁ and |
| | | | X₂ is a stapling amino acid (SEQ |
| | | | ID NO: 274), wherein a side chain |
| | | | of X₁ is cross-linked to a side chain |
| | | | ofX₂ |
| 76 | GX₁RKRX₂RKFRNKIKEKKKKIGQK, | QGL | PKLA (SEQ ID NO: 275) |
| | wherein each of X₁ and X₂ | | |
| | is a stapling amino acid | | |
| | (SEQ ID NO: 268), wherein | | |
| | a side chain of X₁ is | | |
| | cross-linked to a side | | |
| | chain of X₂ | | |
| 77 | G | FSK | KGKKIKNLX₁ISGX₂KG, wherein |
| | | | each of X₁ and X₂ is a stapling |
| | | | amino acid (SEQ ID NO: 276), |
| | | | wherein a side chain of X₁ is cross- |
| | | | linked to a side chain of X₂ |
| 78 | GX₁FSKX₂KGKKIKNL, | ISG | KG |
| | wherein each of X₁ and X₂ | | |
| | is a stapling amino acid | | |
| | (SEQ ID NO: 269), | | |
| | wherein a side chain of X₁ | | |
| | is cross-linked to a side | | |
| | chain of X₂ | | |

In certain instances, the sequences set forth above in Table 3 can have at least one (e.g., 1, 2, 3, 4, 5, 6) amino acid substitution or deletion. The magainin II peptides can include any amino acid sequence described herein.

The tether of Formula (I) can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (I), x is 2, 3, or 6. In some instances of Formula (I), each y is independently an integer between 0 and 15, or 3 and 15. In some instances of Formula (I), R₁ and R₂ are each independently H or C₁-C₆ alkyl. In some instances of Formula (I), R₁ and R₂ are each independently C₁-C₃ alkyl. In some instances or Formula (I), at least one of R₁ and R₂ are methyl. For example, R₁ and R₂ can both be methyl. In some instances of Formula (I), R₃ is alkyl (e.g., C₈ alkyl) and x is 3. In some instances of Formula (I), R₃ is C₁₁ alkyl and x is 6. In some instances of Formula (I), R₃ is alkenyl (e.g., C₈ alkenyl) and x is 3. In some instances of Formula (I), x is 6 and R₃ is C₁₁ alkenyl. In some instances of Formula (I), R₃ is a straight chain alkyl, alkenyl, or alkynyl. In some instances of Formula (I), R₃ is —CH₂—CH₂—CH₂—CH═CH—CH₂—CH₂—CH₂—. In some instances of Formula (I) having an i, i+4 staple, R₃ is —CH₂—CH₂—CH₂—CH═CH—CH₂—CH₂—CH₂—. In some instances of Formula (I) having an i, i+7 staple, R₃ is —(CH₂)₆—CH═CH—(CH₂)₆—.

In some instances of Formula (I),

each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;

each R₃ is independently alkylene, alkenylene, or alkynylene, any of which is substituted or unsubstituted;

z is 1, 2, 3, 4, 5, 6, 7, 8, 9, or 10; and

(a) each [Xaa]_{w} is absent, each [Xaa]ₓ is IGK, and each [Xaa]_{y} is LHSAKKFGKAFVGEIBNS (SEQ ID NO:32) or LHSAKKFGKAFVGEIANS (SEQ ID NO:89);

(b) each [Xaa]_{w} is G, each [Xaa]ₓ is GKF, and each [Xaa]_{y} is HSAKKFGKAFVGEIBNS (SEQ ID NO:122) or HSAKKFGKAFVGEIANS (SEQ ID NO:90);

(c) each [Xaa]_{w} is GI, each [Xaa]ₓ is KFL, and each [Xaa]_{y} is SAKKFGKAFVGEIBNS (SEQ ID NO:76) or SAKKFGKAFVGEIANS (SEQ ID NO:94);

(d) each [Xaa]_{w} is GIGK (SEQ ID NO:39), each [Xaa]ₓ is LHS, and each [Xaa]_{y} is KKFGKAFVGEIBNS (SEQ ID NO:77) or KKFGKAFVGEIANS (SEQ ID NO:95);

(e) each [Xaa]_{w} is GIGKFL (SEQ ID NO:78), each [Xaa]ₓ is SAK, and each [Xaa]_{y} is FGKAFVGEIBNS (SEQ ID NO:79) or FGKAFVGEIANS (SEQ ID NO:61);

(f) each [Xaa]_{w} is GIGKFLHSAKK (SEQ ID NO:80), each [Xaa]ₓ is GKA, and each [Xaa]_{y} is VGEIBNS (SEQ ID NO:81) or VGEIANS (SEQ ID NO:62);

(g) each [Xaa]_{w} is GIGKFLHSAKKFGKA (SEQ ID NO:82), each [Xaa]ₓ is VGE, and each [Xaa]_{y} is BNS or ANS;

(h) each [Xaa]_{w} is GIGKFLHSAKKFGKAF (SEQ ID NO:83), each [Xaa]ₓ is GEI, and each [Xaa]_{y} is NS;

(i) each [Xaa]_{w} is KIGKFLHSAKKFGKA (SEQ ID NO:84), each [Xaa]ₓ is VGE, and each [Xaa]_{y} is BNS or ANS;

(j) each [Xaa]_{w} is GKGKFLHSAKKFGKA (SEQ ID NO:85), each [Xaa]ₓ is VGE, and each [Xaa]_{y} is BNS or ANS;

(k) each [Xaa]_{w} is GIKKFLHSAKKFGKA (SEQ ID NO:86), each [Xaa]ₓ is VGE, and each [Xaa]_{y} is BNS or ANS;

(l) each [Xaa]_{w} is GIGKFLKSAKKFGKA (SEQ ID NO:87), each [Xaa]ₓ is VGE, and each [Xaa]_{y} is BNS or ANS;

(m) each [Xaa]_{w} is GIGKFLHKAKKFGKA (SEQ ID NO:88), each [Xaa]ₓ is VGE, and each [Xaa]_{y} is BNS or ANS;

(n) each [Xaa]_{w} is GIGKFLHSAKKFGKA (SEQ ID NO:82), each [Xaa]ₓ is VGK, and each [Xaa]_{y} is BNS or ANS;

(o) each [Xaa]_{w} is GIGKFLHSAKKFGKA (SEQ ID NO:82), each [Xaa]ₓ is VGE, and each [Xaa]_{y} is BKS or AKS;

(p) each [Xaa]_{w} is GIEKFLHSAKKFGKA (SEQ ID NO:91), each [Xaa]ₓ is VGE, and each [Xaa]_{y} is BNS or ANS;

(q) each [Xaa]_{w} is GIGKFLESAKKFGKA (SEQ ID NO:92), each [Xaa]ₓ is VGE, and each [Xaa]_{y} is BNS or ANS;

(r) each [Xaa]_{w} is GIGKFLHEAKKFGKA (SEQ ID NO:93), each [Xaa]ₓ is VGE, and each [Xaa]_{y} is BNS or ANS;

(s) each [Xaa]_{w} is GIGKFLHSAKKFGKA (SEQ ID NO:82), each [Xaa]ₓ is VEE, and each [Xaa]_{y} is BNS or ANS;

(t) each [Xaa]_{w} is GIGKFLHSAKKFGKA (SEQ ID NO:82), each [Xaa]ₓ is VGE, and each [Xaa]_{y} is BES or AES;

(u) each [Xaa]_{w} is G, each [Xaa]ₓ is GKF, and each [Xaa]_{y} is HSKKKFGKAX₁VGEX₂AKK (SEQ ID NO:96), wherein each of X₁ and X₂ is a stapling amino acid, and wherein a side chain of X₁ is cross-linked to a side chain of X₂; or

(v) each [Xaa]_{w} is GX₁GKFX₂HSKKKFGKA (SEQ ID NO:97), each [Xaa]ₓ is VGE, and each [Xaa]_{y} is AKK, wherein each of X₁ and X₂ is a stapling amino acid, and wherein a side chain of X₁ is cross-linked to a side chain of X₂;

(w) each [Xaa]_{w} is GIGKFLHSAKKFGKA (SEQ ID NO:82), each [Xaa]ₓ is VGE, and each [Xaa]_{y} is BNE or ANE;

(x) each [Xaa]_{w} is absent, each [Xaa]ₓ is KGK, and each [Xaa]_{y} is LHSAKKFGKAX₃VGEX₄BNS (SEQ ID NO:160) or LHSAKKFGKAX₃VGEX₄ANS (SEQ ID NO:161), wherein each of X₃ and X₄ is a stapling amino acid, and wherein a side chain of X₃ is cross-linked to a side chain of X₄;

(y) each [Xaa]_{w} is X₁KGKX₂LHSAKKFGKA, wherein each of X₁ and X₂ is a stapling amino acid (SEQ ID NO:162), and wherein a side chain of X₁ is cross-linked to a side chain of X₂, each [Xaa]ₓ is VGE, and each [Xaa]_{y} is BNS or ANS;

(z) each [Xaa]_{w} is absent, each [Xaa]ₓ is IEK, and each [Xaa]_{y} is LHSAKKFGKAX₃VGEX₄BNS (SEQ ID NO:163) or LHSAKKFGKAX₃VGEX₄ANS (SEQ ID NO:164), wherein each of X₃ and X₄ is a stapling amino acid, and wherein a side chain of X₃ is cross-linked to a side chain of X₄;

(aa) each [Xaa]_{w} is X₁IEKX₂LHSAKKFGKA, wherein each of X₁ and X₂ is a stapling amino acid (SEQ ID NO:165), and wherein a side chain of X₁ is cross-linked to a side chain of X₂, each [Xaa]ₓ is VGE, and each [Xaa]_{y} is BNS or ANS;

(bb) each [Xaa]_{w} is absent, each [Xaa]ₓ is IGK, and each [Xaa]_{y} is LESAKKFGKAX₃VGEX₄BNS (SEQ ID NO:166) or LESAKKFGKAX₃VGEX₄ANS (SEQ ID NO:167), wherein each of X₃ and X₄ is a stapling amino acid, and wherein a side chain of X₃ is cross-linked to a side chain of X₄;

(cc) each [Xaa]_{w} is X₁IGKX₂LESAKKFGKA, wherein each of X₁ and X₂ is a stapling amino acid (SEQ ID NO:191), and wherein a side chain of X₁ is cross-linked to a side chain of X₂, each [Xaa]ₓ is VGE, and each [Xaa]_{y} is BNS or ANS;

(dd) each [Xaa]_{w} is absent, each [Xaa]ₓ is IGK, and each [Xaa]_{y} is LHSAKKFGKAX₃VEEX₄BNS (SEQ ID NO:315) or LHSAKKFGKAX₃VEEX₄ANS (SEQ ID NO:316), wherein each of X₃ and X₄ is a stapling amino acid, and wherein a side chain of X₃ is cross-linked to a side chain of X₄;

(ee) each [Xaa]_{w} is X₁IGKX₂LHSAKKFGKA, wherein each of X₁ and X₂ is a stapling amino acid (SEQ ID NO:178), and wherein a side chain of X₁ is cross-linked to a side chain of X₂, each [Xaa]ₓ is VEE, and each [Xaa]_{y} is BNS or ANS;

(ff) each [Xaa]_{w} is absent, each [Xaa]ₓ is IGK, and each [Xaa]_{y} is LHSAKKFGKAFVGEIBES (SEQ ID NO:168) or LHSAKKFGKAFVGEIAES (SEQ ID NO:169);

(gg) each [Xaa]_{w} is GK, each [Xaa]ₓ is KFL, and each [Xaa]_{y} is SAKKFGKAFVGEIBNS (SEQ ID NO:170) or SAKKFGKAFVGEIANS (SEQ ID NO:171);

(hh) each [Xaa]_{w} is GI, each [Xaa]ₓ is KFL, and each [Xaa]_{y} is SAKKFGKAFVEEIBNS (SEQ ID NO:172) or SAKKFGKAFVEEIANS (SEQ ID NO:173);

(ii) each [Xaa]_{w} is GK, each [Xaa]ₓ is KFL, and each [Xaa]_{y} is SAKKFGKAFVGEIBES (SEQ ID NO:174) or SAKKFGKAFVGEIAES (SEQ ID NO:175);

(jj) each [Xaa]_{w} is absent, each [Xaa]ₓ is IGK, and each [Xaa]_{y} is LHSAKKFGKAX₃VGEX₄BNS (SEQ ID NO:176) or LHSAKKFGKAX₃VGEX₄ANS (SEQ ID NO:177), wherein each of X₃ and X₄ is a stapling amino acid, and wherein a side chain of X₃ is cross-linked to a side chain of X₄;

(kk) each [Xaa]_{w} is X₁IGKX₂LHSAKKFGKA, wherein each of X₁ and X₂ is a stapling amino acid (SEQ ID NO:178), and wherein a side chain of X₁ is cross-linked to a side chain of X₂, each [Xaa]ₓ is VGE, and each [Xaa]_{y} is BNS or ANS;

(ll) each [Xaa]_{w} is GI, each [Xaa]ₓ is KFL, and each [Xaa]_{y} is SAKKFGKAX₃VGEX₄BNS (SEQ ID NO:179) or SAKKFGKAX₃VGEX₄ANS (SEQ ID NO:180), wherein each of X₃ and X₄ is a stapling amino acid, and wherein a side chain of X₃ is cross-linked to a side chain of X₄;

(mm) each [Xaa]_{w} is GIX₁KFLX₂SAKKFGKA, wherein each of X₁ and X₂ is a stapling amino acid (SEQ ID NO:181), and wherein a side chain of X₁ is cross-linked to a side chain of X₂, each [Xaa]ₓ is VGE, and each [Xaa]_{y} is BNS or ANS;

(nn) each [Xaa]_{w} is GI, each [Xaa]ₓ is KFL, and each [Xaa]_{y} is KAKKFGKAX₃VGEX₄BNS (SEQ ID NO:182) or KAKKFGKAX₃VGEX₄ANS (SEQ ID NO:183), wherein each of X₃ and X₄ is a stapling amino acid, and wherein a side chain of X₃ is cross-linked to a side chain of X₄;

(oo) each [Xaa]_{w} is GIX₁KFLX₂KAKKFGKA, wherein each of X₁ and X₂ is a stapling amino acid (SEQ ID NO:184), and wherein a side chain of X₁ is cross-linked to a side chain of X₂, each [Xaa]ₓ is VGE, and each [Xaa]_{y} is BNS or ANS;

(pp) each [Xaa]_{w} is GI, each [Xaa]ₓ is KFL, and each [Xaa]_{y} is SKKKFGKAX₃VGEX₄BNS (SEQ ID NO:185) or SKKKFGKAX₃VGEX₄ANS (SEQ ID NO:186), wherein each of X₃ and X₄ is a stapling amino acid, and wherein a side chain of X₃ is cross-linked to a side chain of X₄;

(qq) each [Xaa]_{w} is GIX₁KFLX₂SKKKFGKA, wherein each of X₁ and X₂ is a stapling amino acid (SEQ ID NO:187), wherein a side chain of X₁ is cross-linked to a side chain of X₂, each [Xaa]ₓ is VGE, and each [Xaa]_{y} is BNS or ANS;

(rr) each [Xaa]_{w} is G, each [Xaa]ₓ is GFK, and each [Xaa]_{y} is HSKKKFGKAX₃VGEX₄BNS (SEQ ID NO:188) or HSKKKFGKAX₃VGEX₄ANS (SEQ ID NO:189), wherein each of X₃ and X₄ is a stapling amino acid, and wherein a side chain of X₃ is cross-linked to a side chain of X₄; or

(ss) each [Xaa]_{w} is GX₁GKFX₂HSKKKFGKA, wherein each of X₁ and X₂ is a stapling amino acid (SEQ ID NO:190), and wherein a side chain of X₁ is cross-linked to a side chain of X₂, each [Xaa]ₓ is VGE, and each [Xaa]_{y} is BNS or ANS;

(tt) each [Xaa]_{w} is GIG, each [Xaa]ₓ is FLH, and each [Xaa]_{y} is AKKFGKAFVGEIBNS (SEQ ID NO:207) or AKKFGKAFVGEIANS (SEQ ID NO:213);

(uu) each [Xaa]_{w} is GIGKFLH (SEQ ID NO:200), each [Xaa]ₓ is AKK, and each [Xaa]_{y} is GKAFVGEIBNS (SEQ ID NO:208) or GKAFVGEIANS (SEQ ID NO:214);

(vv) each [Xaa]_{w} is GIGKFLHS (SEQ ID NO:201), each [Xaa]ₓ is KKF, and each [Xaa]_{y} is KAFVGEIBNS (SEQ ID NO:209) or KAFVGEIANS (SEQ ID NO:215);

(ww) each [Xaa]_{w} is GIGKFLHSA (SEQ ID NO:202), each [Xaa]ₓ is KFG, and each [Xaa]_{y} is AFVGEIBNS (SEQ ID NO:210) or AFVGEIANS (SEQ ID NO:216);

(xx) each [Xaa]_{w} is GIGKFLHSAKKFG (SEQ ID NO:203), each [Xaa]ₓ is AFV, and each [Xaa]_{y} is EIBNS (SEQ ID NO:211) or EIANS (SEQ ID NO:217);

(yy) each [Xaa]_{w} is GIGKFLHSAKKFGK (SEQ ID NO:204), each [Xaa]ₓ is FVG, and each [Xaa]_{y} is IBNS (SEQ ID NO:212) or IANS (SEQ ID NO:218);

(zz) each [Xaa]_{w} is GIGKFLHSAKKFGKAFV (SEQ ID NO:205), each [Xaa]ₓ is EIB, and each [Xaa]_{y} is S;

(aaa) each [Xaa]_{w} is GIGKFLHSAKKFGKAFVG (SEQ ID NO:206), each [Xaa]ₓ is IBN, and each [Xaa]_{y} is absent;

(bbb) each [Xaa]_{w} is absent, each [Xaa]ₓ is IGKFLH (SEQ ID NO:237), and each [Xaa]_{y} is AKKFGKAFVGEIBNS (SEQ ID NO:253) or AKKFGKAFVGEIANS (SEQ ID NO:279);

(ccc) each [Xaa]_{w} is G, each [Xaa]ₓ is GKFLHS (SEQ ID NO:238), and each [Xaa]_{y} is KKFGKAFVGEIBNS (SEQ ID NO:254) or KKFGKAFVGEIANS (SEQ ID NO:280);

(ddd) each [Xaa]_{w} is GI, each [Xaa]ₓ is KFLHSA (SEQ ID NO:239), and each [Xaa]_{y} is KFGKAFVGEIBNS (SEQ ID NO:255) or KFGKAFVGEIANS (SEQ ID NO:281);

(eee) each [Xaa]_{w} is GIG, each [Xaa]ₓ is FLHSAK (SEQ ID NO:240), and each [Xaa]_{y} is FGKAFVGEIBNS (SEQ ID NO:256) or FGKAFVGEIANS (SEQ ID NO:282);

(fff) each [Xaa]_{w} is GIGK (SEQ ID NO:39), each [Xaa]ₓ is LHSAKK (SEQ ID NO:241), and each [Xaa]_{y} is GKAFVGEIBNS (SEQ ID NO:257) or GKAFVGEIANS (SEQ ID NO:283);

(ggg) each [Xaa]_{w} is GIGKF (SEQ ID NO:234), each [Xaa]ₓ is HSAKKF (SEQ ID NO:242), and each [Xaa]_{y} is KAFVGEIBNS (SEQ ID NO:258) or KAFVGEIANS (SEQ ID NO:284);

(hhh) each [Xaa]_{w} is GIGKFL (SEQ ID NO:78), each [Xaa]ₓ is SAKKFG (SEQ ID NO:243), and each [Xaa]_{y} is AFVGEIBNS (SEQ ID NO:259) or AFVGEIANS (SEQ ID NO:285);

(iii) each [Xaa]_{w} is GIGKFLH (SEQ ID NO:200), each [Xaa]ₓ is AKKFGK (SEQ ID NO:244), and each [Xaa]_{y} is FVGEIBNS (SEQ ID NO:260) or FVGEIANS (SEQ ID NO:286);

(jjj) each [Xaa]_{w} is GIGKFLHS (SEQ ID NO:201), each [Xaa]ₓ is KKFGKA (SEQ ID NO:245), and each [Xaa]_{y} is VGEIBNS (SEQ ID NO:261) or VGEIANS (SEQ ID NO:287);

(kkk) each [Xaa]_{w} is GIGKFLHSA (SEQ ID NO:202), each [Xaa]ₓ is KFGKAF (SEQ ID NO:246), and each [Xaa]_{y} is GEIBNS (SEQ ID NO:262) or GEIANS (SEQ ID NO:288);

(lll) each [Xaa]_{w} is GIGKFLHSAK (SEQ ID NO:235), each [Xaa]ₓ is FGKAFV (SEQ ID NO:247), and each [Xaa]_{y} is EIBNS (SEQ ID NO:263) or EIANS (SEQ ID NO:289);

(mmm) each [Xaa]_{w} is GIGKFLHSAKK (SEQ ID NO:80), each [Xaa]ₓ is GKAFVG (SEQ ID NO:248), and each [Xaa]_{y} is IBNS (SEQ ID NO:264) or IANS (SEQ ID NO:290);

(nnn) each [Xaa]_{w} is GIGKFLHSAKKF (SEQ ID NO:236), each [Xaa]ₓ is KAFVGE (SEQ ID NO:249), and each [Xaa]_{y} is BNS or ANS;

(ooo) each [Xaa]_{w} is GIGKFLHSAKKFG (SEQ ID NO:203), each [Xaa]ₓ is AFVGEI (SEQ ID NO:250), and each [Xaa]_{y} is NS;

(ppp) each [Xaa]_{w} is GIGKFLHSAKKFGK (SEQ ID NO:204), each [Xaa]ₓ is FVGEIB (SEQ ID NO:251) or FVGEIA (SEQ ID NO:277), and each [Xaa]_{y} is S;

(qqq) each [Xaa]_{w} is GIGKFLHSAKKFGKA (SEQ ID NO:82), each [Xaa]ₓ is VGEIBN (SEQ ID NO:252) or VGEIAN (SEQ ID NO:278), and each [Xaa]_{y} is absent;

(rrr) each [Xaa]_{w} is G, each [Xaa]ₓ is GSF, and each [Xaa]_{y} is KKKAHVGKHX₁GKAX₂LTHYL, wherein each of X₁ and X₂ is a stapling amino acid (SEQ ID NO:270), wherein a side chain of X₁ is cross-linked to a side chain of X₂;

(sss) each [Xaa]_{w} is GX₁GSFX₂KKKAHVGKH, wherein each of X₁ and X₂ is a stapling amino acid (SEQ ID NO:265), wherein a side chain of X₁ is cross-linked to a side chain of X₂, each [Xaa]ₓ is GKA, and each [Xaa]_{y} is LTHYL (SEQ ID NO:271);

(ttt) each [Xaa]_{w} is G, each [Xaa]ₓ is GSF, and each [Xaa]_{y} is KKAAHVGKHX₁GKAX₂LTHYL, wherein each of X₁ and X₂ is a stapling amino acid (SEQ ID NO:291), wherein a side chain of X₁ is cross-linked to a side chain of X₂;

(uuu) each [Xaa]_{w} is GX₁GSFX₂KKAAHVGKH, wherein each of X₁ and X₂ is a stapling amino acid (SEQ ID NO:266), wherein a side chain of X₁ is cross-linked to a side chain of X₂, each [Xaa]ₓ is GKA, and each [Xaa]_{y} is LTHYL (SEQ ID NO:272);

(vvv) each [Xaa]_{w} is GWGSFFKKAAHV (SEQ ID NO:267), each [Xaa]ₓ is KHV, and each [Xaa]_{y} is KAALTHYL (SEQ ID NO:273);

(www) each [Xaa]_{w} is G, each [Xaa]ₓ is RKR, and each [Xaa]_{y} is RKFRNKIKEKKKKIGQKX₁QGLX₂PKLA, wherein each of X₁ and X₂ is a stapling amino acid (SEQ ID NO:274), wherein a side chain of X₁ is cross-linked to a side chain of X₂;

(xxx) each [Xaa]_{w} is GX₁RKRX₂RKFRNKIKEKKKKIGQK, wherein each of X₁ and X₂ is a stapling amino acid (SEQ ID NO:268), wherein a side chain of X₁ is cross-linked to a side chain of X₂, each [Xaa]ₓ is QGL, and each [Xaa]_{y} is PKLA (SEQ ID NO:275);

(yyy) each [Xaa]_{w} is G, each [Xaa]ₓ is FSK, and each [Xaa]_{y} is KGKKIKNLX₁ISGX₂KG, wherein each of X₁ and X₂ is a stapling amino acid (SEQ ID NO:276), wherein a side chain of X₁ is cross-linked to a side chain of X₂; or

(zzz) each [Xaa]_{w} is GX₁FSKX₂KGKKIKNL, wherein each of X₁ and X₂ is a stapling amino acid (SEQ ID NO:269), wherein a side chain of X₁ is cross-linked to a side chain of X₂, each [Xaa]ₓ is ISG, and each [Xaa]_{y} is KG; and

wherein B is norleucine. In some instances of Formula (I), R₁ is an alkyl. In some instances of Formula (I), R₁ is a methyl group. In some instances of Formula (I), R₂ is an alkyl. In some instances of Formula (I), R₂ is a methyl group. In some instances of Formula (I), R₃ is an alkyl or alkenyl. In some instances of Formula (I), R₃ is —CH2-CH2-CH2-CH═CH—CH2-CH2-CH2-. In some instances of Formula (I), R₁ is a methyl group, R₂ is methyl, and R₃ is a —CH2-CH2-CH2-CH═CH—CH2-CH2-CH2-. In some instances of Formula (I), z is 1.

In another aspect of Formula (I), the two alpha, alpha disubstituted stereocenters are both in the R configuration or S configuration (e.g., i, i+4 cross-link), or one stereocenter is R and the other is S (e.g., i, i+4 cross-link). Thus, where Formula (I) is depicted as:

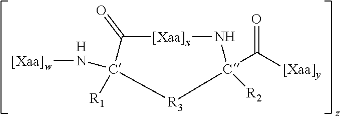

the C′ and C″ disubstituted stereocenters can both be in the R configuration or they can both be in the S configuration, e.g., when x is 3. When x is 6 in Formula (I), the C′ disubstituted stereocenter is in the R configuration and the C″ disubstituted stereocenter is in the S configuration. The R₃ double bond of Formula (I) can be in the E or Z stereochemical configuration.

In some instances of Formula (I), R₃ is [R₄—K—R₄]ₙ; and R₄ is a straight chain alkyl, alkenyl, or alkynyl.

As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some instances, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some instances, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some instances, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some instances, the alkyl portion has 1 to 4, 1 to 3, 1 to 2, or 1 carbon atom(s). In some instances, the alkyl portion is methylene. In some instances, the cycloalkyl portion has 3 to 10 ring members or 3 to 7 ring members. In some instances, the cycloalkyl group is monocyclic or bicyclic. In some instances, the cycloalkyl portion is monocyclic. In some instances, the cycloalkyl portion is a C₃₋₇ monocyclic cycloalkyl group.

As used herein, the term “heteroarylalkyl,” employed alone or in combination with other terms, refers to a group of formula heteroaryl-alkyl-. In some instances, the alkyl portion has 1 to 4, 1 to 3, 1 to 2, or 1 carbon atom(s). In some instances, the alkyl portion is methylene. In some instances, the heteroaryl portion is a monocyclic or bicyclic group having 1, 2, 3, or 4 heteroatoms independently selected from nitrogen, sulfur and oxygen. In some instances, the heteroaryl portion has 5 to 10 carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some instances, halo is F or Cl.

In some instances, the disclosure features structurally-stabilized (e.g., stapled) peptides comprising the amino acid sequence of any one of SEQ ID NOs:1, 63-75, 100, 123-127, and 312-314 (or a modified version thereof), wherein: the side chains of two amino acids separated by two, three, or six amino acids are replaced by an internal staple, the side chains of three amino acids are replaced by an internal stitch, the side chains of four amino acids are replaced by two internal staples, or the side chains of five amino acids are replaced by the combination of an internal staple and an internal stitch. In some instances, the disclosure features structurally-stabilized (e.g., stapled) peptides comprising the amino acid sequence of any one of SEQ ID NOs: 1, 63-75, 100, 123-127, and 312-314 (or a modified version thereof), wherein the side chains of two amino acids separated by two, three, or six amino acids are replaced by an internal staple. In some instances, the disclosure features structurally-stabilized (e.g., stapled) peptides comprising the amino acid sequence of any one of SEQ ID NOs: 1, 63-75, 100, 123-127, and 312-314 (or a modified version thereof), wherein the side chains of two amino acids separated by three amino acids are replaced by an internal staple. In some instances, the disclosure features structurally-stabilized (e.g., stapled) peptides comprising the amino acid sequence of any one of SEQ ID NOs:1, 63-75, 100, 123-127, and 312-314 (or a modified version thereof), wherein the side chains of two amino acids separated by six amino acids are replaced by an internal staple. The stapled or stitched peptide can be 5 or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In a specific instance, the stapled or stitched peptide is 5-30 (i.e., 5, 6, 7, 8, 9, 10, 11, 12, 13, 14, 15, 16, 17, 18, 19, 20, 21, 22, 23, 24, 25, 26, 27, 28, 29, 30) in length. In a specific instance, the stapled or stitched peptide is 10-30 (i.e., 10, 11, 12, 13, 14, 15, 16, 17, 18, 19, 20, 21, 22, 23, 24, 25, 26, 27, 28, 29, 30) amino acids in length. In a specific instance, the stapled or stitched peptide is 15-30 (i.e., 15, 16, 17, 18, 19, 20, 21, 22, 23, 24, 25, 26, 27, 28, 29, 30) amino acids in length. In a specific instance, the stapled or stitched peptide is 5-23 (i.e., 5, 6, 7, 8, 9, 10, 11, 12, 13, 14, 15, 16, 17, 18, 19, 20, 21, 22, 23) in length. In a specific instance, the stapled or stitched peptide is 10-30 (i.e., 10, 11, 12, 13, 14, 15, 16, 17, 18, 19, 20, 21, 22, 23) amino acids in length. In a specific instance, the stapled or stitched peptide is 15-23 (i.e., 15, 16, 17, 18, 19, 20, 21, 22, 23) amino acids in length. In a specific instance, the stapled or stitched peptide is 23-30 (i.e., 23, 24, 25, 26, 27, 28, 29, or 30) amino acids in length. In a specific instance, the stapled or stitched peptide is 23 amino acids in length. Exemplary stapled peptides are shown in FIG. 1, FIG. 5A, FIG. 5B, FIG. 9, FIG. 11, FIG. 16, FIG. 17A, FIG. 17B, and Table 2, and are described in the Formula (I) constructs of Table 3. In one instance, the stapled peptide comprises or consists of a stapled version of the amino acid sequence of any one of SEQ ID NOs: 2-31, 33-38, 40-62, 98, 99, 101-121, and 133-158 (e.g., the product of a ring-closing metathesis reaction(s) performed on a peptide comprising the amino acid sequence of any one of SEQ ID NOs: 2-31, 33-38, 40-62, 98, 99, 101-121, and 133-158, respectively). In one instance, the stapled peptide comprises or consists of a stapled version of the amino acid sequence of any one of SEQ ID NOs: 2-6, 8-11, 13, 15-23, 26, 27, 31, 34, 35, 37, 38, 42, 46, 47, 54, 56, and 58-60 (e.g., the product of a ring-closing metathesis reaction(s) performed on a peptide comprising the amino acid sequence of any one of SEQ ID NOs: 2-6, 8-11, 13, 15-23, 26, 27, 31, 34, 35, 37, 38, 42, 46, 47, 54, 56, and 58-60, respectively). In one instance, the stapled peptide comprises or consists of a stapled version of the amino acid sequence of any one of SEQ ID NOs: 133-145 (e.g., the product of a ring-closing metathesis reaction(s) performed on a peptide comprising the amino acid sequence of any one of SEQ ID NOs: 133-145, respectively). In one instance, the stapled peptide comprises or consists of a stapled version of the amino acid sequence of any one of SEQ ID NOs: 101-121, 128-132, and 192-199 (e.g., the product of a ring-closing metathesis reaction(s) performed on a peptide comprising the amino acid sequence of any one of SEQ ID NOs: 101-121, 128-132, and 192-199, respectively). In one instance, the stapled peptide comprises or consists of a stapled version of the amino acid sequence of any one of SEQ ID NOs: 146-158 (e.g., the product of a ring-closing metathesis reaction(s) performed on a peptide comprising the amino acid sequence of any one of SEQ ID NOs: 146-158, respectively). In one instance, the stapled peptide comprises or consists of a stapled version of an amino acid sequence shown in FIG. 1, FIG. 5A, FIG. 5B, FIG. 9, FIG. 11, FIG. 16, FIG. 17A, or FIG. 17B (e.g., the product of a ring-closing metathesis reaction(s) performed on a peptide comprising the amino acid sequence of any one of SEQ ID NOs: 133-145, respectively).

In one instance, the stapled peptide comprises or consists of a stapled version of the amino acid sequence of SEQ ID NO:2 (e.g., the product of a ring-closing metathesis reaction(s) performed on a peptide comprising the amino acid sequence of SEQ ID NO:2). In one instance, the stapled peptide comprises or consists of a stapled version of the amino acid sequence of SEQ ID NO:3 (e.g., the product of a ring-closing metathesis reaction(s) performed on a peptide comprising the amino acid sequence of SEQ ID NO:3). In one instance, the stapled peptide comprises or consists of a stapled version of the amino acid sequence of SEQ ID NO:4 (e.g., the product of a ring-closing metathesis reaction(s) performed on a peptide comprising the amino acid sequence of SEQ ID NO:4). In one instance, the stapled peptide comprises or consists of a stapled version of the amino acid sequence of SEQ ID NO:5 (e.g., the product of a ring-closing metathesis reaction(s) performed on a peptide comprising the amino acid sequence of SEQ ID NO:5). In one instance, the stapled peptide comprises or consists of a stapled version of the amino acid sequence of SEQ ID NO:6 (e.g., the product of a ring-closing metathesis reaction(s) performed on a peptide comprising the amino acid sequence of SEQ ID NO:6). In one instance, the stapled peptide comprises or consists of a stapled version of the amino acid sequence of SEQ ID NO:8 (e.g., the product of a ring-closing metathesis reaction(s) performed on a peptide comprising the amino acid sequence of SEQ ID NO:8). In one instance, the stapled peptide comprises or consists of a stapled version of the amino acid sequence of SEQ ID NO:9 (e.g., the product of a ring-closing metathesis reaction(s) performed on a peptide comprising the amino acid sequence of SEQ ID NO:9). In one instance, the stapled peptide comprises or consists of a stapled version of the amino acid sequence of SEQ ID NO:10 (e.g., the product of a ring-closing metathesis reaction(s) performed on a peptide comprising the amino acid sequence of SEQ ID NO:10). In one instance, the stapled peptide comprises or consists of a stapled version of the amino acid sequence of SEQ ID NO:11 (e.g., the product of a ring-closing metathesis reaction(s) performed on a peptide comprising the amino acid sequence of SEQ ID NO:11). In one instance, the stapled peptide comprises or consists of a stapled version of the amino acid sequence of SEQ ID NO:13 (e.g., the product of a ring-closing metathesis reaction(s) performed on a peptide comprising the amino acid sequence of SEQ ID NO:13). In one instance, the stapled peptide comprises or consists of a stapled version of the amino acid sequence of SEQ ID NO:15 (e.g., the product of a ring-closing metathesis reaction(s) performed on a peptide comprising the amino acid sequence of SEQ ID NO:15). In one instance, the stapled peptide comprises or consists of a stapled version of the amino acid sequence of SEQ ID NO:16 (e.g., the product of a ring-closing metathesis reaction(s) performed on a peptide comprising the amino acid sequence of SEQ ID NO:16). In one instance, the stapled peptide comprises or consists of a stapled version of the amino acid sequence of SEQ ID NO:17 (e.g., the product of a ring-closing metathesis reaction(s) performed on a peptide comprising the amino acid sequence of SEQ ID NO:17). In one instance, the stapled peptide comprises or consists of a stapled version of the amino acid sequence of SEQ ID NO:18 (e.g., the product of a ring-closing metathesis reaction(s) performed on a peptide comprising the amino acid sequence of SEQ ID NO:18). In one instance, the stapled peptide comprises or consists of a stapled version of the amino acid sequence of SEQ ID NO:19 (e.g., the product of a ring-closing metathesis reaction(s) performed on a peptide comprising the amino acid sequence of SEQ ID NO:19). In one instance, the stapled peptide comprises or consists of a stapled version of the amino acid sequence of SEQ ID NO:20 (e.g., the product of a ring-closing metathesis reaction(s) performed on a peptide comprising the amino acid sequence of SEQ ID NO:20). In one instance, the stapled peptide comprises or consists of a stapled version of the amino acid sequence of SEQ ID NO:21 (e.g., the product of a ring-closing metathesis reaction(s) performed on a peptide comprising the amino acid sequence of SEQ ID NO:21). In one instance, the stapled peptide comprises or consists of a stapled version of the amino acid sequence of SEQ ID NO:22 (e.g., the product of a ring-closing metathesis reaction(s) performed on a peptide comprising the amino acid sequence of SEQ ID NO:22). In one instance, the stapled peptide comprises or consists of a stapled version of the amino acid sequence of SEQ ID NO:23 (e.g., the product of a ring-closing metathesis reaction(s) performed on a peptide comprising the amino acid sequence of SEQ ID NO:23). In one instance, the stapled peptide comprises or consists of a stapled version of the amino acid sequence of SEQ ID NO:26 (e.g., the product of a ring-closing metathesis reaction(s) performed on a peptide comprising the amino acid sequence of SEQ ID NO:26). In one instance, the stapled peptide comprises or consists of a stapled version of the amino acid sequence of SEQ ID NO:27 (e.g., the product of a ring-closing metathesis reaction(s) performed on a peptide comprising the amino acid sequence of SEQ ID NO:27). In one instance, the stapled peptide comprises or consists of a stapled version of the amino acid sequence of SEQ ID NO:31 (e.g., the product of a ring-closing metathesis reaction(s) performed on a peptide comprising the amino acid sequence of SEQ ID NO:31). In one instance, the stapled peptide comprises or consists of a stapled version of the amino acid sequence of SEQ ID NO:34 (e.g., the product of a ring-closing metathesis reaction(s) performed on a peptide comprising the amino acid sequence of SEQ ID NO:34). In one instance, the stapled peptide comprises or consists of a stapled version of the amino acid sequence of SEQ ID NO:35 (e.g., the product of a ring-closing metathesis reaction(s) performed on a peptide comprising the amino acid sequence of SEQ ID NO:35). In one instance, the stapled peptide comprises or consists of a stapled version of the amino acid sequence of SEQ ID NO:37 (e.g., the product of a ring-closing metathesis reaction(s) performed on a peptide comprising the amino acid sequence of SEQ ID NO:37). In one instance, the stapled peptide comprises or consists of a stapled version of the amino acid sequence of SEQ ID NO:38 (e.g., the product of a ring-closing metathesis reaction(s) performed on a peptide comprising the amino acid sequence of SEQ ID NO:38). In one instance, the stapled peptide comprises or consists of a stapled version of the amino acid sequence of SEQ ID NO:42 (e.g., the product of a ring-closing metathesis reaction(s) performed on a peptide comprising the amino acid sequence of SEQ ID NO:42). In one instance, the stapled peptide comprises or consists of a stapled version of the amino acid sequence of SEQ ID NO:46 (e.g., the product of a ring-closing metathesis reaction(s) performed on a peptide comprising the amino acid sequence of SEQ ID NO:46). In one instance, the stapled peptide comprises or consists of a stapled version of the amino acid sequence of SEQ ID NO:47 (e.g., the product of a ring-closing metathesis reaction(s) performed on a peptide comprising the amino acid sequence of SEQ ID NO:47). In one instance, the stapled peptide comprises or consists of a stapled version of the amino acid sequence of SEQ ID NO:54 (e.g., the product of a ring-closing metathesis reaction(s) performed on a peptide comprising the amino acid sequence of SEQ ID NO:54). In one instance, the stapled peptide comprises or consists of a stapled version of the amino acid sequence of SEQ ID NO:56 (e.g., the product of a ring-closing metathesis reaction(s) performed on a peptide comprising the amino acid sequence of SEQ ID NO:56). In one instance, the stapled peptide comprises or consists of a stapled version of the amino acid sequence of SEQ ID NO:58 (e.g., the product of a ring-closing metathesis reaction(s) performed on a peptide comprising the amino acid sequence of SEQ ID NO:58). In one instance, each of X₁ and X₂ is (S)-2-(4′-pentenyl)Alanine.

In one instance, the stapled peptide comprises or consists of a stapled version of the amino acid sequence of SEQ ID NO:60 (e.g., the product of a ring-closing metathesis reaction(s) performed on a peptide comprising the amino acid sequence of SEQ ID NO:60). In one instance, the stapled peptide comprises or consists of a stapled version of the amino acid sequence of SEQ ID NO:59 (e.g., the product of a ring-closing metathesis reaction(s) performed on a peptide comprising the amino acid sequence of SEQ ID NO:59). In one instance, each of X₁, X₂, X₃, and X₄ is (S)-2-(4′-pentenyl)Alanine.

Exemplary stapled peptides are shown in Table 2. In one instance, the stapled peptide comprises or consists of the amino acid sequence of SEQ ID NO:2 or 101. In one instance, the stapled peptide comprises or consists of the amino acid sequence of SEQ ID NO:3 or 102. In one instance, the stapled peptide comprises or consists of the amino acid sequence of SEQ ID 15 NO:4 or 103. In one instance, the stapled peptide comprises or consists of the amino acid sequence of SEQ ID NO:6 or 104. In one instance, the stapled peptide comprises or consists of the amino acid sequence of SEQ ID NO:8 or 105. In one instance, the stapled peptide comprises or consists of the amino acid sequence of SEQ ID NO:13 or 106. In one instance, the stapled peptide comprises or consists of the amino acid sequence of SEQ ID NO:17 or 107. In one instance, the stapled peptide comprises or consists of the amino acid sequence of SEQ ID NO:21 or 109. In one instance, the stapled peptide comprises or consists of the amino acid sequence of SEQ ID NO:22 or 110. In one instance, the stapled peptide comprises or consists of the amino acid sequence of SEQ ID NO:23 or 111. In one instance, the stapled peptide comprises or consists of the amino acid sequence of SEQ ID NO:26 or 112. In one instance, the stapled peptide comprises or consists of the amino acid sequence of SEQ ID NO:27 or 113. In one instance, the stapled peptide comprises or consists of the amino acid sequence of SEQ ID NO:35 or 114. In one instance, the stapled peptide comprises or consists of the amino acid sequence of SEQ ID NO:38 or 115. In one instance, the stapled peptide comprises or consists of the amino acid sequence of SEQ ID NO:42 or 116. In one instance, the stapled peptide comprises or consists of the amino acid sequence of SEQ ID NO:46 or 117. In one instance, the stapled peptide comprises or consists of the amino acid sequence of SEQ ID NO:47 or 118. In one instance, the stapled peptide comprises or consists of the amino acid sequence of SEQ ID NO:56 or 119. In one instance, the stapled peptide comprises or consists of the amino acid sequence of SEQ ID NO:58 or 120. In one instance, the stapled peptide comprises or consists of the amino acid sequence of SEQ ID NO:18 or 108. In one instance, the stapled peptide comprises or consists of the amino acid sequence of SEQ ID NO:60 or 121. In one instance, the stapled peptide comprises or consists of the amino acid sequence of SEQ ID NO:5 or 192. In one instance, the stapled peptide comprises or consists of the amino acid sequence of SEQ ID NO:9 or 193. In one instance, the stapled peptide comprises or consists of the amino acid sequence of SEQ ID NO:10 or 194. In one instance, the stapled peptide comprises or consists of the amino acid sequence of SEQ ID NO:11 or 195. In one instance, the stapled peptide comprises or consists of the amino acid sequence of SEQ ID NO:15 or 196. In one instance, the stapled peptide comprises or consists of the amino acid sequence of SEQ ID NO:16 or 197. In one instance, the stapled peptide comprises or consists of the amino acid sequence of SEQ ID NO:19 or 198. In one instance, the stapled peptide comprises or consists of the amino acid sequence of SEQ ID NO:20 or 199.

In certain instances, the stapled peptide comprises or consists of a variant of the amino acid sequence set forth in any one of SEQ ID NOs: 63-75, 100, 123-127, and 312-314, wherein two amino acids each separated by 3 amino acids (i.e., positions i and i+4) are modified to structurally stabilize the peptide (e.g., by substituting them with non-natural amino acids to permit hydrocarbon stitching, i.e., stapling amino acids). In certain instances, the two amino acids each separated by three amino acids are at the amino acid positions in the magainin II peptide corresponding to positions 1 and 5 of the amino acid sequence of SEQ ID NO:1. In certain instances, the two amino acids each separated by three amino acids are at the amino acid positions in the magainin II peptide corresponding to positions 2 and 6 of the amino acid sequence of SEQ ID NO:1. In certain instances, the two amino acids each separated by three amino acids are at the amino acid positions in the magainin II peptide corresponding to positions 3 and 7 of the amino acid sequence of SEQ ID NO:1. In certain instances, the two amino acids each separated by three amino acids are at the amino acid positions in the magainin II peptide corresponding to positions 4 and 8 of the amino acid sequence of SEQ ID NO:1. In certain instances, the two amino acids each separated by three amino acids are at the amino acid positions in the magainin II peptide corresponding to positions 5 and 9 of the amino acid sequence of SEQ ID NO:1. In certain instances, the two amino acids each separated by three amino acids are at the amino acid positions in the magainin II peptide corresponding to positions 7 and 11 of the amino acid sequence of SEQ ID NO:1. In certain instances, the two amino acids each separated by three amino acids are at the amino acid positions in the magainin II peptide corresponding to positions 8 and 12 of the amino acid sequence of SEQ ID NO:1. In certain instances, the two amino acids each separated by three amino acids are at the amino acid positions in the magainin II peptide corresponding to positions 9 and 13 of the amino acid sequence of SEQ ID NO:1. In certain instances, the two amino acids each separated by three amino acids are at the amino acid positions in the magainin II peptide corresponding to positions 10 and 14 of the amino acid sequence of SEQ ID NO:1. In certain instances, the two amino acids each separated by three amino acids are at the amino acid positions in the magainin II peptide corresponding to positions 12 and 16 of the amino acid sequence of SEQ ID NO:1. In certain instances, the two amino acids each separated by three amino acids are at the amino acid positions in the magainin II peptide corresponding to positions 14 and 18 of the amino acid sequence of SEQ ID NO:1. In certain instances, the two amino acids each separated by three amino acids are at the amino acid positions in the magainin II peptide corresponding to positions 15 and 19 of the amino acid sequence of SEQ ID NO:1. In certain instances, the two amino acids each separated by three amino acids are at the amino acid positions in the magainin II peptide corresponding to positions 16 and 20 of the amino acid sequence of SEQ ID NO:1. In certain instances, the two amino acids each separated by three amino acids are at the amino acid positions in the magainin II peptide corresponding to positions 17 and 21 of the amino acid sequence of SEQ ID NO:1. In certain instances, the two amino acids each separated by three amino acids are at the amino acid positions in the magainin II peptide corresponding to positions 18 and 22 of the amino acid sequence of SEQ ID NO:1. In certain instances, the two amino acids each separated by three amino acids are at the amino acid positions in the magainin II peptide corresponding to positions 19 and 23 of the amino acid sequence of SEQ ID NO:1.

In certain instances, the stapled peptide comprises or consists of a variant of the amino acid sequence set forth in any one of SEQ ID NOs: 63-75, 100, 123-127, and 312-314, wherein two amino acids each separated by 6 amino acids (i.e., positions i and i+7) are modified to structurally stabilize the peptide (e.g., by substituting them with non-natural amino acids to permit hydrocarbon stapling/stitching, i.e., stapling amino acids).

In certain instances, the stapled peptide comprises or consists of a variant of the amino acid sequence set forth in SEQ ID NO:75, wherein two pairs of amino acids are modified to structurally stabilize the peptide (e.g., by substituting them with non-natural amino acids to permit hydrocarbon stitching, i.e., stapling amino acids), wherein the two amino acids of each pair are separated by 3 amino acids (i.e., positions i and i+4). In certain instances, the four amino acids are at the amino acid positions in the magainin II peptide corresponding to positions 2, 6, 16, and 20 of the amino acid sequence of SEQ ID NO:1. In certain instances, the four amino acids are at the amino acid positions in the magainin II peptide corresponding to positions 1, 5, 16, and 20 of the amino acid sequence of SEQ ID NO:1. In certain instances, the four amino acids are at the amino acid positions in the magainin II peptide corresponding to positions 3, 7, 16, and 20 of the amino acid sequence of SEQ ID NO:1.

In some instances, the structurally-stabilized (e.g., stapled) oncolytic peptides described herein specifically lyse cancer cells (e.g., hematological cancer cells, e.g., leukemia cells, lymphoma cells, multiple myeloma cells). In some instances, the structurally-stabilized peptides specifically lyse hematological cancer cells. In some instances, the structurally-stabilized peptides specifically lyse leukemia cells. In some instances, the structurally-stabilized peptides specifically lyse lymphoma cells. In some instances, the structurally-stabilized peptides specifically lyse multiple myeloma cells. A structurally-stabilized oncolytic peptide specifically lyses cancer cells if it is selective for lysing cancer cell membranes versus lysing non-cancerous mammalian cell (e.g., red blood cell or kidney cell) membranes. In other words, a structurally-stabilized peptide specifically lyses cancer cells if it is able to lyse or inhibit the growth of a cancer cell while also having a relatively low ability to lyse or inhibit the growth of a non-cancerous mammalian cell (e.g., a red blood cell or a kidney cell). In some instances, a structurally-stabilized peptide specifically lyses cancer cells if it lyses at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher cancer cells at a particular concentration of structurally-stabilized peptide than non-cancerous mammalian cells (e.g., red blood cells or kidney cells) at the same concentration of the structurally-stabilized peptide. An anti-cancer (e.g., anti-leukemia, anti-leukemia) peptide selective for cancer cell membranes versus non-cancerous mammalian cell membranes (e.g., red blood cell or kidney cell membranes) can have a therapeutic concentration of, e.g., about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 12 μg/mL, about 14 μg/mL, about 16 μg/mL, about 18 μg/mL, about 20 μg/mL, about 22 μg/mL, about 24 μg/mL, about 26 μg/mL, about 28 μg/mL, or about 30 μg/mL. An anti-cancer peptide selective for cancer cell membranes versus non-cancerous mammalian cell (e.g., red blood cell or kidney cell) membranes may lyse, e.g., less than about 50%, less than about 40%, less than about 30%, less than about 25%, less than about 20%, less than about 15%, less than about 10%, less than about 5%, less than about 2.5%, less than about 2%, or less than about 1% of red blood cells (RBCs) in a RBC hemolytic activity assay when administered at a concentration, e.g., greater than or approximately equal to 1-fold, 1.5-fold, 2-fold, 2.5-fold, 3-fold, 4-fold, 5-fold, 6-fold, 7-fold, 8-fold, 9-fold, or 10-fold of the concentration required to lyse 50% of cancer cells. An anti-cancer peptide selective for cancer cell membranes versus non-cancerous mammalian cell (e.g., red blood cell or kidney cell) membranes may lyse, e.g., less than about 10% of RBCs in a RBC hemolytic activity assay when administered at a concentration of 100 μg/mL.

In some instances, the structurally-stabilized (e.g., stapled) oncolytic peptides described herein also specifically lyse bacterial cells. A structurally-stabilized oncolytic peptide specifically lyses bacterial cells if it is selective for lysing bacterial cell membranes versus lysing non-cancerous mammalian cell (e.g., red blood cell or kidney cell) membranes. In other words, a structurally-stabilized peptide specifically lyses bacterial cells if it is able to lyse or inhibit the growth of a bacterial cell while also having a relatively low ability to lyse or inhibit the growth of a non-cancerous mammalian cell (e.g., a red blood cell or a kidney cell). In some instances, a structurally-stabilized peptide specifically lyses bacterial cells if it lyses at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher cancer cells at a particular concentration of structurally-stabilized peptide than non-cancerous mammalian cells (e.g., red blood cells or kidney cells) at the same concentration of the structurally-stabilized peptide. A structurally-stabilized oncolytic peptide selective for bacterial cell membranes versus non-cancerous mammalian cell membranes (e.g., red blood cell or kidney cell membranes) can have a therapeutic concentration of, e.g., about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 12 μg/mL, about 14 μg/mL, about 16 μg/mL, about 18 μg/mL, about 20 μg/mL, about 22 μg/mL, about 24 μg/mL, about 26 μg/mL, about 28 μg/mL, or about 30 μg/mL. A structurally-stabilized oncolytic peptide selective for bacterial cell membranes versus non-cancerous mammalian cell (e.g., red blood cell or kidney cell) membranes may lyse, e.g., less than about 50%, less than about 40%, less than about 30%, less than about 25%, less than about 20%, less than about 15%, less than about 10%, less than about 5%, less than about 2.5%, less than about 2%, or less than about 1% of RBCs in a RBC hemolytic activity assay when administered at a concentration, e.g., greater than or approximately equal to 1-fold, 1.5-fold, 2-fold, 2.5-fold, 3-fold, 4-fold, 5-fold, 6-fold, 7-fold, 8-fold, 9-fold, or 10-fold of the concentration required to lyse 50% of bacterial cells. A structurally-stabilized oncolytic peptide selective for bacterial cell membranes versus non-cancerous mammalian cell (e.g., red blood cell or kidney cell) membranes may lyse, e.g., less than about 10% of RBCs in a RBC hemolytic activity assay when administered at a concentration of 100 μg/mL.

To avoid mammalian cell lytic properties and generate cancer-selective (e.g., hematological cancer-selective, e.g., leukemia-selective, lymphoma-selective, multiple myeloma-selective) stabilized (e.g., stapled) AMPs, any of the stabilized (e.g., stapled) AMPs of this document can include an α-helical region that contains a first surface hydrophobic patch. In these stabilized AMPs the replacement of a relevant pair of amino acids by a linking group (e.g., R3 in Formula (I)) results in discontinuity between the first surface hydrophobic patch and an additional one or more (e.g., 2, 3, 4, 5, 6, 8, or 10) surface hydrophobic patches in α-helical region. Referring to Formula (I), such stabilized AMPs can be made, e.g., by determining the location of an established surface hydrophobic patch in an α-helical region of the stabilized AMP, and selecting integers w and y such that all amino acids [Xaa]x are located within the established surface hydrophobic patch. In an alternative method for generating cancer-selective structurally-stabilized AMPs, again referring to Formula (I), the location of two or more (e.g., 3, 4, 5, 6, 8, or 10) established surface hydrophobic patches in an α-helical region of the stabilized AMP is determined and integers w and y are selected such that amino acids [Xaa]x do not connect two or more (e.g., 3, 4, 5, 6, 8, or 10) established surface hydrophobic patches in the α-helical region of the stabilized AMP.

While hydrocarbon tethers are common, other tethers can also be employed in the structurally-stabilized magainin II peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the magainin II peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc., 113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U.S.A, 97:3803-3808 (2000); Double-click stapling: Lau et al., Chem. Sci., 5:1804-1809 (2014); Bis-lactam stapling: J. C. Phelan et al., J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3, i to i+4, and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

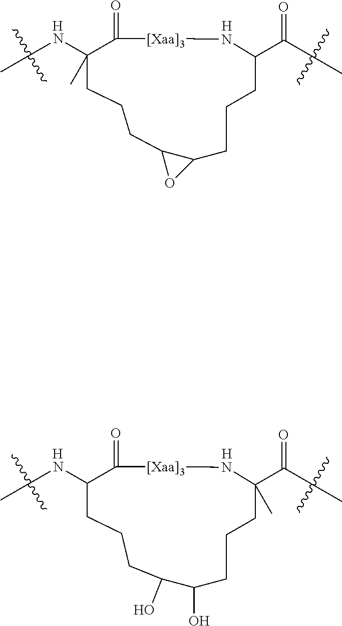

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the peptide or via the amino acid side chain. Other agents can be attached to the functionalized tether, e.g., an agent that facilitates entry of the peptide into cells.

In some instances, α-methyl, α-alkenyl non-natural amino acids are used in the peptide to improve the stability of the alpha helical secondary structure. However, α-methyl, α-alkenyl non-natural amino acids are not required, and instances using mono-alpha substituents (e.g., in the tethered amino acids) are also envisioned.

The structurally-stabilized (e.g., stapled) peptides can include a drug, a toxin, a derivative of polyethylene glycol; a second peptide; a carbohydrate, etc. Where a polymer or other agent is linked to the structurally-stabilized (e.g., stapled) peptide, it can be desirable for the composition to be substantially homogeneous.

The addition of polyethelene glycol (PEG) molecules can improve the pharmacokinetic and pharmacodynamic properties of the peptide. For example, PEGylation can reduce renal clearance and can result in a more stable plasma concentration. PEG is a water soluble polymer and can be represented as linked to the peptide as formula:

XO—(CH₂CH₂O)ₙ—CH₂CH₂—Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the peptide. Y may also be a maleimide linkage to a thiol group (including but not limited to, the thiol group of cysteine). Other methods for linking PEG to a peptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain instances, macromolecular polymer (e.g., PEG) is attached to a structurally-stabilized (e.g., stapled) peptide described herein through an intermediate linker. In certain instances, the linker is made up of from 1 to 20 amino acids linked by peptide bonds, wherein the amino acids are selected from the 20 naturally occurring amino acids. Some of these amino acids may be glycosylated, as is well understood by those in the art. In other instances, the 1 to 20 amino acids are selected from glycine, alanine, proline, asparagine, glutamine, and lysine. In other instances, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

The structurally-stabilized (e.g., stapled) peptides can also be modified, e.g., to further facilitate cellular uptake or increase in vivo stability, in some instances. For example, acylating or PEGylating a structurally-stabilized peptide facilitates cellular uptake, increases bioavailability, increases blood circulation, alters pharmacokinetics, decreases immunogenicity and/or decreases the needed frequency of administration.

In some instances, the structurally-stabilized (e.g., stapled) peptides disclosed herein have an enhanced ability to penetrate cell membranes (e.g., relative to non-stabilized peptides). See, e.g., International Publication No. WO 2017/147283, which is incorporated by reference herein in its entirety.

Methods of synthesizing the structurally-stabilized (e.g., stapled) peptides described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this invention can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH2 protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech. Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The peptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C₁-C₂₀ straight or branched alkyl groups); fatty acid radicals; and combinations thereof. α-methyl, α-alkenyl non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). For peptides where an i linked to i+4 staple is used (two turns of the helix stabilized) either: two (S)-2-(4′-pentenyl)Alanine amino acids are used. Inhibitors are synthesized on a solid support using solid-phase peptide synthesis (SPPS) on MBHA resin (see, e.g., WO 2010/148335).

Fmoc-protected α-amino acids (other than the olefinic amino acids N-Fmoc-α,α-Bis(4′-pentenyl)glycine, (S)—N-Fmoc-α-(4′-pentenyl)alanine, (R)—N-Fmoc-α-(7′-octenyl)alanine, (R)—N-Fmoc-α-(7′-octenyl)alanine, and (R)—N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, Calif.). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stapling or stitching, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some instances, the structurally-stabilized (e.g., stapled) peptides are substantially free of non-structurally-stabilized peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

Also provided herein is a method of producing a structurally-stabilized (e.g., stapled) peptide comprising: (a) stapling or stitching a magainin II peptide (or variant thereof); and (b) isolating the stapled or stitched peptide.

Properties of the stabilized (e.g., stapled) peptides of the invention can be assayed, for example, using the methods described below and in the Examples.

Assays to Determine α-Helicity: Compounds are dissolved in an aqueous solution (e.g., 5 mM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g., temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol. 130:208 (1986)).

Assays to Determine Melting Temperature (Tm): Cross-linked or the unmodified template peptides are dissolved in distilled H₂O or other buffer or solvent (e.g., at a final concentration of 50 μM) and Tm is determined by measuring the change in ellipticity over a temperature range (e.g., 4 to 95° C.) on a spectropolarimeter (e.g., Jasco J-710, Aviv) using standard parameters (e.g., wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In vitro Protease Resistance Assays: The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The stabilized peptides of the present invention may be subjected to in vitro enzymatic proteolysis (e.g., trypsin, chymotrypsin, pepsin) to assess for any change in degradation rate compared to a corresponding unstabilized or alternatively stapled or stitched peptide. For example, the stabilized peptide and a corresponding unstabilized peptide are incubated with trypsin agarose and the reactions quenched at various time points by centrifugation and subsequent HPLC injection to quantitate the residual substrate by ultraviolet absorption at 280 nm. Briefly, the stabilized peptide and its precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Stabilized peptides and/or a corresponding unstabilized peptide can be each incubated with fresh mouse, rat and/or human serum (e.g., 1-2 mL) at 37° C. for, e.g., 0, 1, 2, 4, 8, and 24 hours. Samples of differing stabilized peptide concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N2<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of stabilized peptides in serum.

In vivo Protease Resistance Assays: A key benefit of peptide stapling or stitching is the translation of in vitro protease resistance into markedly improved pharmacokinetics in vivo. Assays for analyzing in vivo protease resistance of a peptide described herein are known in the art.

RBC hemolysis assay: To assess the hemolytic activity of a structurally-stabilized peptide described herein, a red blood cell (RBV) hemolysis assay can be used. Human blood samples are centrifuged to isolate RBCs, which are then washed and suspended in phosphate-buffered saline to yield a 1% (v/v) suspension. The suspension is added to serial dilutions of peptide stocks in water in clear round-bottom polypropylene 96-well plates and the plates incubated for 1 hour at 37° C. The plates are then centrifuged and the supernatant isolated to determine the amount of hemoglobin released using a spectrophotometer (570 nm). Percent hemolysis is calculated as: ([Treated Absorbance−Untreated Control Absorbance]×100)/(1% Triton X-100 Treated Absorbance−Untreated Control Absorbance).

LDH release assay: To assess the release of lactate dehydrogenase (LDH) upon treatment with a structurally-stabilized peptide described herein, an LDH release assay can be used. Cultured cells, including, e.g., cancer cells and HUVEC cells, are plated in 96-well format (2×10⁴ cells per well; including overnight incubation for adherent cells) and then treated with serial dilutions of structurally-stabilized peptides in a final volume in a final volume of 100 μL and incubated at 37° C. for the indicated time period (e.g., 90 minutes). The plates are spun down at 1500 rpm for 5 minutes at 4° C., and 80 μL of cell culture media is transferred to a clear plate (Corning), incubated with 80 μL of LDH reagent (Roche) for 15 minutes while shaking, and absorbance is measured at 490 nm on a microplate reader (SpectraMax M5 Microplate Reader, Molecular Devices). LDH release assays, as tailored to specific cancer cell types (OCI-AML3 cells and primary human pediatric B-ALL cells), are exemplified below.

LDH release assay of OCI-AML3 cells: OCI-AML3 cells are seeded in 96-well plates (2×10⁴ cells per well) in RPMI medium containing 5% FBS and treated with structurally-stabilized peptides for 90 minutes. LDH release is quantified by incubating centrifuged (e.g., at 1500 rpm) cell culture medium 1:1 with LDH reagent (Roche), followed by absorbance measurement 490 nm on a microplate reader (Spectramax M5). Percent LDH release was normalized to 1% Triton.

LDH release assay of primary human pediatric B-ALL cells: Peripheral blood mononuclear cells (PBMCs) are purified from primary human peripheral blood from a pediatric B-ALL patient by density-gradient centrifugation (Ficoll) and plated in 96-well plates (5×10⁴ cells per well). Cells are treated with structurally-stabilized peptide in RPMI medium, containing 5% FBS for 90 minutes. LDH release is quantified by incubating centrifuged (e.g., at 1500 rpm) cell culture medium 1:1 with LDH reagent (Roche), followed by absorbance measurement 490 nm on a microplate reader (Spectramax M5). Percent LDH release was normalized to 1% Triton.

IXM Live/Dead Imaging Assay: An alternative approach to assessing the rapid-onset cytotoxic activity of structurally-stabilized peptides involves high content imaging by epiflorescence microscopy and Image Xpress processing. Cells (e.g., HeLa cells) are plated in 384-well plates (e.g., 3×10³ cells per well) in medium (e.g., DMEM medium containing 5% FBS) along with DRAQ7 (stains only permeabilized, identifies non-viable cells; 0.1 μM) and Hoechst 33342 (stains all nuclei, provides total cell count; 1 μM) and treated with a structurally-stabilized peptide at various concentrations for 120 minutes. Cells are imaged on an ImageXpress Micro (Molecular Devices). Percent dead is plotted by computing DRAQ7-positive cells over total cell count (Hoechst). Treatment with 0.02% or 0.04% Triton leads to all cells being DRAQ7 positive and thus provides the positive control for cell lysis.

PRISM Analysis: To broadly determine the cytotoxicity of structurally-stabilized peptides across human cancer classes and subtypes, Profiling Relative Inhibition Simultaneously in Mixtures (PRISM) analysis is performed as described in Yu et al., Nat. Biotechnol. 2016, 34(4):419-423, which is incorporated by reference herein in its entirety. Compounds are evaluated in an 8-point 3-fold dilution series with a top dose of 40 μM in 384-well plates. Cell sets (PR500 and PR300+) are treated for 3 days, followed by lysis, barcode amplification and detection using a bead-based Luminex system (see Yu et al., Nat. Biotechnol. 2016, 34(4):419-423; Corsello et al., Nat Cancer, 2020, 1: 235-248; Li et al., Nature Medicine, 2019, 25(5):850-860; McFarland et al., Nature Communications, 2020, 11(1):4296; Schauer et al., Nat Chem Biol, 2019, 15: 681-689; Ling et al., Pharmacology & Therapeutics, 2018, 191:178-189; Oberlick et al., Cell Reports, 2019, 28(9):2331-2344).

### Structurally-Stabilized Oncolytic Peptide Variants

In some instances, structurally-stabilized (e.g., stapled) peptides can be made by modifying (e.g., by amino acid substitution) a peptide comprising or consisting of the amino acid sequence of any one of SEQ ID NOs:1, 63-75, 100, 123-127, 219-221, 227-232, and 312-314, or a modified version thereof. In some instances, an internal staple replaces the side chains of 2 amino acids, i.e., each staple is between two amino acids separated by, for example, 2, 3, or 6 amino acids. In some instances, an internal stitch replaces the side chains of 3 amino acids, i.e., the stitch is a pair of crosslinks between three amino acids separated by, for example, 2, 3, or 6 amino acids. In some instances, the internal stitch replaces the side chain of a first amino acid and a second and a third amino acid thereby cross-linking the first amino acid (which lies between the second and third amino acids) to the second and third amino acid via an internal cross-link, wherein the first and second amino acid are separated by two, three, or six amino acids, the first and the third amino acids are separated by three or six amino acids, and the second and third amino acids are distinct amino acids.

The structurally-stabilized (e.g., stapled) peptide comprises at least two modified amino acids (relative to an antimicrobial peptide (e.g., magainin II, pleurocidin, CAP18, esculentin peptide)) joined by an internal intramolecular cross-link (or “staple”), wherein the at least two amino acids are separated by 2, 3, or 6 amino acids. Structurally-stabilized peptides herein include stapled peptides, including peptides having two staples and/or stitched peptides. The at least two modified amino acids can be non-natural alpha-amino acids (including, but not limited to α-methyl, α-alkenyl and N-terminal alkylated amino acids). There are many known non-natural amino acids that may be used as stapling amino acids or stitching amino acids, any of which may be included in the peptides of the present invention. One example of a non-natural amino acid that may be used as a stapling amino acid is an α-methyl, α-alkenyl non-natural amino acid. Some additional examples of non-natural amino acids that may be used as stapling amino acids or stitching amino acids are: (R)-2-(7′-octenyl)Alanine, (S)-2-(7′-octenyl)Alanine, (S)-2-(4′-pentenyl)Alanine, (R)-2-(4′-pentenyl)Alanine, and 2,2-Bis(4′-pentenyl)glycine. In some instances, the amino acids forming the staple (also referred to as the “stapling amino acids”) are (R)-2-(2′-propenyl)alanine and (S)-2-(4′-pentenyl)Alanine at positions i and i+3, respectively, of the staple. In some instances, the amino acids forming the staple are (S)-2-(4′-pentenyl)Alanine and (R)-2-(2′-propenyl)alanine at positions i and i+3, respectively, of the staple. In some instances, the amino acids forming the staple are (R)-2-(4′-pentenyl)Alanine and (S)-2-(4′-pentenyl)Alanine at positions i and i+3, respectively, of the staple. In some instances, the amino acids forming the staple are (S)-2-(4′-pentenyl)Alanine and (R)-2-(4′-pentenyl)Alanine at positions i and i+3, respectively, of the staple. In some instances, the amino acids forming the staple are (S)-2-(4′-pentenyl)Alanine at each of positions i and i+4 of the staple. In some instances, the amino acids forming the staple are (R)-2-(4′-pentenyl)Alanine at each of positions i and i+4 of the staple. In some instances, the amino acids forming the staple or stitch are (R)-2-(7′-octenyl)Alanine and (S)-2-(4′-pentenyl)Alanine at positions i and i+7, respectively, of the staple. In some instances, the amino acids forming the staple or stitch are (S)-2-(7′-octenyl)Alanine and (R)-2-(4′-pentenyl)Alanine at positions i and i+7, respectively, of the staple. In certain instances when a stitch is at the i, i+4, and i+11 residues, (S)-2-(4′-pentenyl)Alanine, 2,2-Bis(4′-pentenyl)glycine, and (S)-2-(7′-octenyl)Alanine are substituted for the amino acids at i, i+4, and i+11 positions, respectively. In certain instances when a stitch is at the i, i+7, and i+11 residues, (R)-2-(7′-octenyl)Alanine, 2,2-Bis(4′-pentenyl)glycine, and (S)-2-(4′-pentenyl)Alanine are substituted for the amino acids at i, i+7, and i+11 positions, respectively. In certain instances when a stitch is at the i, i+7, and i+14 residues, (R)-2-(7′-octenyl)Alanine, 2,2-Bis(4′-pentenyl)glycine, and (S)-2-(7′-octenyl)Alanine are substituted for the amino acids at i, i+7, and i+14, respectively. In certain instances when a stitch is at the i, i+7, and i+14 residues, (S)-2-(7′-octenyl)Alanine, 2,2-Bis(4′-pentenyl)glycine, and (R)-2-(7′-octenyl)Alanine are substituted for the amino acids at i, i+7, and i+14, respectively. In certain instances when a stitch is at the i, i+7, and i+14 residues, (R)-2-(4′-pentenyl)Alanine, 2,2-Bis(7′-octenyl)glycine, and (S)-2-(4′-pentenyl)Alanine are substituted for the amino acids at i, i+7, and i+14, respectively. In certain instances when a stitch is at the i, i+7, and i+14 residues, (S)-2-(4′-pentenyl)Alanine, 2,2-Bis(7′-octenyl)glycine, and (R)-2-(4′-pentenyl)Alanine are substituted for the amino acids at i, i+7, and i+14, respectively.

In some instances, structurally-stabilized (e.g., stapled) peptide variants of the disclosure are prepared from a peptide comprising or consisting of the amino acid sequence of any one of SEQ ID NOs:1, 63-75, 100, 123-127, 219-221, 227-232, and 312-314, and having e.g., 1, 2, 3, 4, 5, 6, 7, 8, or 9 amino acid substitutions (e.g., 1, 2, 3, 4, 5, 6, 7, 8, or 9 amino acids are conservatively or non-conservatively substituted) and/or having, e.g., 1, 2, 3, 4, 5, 6, 7, 8, or 9 amino acid deletions from the N- and/or C-terminus (e.g., 1, 2, 3, 4, 5, 6, 7, 8, or 9 amino acids from the N- and/or C-terminus are deleted). Exemplary magainin II peptides, including variants, are provided in Table 1 and in the amino acid sequence of SEQ ID NO:1. For example, in certain instances, the structurally-stabilized peptide variants of this disclosure can have 1, 2, 3, 4, 5, 6, 7, 8, 9, 10, 11, 12, or 13 amino acid substitutions in the amino acid sequence of any one of SEQ ID NOs: 1, 63-75, 100, 123-127, and 312-314 (e.g., 1, 2, 3, 4, 5, 6, 7, 8, 9, 10, 11, 12, or 13 amino acids are conservatively or non-conservatively substituted). Exemplary stabilized pleurocidin peptides are provided in SEQ ID NOs.: 222-224. An exemplary stabilized CAP18 peptide is provided in SEQ ID NOs.:225. An exemplary stabilized esculentin peptide is provided in SEQ ID NOs.:226. In some instances, one to three amino acids of the amino acid sequence of any one of SEQ ID NOs:1, 63-75, 100, 123-127, 219-221, 227-232, and 312-314 are substituted. In some instances, the substitution(s) is/are a conservative amino acid substitution. In other instances, the substitution(s) is/are a non-conservative amino acid substitution. In some instances, where there are more than one amino acid substitutions, the substitutions are both conservative and non-conservative amino acid substitutions. In some instances, where there are more than one amino acid substitutions, each of the substitutions are conservative amino acid substitutions. In certain instances, the substituted amino acid(s) are selected from the group consisting of L-Ala, D-Ala, Aib, Sar, Ser, a substituted alanine, or a substituted glycine derivative.

In certain instances, the structurally-stabilized (e.g., stapled) variant peptides of this disclosure can have 1, 2, 3, 4, or 5, amino acids removed/deleted from the C-terminus of the sequence set forth in any one of SEQ ID NOs: 1, 63-75, 100, 123-127, 219-221, 227-232, and 312-314. In certain instances, the structurally-stabilized (e.g., stapled) variant peptides of this disclosure can have 1, 2, 3, 4, or 5, amino acid removed/deleted from the N-terminus of the sequence set forth in any one of SEQ ID NOs:1, 63-75, 100, 123-127, 219-221, 227-232, and 312-314. In certain instances, these removed amino acids can be replaced with 1-6 (e.g., 1, 2, 3, 4, 5, or 6) amino acids selected from the group consisting of L-Ala, D-Ala, Aib, Sar, Ser, a substituted alanine, or a substituted glycine derivative.

In some instances, the structurally-stabilized (e.g., stapled) variant peptides of this disclosure can have one or more (e.g., 1, 2, 3, 4, 5, 6, 7, 8) amino acids corresponding to positions 2, 5, 6, 12, 16, 17, 20, and 21 of SEQ ID NO:1 (i.e., corresponding to Ile2, Phe5, Leu6, Phe12, Phe16, Val17, Ile20, and Met21 of SEQ ID NO:1) substituted with a different hydrophobic amino acid. For example, in some instances, the amino acid corresponding to Ile2 of SEQ ID NO:1 is substituted with phenylalanine (Phe, F). These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

In some instances, the structurally-stabilized (e.g., stapled) variant peptides of this disclosure can have one or more (e.g., 1, 2, 3, 4) amino acids corresponding to positions 4, 10, 11, and 14 of SEQ ID NO:1 (i.e., corresponding to Lys4, Lys10, Lys11, and Lys14 of SEQ ID NO:1) substituted with a different positively charged amino acid. For example, in some instances, the amino acid corresponding to Lys4 of SEQ ID NO:1 is substituted with Histidine (His, H), or Arginine (Arg, R), or Ornithine. These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

In some instances, the structurally-stabilized (e.g., stapled) variant peptides of this disclosure can have one or more (e.g., 1, 2, 3, 4) amino acids corresponding to positions 7, 8, 22, and 23 of SEQ ID NO:1 (i.e., corresponding to His7, Ser8, Asn22, and Ser23 of SEQ ID NO:1) substituted with a different hydrophilic amino acid. For example, in some instances, the amino acid corresponding to His7 of SEQ ID NO:1 is substituted with Serine (Ser, S). These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

In some instances, the structurally-stabilized (e.g., stapled) variant peptides of this disclosure can have the amino acid corresponding to position 19 of SEQ ID NO:1 (i.e., corresponding to Glu19 of SEQ ID NO:1) substituted with another negatively charged amino acid. For example, in some instances, the amino acid corresponding to Glu19 of SEQ ID NO:1 is substituted with aspartate (Asp, D). These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

The structurally-stabilized (e.g., stapled) peptide variants described herein can be optimized for therapeutic use. For example, if any of the above-described structurally-stabilized (e.g., stapled) peptide variants cause red blood cell membrane disruption (cell lysis), the peptides can be optimized by lowering the overall peptide hydrophobicity. This can for example be achieved by substituting especially hydrophobic residues with an amino acid with lower hydrophobicity (e.g., lysine or glutamate). Red blood cell membrane disruption can also be lowered by reducing the overall positive charge of the peptide. This can be accomplished by substituting basic residues with uncharged or acidic residues. In certain instances, both the overall peptide hydrophobicity and the overall positive charge of the peptide are lowered. In some instances, the minimum overall positive charge of the peptide is +3.

In certain instances, the structurally-stabilized (e.g., stapled) peptide variants described herein are 5 or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In a specific instance, the structurally-stabilized (e.g., stapled) peptide variants described herein are 5-30 (i.e., 5, 6, 7, 8, 9, 10, 11, 12, 13, 14, 15, 16, 17, 18, 19, 20, 21, 22, 23, 24, 25, 26, 27, 28, 29, 30) in length. In a specific instance, the structurally-stabilized (e.g., stapled) peptide variants described herein are 10-30 (i.e., 10, 11, 12, 13, 14, 15, 16, 17, 18, 19, 20, 21, 22, 23, 24, 25, 26, 27, 28, 29, 30) amino acids in length. In a specific instance, the structurally-stabilized (e.g., stapled) peptide variants described herein are 15-30 (i.e., 15, 16, 17, 18, 19, 20, 21, 22, 23, 24, 25, 26, 27, 28, 29, 30) amino acids in length. In a specific instance, the structurally-stabilized (e.g., stapled) peptide variants described herein are 5-23 (i.e., 5, 6, 7, 8, 9, 10, 11, 12, 13, 14, 15, 16, 17, 18, 19, 20, 21, 22, 23) in length. In a specific instance, the structurally-stabilized (e.g., stapled) peptide variants described herein are 10-30 (i.e., 10, 11, 12, 13, 14, 15, 16, 17, 18, 19, 20, 21, 22, 23) amino acids in length. In a specific instance, the structurally-stabilized (e.g., stapled) peptide variants described herein are 15-23 (i.e., 15, 16, 17, 18, 19, 20, 21, 22, 23) amino acids in length. In a specific instance, the structurally-stabilized (e.g., stapled) peptide variants described herein are 23-30 (i.e., 23, 24, 25, 26, 27, 28, 29, or 30) amino acids in length. In certain instances, the structurally-stabilized (e.g., stapled) peptide variants described herein are 23 amino acids in length.

In certain instances, the structurally-stabilized (e.g., stapled) peptide variant comprises or consists of the amino acid sequence set forth in Table 2. In certain instances, the structurally-stabilized (e.g., stapled) peptide variant comprises or consists of a stapled form of an amino acid sequence set forth in Table 2. In certain instances, the structurally-stabilized (e.g., stapled) peptide variant comprises or consists of a stapled form of an amino acid sequence set forth in FIG. 5A, FIG. 5B, FIG. 9, FIG. 11, FIG. 16, FIG. 17A, or FIG. 17B. In certain instances, the structurally-stabilized (e.g., stapled) peptide variant comprises or consists of any one of constructs 1-22 or 46-53 of Table 3. In certain instances, the structurally-stabilized (e.g., stapled) peptide variant comprises or consists of any one of constructs 23-45 of Table 3. In certain instances, the structurally-stabilized (e.g., stapled) peptide variant comprises or consists of any one of constructs 54-78 of Table 3.

In some instances, this disclosure features stabilized magainin peptide variants comprising the amino acid sequence set forth in SEQ ID NO:1 or 100 with 2 to 10 (e.g., 2, 3, 4, 5, 6, 7, 8, 9, 10) amino acid substitutions. These variants can either lyse leukemia and/or lymphoma cells; or lyse leukemia and/or lymphoma cells and bacterial cells better than the unstapled magainin peptide of SEQ ID NO:1 or 100. In some instances, these variants have stapling amino acids (e.g., a, a, di-substituted non-natural amino acids with olefinic side chains) at positions: 1 and 5; 2 and 6; 3 and 7; 4 and 8; 5 and 9; 7 and 11; 8 and 12; 9 and 13; 10 and 14; 12 and 16; 14 and 18, 15 and 19; 16 and 20; 17 and 21; 18 and 22; or 19 and 23 of SEQ ID NO:1 or 100. In some instances, these variants have stapling amino acids (e.g., a, a, di-substituted non-natural amino acids with olefinic side chains) at positions: 1 and 5 and 16 and 20 of SEQ ID NO:1 or 100. In some instances, these variants have stapling amino acids (e.g., a, a, di-substituted non-natural amino acids with olefinic side chains) at positions: 2 and 6 and 16 and 20 of SEQ ID NO:1 or 100. In some instances, these variants have stapling amino acids (e.g., a, a, di-substituted non-natural amino acids with olefinic side chains) at positions: 3 and 7 and 16 and 20 of SEQ ID NO:1 or 100. In some instances, the above peptides also have one or more (e.g., 1, 2, 3, 4, 5, 6, 7, 8, 9) of the following positions substituted with a lysine: positions, 1, 2, 3, 7, 8, 9, 19, 22, and 23 of SEQ ID NO:1. In some instances, the above peptides have one or more (e.g., 1, 2, 3, 4, 5, 6, 7, 8, 9) of the following positions substituted with a glutamic acid: positions, 3, 7, 8, 18, and 22 of SEQ ID NO:1. In certain instances, these peptides have an alanine at position 21 of SEQ ID NO:1 or 100. In some instances, the above peptides have one or more (e.g., 1, 2, 3, 4, 5, 6, 7, 8, 9) positions (e.g., positions 1, 3, 7, 8, 18, 19, 21, 22, 23 of SEQ ID NO:1 or 100) substituted with a conservative amino acid substitution. In certain cases, these variants kill AML cells. In certain cases, these variants kill MLL cells. In certain cases, these variants kill histiocytic lymphoma cells. In certain cases, these variants kill both cancer cells and bacterial cells.

In some instances, this disclosure features stabilized magainin peptide variants comprising the amino acid sequence set forth in SEQ ID NO:60. In some instances the variants have 1 to 7 (e.g., 1, 2, 3, 4, 5, 6, 7) amino acid substitutions in SEQ ID NO:60. In some cases, the substitutions are at one or more (e.g., 1, 2, 3, 4, 5, 6, 7) of positions 1, 3, 7, 8, 18, 19, and 22 of SEQ ID NO:60. In some cases, these positions can be substituted with a conservative amino acid substitution. In other cases, one or more of positions 1, 3, 7, 8, and/or 19 of SEQ ID NO:60 can be substituted with a lysine. In some cases, one or more of positions 3, 7, 8, 18, and 22 of SEQ ID NO:60 can be substituted with a glutamic acid. In certain cases, these variants kill AML cells. In certain cases, these variants kill MLL cells. In certain cases, these variants kill histiocytic lymphoma cells. In certain cases, these variants kill both cancer cells and bacterial cells.

In some instances, this disclosure features stabilized magainin peptide variants comprising the amino acid sequence set forth in SEQ ID NO:22. In some instances the variants have 1 to 4 (e.g., 1, 2, 3, 4) amino acid substitutions in SEQ ID NO:22. In some cases, the substitutions are at one or more (e.g., 1, 2, 3, 4) of positions 3, 7, 18, and 22 of SEQ ID NO:22. In some cases, these positions can be substituted with a conservative amino acid substitution. In some cases, one or more of positions 3, 7, 8, 18, and 22 of SEQ ID NO:22 can be substituted with a glutamic acid. In certain cases, these variants kill AML cells. In certain cases, these variants kill MLL cells. In certain cases, these variants kill histiocytic lymphoma cells. In certain cases, these variants kill both cancer cells and bacterial cells.

In some instances, this disclosure features stabilized magainin peptide variants comprising the amino acid sequence set forth in SEQ ID NO:17. In some instances the variants have 1 to 4 (e.g., 1, 2, 3, 4) amino acid substitutions in SEQ ID NO:17. In some cases, the substitutions are at one or more (e.g., 1, 2, 3, 4) of positions 1, 5, 8, or 9 of SEQ ID NO:17. In some cases, these positions can be substituted with a conservative amino acid substitution. In some cases, positions 1 and 5 of SEQ ID NO:17 can be substituted with stapling amino acids (e.g., α-methyl, α-alkenyl non-natural amino acids with olefinic side chains). In some cases, one or more of positions 8, and 9 of SEQ ID NO:17 can be substituted with a lysine. In certain cases, these variants kill AML cells. In certain cases, these variants kill MLL cells. In certain cases, these variants kill histiocytic lymphoma cells. In certain cases, these variants kill both cancer cells and bacterial cells.

### Methods of Making Structurally-Stabilized Peptides

Also provided herein are methods of making structurally-stabilized peptides (e.g., a structurally-stabilized peptide described herein. In some instances, the method comprises (a) providing a peptide described herein, wherein the peptide comprises two or more stapling amino acids, and (b) performing a ring-closing metathesis reaction. In some instances, the method comprising: (a) providing a peptide having the sequence set forth in any one of SEQ ID NOs: 37, 98, 99, 133-145 and 222-226, and (b) cross-linking the peptide. In some instances in which the peptide has the sequence set forth in any one of SEQ ID NOs: 37, 98, 99, and 133-145, X₁ and X₂ in SEQ ID NO: 37, 137, 138, 139, or 140 is (S)-2-(4′-pentenyl)Alanine and wherein X₁, X₂, X₃, and X₄ in SEQ ID NO: 98, 99, 133, 134, 135, 136, 141, 142, 143, 144, or 145 is (S)-2-(4′-pentenyl)Alanine. In some instances, the method further comprises formulating the stapled peptide as a pharmaceutical composition. In some instances, the pharmaceutical composition is a sterile pharmaceutical composition.

Fmoc-based solid-phase peptide synthesis may be used to synthesize the structurally stabilized peptides described herein (e.g., in accordance with reported methods for generating all-hydrocarbon stapled peptides, e.g., Bird, G. H., Crannell, W. C. & Walensky, L. D. Chemical synthesis of hydrocarbon-stapled peptides for protein interaction research and therapeutic targeting. Curr. Protoc. Chem. Biol. 3, 99-117 (2011)). For example, to achieve the various staple lengths, α-methyl, α-alkenyl amino acids may be installed at i, i+4 positions using two (S)-pentenyl alanine residues (S5) and at i, i+7 positions by inserting (R)-octenyl alanine (R8) at the i position and S5 at the i+7 position. For the stapling reaction, Grubbs first-generation ruthenium catalyst dissolved in dichloroethane is added to the resin-bound peptides. To ensure maximal conversion, 3-5 rounds of stapling may be performed. The peptides are then cleaved off of the resin using, e.g., trifluoroacetic acid, precipitated using, e.g., a hexane:ether (1:1) mixture, air dried and purified by, e.g., LC-MS. Peptides may be quantified by amino acid analysis. TFA-HCl exchange may be performed on peptides to be used in animal studies.

### Exemplary Structurally-Stabilized Oncolytic Peptide Variants

In a specific aspect, the structurally-stabilized (e.g., stapled) peptide is based on the amino acid sequence of SEQ ID NO:100. In a specific aspect, the structurally-stabilized (e.g., stapled) peptide comprises the amino acid sequence of SEQ ID NO:100 with: (i) two or more amino acid substitutions with stapling amino acids, and (ii) 0 to 6 (i.e., 0, 1, 2, 3, 4, 5, 6) additional amino acid substitutions, insertions, and/or deletions. In a specific aspect, the structurally-stabilized (e.g., stapled) peptide consists of the amino acid sequence of SEQ ID NO:100 with: (i) two or more amino acid substitutions with stapling amino acids, and (ii) 0 to 6 (i.e., 0, 1, 2, 3, 4, 5, 6) additional amino acid substitutions, insertions, and/or deletions. These magainin II peptides or variants kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell. In certain aspects, the structurally-stabilized (e.g., stapled) peptide is based on the amino acid sequence of SEQ ID NO:100 with 0 to 3 amino acid deletions from the N-terminus. In certain aspects, the stabilized peptide is based on the amino acid sequence of SEQ ID NO:100 with 0 to 3 amino acid deletions from the C-terminus. In a specific aspect, the structurally-stabilized (e.g., stapled) peptide is based on the amino acid sequence of GIGKFLHSAKKFGKAX₁VGEX₂BNS, wherein each of X₁ and X₂ is independently a stapling amino acid, and wherein B is norleucine (SEQ ID NO:17). In a specific aspect, the structurally-stabilized (e.g., stapled) peptide comprises the amino acid sequence of GIGKFLHSAKKFGKAX₁VGEX₂BNS, wherein each of X₁ and X₂ is independently a stapling amino acid, and wherein B is norleucine (SEQ ID NO:17), and has 0 to 6 (i.e., 0, 1, 2, 3, 4, 5, 6) amino acid substitutions, insertions, and/or deletions relative to the amino acid sequence of SEQ ID NO:17 at position(s) other than X₁ and X₂. In a specific aspect, the structurally-stabilized (e.g., stapled) peptide consists of the amino acid sequence of GIGKFLHSAKKFGKAX₁VGEX₂BNS, wherein each of X₁ and X₂ is independently a stapling amino acid, and wherein B is norleucine (SEQ ID NO:17), and has 0 to 6 (i.e., 0, 1, 2, 3, 4, 5, 6) amino acid substitutions, insertions, and/or deletions relative to the amino acid sequence of SEQ ID NO:17 at position(s) other than X₁ and X₂. In certain aspects, the structurally-stabilized (e.g., stapled) peptide is based on the amino acid sequence of SEQ ID NO:17 with 0 to 3 amino acid deletions from the N-terminus. In certain aspects, the structurally-stabilized (e.g., stapled) peptide is based on the amino acid sequence of SEQ ID NO:17 with 0 to 3 amino acid deletions from the C-terminus. In a specific aspect, the structurally-stabilized (e.g., stapled) peptide comprises the amino acid sequence of GIGKFLHSAKKFGKAX₁VGEX₂BNS, wherein each of X₁ and X₂ is (S)-2-(4′-pentenyl)Alanine, and wherein B is norleucine (SEQ ID NO:107), and has 0 to 6 (i.e., 0, 1, 2, 3, 4, 5, 6) amino acid substitutions, insertions, and/or deletions relative to the amino acid sequence of SEQ ID NO:107 at position(s) other than X₁ and X₂. In a specific aspect, the structurally-stabilized (e.g., stapled) peptide consists of the amino acid sequence of GIGKFLHSAKKFGKAX₁VGEX₂BNS, wherein each of X₁ and X₂ is (S)-2-(4′-pentenyl)Alanine, and wherein B is norleucine (SEQ ID NO:107), and has 0 to 6 (i.e., 0, 1, 2, 3, 4, 5, 6) amino acid substitutions, insertions, and/or deletions relative to the amino acid sequence of SEQ ID NO:107 at position(s) other than X₁ and X₂. In certain aspects, the structurally-stabilized (e.g., stapled) peptide is based on the amino acid sequence of SEQ ID NO:107 with 0 to 3 amino acid deletions from the N-terminus. In certain aspects, the structurally-stabilized (e.g., stapled) peptide is based on the amino acid sequence of SEQ ID NO:107 with 0 to 3 amino acid deletions from the C-terminus. In certain aspects, the 1 to 3 amino acid in the amino acid sequence of SEQ ID NO:100, 17, or 107 that are removed from the N-terminus are replaced with 1 to 6 amino acids from the croup consisting of alanine, D-alanine, α-aminoisobutyric acid, N-methyl glycine, serine, a substituted alanine, and a glycine derivative. In certain aspects, the 1 to 3 amino acid in the amino acid sequence of SEQ ID NO:100, 17, or 107 that are removed from the C-terminus are replaced with 1 to 6 amino acids from the croup consisting of alanine, D-alanine, α-aminoisobutyric acid, N-methyl glycine, serine, a substituted alanine, and a glycine derivative. In certain instances, one or more (e.g., 1, 2, 3, 4, 5, 6, 7, 8) amino acids corresponding to positions 2, 5, 6, 12, 16, 17, 20, and 21 of SEQ ID NO:100, 17, or 107 (i.e., corresponding to Ile2, Phe5, Leu6, Phe12, Phe16, Val17, Ile20, and Met21 of SEQ ID NO:1) is substituted with a different hydrophobic amino acid. In some instances, the structurally-stabilized (e.g., stapled) variant peptides of this disclosure can have one or more (e.g., 1, 2, 3, 4) amino acids corresponding to positions 4, 10, 11, and 14 of SEQ ID NO: 100, 17, or 107 (i.e., corresponding to Lys4, Lys10, Lys11, and Lys14 of SEQ ID NO:1) is substituted with a different positively charged amino acid. In some instances, the structurally-stabilized (e.g., stapled) variant peptides of this disclosure can have one or more (e.g., 1, 2, 3, 4) amino acids corresponding to positions 7, 8, 22, and 23 of SEQ ID NO:100, 17, or 107 (i.e., corresponding to His7, Ser8, Asn22, and Ser23 of SEQ ID NO:1) is substituted with a different hydrophilic amino acid. In some instances, the structurally-stabilized (e.g., stapled) variant peptides of this disclosure can have the amino acid corresponding to position 19 of SEQ ID NO:100, 17, or 107 (i.e., corresponding to Glu19 of SEQ ID NO:1) is substituted with another negatively charged amino acid. These peptides kill human hematological cancer cells (e.g., leukemia, lymphoma, multiple myeloma cells) or human cells with a cell membrane comprising an anionic outer leaflet or with a cell membrane comprising an outer leaflet having an increased negative charge relative to the outer leaflet of a cell membrane of a normal counterpart human cell.

In a specific aspect, the structurally-stabilized (e.g., stapled) peptide comprises an amino acid sequence of Formula (I), wherein [Xaa]_{w} is GIGKFLHSAKKFGKA (SEQ ID NO:82), [Xaa]ₓ is VGE, and [Xaa]_{y} is BNS, wherein B is norleucine, R₁ is a methyl group, R₃ is an alkenyl group, R₂ is a methyl group, and z is 1.

In a specific aspect, the structurally-stabilized (e.g., stapled) peptide comprises a stapled form of the amino acid sequence of SEQ ID NO:17 (e.g., the product of a ring-closing metathesis reaction on a peptide comprising the amino acid sequence of SEQ ID NO:17). In a specific aspect, the structurally-stabilized (e.g., stapled) peptide consists of a stapled form of the amino acid sequence of the amino acid sequence of SEQ ID NO:17 (e.g., the product of a ring-closing metathesis reaction on a peptide consisting of the amino acid sequence of SEQ ID NO:17). In a specific aspect, the structurally-stabilized (e.g., stapled) peptide comprises a stapled form of the amino acid sequence of SEQ ID NO:107 (e.g., the product of a ring-closing metathesis reaction on a peptide comprising the amino acid sequence of SEQ ID NO:107). In a specific aspect, the structurally-stabilized (e.g., stapled) peptide consists of a stapled form of the amino acid sequence of SEQ ID NO:107 (e.g., the product of a ring-closing metathesis reaction on a peptide consisting of the amino acid sequence of SEQ ID NO:107).

### Methods of Use

Provided herein is a method of treating a cancer (e.g., a hematological cancer) using a stapled antimicrobial peptide. The disclosure features methods of using a structurally-stabilized oncolytic peptide (e.g., any of the structurally-stabilized (e.g., stapled) peptides (or pharmaceutical compositions comprising said structurally-stabilized peptides) described herein) for the prevention and/or treatment of a cancer (e.g. hematological cancer, e.g., leukemia, lymphoma, multiple myeloma) in a subject (e.g., human) in need thereof. In some instances, use of the structurally-stabilized oncolytic peptide in the prevention and/or treatment of a cancer in a subject (e.g., human) avoids toxicity to normal (non-cancerous) tissue in the subject. In some instances, the disclosure provides a method of killing human cells having a cell membrane comprising an anionic outer leaflet or having a cell membrane comprising an outer leaflet having an increased net negative charge relative to the outer leaflet of a cell membrane of a normal counterpart human cell, by contacting the cell with a StOP disclosed herein. The terms “treat” or “treating,” as used herein, refers to alleviating, inhibiting, or ameliorating the disease or infection from which the subject is suffering. In one instance, the method of treating cancer (e.g., hematological cancer, e.g., leukemia, lymphoma, multiple myeloma) in a subject (e.g., human) in need thereof comprises administering to the subject a therapeutically effective amount of a structurally-stabilized peptide, wherein the structurally-stabilized peptide is 5 to 50 amino acids in length and has at least 60% (at least 65%, at least 70%, at least 75%, at least 80%, at least 85%, or at least 90%) identity to the amino acid sequence GIGKFLHSAKKFGKAFVGEUVINS (SEQ ID NO:1) or GIGKFLHSAKKFGKAFVGEIBNS (SEQ ID NO:100) (over the full length of the sequence). In certain instances, the cancer is a hematological cancer. In certain instances, the cancer is leukemia. In certain instances, the leukemia is acute myeloid leukemia. In certain instances, the leukemia is mixed lineage leukemia. In certain cases, the cancer is a lymphoma. In certain cases, the lymphoma is a histiocytic lymphoma. In certain instances, the cancer is multiple myeloma. In certain instances, the structurally-stabilized peptide is a structurally-stabilized peptide described herein. In some instances, the structurally-stabilized peptide is the amino acid sequence set forth in one of SEQ ID NO: 2, 22, 42, 46, 56, 58, or 60, or a variant thereof (e.g., having 1 to 8 amino acid substitutions). In some instances, the structurally-stabilized peptide is the amino acid sequence set forth in one of SEQ ID NO: 2, 4, 17, 22, 27, 28, 42, 46, 56, 58, 60, 98, 99, 133-145, or 222-226, or a variant thereof (e.g., having 1 to 8 amino acid substitutions). In some instances, the structurally-stabilized peptide is the amino acid sequence set forth in one of SEQ ID NO: 2-4, 6, 8, 13, 17, 18, 21-23, 26, 27, 31, 34, 35, 37, 38, 42, 46, 47, 54, 56, or 58-60, or a variant thereof (e.g., having 1 to 8 amino acid substitutions). In some instances, the structurally-stabilized peptide is the amino acid sequence set forth in one of SEQ ID NO: 133-145, or a variant thereof (e.g., having 1 to 8 amino acid substitutions). In some instances, the structurally-stabilized peptide is the amino acid sequence set forth in one of SEQ ID NO: 101-121 or 128-132, or a variant thereof (e.g., having 1 to 8 amino acid substitutions). In some instances, the structurally-stabilized peptide is the amino acid sequence set forth in one of SEQ ID NO: 146-158, or a variant thereof (e.g., having 1 to 8 amino acid substitutions). In some instances, the structurally-stabilized peptide is the amino acid sequence set forth in one of SEQ ID NO: 222-226, or a variant thereof (e.g., having 1 to 8 amino acid substitutions). In some instances, the structurally-stabilized peptide is the amino acid sequence set forth in SEQ ID NO:233.

This disclosure also features methods of using a structurally-stabilized oncolytic peptide (e.g., any of the structurally-stabilized (e.g., stapled) peptides (or pharmaceutical compositions comprising said structurally-stabilized peptides) described herein) for the treatment of a cancer (e.g. a hematological cancer, e.g., leukemia, lymphoma, multiple myeloma) while also prophylaxing or treating bacterial infections (e.g., due to immune suppression as a consequence of chemotherapy and/or radiotherapy) in a subject (e.g., human) in need thereof. In one instance, the method of treatment of a subject (e.g., human) in need thereof comprises administering to the subject a therapeutically effective amount of a structurally-stabilized peptide, wherein the structurally-stabilized peptide is 5 to 50 amino acids in length and has at least 60% (at least 65%, at least 70%, at least 75%, at least 80%, at least 85%, or at least 90%) identity to the amino acid sequence GIGKFLHSAKKFGKAFVGEIMNS (SEQ ID NO:1) or GIGKFLHSAKKFGKAFVGEIBNS (SEQ ID NO:100). In certain instances, the structurally-stabilized peptide is a structurally stabilized peptide described herein. In some instances, the structurally-stabilized peptide is one of SEQ ID NO:17, 27, 98, or 99, or a variant thereof (e.g., having 1 to 8 amino acid substitutions). In certain instances, the cancer is a hematological cancer. In certain instances, the cancer is leukemia. In certain instances, the leukemia is acute myeloid leukemia. In certain instances, the leukemia is mixed lineage leukemia. In certain cases, the cancer is a lymphoma. In certain cases, the lymphoma is a histiocytic lymphoma. In certain instances, the cancer is multiple myeloma. In certain instances the bacterial infection is a Gram-negative bacterial infection. In other instances, the bacterial infection is a Gram-positive bacterial infection. In other instances, the bacterial infection is an antibiotic-resistant bacterial infection. In some instances, the structurally-stabilized peptide is the amino acid sequence set forth in one of SEQ ID NO: 2-4, 6, 8, 13, 17, 18, 21-23, 26, 27, 31, 34, 35, 37, 38, 42, 46, 47, 54, 56, or 58-60, or a variant thereof (e.g., having 1 to 8 amino acid substitutions). In some instances, the structurally-stabilized peptide is the amino acid sequence set forth in one of SEQ ID NO: 133-145, or a variant thereof (e.g., having 1 to 8 amino acid substitutions). In some instances, the structurally-stabilized peptide is the amino acid sequence set forth in one of SEQ ID NO: 101-121 or 128-132, or a variant thereof (e.g., having 1 to 8 amino acid substitutions). In some instances, the structurally-stabilized peptide is the amino acid sequence set forth in one of SEQ ID NO: 146-158, or a variant thereof (e.g., having 1 to 8 amino acid substitutions). In some instances, the structurally-stabilized peptide is the amino acid sequence set forth in one of SEQ ID NO: 222-226, or a variant thereof (e.g., having 1 to 8 amino acid substitutions). In some instances, the structurally-stabilized peptide is the amino acid sequence set forth in SEQ ID NO:233.

The disclosure also features methods using a structurally-stabilized oncolytic peptide (e.g., any of the structurally-stabilized (e.g., stapled) peptides (or pharmaceutical compositions comprising said structurally-stabilized peptides) described herein) for inhibiting proliferation of a cancer cell in a subject (e.g., human) in need thereof. In some instances, the method of inhibiting proliferation of a cancer cell in a subject (e.g., human) in need thereof comprises administering to the subject a therapeutically effective amount of a structurally-stabilized peptide, wherein the structurally-stabilized peptide is 5 to 50 amino acids in length and has at least 60% (at least 65%, at least 70%, at least 75%, at least 80%, at least 85%, or at least 90%) identity to the amino acid sequence GIGKFLHSAKKFGKAFVGEIMNS (SEQ ID NO:1) or GIGKFLHSAKKFGKAFVGEIBNS (SEQ ID NO:100) (over the full length of the sequence), wherein the structurally-stabilized peptide specifically lyses the cancer cells. In some instances, the cancer cell is a hematological cancer cell. In some instances, the cancer cell is a leukemia cancer cell. In some instances, the cancer cell is an acute myeloid leukemia cancer cell. In some instances, the cancer cell is a mixed lineage leukemia cancer cell. In some instances, the cancer cell is a lymphoma cancer cell. In certain cases, the lymphoma cell is a histiocytic lymphoma cell. In certain cases, the cancer cell is multiple myeloma cell. In some instances, the structurally-stabilized peptide is the amino acid sequence set forth in one of SEQ ID NO: 2, 22, 42, 46, 56, 58, or 60, or a variant thereof (e.g., having 1 to 8 amino acid substitutions). In some instances, the structurally-stabilized peptide is the amino acid sequence set forth in one of SEQ ID NO: 2, 4, 17, 22, 27, 28, 42, 46, 56, 58, 60, 98, 99, or 133-145, or a variant thereof (e.g., having 1 to 8 amino acid substitutions). In some instances, the structurally-stabilized peptide is the amino acid sequence set forth in one of SEQ ID NO: 2-4, 6, 8, 13, 17, 18, 21-23, 26, 27, 31, 34, 35, 37, 38, 42, 46, 47, 54, 56, or 58-60, or a variant thereof (e.g., having 1 to 8 amino acid substitutions). In some instances, the structurally-stabilized peptide is the amino acid sequence set forth in one of SEQ ID NO: 133-145, or a variant thereof (e.g., having 1 to 8 amino acid substitutions). In some instances, the structurally-stabilized peptide is the amino acid sequence set forth in one of SEQ ID NO: 101-121 or 128-132, or a variant thereof (e.g., having 1 to 8 amino acid substitutions). In some instances, the structurally-stabilized peptide is the amino acid sequence set forth in one of SEQ ID NO: 146-158, or a variant thereof (e.g., having 1 to 8 amino acid substitutions). In some instances, the structurally-stabilized peptide is the amino acid sequence set forth in one of SEQ ID NO: 222-226, or a variant thereof (e.g., having 1 to 8 amino acid substitutions). In some instances, the structurally-stabilized peptide is the amino acid sequence set forth in SEQ ID NO:233. In some instances, the structurally-stabilized peptide specifically lyses bacteria cells in addition to the cancer cells.

In certain instances, the structurally-stabilized peptide used in a method described herein is a structurally stabilized peptide described herein. The structurally-stabilized peptides disclosed herein are capable of specifically lysing cancer cells (e.g., hematological cancer cells, e.g., leukemia cells, lymphoma cells, multiple myeloma cells). In certain instances, the structurally-stabilized peptides disclosed herein are capable of specifically lysing hematological cancer cells. In certain instances, the structurally-stabilized peptides disclosed herein are capable of specifically lysing leukemia cells. In certain instances, the structurally-stabilized peptides disclosed herein are capable of specifically lysing lymphoma cells. In certain instances, the structurally-stabilized peptides disclosed herein are capable of specifically lysing multiple myeloma cells. In certain instances, the structurally-stabilized peptides disclosed herein for use in a method of treating cancer or in a method of inhibiting proliferation of a cancer cell are capable of specifically lysing cancer cells of the type of cancer to be treated or of the type of cancer cell for which proliferation is to be inhibited. For example, if the method comprises treating leukemia/lymphoma, the structurally-stabilized peptide (or composition comprising the structurally-stabilized peptide) used in the method is capable of specifically lysing leukemia/lymphoma cells. In another example, if the method comprises inhibiting leukemia/lymphoma cancer cells, the structurally-stabilized peptide (or composition comprising the structurally-stabilized peptide) used in the method is capable of specifically lysing leukemia/lymphoma cells. It follows that a structurally-stabilized peptide that is incapable of specifically lysing cells of a particular cancer type is not used in a method of treating that cancer type or in a method of inhibiting proliferation of a cancer cell of that cancer type. For example, a structurally-stabilized peptide that is incapable of specifically lysing, e.g., breast cancer cells, would not be used in a method of treating breast cancer in a subject in need thereof. As another example, a structurally-stabilized peptide that is incapable of specifically lysing, e.g., breast cancer cells, would not be used in a method of inhibiting proliferation of a breast cancer cell in a subject in need thereof. Methods for determining if a structurally-stabilized peptide is capable of specifically lysing cancer cells are known in the art and described herein.

The structurally-stabilized (e.g., stapled) peptides (or compositions comprising the peptides) described herein can be useful for treating a hematological cancer (e.g., a leukemia, a lymphoma, and/or multiple myeloma) in a human subject. The peptides (or compositions comprising the peptides) described herein can also be useful for preventing cancer, e.g., a hematological cancer, e.g., a leukemia or a lymphoma or multiple myeloma, from developing in a human subject. Thus, provided herein are methods of treating a hematological cancer (e.g., a leukemia or a lymphoma or multiple myeloma) in a human subject in need thereof, comprising administering to the subject a therapeutically effective amount of a structurally-stabilized peptide described herein (or a composition comprising the structurally-stabilized peptide). Also provided herein are methods of preventing a hematological cancer (e.g., a leukemia or a lymphoma or multiple myeloma) in a human subject in need thereof, comprising administering to the subject a therapeutically effective amount of a structurally-stabilized peptide described herein (or a composition comprising the structurally-stabilized peptide).

The stabilized peptides described herein can be used to lyse cancer cells (e.g., hematological cancer cells, e.g., leukemia and/or lymphoma and/or multiple myeloma cells) and to kill cancer cells. These peptides can also be used to kill drug-resistant cancer cells. The killing is specific in that cancer cells are lysed but non-cancerous cells are spared (e.g., less than 20%, less than 15%, less than 10%, less than 5%, less than 4%, less than 3%, or less than 1% of non-cancerous cells are killed).

In certain instances, the human subject in need thereof is administered a structurally-stabilized (e.g., stapled) peptide described in Table 2, FIG. 1, FIG. 5A, FIG. 5B, FIG. 9, FIG. 11, FIG. 16, FIG. 17A, or FIG. 17B, or a construct of Formula (I) described in Table 3. In certain instances, the stapled peptide comprises the amino acid sequence set forth in any one of SEQ ID NOs: 2-4, 6, 8, 13, 17, 18, 21-23, 26, 27, 35, 38, 42, 46, 47, 56, 58, and 60 or a modified version thereof. In certain instances, the stapled peptide consists of the amino acid sequence set forth in any one of SEQ ID NOs: 2-4, 6, 8, 13, 17, 18, 21-23, 26, 27, 35, 38, 42, 46, 47, 56, 58, and 60 or a modified version thereof. In certain instances, the stapled peptide comprises the amino acid sequence set forth in any one of SEQ ID NOs:101-121 or a modified version thereof. In certain instances, the stapled peptide consists of the amino acid sequence set forth in any one of SEQ ID NOs:101-121 or a modified version thereof. In certain instances, the stapled peptide comprises a stapled form of amino acid sequence set forth in any one of SEQ ID NOs:2-4, 6, 8, 13, 17, 18, 21-23, 26, 27, 35, 38, 42, 46, 47, 56, 58, and 60 or a modified version thereof. In certain instances, the stapled peptide consists of a stapled form of amino acid sequence set forth in any one of SEQ ID NOs:2-4, 6, 8, 13, 17, 18, 21-23, 26, 27, 35, 38, 42, 46, 47, 56, 58, and 60 or a modified version thereof. In certain instances, the stapled peptide comprises a stapled form of amino acid sequence set forth in any one of SEQ ID NOs:101-121 or a modified version thereof. In certain instances, the stapled peptide consists of a stapled form of amino acid sequence set forth in any one of SEQ ID NOs:101-121 or a modified version thereof. In some instances, the structurally-stabilized peptide is the amino acid sequence set forth in one of SEQ ID NO: 2-4, 6, 8, 13, 17, 18, 21-23, 26, 27, 31, 34, 35, 37, 38, 42, 46, 47, 54, 56, or 58-60, or a variant thereof (e.g., having 1 to 8 amino acid substitutions). In some instances, the structurally-stabilized peptide is the amino acid sequence set forth in one of SEQ ID NO: 133-145, or a variant thereof (e.g., having 1 to 8 amino acid substitutions). In some instances, the structurally-stabilized peptide is the amino acid sequence set forth in one of SEQ ID NO: 101-121 or 128-132, or a variant thereof (e.g., having 1 to 8 amino acid substitutions). In some instances, the structurally-stabilized peptide is the amino acid sequence set forth in one of SEQ ID NO: 146-158, or a variant thereof (e.g., having 1 to 8 amino acid substitutions). In some instances, the structurally-stabilized peptide is the amino acid sequence set forth in one of SEQ ID NO: 222-226, or a variant thereof (e.g., having 1 to 8 amino acid substitutions). In some instances, the structurally-stabilized peptide is the amino acid sequence set forth in SEQ ID NO:233.

In certain instances, the human subject in need thereof is administered any one of constructs 1-22 or 46-53 described in Table 3. In certain instances, the human subject in need thereof is administered any one of constructs 23-45 described in Table 3. In certain instances, the human subject in need thereof is administered construct 21 described in Table 3. In certain instances, the human subject in need thereof is administered construct 22 described in Table 3. In certain instances, the human subject in need thereof is administered any one of constructs 54-78 described in Table 3.

In certain instances, the cancer is a cancer originating from bone, prostate, stomach, urinary tract, CNS, peripheral nerve, hematopoietic, kidney, thyroid, skin, soft tissue, salivary, ovary, testis, lung, pleura, endometrium, pancreas, breast, upper digestive, large intestine, autonomic ganglia, oesophagus, liver, and biliary tissue.

In certain instances, the cancer is a hematological cancer. Examples of hematological cancers include leukemias, lymphomas, and myelomas.

In some instances, the hematological cancer is a leukemia. In some instances, the leukemia is a lymphoblastic leukemia. In some instances, the leukemia is a myeloid leukemia. In some instances, the leukemia is a mixed lineage leukemia. Non-limiting examples of leukemias include acute myeloid leukemia (AML), acute lymphocytic leukemia (ALL), chronic lymphocytic leukemia, chronic myeloid leukemia, chronic myelomonocytic leukemia, hairy cell leukemia, myelodysplastic syndromes, acute promyelocytic leukemia, myeloproliferative neoplasms, and systemic mastocytosis. Thus, in some instances, the hematological cancer is acute myeloid leukemia (AML), acute lymphocytic leukemia (ALL), chronic lymphocytic leukemia, chronic myeloid leukemia, chronic myelomonocytic leukemia, hairy cell leukemia, myelodysplastic syndromes, acute promyelocytic leukemia, myeloproliferative neoplasms, or systemic mastocytosis. In some instances, the hematological cancer is acute myeloid leukemia.

In some instances, the hematological cancer is a lymphoma. Non-limiting examples of lymphomas include non-Hodgkin lymphoma, a Hodgkin's lymphoma (e.g., lymphocyte-depleted Hodgkin's disease, lymphocyte-rich Hodgkin's disease, mixed cellularity Hodgkin's lymphoma, nodular lymphocyte-predominant Hodgkin's disease, and nodular sclerosis Hodgkin's lymphoma), histiocytic lymphoma, a B-cell lymphoma (e.g., diffuse large B-cell lymphoma), a T-cell lymphoma (e.g., cutaneous T-cell lymphoma (e.g., mycosis fungoides and Sezary syndrome)), angioimmunoblastic lymphoma, anaplastic large cell lymphoma, precursor T-lymphoblastic lymphoma/leukemia, peripheral T-cell lymphoma-unspecified, adult T cell lymphoma, extranodal natural killer/T-cell lymphoma nasal type, enteropathy-associated intestinal T-cell lymphoma, and lymphoblastic lymphoma), Burkitt's lymphoma, follicular lymphoma, mantle cell lymphoma, primary mediastinal B cell lymphoma, small lymphocytic lymphoma, and Waldenstrom macroglobulinemia. Thus, in some instances, the hematological cancer is non-Hodgkin lymphoma, a Hodgkin's lymphoma (e.g., lymphocyte-depleted Hodgkin's disease, lymphocyte-rich Hodgkin's disease, mixed cellularity Hodgkin's lymphoma, nodular lymphocyte-predominant Hodgkin's disease, and nodular sclerosis Hodgkin's lymphoma), histiocytic lymphoma, a B-cell lymphoma (e.g., diffuse large B-cell lymphoma), a T-cell lymphoma (e.g., cutaneous T-cell lymphoma (e.g., mycosis fungoides and Sezary syndrome)), angioimmunoblastic lymphoma, anaplastic large cell lymphoma, precursor T-lymphoblastic lymphoma/leukemia, peripheral T-cell lymphoma-unspecified, adult T cell lymphoma, extranodal natural killer/T-cell lymphoma nasal type, enteropathy-associated intestinal T-cell lymphoma, and lymphoblastic lymphoma), Burkitt's lymphoma, follicular lymphoma, mantle cell lymphoma, primary mediastinal B cell lymphoma, small lymphocytic lymphoma, or Waldenstrom macroglobulinemia.

In some instances the hematological cancer is multiple myeloma (e.g., a hyperdiploid multiple myeloma or a hypodiploid multiple myeloma). Non-limiting examples of multiple myeloma include light chain myeloma, non-secretory myeloma, solitary plasmacytoma, extramedullary plasmacytoma, monoclonal gammopathy of undetermined significance, smoldering multiple myeloma, IgG myeloma, IgA myeloma, IgM myeloma, IgD myeloma, and IgE myeloma. Thus, in some instances, the hematological cancer is light chain myeloma, non-secretory myeloma, solitary plasmacytoma, extramedullary plasmacytoma, monoclonal gammopathy of undetermined significance, smoldering multiple myeloma, IgG myeloma, IgA myeloma, IgM myeloma, IgD myeloma, or IgE myeloma.

In general, methods include selecting a subject and administering to the subject an effective amount of one or more of the structurally-stabilized (e.g., stapled) peptide herein, e.g., in or as a pharmaceutical composition, and optionally repeating administration as required for the method (e.g., the prevention or treatment of cancer (e.g., a hematological cancer cell, e.g., leukemia, lymphoma, multiple myeloma) or the inhibition of proliferation of a cancer cell) and can be administered orally, intravenously or topically. A subject can be selected for treatment based on, e.g., determining that the subject has cancer (e.g., a hematological cancer, e.g., leukemia, lymphoma, multiple myeloma).

Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds (structurally-stabilized peptides) selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### Pharmaceutical Compositions

One or more of any of the structurally-stabilized (e.g., stapled) peptides described herein can be formulated for use as or in pharmaceutical compositions. The pharmaceutical compositions may be used in the methods of use described herein (see above). In certain instances, the pharmaceutical composition comprises a structurally-stabilized (e.g., stapled) peptide comprising or consisting of an amino acid sequence that is identical to an amino acid sequence set forth in Table 2 or in SEQ ID NOs.: 222-226, except for 1 to 10, 1 to 9, 1 to 8, 1 to 7, 1 to 6, 1 to 5, 1 to 4, 1 to 3, 1 to 2, or 1 amino acid substitution, insertion, or deletion. In certain instances, the pharmaceutical composition comprises a structurally-stabilized (e.g., stapled) peptide comprising or consisting of an amino acid sequence that is identical to an amino acid sequence set forth in FIG. 1, FIG. 5A, FIG. 5B, FIG. 9, FIG. 11, FIG. 16, FIG. 17A, or FIG. 17B, except for 1 to 10, 1 to 9, 1 to 8, 1 to 7, 1 to 6, 1 to 5, 1 to 4, 1 to 3, 1 to 2, or 1 amino acid substitution, insertion, or deletion. In certain instances, the pharmaceutical composition comprises a structurally-stabilized (e.g., stapled) peptide comprising or consisting of any one of constructs 1-22 or 46-53 of Formula (I) described in Table 3, except for 1 to 10, 1 to 9, 1 to 8, 1 to 7, 1 to 6, 1 to 5, 1 to 4, 1 to 3, 1 to 2, or 1 amino acid substitution, insertion, or deletion. In certain instances, the pharmaceutical composition comprises a structurally-stabilized (e.g., stapled) peptide comprising or consisting of any one of constructs 23-45 of Formula (I) described in Table 3, except for 1 to 10, 1 to 9, 1 to 8, 1 to 7, 1 to 6, 1 to 5, 1 to 4, 1 to 3, 1 to 2, or 1 amino acid substitution, insertion, or deletion. In certain instances, the pharmaceutical composition comprises a structurally-stabilized (e.g., stapled) peptide comprising or consisting of any one of constructs 54-78 of Formula (I) described in Table 3, except for 1 to 10, 1 to 9, 1 to 8, 1 to 7, 1 to 6, 1 to 5, 1 to 4, 1 to 3, 1 to 2, or 1 amino acid substitution, insertion, or deletion. Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays and/or solutions).

In some instances, pharmaceutical compositions can include an effective amount of one or more structurally-stabilized (e.g., stapled) peptides. The terms “effective amount” and “effective to treat,” as used herein, refer to an amount or a concentration of one or more structurally-stabilized (e.g., stapled) peptides or a pharmaceutical composition described herein utilized for a period of time (including acute or chronic administration and periodic or continuous administration) that is effective within the context of its administration for causing an intended effect or physiological outcome (e.g., treatment of cancer).

Pharmaceutical compositions of this invention can include one or more structurally-stabilized (e.g., stapled) peptides described herein and any pharmaceutically acceptable carrier and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient, together with a structurally-stabilized peptide of this disclosure, and which does not destroy the pharmacological activity thereof and is nontoxic when administered in doses sufficient to deliver a therapeutic amount of the compound.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

In some instances, one or more structurally-stabilized (e.g., stapled) peptides disclosed herein can be conjugated, for example, to a carrier protein. Such conjugated compositions can be monovalent or multivalent. For example, conjugated compositions can include one structurally-stabilized (e.g., stapled) peptide disclosed herein conjugated to a carrier protein. Alternatively, conjugated compositions can include two or more structurally-stabilized (e.g., stapled) peptides disclosed herein conjugated to a carrier.

As used herein, when two entities are “conjugated” to one another they are linked by a direct or indirect covalent or non-covalent interaction. In certain instances, the association is covalent. In other instances, the association is non-covalent. Non-covalent interactions include hydrogen bonding, van der Waals interactions, hydrophobic interactions, magnetic interactions, electrostatic interactions, etc. An indirect covalent interaction is when two entities are covalently connected, optionally through a linker group.

Carrier proteins can include any protein that increases or enhances immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561, 1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: A Staple Scanning Library of an Antimicrobial Peptide Yields Selectively Oncolytic Peptides

In designing compounds that could specifically lyse cancer cells, a helical wheel analysis was performed on the sequence of magainin II (SEQ ID NO: 1) (FIG. 1A and FIG. 1B). This analysis determined which residues would be amenable to substitution for hydrocarbon cross-linking amino acids for peptide stapling. It is noted that the hydrophobic residues are aligned on the left (hydrophobic face) and the positively charged residues are aligned on the right (i.e. the nature of this amphipathic alpha-helical AMP) (FIG. 1B).

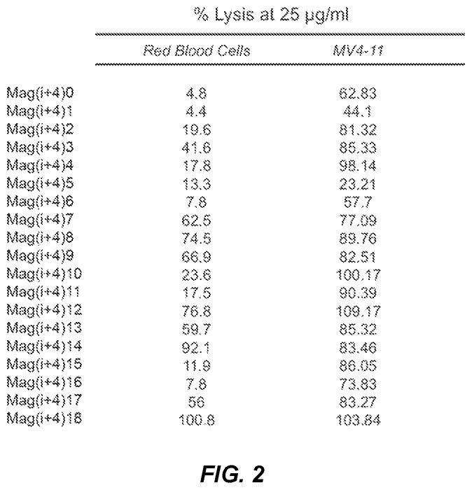

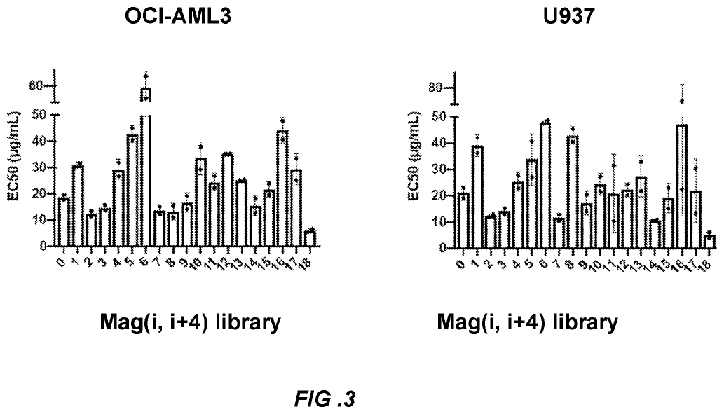

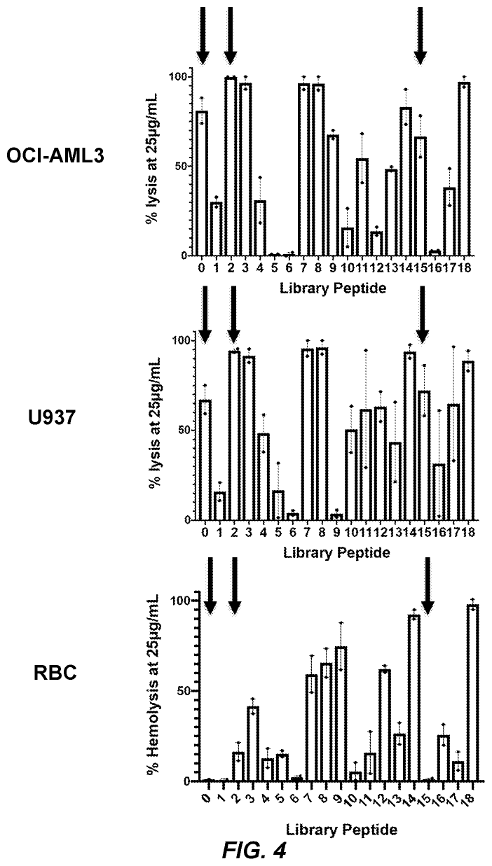

The i, i+4 staple scanning library of magainin II (Mag(i+4) library) was synthesized and screened against MV4;11 MLL leukemia cells and RBCs to assess for relatively lytic activity. The comparative screen demonstrated that the MV4;11 cells were overall more sensitive to the lytic activity of the Mag(i+4) library, with specific stapled peptides showing a broad window of specificity for cancer cell lysis activity over hemolytic activity (e.g. Mag(i+4) compounds 0, 1, 2, 4, 6, 11, 15, and 16) (FIG. 2). In particular, select stapled peptides were found to be selectively toxic (lytic) to leukemia cells with relatively little to no effect on red blood cells. The stapled peptides that showed a window of specificity for leukemia cells over red blood cells (i.e., selectively lyse leukemia cells) include Mag(i+4) 0, 1, 2, 4, 6, 11, 15 and 16 (SEQ ID NOs:2-4, 6, 8, 13, 17, and 18). In applying the library to OCI-AML3 and U937 cells, staple location was found to dictate the degree of cancer cell cytotoxicity, with select stapled peptides (SEQ ID NOs: 2, 4, 5, 9, 10, 11, 16, and 20) showing especially potent cancer killing activity (FIG. 3). In comparing the membrane lytic activity of the Mag(i+4) library in OCI-AML3, U937, and RBCs, a distinct pattern of lysis emerged between the leukemia cells and RBCs (FIG. 4). Whereas stapled peptides such as Mag(i+4)18 were lytic to all three cell types and Mag(i+4)6 had no effect on any cell type, several examples of striking cancer cell selectivity emerged. Mag(i+4) peptides with staples 0, 2, and 15 were profoundly lytic for both leukemia cell lines but had little to no effect on RBCs (FIG. 4, arrows), highlighting the discovery of cancer cell specific oncolytic peptides.

### Example 2: Lysine and Glutamate Scans Identify Stapled Oncolytic Peptides with an Expanded Window of Specificity

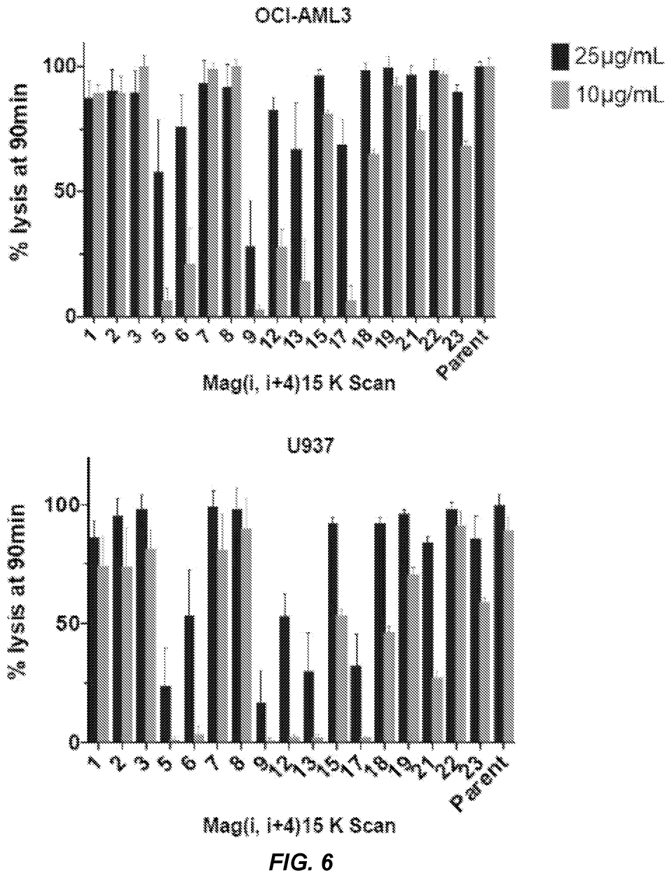

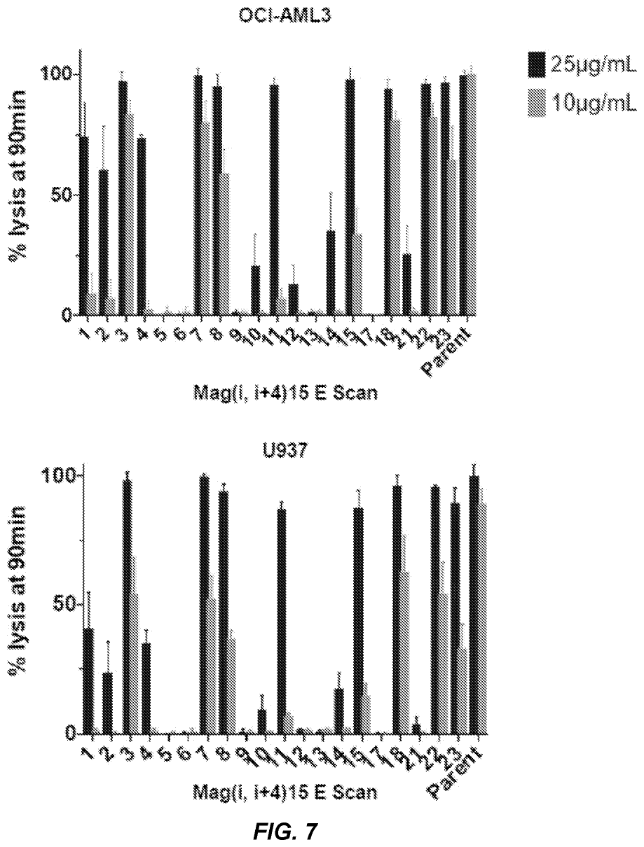

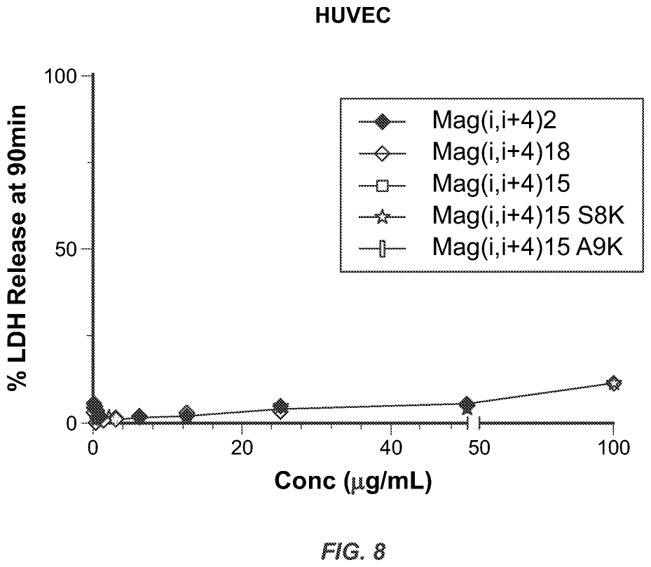

Stapled antimicrobial peptides incorporating lysine residues that disrupt the continuity of hydrophobic surfaces can substantially reduce non-specific mammalian cell lysis and also decrease antibacterial activity. Glutamate mutagenesis can even further reduce non-specific mammalian cell lysis and antibacterial activity by both disrupting continuous hydrophobic surfaces and reducing overall positive charge. Thus, selective oncolytic peptides that retain potent cancer cell membrane lytic activity upon lysine and glutamate mutagenesis may exhibit an even greater therapeutic window of membrane specificity. To identify such constructs, lysine and glutamate scanning libraries were synthesized based on Mag(i+4)15 (FIG. 5), a stapled peptide shown to have cancer cell-specific lytic activity (FIG. 4). These libraries were generated to identify stapled peptides that could retain cancer cell killing while further reducing non-cancer cell lysis by lysine and glutamate mutagenesis. Notably, it was found that a substantial portion of lysine mutants retain substantial, if not all, oncolytic activity (e.g. positions 1, 2, 3, 7, 8, 15, 18, 19, 21, 22, 23), as measured in OCI-AML3 and U937 cells by LDH release at 10 and 25 μg/mL dosing at 90 minutes (FIG. 6). The parent compound, Mag(i+4)15 served as the control compound. A series of lysine mutants retained substantial or all cancer cell killing activity (e.g. amino acid positions 1, 2, 3, 7, 8, 15, 18, 19, 21, 22, 23). Notably, G1K and I2K mutagenesis substantially impaired Mag(i+4)15 killing of B. cereus and S. aureus compared to the parent compound, whereas no such reduction of toxicity was observed for the leukemia cells treated with the same mutant constructs. Glutamate mutagenesis is known to reduce the membrane perturbing effects of AMPs even further, yet it was identified that a significant number of glutamate mutants of Mag(i+4)15 that also retained potent membrane lysis of OCI-AML and U937 cells (e.g. positions 3, 7, 8, 18, 22), as assessed by LDH release at 10 and 25 μg/mL dosing at 90 minutes (FIG. 7). Once again, the parent compound Mag(i+4)15 served as the control compound. A series of glutamate mutants retained substantial or all cancer cell killing activity (e.g. amino acid positions 3, 7, 8, 15, 18, 22, 23). Importantly, a selection of oncolytic peptides and their mutants had little to no effect on non-transformed cells, such as HUVECs, with no membrane lysis observed in the dose range that effectively lysed the leukemia cells (FIG. 8). These data highlight that stapled oncolytic peptides can be tuned by mutagenesis to enhance cancer cell selectivity even further.

### Example 3: Stapled Peptides with Dual Oncolytic and Antibacterial Activities

A lead double-stapled magainin II peptide was successfully developed with stabilized alpha-helical structure, proteolytic resistance and potent antibacterial activity in vitro and in vivo. This construct, named Mag(i+4)1,15 (A9K, B21A, N22K, S23K) (FIG. 9, top), was further tested for oncolytic properties and was found to lyse a panel of leukemia cells with EC50s between 15.8 and 36.6 at 90 minutes and 11.5 and 28.2 at 24 hours, as measured by LDH release assay (FIG. 9, bottom). To further probe membrane-lytic specificity within cancer subtypes, MCF-7 breast cancer cells were further tested and SJSA1 osteosarcoma cells with Mag(i+4)1,15 (A9K, B21A, N22K, S23K), and observed little to no lytic activity in the effective dose range for leukemia cells at either 90 minutes or 24 hours (FIG. 10). Mag(i+4)1,15(A9K, B21A, N22K, S23K), which can effectively lyse leukemia cells with EC50s<36.6 μg/mL, showed little to no effect at 24 hour on the membranes of MCF-7 breast cancer and SJSA1 osteosarcoma cells over a relatively broad dose range. Time-dependent lysis was observed in the osteosarcoma and breast cancer lines at the two highest doses of 100 and 200 μg/mL at 24 hours. These data demonstrate that select stapled peptides can be both oncolytic and bacteriolytic, and also discriminate between different types of cancer cell membranes to yield cancer cell type-specific lytic activity (FIG. 9, FIG. 10).

### Example 4: Design of Differentially Stapled and Mutated Oncolytic Peptides to Achieve Oncolytic and Dual Oncolytic/Bacteriolytic Stapled Peptides

By integrating the lysis data from staple scanning, lysine scanning, and glutamate scanning libraries, stapled oncolytic magainin peptides can be designed with tunable membrane selectivities, including compounds that are cancer cell type specific or capable of targeting both cancer cells and bacteria (FIG. 11). The latter dual action can be an important advantage in treating cancer patients who are especially susceptible to infection due to immune suppression, including chemotherapy induced neutropenia. Stapling strategies that yield oncolytic peptides include single stapling, double-stapling, and stitching, which can incorporate distinct non-natural amino acid pairs to yield [i, i+3], [i, i+4], and [i, i+7] internal crosslinks of various combinations, as schematized in FIGS. 12, 13, and 14. Such constructs can be further iterated by mutagenesis scans, as exemplified in FIG. 5, to yield integrated constructs with optimal therapeutic properties (FIG. 11, FIG. 15, FIG. 16).

### Example 5: Testing of Differentially Stapled and Mutated StOPs to Achieve Potent and Selective Oncolytic Stapled Peptides Across the Diversity of Human Cancer Cell Types, Including Primary Patient Specimens

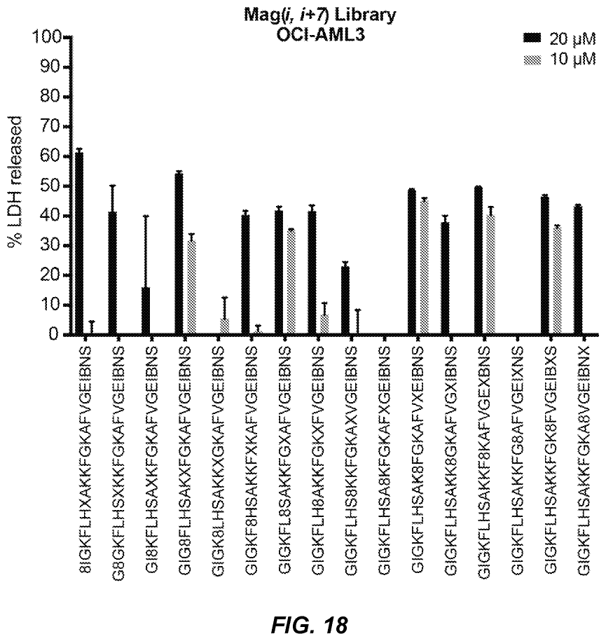

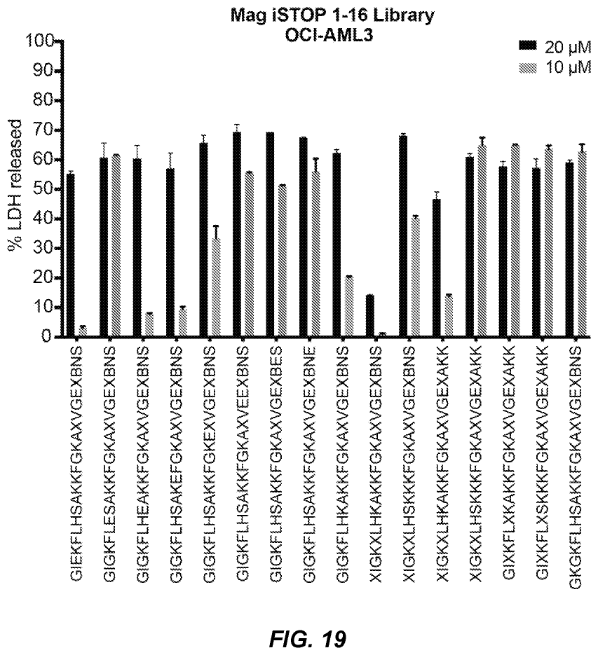

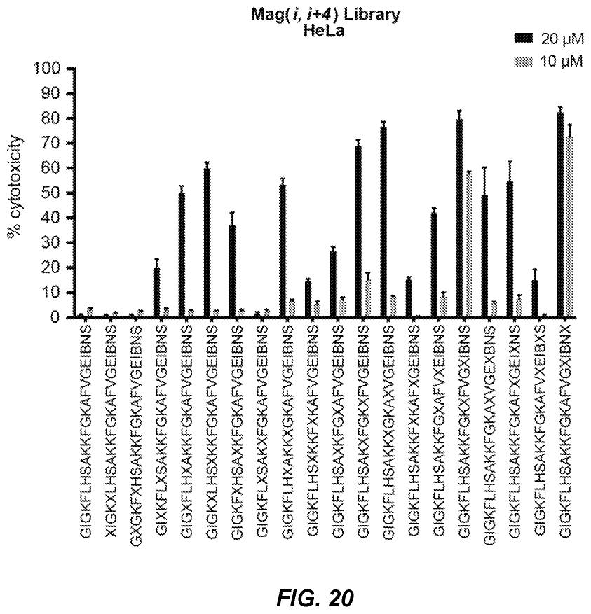

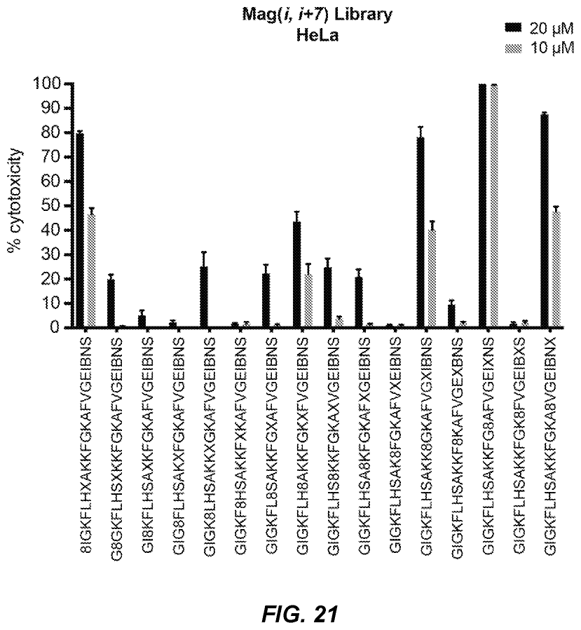

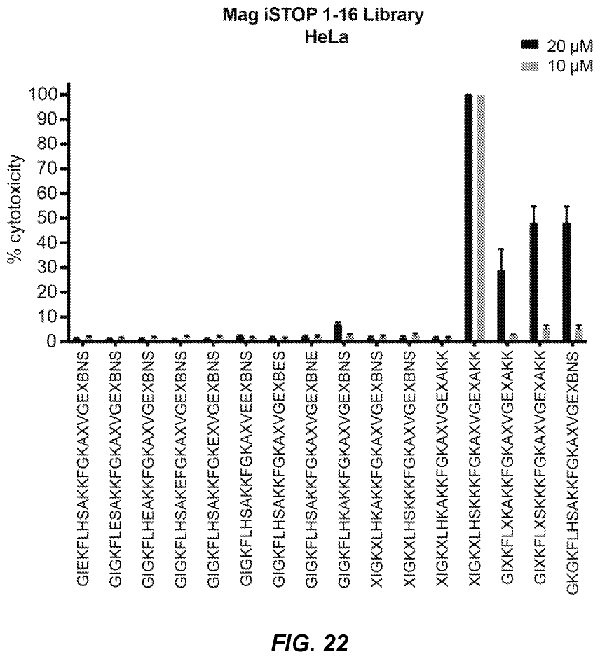

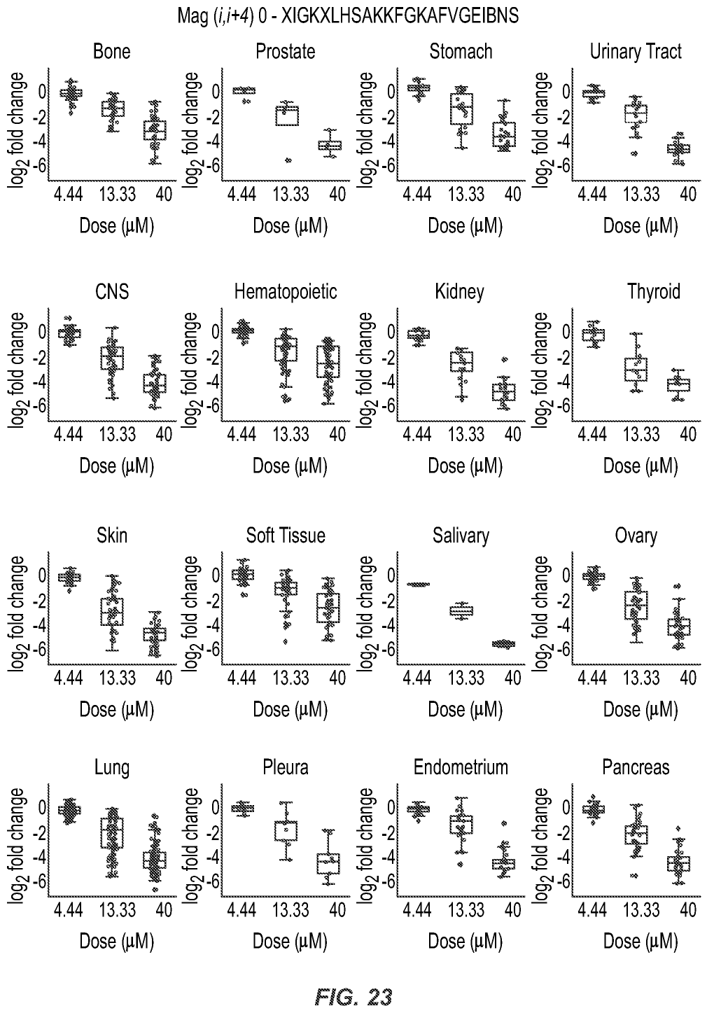

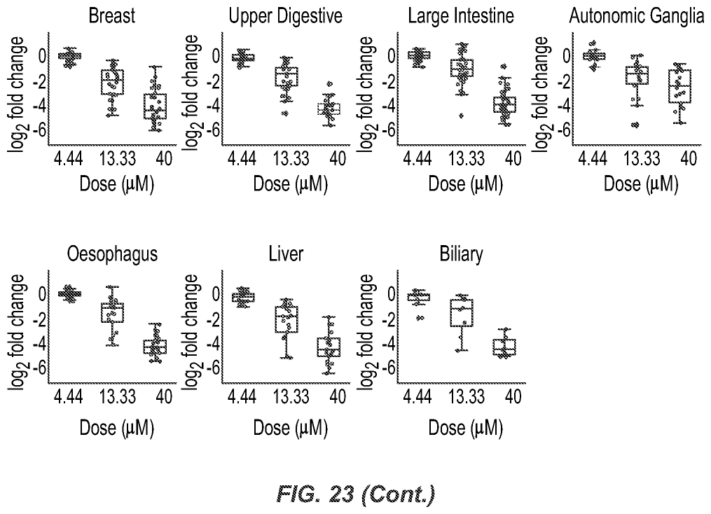

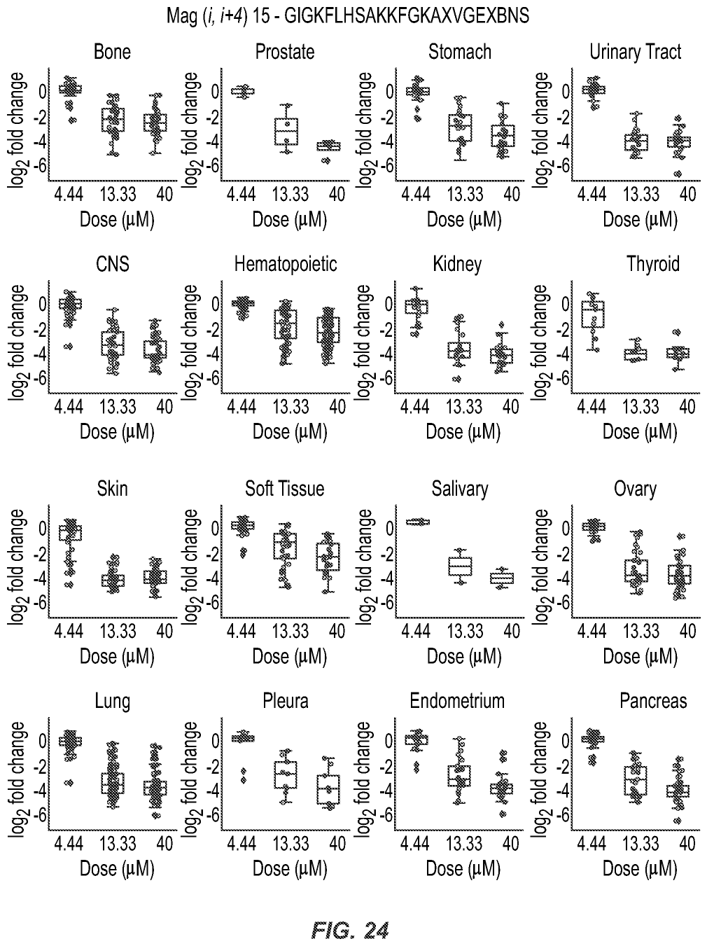

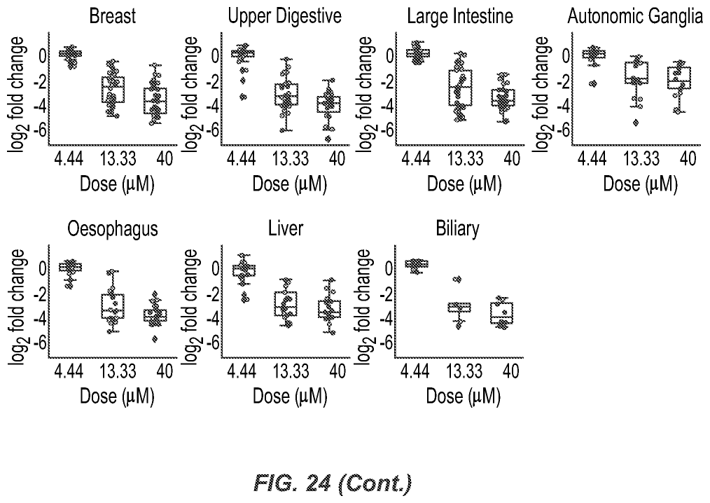

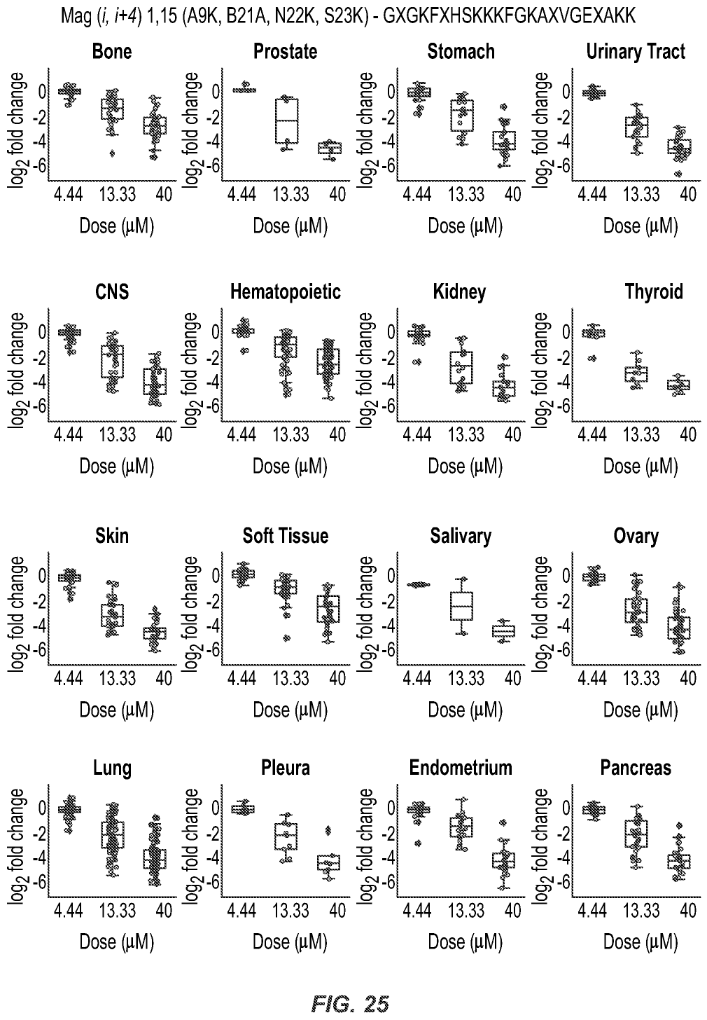

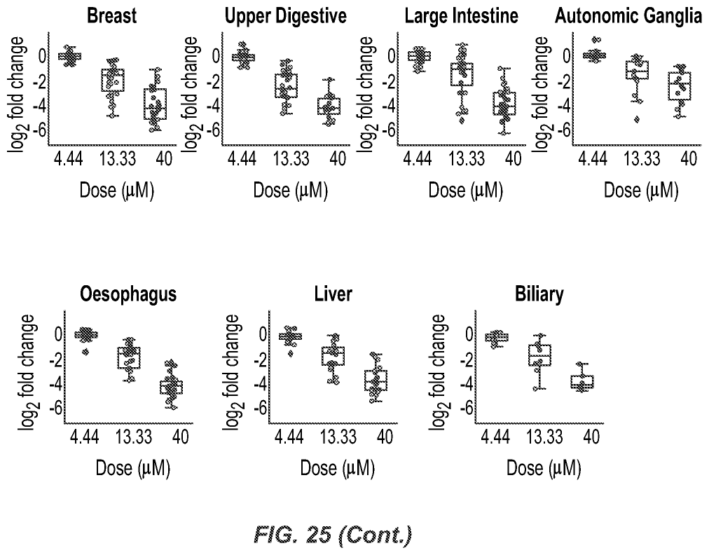

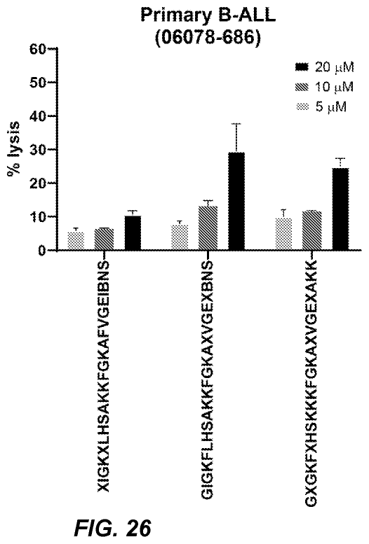

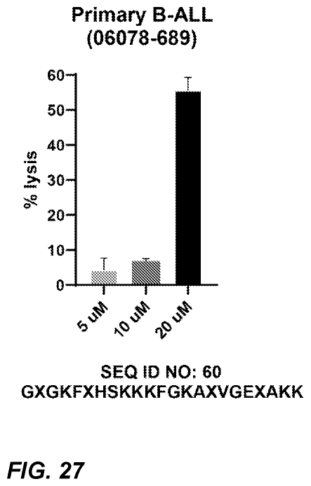

A series of stapled Magainin II peptides were generated by incorporating single i, i+7 staples, double i, i+4 staples, and point mutation(s) to iteratively assess and tune potency and selectivity (FIG. 17A and FIG. 17B). The compounds were tested in a series of cancer cell lines, revealing both selectivity of individually designed stapled oncolytic peptides for particular cancer cell types, and also a breadth of cytotoxic activity across a comprehensive battery of human cancer cell lines representing 45 lineages classified across 23 tissues of origin. Single i, i+4, single i, i+7 and an integrated stapled oncolytic peptide (iStOP) libraries were tested in OCI-AML3 cells (FIG. 4, FIG. 18, FIG. 19) and HeLa cervical cancer cells (FIG. 20, FIG. 21, FIG. 22) and showed differential cell lysis and cytotoxic activity depending on staple type, location, and combination of staple type(s) and mutants, with overlapping and nonoverlapping cancer cell susceptibility results. For example, comparing iStOPs 1-16 in OCI-AML3 and HeLa cells, there is (1) generally more susceptibility in the leukemia than cervical cancer cell line (e.g., iStOPs 1-12 show little activity in HeLa cells but differential potency in OCI-AML3 cells with select compounds showing especially potent and selective activity in OCI-AML3, e.g., iStOPs 2, 5, 6, 7, 8, 11), (2) differential susceptibility based on sequence/staple/mutational composition within each cancer cell line, and (3) compositions capable of killing both cancer cell lines (e.g., iStOPs 13-16). These constructs illustrate how hydrocarbon-stapling and mutagenesis can be integrated to yield optimal oncolytic peptides for therapeutic applications. A subpanel of constructs shown to be selective for lysing cancer cells and/or bacterial cells but not normal mammalian cells were then tested across more than 750 cancer cell lines representing more than 45 cancer cell lineages and classified by 23 tissues of origin. Dose-responsive cancer cell killing was observed across the full spectrum of human cancer cells, reflecting a remarkable breadth of cancer cell killing activity by StOPs of SEQ ID NO:2 (FIG. 23), SEQ ID NO:17 (FIG. 24), and SEQ ID NO:60 (FIG. 25). These results were next extended to primary human leukemia cells. StOPs corresponding to SEQ ID NOs: 2, 17, and 60 induced dose-responsive B-ALL lysis (patient sample 06078-686, 60% blasts) with differential potencies based on single-dose treatment and LDH release evaluation after only 90 minutes (FIG. 26). The StOP having the amino acid sequence of SEQ ID NO:60, which has been used to effectively treat bacterial infection in mice without gross toxicity, red cell hemolysis or damage to kidney epithelium (Mourtada et al., Nature Biotech, 2019), was the most potent and was again shown to have a dose-responsive lytic profile upon treatment of a distinct primary human B-ALL cell specimen (patient sample 06078-689, 70% blasts) (FIG. 27). Taken together, these data demonstrate that StOPs can be iteratively synthesized and tested to identify, through experimentation, those compositions capable of lysing human cancer cells, both broadly and in subtype-specific fashion (as exemplified by designs that lyse AML cells but not HeLa cells, both AML cells and HeLa cells, a broad spectrum of human cancer cells, and primary B-ALL cells), with iterative incorporation of staple type(s), staple placement, and point mutation(s) to arrive at lead constructs for desired applications (e.g., broad cancer targeting, selective cancer type targeting, and concomitant avoidance of toxicity to normal mammalian tissues).

### Materials and Methods Used in Examples 1-5:

Stapled oncolytic peptide synthesis: Fmoc-based solid-phase peptide synthesis was used to synthesize stapled antimicrobial peptides in accordance with previously reported methods for generating all-hydrocarbon stapled peptides. To achieve the various staple lengths, α-methyl, α-alkenyl amino acids were installed at i, i+4 positions using two S-pentenyl alanine residues (S5). For the stapling reaction, Grubbs 1st generation ruthenium catalyst dissolved in dichloroethane was added to the resin-bound peptides. To ensure maximal conversion, three to five rounds of stapling were performed. The peptides were then cleaved off of the resin using trifluoroacetic acid, precipitated using a hexane:ether (1:1) mixture, air dried, and purified by LC-MS. All peptides were quantified by amino acid analysis.
RBC hemolysis assay: Human blood samples were centrifuged to isolate red blood cells (RBCs), which were then washed and suspended in phosphate-buffered saline to yield a 1% (v/v) suspension. The suspension was added to serial dilutions of peptide stocks in water in clear round-bottom polypropylene 96-well plates and the plates incubated for 1 hour at 37° C. The plates were then centrifuged and the supernatant isolated to determine the amount of hemoglobin released using a spectrophotometer (570 nm). Percent hemolysis was calculated as: ([Treated Absorbance−Untreated Control Absorbance]×100)/(1% Triton X-100 Treated Absorbance−Untreated Control Absorbance).

LDH release assay: Cultured cells, including cancer cells and HUVEC cells, were plated in 96-well format (2×10⁴ cells per well; including overnight incubation for adherent cells) and then treated with serial dilutions of Mag(i+4) peptides in a final volume in a final volume of 100 L and incubated at 37° C. for the indicated time period. The plates were spun down at 1500 rpm for 5 min at 4° C., and 80 μL of cell culture media was transferred to a clear plate (Corning), incubated with 80 μL of LDH reagent (Roche) for 15 min while shaking, and absorbance measured at 490 nm on a microplate reader (SpectraMax M5 Microplate Reader, Molecular Devices). LDH release assays, as tailored to specific cancer cell types, are exemplified below.

LDH release assay of OCI-AML3 cells: OCI-AML3 cells were seeded in 96-well plates (2×10⁴ cells per well) in RPMI medium containing 5% FBS and treated with peptides having the indicated amino acid sequences for 90 minutes. LDH release was quantified by incubating centrifuged cell culture medium 1:1 with LDH reagent (Roche), followed by absorbance measurement 490 nm on a microplate reader (Spectramax M5). Percent LDH release was normalized to 1% Triton. Data was pooled from 2 technical replicates.

LDH release assay of primary human pediatric B-ALL cells: Primary human peripheral blood from a pediatric B-ALL patient was acquired under Dana-Farber Cancer Institute study protocol 06-078. Peripheral blood mononuclear cells (PBMCs) were purified by density-gradient centrifugation (Ficoll) and plated in 96-well plates (5×10⁴ cells per well). Cells were treated with StOP in RPMI medium, containing 5% FBS for 90 minutes. LDH release was quantified by incubating centrifuged cell culture medium 1:1 with LDH reagent (Roche), followed by absorbance measurement 490 nm on a microplate reader (Spectramax M5). Percent LDH release was normalized to 1% Triton.

IXM Live/Dead Imaging Assay: HeLa Cells: An alternative approach to assessing the rapid-onset cytotoxic activity of StOPs involves high content imaging by epiflorescence microscopy and Image Xpress processing. HeLa cells were plated in 384-well plates (3×10³ cells per well) in DMEM medium containing 5% FBS along with DRAQ7 (stains only permeabilized, identifies non-viable cells; 0.1 μM) and Hoechst 33342 (stains all nuclei, provides total cell count; 1 μM) and treated with the peptides having the indicated amino acid sequences at the indicated concentrations for 120 minutes. Cells were imaged on an ImageXpress Micro (Molecular Devices). Percent dead was plotted by computing DRAQ7-positive cells over total cell count (Hoechst). Treatment with 0.02% or 0.04% Triton led to all cells being DRAQ7 positive and thus provides the positive control for cell lysis. Data is pooled from 2 biological replicates.

PRISM Analysis: To broadly determine the cytotoxicity of StOPs across human cancer classes and subtypes, Profiling Relative Inhibition Simultaneously in Mixtures (PRISM) analysis of StOPs of SEQ ID NOs: 2, 17 and 60 was performed as described in Yu et al., Nat. Biotechnol. 2016, 34(4):419-423, which is incorporated by reference herein in its entirety. Compounds were evaluated in an 8-point 3-fold dilution series with a top dose of 40 μM in 384-well plates. Data for the top three doses (40 μM, 13.33 μM, and 4.44 μM) are presented in FIGS. 23-25. Cell sets (PR500 and PR300+) were treated for 3 days, followed by lysis, barcode amplification and detection using a bead-based Luminex system (see Yu et al., Nat. Biotechnol. 2016, 34(4):419-423).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of killing a cancer cell or inhibiting proliferation of a cancer cell in a human subject in need thereof, the method comprising administering to the human subject a therapeutically effective amount of a structurally-stabilized peptide, wherein the structurally-stabilized peptide is 18 to 50 amino acids in length and has at least 65% identity over the full length of the amino acid sequence GIGKFLHSAKKFGKAFVGEIMNS (SEQ ID NO:1) or the amino acid sequence set forth in any one of SEQ ID NOs: 219-221, 228, 231, and 232, wherein the structurally-stabilized peptide specifically lyses the cancer cell.

2.-4. (canceled)

5. A method of treating a cancer in a human subject in need thereof, the method comprising administering to the human subject a therapeutically effective amount of a structurally-stabilized peptide, wherein the structurally-stabilized peptide is 18 to 50 amino acids in length and has at least 65% identity over the full length of the amino acid sequence GIGKFLHSAKKFGKAFVGEIMNS (SEQ ID NO:1) or the amino acid sequence set forth in any one of SEQ ID NOs: 219-221, 228, 231, and 232, wherein the structurally-stabilized peptide specifically lyses a cancer cell of the human subject.

6. (canceled)

7. The method of claim 5, wherein cancer is a hematological cancer.

8. The method of claim 5, wherein the hematological cancer is leukemia, lymphoma, or multiple myeloma.

9.-12. (canceled)

13. The method of claim 5, wherein the structurally-stabilized peptide comprises the amino acid sequence:
(a) GX₁GKFX₂HSKKKFGKAX₃VGEX₄AKK, wherein each of X₁, X₂, X₃, and X₄ is independently a stapling amino acid (SEQ ID NO:60), and wherein a side chain of X₁ is cross-linked to a side chain of X₂ and a side chain of X₃ is cross-linked to a side chain of X₄.
(b) X₁IGKX₂LHSAKKFGKAX₃VGEX₄BNS, wherein each of X₁, X₂, X₃, and X₄ is independently a stapling amino acid (SEQ ID NO:141), and wherein a side chain of X₁ is cross-linked to a side chain of X₂ and a side chain of X₃ is cross-linked to a side chain of X₄;
(c) GIX₁KFLX₂SAKKFGKAX₃VGEX₄BNS, wherein each of X₁, X₂, X₃, and X₄ is independently a stapling amino acid (SEQ ID NO:142), and wherein a side chain of X₁ is cross-linked to a side chain of X₂ and a side chain of X₃ is cross-linked to a side chain of X₄;
(d) GIX₁KFLX₂KAKKFGKAX₃VGEX₄BNS, wherein each of X₁, X₂, X₃, and X₄ is independently a stapling amino acid (SEQ ID NO:143), and wherein a side chain of X₁ is cross-linked to a side chain of X₂ and a side chain of X₃ is cross-linked to a side chain of X₄;
(e) GIX₁KFLX₂SKKKFGKAX₃VGEX₄BNS, wherein each of X₁, X₂, X₃, and X₄ is independently a stapling amino acid (SEQ ID NO:144), and wherein a side chain of X₁ is cross-linked to a side chain of X₂ and a side chain of X₃ is cross-linked to a side chain of X₄;
(f) GX₁GKFX₂HSKKKFGKAX₃VGEX₄BNS, wherein each of X₁, X₂, X₃, and X₄ is independently a stapling amino acid (SEQ ID NO:145), and wherein a side chain of X₁ is cross-linked to a side chain of X₂ and a side chain of X₃ is cross-linked to a side chain of X₄;
(g) GIGKFLHSAKKFGKAX₁VGEX₂BNS, wherein each of X₁ and X₂ is independently a stapling amino acid (SEQ ID NO:17), and wherein a side chain of X₁ is cross-linked to a side chain of X₂;
(h) GIGKFLHKAKKFGKAX₁VGEX₂BNS, wherein each of X₁ and X₂ is independently a stapling amino acid (SEQ ID NO:27), and wherein a side chain of X₁ is cross-linked to a side chain of X₂;
(i) X₁IGKX₂LHKAKKFGKAX₃VGEX₄BNS, wherein each of X₁, X₂, X₃, and X₄ is independently a stapling amino acid (SEQ ID NO:98), and wherein a side chain of X₁ is cross-linked to a side chain of X₂ and a side chain of X₃ is cross-linked to a side chain of X₄;
(i) X₁IGKX₂LHSKKKFGKAX₃VGEX₄BNS, wherein each of X₁, X₂, X₃, and X₄ is independently a stapling amino acid (SEQ ID NO:99), and wherein a side chain of X₁ is cross-linked to a side chain of X₂ and a side chain of X₃ is cross-linked to a side chain of X₄;
(k) GIGKFLHSKKKFGKAX₁VGEX₂BNS, wherein each of X₁ and X₂ is independently a stapling amino acid (SEQ ID NO:28), and wherein a side chain of X₁ is cross-linked to a side chain of X₂;
(l) X₁KGKX₂LHSAKKFGKAX₃VGEX₄BNS, wherein each of X₁, X₂, X₃, and X₄ is independently a stapling amino acid (SEQ ID NO:133), and wherein a side chain of X₁ is cross-linked to a side chain of X₂ and a side chain of X₃ is cross-linked to a side chain of X₄;
(m) X₁IEKX₂LHSAKKFGKAX₃VGEX₄BNS, wherein each of X₁, X₂, X₃, and X₄ is independently a stapling amino acid (SEQ ID NO:134), and wherein a side chain of X₁ is cross-linked to a side chain of X₂ and a side chain of X₃ is cross-linked to a side chain of X₄;
(n) X₁IGKX₂LESAKKFGKAX₃VGEX₄BNS, wherein each of X₁, X₂, X₃, and X₄ is independently a stapling amino acid (SEQ ID NO:135), and wherein a side chain of X₁ is cross-linked to a side chain of X₂ and a side chain of X₃ is cross-linked to a side chain of X₄;
(o) X₁IGKX₂LHSAKKFGKAX₃VEEX₄BNS, wherein each of X₁, X₂, X₃, and X₄ is independently a stapling amino acid (SEQ ID NO:136), and wherein a side chain of X₁ is cross-linked to a side chain of X₂ and a side chain of X₃ is cross-linked to a side chain of X₄;
(p) X₁IGKX₂LHSAKKFGKAFVGEIBES, wherein each of X₁, and X₂ is independently a stapling amino acid (SEQ ID NO:137), and wherein a side chain of X₁ is cross-linked to a side chain of X₂;
(q) GKX₁KFLX₂SAKKFGKAFVGEIBNS, wherein each of X₁, and X₂ is independently a stapling amino acid (SEQ ID NO:138), and wherein a side chain of X₁ is cross-linked to a side chain of X₂;
(r) GIX₁KFLX₂SAKKFGKAFVEEIBNS, wherein each of X₁ and X₂ is independently a stapling amino acid (SEQ ID NO:139), and wherein a side chain of X₁ is cross-linked to a side chain of X₂;
(s) GKX₁KFLX₂SAKKFGKAFVGEIBES, wherein each of X₁ and X₂ is independently a stapling amino acid (SEQ ID NO:140), and wherein a side chain of X₁ is cross-linked to a side chain of X₂;
(t) X₁IGKX₂LHSAKKFGKAFVGEIBNS, wherein each of X₁ and X₂ is independently a stapling amino acid (SEQ ID NO:2), and wherein a side chain of X₁ is cross-linked to a side chain of X₂;
(u) GX₁GKFX₂HSAKKFGKAFVGEIBNS, wherein each of X₁ and X₂ is independently a stapling amino acid (SEQ ID NO:3), and wherein a side chain of X₁ is cross-linked to a side chain of X₂;
(v) GIX₁KFLX₂SAKKFGKAFVGEIBNS, wherein each of X₁ and X₂ is independently a stapling amino acid (SEQ ID NO:4), and wherein a side chain of X₁ is cross-linked to a side chain of X₂;
(w) GIGX₁FLHX₂AKKFGKAFVGEIBNS, wherein each of X₁ and X₂ is individually a stapling amino acid (SEQ ID NO:5), and wherein a side chain of X₁ is cross-linked to a side chain of X₂;
(x) GIGKX₁LHSX₂KKFGKAFVGEIBNS, wherein each of X₁ and X₂ is independently a stapling amino acid (SEQ ID NO:6), and wherein a side chain of X₁ is cross-linked to a side chain of X₂;
(y) GIGKFLX₁SAKX₂FGKAFVGEIBNS, wherein each of X₁ and X₂ is independently a stapling amino acid (SEQ ID NO:8), and wherein a side chain of X₁ is cross-linked to a side chain of X₂;
(z) GIGKFLHX₁AKKX₂GKAFVGEIBNS, wherein each of X₁ and X₂ is independently a stapling amino acid (SEQ ID NO:9), and wherein a side chain of X₁ is cross-linked to a side chain of X₂;
(aa) GIGKFLHSX₁KKFX₂KAFVGEIBNS, wherein each of X₁ and X₂ is independently a stapling amino acid (SEQ ID NO:10), and wherein a side chain of X₁ is cross-linked to a side chain of X₂;
(bb) GIGKFLHSAX₁KFGX₂AFVGEIBNS, wherein each of X₁ and X₂ is independently a stapling amino acid (SEQ ID NO:11), and wherein a side chain of X₁ is cross-linked to a side chain of X₂;
(cc) GIGKFLHSAKKX₁GKAX₂VGEIBNS, wherein each of X₁ and X₂ is independently a stapling amino acid (SEQ ID NO:13), and wherein a side chain of X₁ is cross-linked to a side chain of X₂;
(dd) GIGKFLHSAKKFGX₁AFVX₂EIBNS, wherein each of X₁ and X₂ is independently a stapling amino acid (SEQ ID NO:15), and wherein a side chain of X₁ is cross-linked to a side chain of X₂;
(ee) GIGKFLHSAKKFGKX₁FVGX₂IBNS, wherein each of X₁ and X₂ is independently a stapling amino acid (SEQ ID NO:16), and wherein a side chain of X₁ is cross-linked to a side chain of X₂;
(ff) GIGKFLHSAKKFGKAFX₁GEIX₂NS, wherein each of X₁ and X₂ is independently a stapling amino acid (SEQ ID NO:18), and wherein a side chain of X₁ is cross-linked to a side chain of X₂;
(gg) GIGKFLHSAKKFGKAFVX₁EIBX₂S, wherein each of X₁ and X₂ is independently a stapling amino acid (SEQ ID NO:19), and wherein a side chain of X₁ is cross-linked to a side chain of X₂;
(hh) GIGKFLHSAKKFGKAFVGX₁IBNX₂, wherein each of X₁ and X₂ is independently a stapling amino acid (SEQ ID NO:20), and wherein a side chain of X₁ is cross-linked to a side chain of X₂;
(ii) KIGKFLHSAKKFGKAX₁VGEX₂BNS, wherein each of X₁ and X₂ is independently a stapling amino acid (SEQ ID NO:21), and wherein a side chain of X₁ is cross-linked to a side chain of X₂;
(ii) GKGKFLHSAKKFGKAX₁VGEX₂BNS, wherein each of X₁ and X₂ is independently a stapling amino acid (SEQ ID NO:22), and wherein a side chain of X₁ is cross-linked to a side chain of X₂;
(kk) GIKKFLHSAKKFGKAX₁VGEX₂BNS, wherein each of X₁ and X₂ is independently a stapling amino acid (SEQ ID NO:23), and wherein a side chain of X₁ is cross-linked to a side chain of X₂;
(ll) GIGKFLKSAKKFGKAX₁VGEX₂BNS, wherein each of X₁ and X₂ is independently a stapling amino acid (SEQ ID NO:26), and wherein a side chain of X₁ is cross-linked to a side chain of X₂;
(mm) GIGKFLHSAKKFGKKX₁VGEX₂BNS, wherein each of X₁ and X₂ is independently a stapling amino acid (SEQ ID NO:31), and wherein a side chain of X₁ is cross-linked to a side chain of X₂;
(nn) GIGKFLHSAKKFGKAX₁VKEX₂BNS, wherein each of X₁ and X₂ is independently a stapling amino acid (SEQ ID NO:34), and wherein a side chain of X₁ is cross-linked to a side chain of X₂;
(oo) GIGKFLHSAKKFGKAX₁VGKX₂BNS, wherein each of X₁ and X₂ is independently a stapling amino acid (SEQ ID NO:35), and wherein a side chain of X₁ is cross-linked to a side chain of X₂;
(pp) GIGKFLHSAKKFGKAX₁VGEX₂KNS, wherein each of X₁ and X₂ is independently a stapling amino acid (SEQ ID NO:37), and wherein a side chain of X₁ is cross-linked to a side chain of X₂;
(qq) GIGKFLHSAKKFGKAX₁VGEX₂BKS, wherein each of X₁ and X₂ is independently a stapling amino acid (SEQ ID NO:38), and wherein a side chain of X₁ is cross-linked to a side chain of X₂;
(rr) GIEKFLHSAKKFGKAX₁VGEX₂BNS, wherein each of X₁ and X₂ is independently a stapling amino acid (SEQ ID NO:42), and wherein a side chain of X₁ is cross-linked to a side chain of X₂;
(ss) GIGKFLESAKKFGKAX₁VGEX₂BNS, wherein each of X₁ and X₂ is independently a stapling amino acid (SEQ ID NO:46), and wherein a side chain of X₁ is cross-linked to a side chain of X₂;
(tt) GIGKFLHEAKKFGKAX₁VGEX₂BNS, wherein each of X₁ and X₂ is independently a stapling amino acid (SEQ ID NO:47), and wherein a side chain of X₁ is cross-linked to a side chain of X₂;
(uu) GIGKFLHSAKKFGKEX₁VGEX₂BNS, wherein each of X₁ and X₂ is independently a stapling amino acid (SEQ ID NO:54), and wherein a side chain of X₁ is cross-linked to a side chain of X₂;
(vv) GIGKFLHSAKKFGKAX₁VEEX₂BNS, wherein each of X₁ and X₂ is independently a stapling amino acid (SEQ ID NO:56), and wherein a side chain of X₁ is cross-linked to a side chain of X₂;
(ww) GIGKFLHSAKKFGKAX₁VGEX₂BES, wherein each of X₁ and X₂ is independently a stapling amino acid (SEQ ID NO:58), and wherein a side chain of X₁ is cross-linked to a side chain of X₂; or
(xx) GIGKFLHSAKKFGKAX₁VGEX₂BNE, wherein each of X₁ and X₂ is independently a stapling amino acid (SEQ ID NO:59), and wherein a side chain of X₁ is cross-linked to a side chain of X₂,
wherein B in the above recited sequences is norleucine.

14. The method of claim 5, wherein the structurally-stabilized peptide comprises the formula: 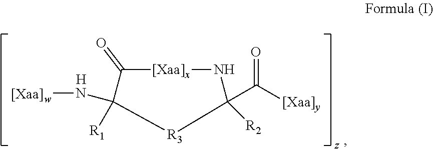
or a pharmaceutically acceptable salt thereof, wherein:
each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
each R₃ is independently alkylene, alkenylene, or alkynylene, any of which is substituted or unsubstituted;
z is 1, 2, 3, 4, 5, 6, 7, 8, 9, or 10; and
(a) each [Xaa]_{w} is absent, each [Xaa]ₓ is IGK, and each [Xaa]_{y} is LHSAKKFGKAFVGEIBNS (SEQ ID NO:32) or LHSAKKFGKAFVGEIANS (SEQ ID NO:89);
(b) each [Xaa]_{w} is G, each [Xaa]ₓ is GKF, and each [Xaa]_{y} is HSAKKFGKAFVGEIBNS (SEQ ID NO:122) or HSAKKFGKAFVGEIANS (SEQ ID NO:90);
(c) each [Xaa]_{w} is GI, each [Xaa]ₓ is KFL, and each [Xaa]_{y} is SAKKFGKAFVGEIBNS (SEQ ID NO:76) or SAKKFGKAFVGEIANS (SEQ ID NO:94);
(d) each [Xaa]_{w} is GIGK (SEQ ID NO:39), each [Xaa]ₓ is LHS, and each [Xaa]_{y} is KKFGKAFVGEIBNS (SEQ ID NO:77) or KKFGKAFVGEIANS (SEQ ID NO:95);
(e) each [Xaa]_{w} is GIGKFL (SEQ ID NO:78), each [Xaa]ₓ is SAK, and each [Xaa]_{y} is FGKAFVGEIBNS (SEQ ID NO:79) or FGKAFVGEIANS (SEQ ID NO:61);
(f) each [Xaa]_{w} is GIGKFLHSAKK (SEQ ID NO:80), each [Xaa]ₓ is GKA, and each [Xaa]_{y} is VGEIBNS (SEQ ID NO:81) or VGEIANS (SEQ ID NO: 62);
(g) each [Xaa]_{w} is GIGKFLHSAKKFGKA (SEQ ID NO: 82), each [Xaa]ₓ is VGE, and each [Xaa]_{y} is BNS or ANS;
(h) each [Xaa]_{w} is GIGKFLHSAKKFGKAF (SEQ ID NO:83), each [Xaa]ₓ is GEI, and each [Xaa]_{y} is NS;
(i) each [Xaa]_{w} is KIGKFLHSAKKFGKA (SEQ ID NO:84), each [Xaa]ₓ is VGE, and each [Xaa]_{y} is BNS or ANS;
(j) each [Xaa]_{w} is GKGKFLHSAKKFGKA (SEQ ID NO:85), each [Xaa]ₓ is VGE, and each [Xaa]_{y} is BNS or ANS;
(k) each [Xaa]_{w} is GIKKFLHSAKKFGKA (SEQ ID NO:86), each [Xaa]ₓ is VGE, and each [Xaa]_{y} is BNS or ANS;
(l) each [Xaa]_{w} is GIGKFLKSAKKFGKA (SEQ ID NO:87), each [Xaa]ₓ is VGE, and each [Xaa]_{y} is BNS or ANS;
(m) each [Xaa]_{w} is GIGKFLHKAKKFGKA (SEQ ID NO:88), each [Xaa]ₓ is VGE, and each [Xaa]_{y} is BNS or ANS;
(n) each [Xaa]_{w} is GIGKFLHSAKKFGKA (SEQ ID NO:82), each [Xaa]ₓ is VGK, and each [Xaa]_{y} is BNS or ANS;
(o) each [Xaa]_{w} is GIGKFLHSAKKFGKA (SEQ ID NO:82), each [Xaa]ₓ is VGE, and each [Xaa]_{y} is BKS or AKS;
(p) each [Xaa]_{w} is GIEKFLHSAKKFGKA (SEQ ID NO:91), each [Xaa]ₓ is VGE, and each [Xaa]_{y} is BNS or ANS;
(q) each [Xaa]_{w} is GIGKFLESAKKFGKA (SEQ ID NO:92), each [Xaa]ₓ is VGE, and each [Xaa]_{y} is BNS or ANS;
(r) each [Xaa]_{w} is GIGKFLHEAKKFGKA (SEQ ID NO:93), each [Xaa]ₓ is VGE, and each [Xaa]_{y} is BNS or ANS;
(s) each [Xaa]_{w} is GIGKFLHSAKKFGKA (SEQ ID NO:82), each [Xaa]ₓ is VEE, and each [Xaa]_{y} is BNS or ANS;
(t) each [Xaa]_{w} is GIGKFLHSAKKFGKA (SEQ ID NO:82), each [Xaa]ₓ is VGE, and each [Xaa]_{y} is BES or AES;
(u) each [Xaa]_{w} is G, each [Xaa]ₓ is GKF, and each [Xaa]_{y} is HSKKKFGKAX₁VGEX₂AKK (SEQ ID NO:96), wherein each of X₁ and X₂ is a stapling amino acid, and wherein a side chain of X₁ is cross-linked to a side chain of X₂; or
(v) each [Xaa]_{w} is GX₁GKFX₂HSKKKFGKA (SEQ ID NO:97), each [Xaa]ₓ is VGE, and each [Xaa]_{y} is AKK, wherein each of X₁ and X₂ is a stapling amino acid, and wherein a side chain of X₁ is cross-linked to a side chain of X₂;
(w) each [Xaa]_{w} is GIGKFLHSAKKFGKA (SEQ ID NO:82), each [Xaa]ₓ is VGE, and each [Xaa]_{y} is BNE or ANE;
(x) each [Xaa]_{w} is absent, each [Xaa]ₓ is KGK, and each [Xaa]_{y} is LHSAKKFGKAX₃VGEX₄BNS (SEQ ID NO:160) or LHSAKKFGKAX₃VGEX₄ANS (SEQ ID NO:161), wherein each of X₃ and X₄ is a stapling amino acid, and wherein a side chain of X₃ is cross-linked to a side chain of X₄;
(y) each [Xaa]_{w} is X₁KGKX₂LHSAKKFGKA, wherein each of X₁ and X₂ is a stapling amino acid (SEQ ID NO:162), and wherein a side chain of X₁ is cross-linked to a side chain of X₂, each [Xaa]ₓ is VGE, and each [Xaa]_{y} is BNS or ANS;
(z) each [Xaa]_{w} is absent, each [Xaa]ₓ is IEK, and each [Xaa]_{y} is LHSAKKFGKAX₃VGEX₄BNS (SEQ ID NO:163) or LHSAKKFGKAX₃VGEX₄ANS (SEQ ID NO:164), wherein each of X₃ and X₄ is a stapling amino acid, and wherein a side chain of X₃ is cross-linked to a side chain of X₄;
(aa) each [Xaa]_{w} is X₁IEKX₂LHSAKKFGKA, wherein each of X₁ and X₂ is a stapling amino acid (SEQ ID NO:165), and wherein a side chain of X₁ is cross-linked to a side chain of X₂, each [Xaa]ₓ is VGE, and each [Xaa]_{y} is BNS or ANS;
(bb) each [Xaa]_{w} is absent, each [Xaa]ₓ is IGK, and each [Xaa]_{y} is LESAKKFGKAX₃VGEX₄BNS (SEQ ID NO:166) or LESAKKFGKAX₃VGEX₄ANS (SEQ ID NO:167), wherein each of X₃ and X₄ is a stapling amino acid, and wherein a side chain of X₃ is cross-linked to a side chain of X₄;
(cc) each [Xaa]_{w} is X₁IGKX₂LESAKKFGKA, wherein each of X₁ and X₂ is a stapling amino acid (SEQ ID NO:191), and wherein a side chain of X₁ is cross-linked to a side chain of X₂, each [Xaa]ₓ is VGE, and each [Xaa]_{y} is BNS or ANS;
(dd) each [Xaa]_{w} is absent, each [Xaa]ₓ is IGK, and each [Xaa]_{y} is LHSAKKFGKAX₃VEEX₄BNS (SEQ ID NO:315) or LHSAKKFGKAX₃VEEX₄ANS (SEQ ID NO:316), wherein each of X₃ and X₄ is a stapling amino acid, and wherein a side chain of X₃ is cross-linked to a side chain of X₄;
(ee) each [Xaa]_{w} is X₁IGKX₂LHSAKKFGKA, wherein each of X₁ and X₂ is a stapling amino acid (SEQ ID NO:178), and wherein a side chain of X₁ is cross-linked to a side chain of X₂, each [Xaa]ₓ is VEE, and each [Xaa]_{y} is BNS or ANS;
(ff) each [Xaa]_{w} is absent, each [Xaa]ₓ is IGK, and each [Xaa]_{y} is LHSAKKFGKAFVGEIBES (SEQ ID NO:168) or LHSAKKFGKAFVGEIAES (SEQ ID NO:169);
(gg) each [Xaa]_{w} is GK, each [Xaa]ₓ is KFL, and each [Xaa]_{y} is SAKKFGKAFVGEIBNS (SEQ ID NO:170) or SAKKFGKAFVGEIANS (SEQ ID NO:171);
(hh) each [Xaa]_{w} is GI, each [Xaa]ₓ is KFL, and each [Xaa]_{y} is SAKKFGKAFVEEIBNS (SEQ ID NO:172) or SAKKFGKAFVEEIANS (SEQ ID NO:173);
(ii) each [Xaa]_{w} is GK, each [Xaa]ₓ is KFL, and each [Xaa]_{y} is SAKKFGKAFVGEIBES (SEQ ID NO:174) or SAKKFGKAFVGEIAES (SEQ ID NO:175);
(jj) each [Xaa]_{w} is absent, each [Xaa]ₓ is IGK, and each [Xaa]_{y} is LHSAKKFGKAX₃VGEX₄BNS (SEQ ID NO:176) or LHSAKKFGKAX₃VGEX₄ANS (SEQ ID NO:177), wherein each of X₃ and X₄ is a stapling amino acid, and wherein a side chain of X₃ is cross-linked to a side chain of X₄;
(kk) each [Xaa]_{w} is X₁IGKX₂LHSAKKFGKA, wherein each of X₁ and X₂ is a stapling amino acid (SEQ ID NO:178), and wherein a side chain of X₁ is cross-linked to a side chain of X₂, each [Xaa]ₓ is VGE, and each [Xaa]_{y} is BNS or ANS;
(ll) each [Xaa]_{w} is GI, each [Xaa]ₓ is KFL, and each [Xaa]_{y} is SAKKFGKAX₃VGEX₄BNS (SEQ ID NO:179) or SAKKFGKAX₃VGEX₄ANS (SEQ ID NO:180), wherein each of X₃ and X₄ is a stapling amino acid, and wherein a side chain of X₃ is cross-linked to a side chain of X₄;
(mm) each [Xaa]_{w} is GIX₁KFLX₂SAKKFGKA, wherein each of X₁ and X₂ is a stapling amino acid (SEQ ID NO:181), and wherein a side chain of X₁ is cross-linked to a side chain of X₂, each [Xaa]ₓ is VGE, and each [Xaa]_{y} is BNS or ANS;
(nn) each [Xaa]_{w} is GI, each [Xaa]ₓ is KFL, and each [Xaa]_{y} is KAKKFGKAX₃VGEX₄BNS (SEQ ID NO:182) or KAKKFGKAX₃VGEX₄ANS (SEQ ID NO:183), wherein each of X₃ and X₄ is a stapling amino acid, and wherein a side chain of X₃ is cross-linked to a side chain of X₄;
(oo) each [Xaa]_{w} is GIX₁KFLX₂KAKKFGKA, wherein each of X₁ and X₂ is a stapling amino acid (SEQ ID NO:184), and wherein a side chain of X₁ is cross-linked to a side chain of X₂, each [Xaa]ₓ is VGE, and each [Xaa]_{y} is BNS or ANS;
(pp) each [Xaa]_{w} is GI, each [Xaa]ₓ is KFL, and each [Xaa]_{y} is SKKKFGKAX₃VGEX₄BNS (SEQ ID NO:185) or SKKKFGKAX₃VGEX₄ANS (SEQ ID NO:186), wherein each of X₃ and X₄ is a stapling amino acid, and wherein a side chain of X₃ is cross-linked to a side chain of X₄;
(qq) each [Xaa]_{w} is GIX₁KFLX₂SKKKFGKA, wherein each of X₁ and X₂ is a stapling amino acid (SEQ ID NO:187), wherein a side chain of X₁ is cross-linked to a side chain of X₂, each [Xaa]ₓ is VGE, and each [Xaa]_{y} is BNS or ANS;
(rr) each [Xaa]_{w} is G, each [Xaa]ₓ is GFK, and each [Xaa]_{y} is HSKKKFGKAX₃VGEX₄BNS (SEQ ID NO:188) or HSKKKFGKAX₃VGEX₄ANS (SEQ ID NO:189), wherein each of X₃ and X₄ is a stapling amino acid, and wherein a side chain of X₃ is cross-linked to a side chain of X₄; or
(ss) each [Xaa]_{w} is GX₁GKFX₂HSKKKFGKA, wherein each of X₁ and X₂ is a stapling amino acid (SEQ ID NO:190), and wherein a side chain of X₁ is cross-linked to a side chain of X₂, each [Xaa]ₓ is VGE, and each [Xaa]_{y} is BNS or ANS;
(tt) each [Xaa]_{w} is GIG, each [Xaa]ₓ is FLH, and each [Xaa]_{y} is AKKFGKAFVGEIBNS (SEQ ID NO:207) or AKKFGKAFVGEIANS (SEQ ID NO:213);
(uu) each [Xaa]_{w} is GIGKFLH (SEQ ID NO:200), each [Xaa]ₓ is AKK, and each [Xaa]_{y} is GKAFVGEIBNS (SEQ ID NO:208) or GKAFVGEIANS (SEQ ID NO:214);
(vv) each [Xaa]_{w} is GIGKFLHS (SEQ ID NO:201), each [Xaa]ₓ is KKF, and each [Xaa]_{y} is KAFVGEIBNS (SEQ ID NO:209) or KAFVGEIANS (SEQ ID NO:215);
(ww) each [Xaa]_{w} is GIGKFLHSA (SEQ ID NO:202), each [Xaa]ₓ is KFG, and each [Xaa]_{y} is AFVGEIBNS (SEQ ID NO:210) or AFVGEIANS (SEQ ID NO:216);
(xx) each [Xaa]_{w} is GIGKFLHSAKKFG (SEQ ID NO:203), each [Xaa]ₓ is AFV, and each [Xaa]_{y} is EIBNS (SEQ ID NO:211) or EIANS (SEQ ID NO:217);
(yy) each [Xaa]_{w} is GIGKFLHSAKKFGK (SEQ ID NO:204), each [Xaa]ₓ is FVG, and each [Xaa]_{y} is IBNS (SEQ ID NO:212) or IANS (SEQ ID NO:218);
(zz) each [Xaa]_{w} is GIGKFLHSAKKFGKAFV (SEQ ID NO:205), each [Xaa]ₓ is EIB, and each [Xaa]_{y} is S;
(aaa) each [Xaa]_{w} is GIGKFLHSAKKFGKAFVG (SEQ ID NO:206), each [Xaa]ₓ is IBN, and each [Xaa]_{y} is absent;
(bbb) each [Xaa]_{w} is absent, each [Xaa]ₓ is IGKFLH (SEQ ID NO:237), and each [Xaa]_{y} is AKKFGKAFVGEIBNS (SEQ ID NO:253) or AKKFGKAFVGEIANS (SEQ ID NO:279);
(ccc) each [Xaa]_{w} is G, each [Xaa]ₓ is GKFLHS (SEQ ID NO:238), and each [Xaa]_{y} is KKFGKAFVGEIBNS (SEQ ID NO:254) or KKFGKAFVGEIANS (SEQ ID NO:280);
(ddd) each [Xaa]_{w} is GI, each [Xaa]ₓ is KFLHSA (SEQ ID NO:239), and each [Xaa]_{y} is KFGKAFVGEIBNS (SEQ ID NO:255) or KFGKAFVGEIANS (SEQ ID NO:281);
(eee) each [Xaa]_{w} is GIG, each [Xaa]ₓ is FLHSAK (SEQ ID NO:240), and each [Xaa]_{y} is FGKAFVGEIBNS (SEQ ID NO:256) or FGKAFVGEIANS (SEQ ID NO:282);
(fff) each [Xaa]_{w} is GIGK (SEQ ID NO:39), each [Xaa]ₓ is LHSAKK (SEQ ID NO:241), and each [Xaa]_{y} is GKAFVGEIBNS (SEQ ID NO:257) or GKAFVGEIANS (SEQ ID NO:283);
(ggg) each [Xaa]_{w} is GIGKF (SEQ ID NO:234), each [Xaa]ₓ is HSAKKF (SEQ ID NO:242), and each [Xaa]_{y} is KAFVGEIBNS (SEQ ID NO:258) or KAFVGEIANS (SEQ ID NO:284);
(hhh) each [Xaa]_{w} is GIGKFL (SEQ ID NO:78), each [Xaa]ₓ is SAKKFG (SEQ ID NO:243), and each [Xaa]_{y} is AFVGEIBNS (SEQ ID NO:259) or AFVGEIANS (SEQ ID NO:285);
(iii) each [Xaa]_{w} is GIGKFLH (SEQ ID NO:200), each [Xaa]ₓ is AKKFGK (SEQ ID NO:244), and each [Xaa]_{y} is FVGEIBNS (SEQ ID NO:260) or FVGEIANS (SEQ ID NO:286);
(jjj) each [Xaa]_{w} is GIGKFLHS (SEQ ID NO:201), each [Xaa]ₓ is KKFGKA (SEQ ID NO:245), and each [Xaa]_{y} is VGEIBNS (SEQ ID NO:261) or VGEIANS (SEQ ID NO:287);
(kkk) each [Xaa]_{w} is GIGKFLHSA (SEQ ID NO:202), each [Xaa]ₓ is KFGKAF (SEQ ID NO:246), and each [Xaa]_{y} is GEIBNS (SEQ ID NO:262) or GEIANS (SEQ ID NO:288);
(lll) each [Xaa]_{w} is GIGKFLHSAK (SEQ ID NO:235), each [Xaa]ₓ is FGKAFV (SEQ ID NO:247), and each [Xaa]_{y} is EIBNS (SEQ ID NO:263) or EIANS (SEQ ID NO:289);
(mmm) each [Xaa]_{w} is GIGKFLHSAKK (SEQ ID NO:80), each [Xaa]ₓ is GKAFVG (SEQ ID NO:248), and each [Xaa]_{y} is IBNS (SEQ ID NO:264) or IANS (SEQ ID NO:290);
(nnn) each [Xaa]_{w} is GIGKFLHSAKKF (SEQ ID NO:236), each [Xaa]ₓ is KAFVGE (SEQ ID NO:249), and each [Xaa]_{y} is BNS or ANS;
(ooo) each [Xaa]_{w} is GIGKFLHSAKKFG (SEQ ID NO:203), each [Xaa]ₓ is AFVGEI (SEQ ID NO:250), and each [Xaa]_{y} is NS;
(ppp) each [Xaa]_{w} is GIGKFLHSAKKFGK (SEQ ID NO:204), each [Xaa]ₓ is FVGEIB (SEQ ID NO:251) or FVGEIA (SEQ ID NO:277), and each [Xaa]_{y} is S; or
(qqq) each [Xaa]_{w} is GIGKFLHSAKKFGKA (SEQ ID NO:82), each [Xaa]ₓ is VGEIBN (SEQ ID NO:252) or VGEIAN (SEQ ID NO:278), and each [Xaa]_{y} is absent; and
wherein B is norleucine.

15. The method of claim 14, wherein R₁ is an alkyl, R₂ is an alkyl, and R₃ is an alkenyl.

16. The method of claim 14, wherein R₁ is a methyl group, R₂ is a methyl group, and R₃ is —CH2-CH2-CH2-CH═CH—CH2-CH2-CH2- or —(CH₂)₆—CH═CH—(CH₂)₆—.

17.-22. (canceled)

23. The method of claim 14, wherein z is 1.

24. The method of claim 5, wherein the structurally-stabilized peptide comprises the amino acid sequence set forth in any one of SEQ ID NOs: 2-6, 8-11, 13, 15-23, 26, 27, 31, 34, 35, 37, 38, 42, 46, 47, 54, 56, 58-60, 101-121, 128-132, and 133-158.

25.-28. (canceled)

29. The method of claim 5, wherein the structurally-stabilized peptide specifically lyses bacterial cells in addition to the cancer cells.

30. The method of claim 5, wherein the method further comprises treating a bacterial infection in a human subject in need thereof, wherein the structurally stabilized peptide comprises the amino acid sequence set forth in any one of SEQ ID NOs: 17, 27, 28, 98, 99, 141-145, and 60.

31. The method of claim 30, wherein the cancer is a leukemia, a lymphoma, or a melanoma.

32.-34. (canceled)

35. A peptide comprising an amino acid sequence:
(a) X₁IGKX₂LHKAKKFGKAX₃VGEX₄BNS, wherein each of X₁, X₂, X₃, and X₄ is independently a stapling amino acid (SEQ ID NO:98) with 0 to 4 amino acid substitutions;
(b) X₁IGKX₂LHSKKKFGKAX₃VGEX₄BNS, wherein each of X₁, X₂, X₃, and X₄ is independently a stapling amino acid (SEQ ID NO:99) with 0 to 4 amino acid substitutions;
(c) GIGKFLHSAKKFGKAX₁VGEX₂KNS, wherein each of X₁ and X₂ is independently a stapling amino acid (SEQ ID NO:37) with 0 to 4 amino acid substitutions;
(d) X₁KGKX₂LHSAKKFGKAX₃VGEX₄BNS, wherein each of X₁, X₂, X₃, and X₄ is independently a stapling amino acid (SEQ ID NO:133) with 0 to 4 amino acid substitutions;
(e) X₁IEKX₂LHSAKKFGKAX₃VGEX₄BNS, wherein each of X₁, X₂, X₃, and X₄ is independently a stapling amino acid (SEQ ID NO:134) with 0 to 4 amino acid substitutions;
(f) X₁IGKX₂LESAKKFGKAX₃VGEX₄BNS, wherein each of X₁, X₂, X₃, and X₄ is independently a stapling amino acid (SEQ ID NO:135) with 0 to 4 amino acid substitutions;
(g) X₁IGKX₂LHSAKKFGKAX₃VEEX₄BNS, wherein each of X₁, X₂, X₃, and X₄ is independently a stapling amino acid (SEQ ID NO:136) with 0 to 4 amino acid substitutions;
(h) X₁IGKX₂LHSAKKFGKAFVGEIBES, wherein each of X₁ and X₂ is independently a stapling amino acid (SEQ ID NO:137) with 0 to 4 amino acid substitutions;
(i) GKX₁KFLX₂SAKKFGKAFVGEIBNS, wherein each of X₁ and X₂ is independently a stapling amino acid (SEQ ID NO:138) with 0 to 4 amino acid substitutions;
(j) GIX₁KFLX₂SAKKFGKAFVEEIBNS, wherein each of X₁ and X₂ is independently a stapling amino acid (SEQ ID NO:139) with 0 to 4 amino acid substitutions;
(k) GKX₁KFLX₂SAKKFGKAFVGEIBES, wherein each of X₁ and X₂ is independently a stapling amino acid (SEQ ID NO:140) with 0 to 4 amino acid substitutions;
(l) X₁IGKX₂LHSAKKFGKAX₃VGEX₄BNS, wherein each of X₁, X₂, X₃, and X₄ is independently a stapling amino acid (SEQ ID NO:141) with 0 to 4 amino acid substitutions;
(m) GIX₁KFLX₂SAKKFGKAX₃VGEX₄BNS, wherein each of X₁, X₂, X₃, and X₄ is independently a stapling amino acid (SEQ ID NO:142) with 0 to 4 amino acid substitutions;
(n) GIX₁KFLX₂KAKKFGKAX₃VGEX₄BNS, wherein each of X₁, X₂, X₃, and X₄ is independently a stapling amino acid (SEQ ID NO:143) with 0 to 4 amino acid substitutions;
(o) GIX₁KFLX₂SKKKFGKAX₃VGEX₄BNS, wherein each of X₁, X₂, X₃, and X₄ is independently a stapling amino acid (SEQ ID NO:144) with 0 to 4 amino acid substitutions; or
(p) GX₁GKFX₂HSKKKFGKAX₃VGEX₄BNS, wherein each of X₁, X₂, X₃, and X₄ is independently a stapling amino acid (SEQ ID NO:145) with 0 to 4 amino acid substitutions;
wherein B is norleucine; and
wherein the peptide specifically lyses cancer cells.

36. (canceled)

37. A structurally-stabilized peptide comprising the peptide of claim 35,
wherein the peptide is stapled.

38.-39. (canceled)

40. A pharmaceutical composition comprising: (i) the peptide of claim 35; and (ii) a pharmaceutically acceptable carrier.

41. A method of making the structurally-stabilized peptide of claim 37, the method comprising: (a) providing a peptide having the sequence set forth in any one of SEQ ID NOs: 37, 98, 99, 133-145, and 308-311, and (b) cross-linking the peptide.

42.-46. (canceled)

47. A pharmaceutical composition comprising:
(a) a means for selectively killing cancer cells or for killing both cancer cells and bacterial cells; and
(b) a pharmaceutically acceptable carrier.

48.-58. (canceled)

59. A pharmaceutical composition comprising: (i) the structurally-stabilized peptide of claim 37; and (ii) a pharmaceutically acceptable carrier.
